(19) United States
(12) Patent Application Publication
(10) Pub. No.: US 2022/0396796 A1
(43) Pub. Date: Dec. 15, 2022

(54) RNA FOR CANCER THERAPY

(71) Applicant: CureVac AG, Tübingen (DE)

(72) Inventors: Mariola FOTIN-MLECZEK, Sindelfingen (DE); Aleksandra KOWALCZYK, Stuttgart (DE); Regina HEIDENREICH, Tübingen (DE); Ulrike GNAD-VOGT, Mühltal (DE); Ute KLINKHARDT, Frankfurt (DE); Katja FIEDLER, Bad Urach (DE)

(73) Assignee: CureVac AG, Tübingen (DE)

(21) Appl. No.: 17/571,893

(22) Filed: Jan. 10, 2022

Related U.S. Application Data
(63) Continuation of application No. 16/326,281, filed on Feb. 18, 2019, now abandoned.

(30) Foreign Application Priority Data
Aug. 19, 2016 (EP) PCT/EP2016/069753
Jun. 13, 2017 (EP) PCT/EP2017/064463

(51) Int. Cl. C12N 15/117; A61K 38/20; A61K 39/00; A61P 35/00

(52) U.S. Cl. CPC C12N 15/117; A61K 38/208; A61K 39/001186; A61K 39/001152; A61K 39/001119; A61K 39/001122; A61K 39/001166; A61K 39/001182; A61K 39/001104; A61K 39/001168; A61K 39/001184; A61K 39/001106; A61P 35/00; C12N 2310/531; C12N 2320/32; A61K 2039/53; A61K 2039/54

(57) ABSTRACT

The present invention relates to RNA, particularly an immunostimulatory RNA (isRNA), a coding RNA or a combination thereof, for use in the treatment or prophylaxis of a disease, in particular a tumor and/or cancer disease. The present invention also provides pharmaceutical compositions, and a kit comprising the RNA(s). Further, the invention also comprises medical uses of the RNA(s) and compositions comprising the RNA(s).

### CROSS-REFERENCE TO RELATED APPLICATIONS

This application is a continuation of U.S. application Ser. No. 16/326,281, filed Feb. 18, 2019, which is a national phase application under 35 U.S.C. § 371 of International Application No. PCT/EP2017/025230, filed Aug. 14, 2017, which claims benefit of International Application No. PCT/EP2017/064463, filed Jun. 13, 2017, and International Application No. PCT/EP2016/069753, filed Aug. 19, 2016, the entire contents of each of which are hereby incorporated by reference.

### BACKGROUND OF THE INVENTION

The present invention relates to RNA, particularly an immunostimulatory RNA (isRNA), a coding RNA or a combination thereof, for use in the treatment or prophylaxis of a disease, in particular a tumor and/or cancer disease. The present invention also provides pharmaceutical compositions, and a kit comprising the RNA(s). Further, the invention also comprises medical uses of the RNA(s) and compositions comprising the RNA(s).

Cancer diseases, also known as malignant tumors, are a group of diseases involving abnormal cell growth with the potential to invade or spread to other parts of the body. In 2012, about 14.1 million new cases of cancer occurred globally (not including skin cancer other than melanoma).

The standard treatments of cancer include chemotherapy, radiation and surgery, or immunotherapy wherein these treatments are applied individually or in combination. Cancer immunotherapy which is focused on stimulating the immune system through vaccination, adoptive cellular immunotherapy, immune checkpoint blockade or other immunostimulants or immunomodulators to elicit an anti-tumor response.

Some approaches use gene therapy and genetic vaccination for treatment of cancer or other tumor diseases. Gene therapy and genetic vaccination are molecular medicine methods, which are based on the introduction of a nucleic acid into cells or into tissues of a patient. Subsequently the information encoded by the nucleic acid introduced is processed in the organism, i.e. resulting in expression of a therapeutic peptide or protein or in expression of an antigen, which is coded by the nucleic acid.

Conventional gene therapeutic methods, including gene therapy and genetic vaccination are based on the use of DNA molecules in order to transfer the desired genetic information into the cell. Various methods have been developed for introducing DNA into cells, such as calcium phosphate transfection, polybrene transfection, protoplast fusion, electroporation, microinjection and lipofection. DNA viruses may likewise be used as a DNA vehicle achieving a very high transfection rate. The use of DNA entails the risk of the DNA being inserted into an intact gene of the host cell's genome by e.g. recombination. In this case the affected gene may be mutated and inactivated or may give rise to misinformation. Another risk of using DNA as a pharmaceutical agent is the risk of inducing pathogenic anti-drug antibodies (anti-DNA antibodies) in the patient, which may result in autoimmune adverse effects.

The use of RNA as a gene therapeutic agent or genetic vaccine is substantially safer, because RNA does not involve the risk of being integrated into the genome inducing an undesired pathogenic induction of anti-drug antibodies.

Thus RNA expression systems have considerable advantages over DNA expression systems in gene therapy and in genetic vaccination although it has been assumed for a long time that the instability of mRNA or of RNA in general may pose a serious problem for medical methods based on RNA expression systems.

The instability of RNA is due, in particular, to RNA-degrading enzymes (ribonucleases—RNases). There are also many further processes that destabilize RNA, wherein interaction between the RNA and proteins often appears to play a crucial role. Some measures for increasing the stability of RNA have been proposed, thus enabling the use thereof as a gene therapy agent or RNA vaccine.

With respect to the ex vivo stability of RNA, European patent application EP 1 083 232 A1 describes a method for introducing RNA, in particular mRNA, into cells and organisms, wherein the RNA forms a complex with a cationic peptide or protein.

The application of mRNA for the treatment and/or prophylaxis of cancer is known. For example, international patent application WO 03/051401 A2 describes a pharmaceutical composition comprising at least one mRNA, which contains at least one region encoding an antigen from a tumor, combined with an aqueous solvent and preferably with a cytokine e.g. GM-CSF. The pharmaceutical composition is proposed for use in therapy and/or prophylaxis of cancer.

International patent application WO 2006/008154 A1 discloses an mRNA mixture for vaccination against tumor diseases, wherein at least one type of mRNA contains at least one tumor antigen-encoding region. At least one other mRNA contains at least one type of an immunogenic protein-coding region. Nevertheless there is still a need for an effective treatment of cancer or tumor diseases. Therefore it is the object of the underlying invention to provide an approach for effective treatment of tumor diseases wherein tumor tissue and cancer cells are specifically destroyed.

This object is solved by the subject-matter of the claims. Particularly, the object underlying the present invention is solved by isRNA, coding RNA or a combination thereof for use in the treatment or prophylaxis of tumor and/or cancer diseases. According to further aspects of the invention, the object is solved by a pharmaceutical composition, by a kit or kit of parts, and by a method of treatment of tumor or cancer diseases.

For the sake of clarity and readability the following definitions are provided. Any technical feature mentioned for these definitions may be read on each and every embodiment of the invention. Additional definitions and explanations may be specifically provided in the context of these embodiments.

Immune system: The immune system may protect organisms from infection. If a pathogen breaks through a physical barrier of an organism and enters this organism, the innate immune system provides an immediate, but non-specific response. If pathogens evade this innate response, vertebrates possess a second layer of protection, the adaptive immune system. Here, the immune system adapts its response during an infection to improve its recognition of the pathogen. Additionally to infections of pathogens this response can also be directed against malignant tumor cells of the body. The improved response is then retained after the pathogen or tumor cell has been eliminated, in the form of an immunological memory, and allows the adaptive immune system to mount faster and stronger attacks each time this pathogen is encountered. According to this, the immune system comprises the innate and the adaptive immune system. Each of these two parts contains so called humoral and cellular components.

Immune response: An immune response may typically either be a specific reaction of the adaptive immune system to an antigen, with antigens being tumor derived (so called specific or adaptive immune response) or an unspecific reaction of the innate immune system (so called unspecific or innate immune response).

Adaptive immune system: The adaptive immune system is composed of highly specialized, systemic cells and processes that eliminate or prevent pathogenic growth. The adaptive immune response provides the vertebrate immune system with the ability to recognize and remember specific pathogens (to generate immunity), and to mount stronger attacks each time the pathogen is encountered. The system is highly adaptable because of somatic hypermutation (a process of increased frequency of somatic mutations), and V(D)J recombination (an irreversible genetic recombination of antigen receptor gene segments). This mechanism allows a small number of genes to generate a vast number of different antigen receptors, which are then uniquely expressed on each individual lymphocyte. Because the gene rearrangement leads to an irreversible change in the DNA of each cell, all of the progeny (offspring) of that cell will then inherit genes encoding the same receptor specificity, including the Memory B cells and Memory T cells that are the keys to long-lived specific immunity. Immune network theory is a theory of how the adaptive immune system works, that is based on interactions between the variable regions of the receptors of T cells, B cells and of molecules made by T cells and B cells that have variable regions.

Adaptive immune response: The adaptive immune response is typically understood to be antigen-specific. Antigen specificity allows for the generation of responses that are tailored to specific antigens, pathogens or pathogen-infected cells. The ability to mount these tailored responses is maintained in the body by “memory cells”. Should a pathogen infect the body more than once, these specific memory cells are used to quickly eliminate it. In this context, the first step of an adaptive immune response is the activation of naïve antigen-specific T cells or different immune cells able to induce an antigen-specific immune response by antigen-presenting cells. This occurs in the lymphoid tissues and organs through which naïve T cells are constantly passing. Cell types that can serve as antigen-presenting cells are inter alia dendritic cells, macrophages, and B cells. Each of these cells has a distinct function in eliciting immune responses. Dendritic cells take up antigens by phagocytosis and macropinocytosis and are stimulated by contact with e.g. a foreign antigen to migrate to the local lymphoid tissue, where they differentiate into mature dendritic cells. Macrophages ingest particulate antigens such as bacteria and are induced by infectious agents or other appropriate stimuli to express MHC molecules. The unique ability of B cells to bind and internalize soluble protein antigens via their receptors may also be important to induce T cells. Presenting the antigen on MHC molecules leads to activation of T cells which induces their proliferation and differentiation into armed effector T cells. The most important function of effector T cells is the killing of infected cells by CD8+ cytotoxic T cells and the activation of macrophages by Th1 cells which together make up cell-mediated immunity, and the activation of B cells by both Th2 and Th1 cells to produce different classes of antibody, thus driving the humoral immune response. T cells recognize an antigen by their T cell receptors which do not recognize and bind antigen directly, but instead recognize short peptide fragments e.g. of pathogen-derived protein antigens, which are bound to MHC molecules on the surfaces of other cells.

Cellular immunity/cellular immune response: Cellular immunity relates typically to the activation of macrophages, natural killer cells (NK), antigen-specific cytotoxic T-lymphocytes, and the release of various cytokines in response to an antigen. In a more general way, cellular immunity is not related to antibodies but to the activation of cells of the immune system. A cellular immune response is characterized e.g. by activating antigen-specific cytotoxic T-lymphocytes that are able to induce apoptosis in body cells displaying epitopes of an antigen on their surface, such as virus-infected cells, cells with intracellular bacteria, and cancer cells displaying tumor antigens; activating macrophages and natural killer cells, enabling them to destroy pathogens; and stimulating cells to secrete a variety of cytokines that influence the function of other cells involved in adaptive immune responses and innate immune responses.

Humoral immunity/humoral immune response: Humoral immunity refers typically to antibody production and the accessory processes that may accompany it. A humoral immune response may be typically characterized, e.g., by Th2 activation and cytokine production, germinal center formation and isotype switching, affinity maturation and memory cell generation. Humoral immunity also typically may refer to the effector functions of antibodies, which include pathogen and toxin neutralization, classical complement activation, and opsonin promotion of phagocytosis and pathogen elimination.

Innate immune system: The innate immune system, also known as non-specific immune system, comprises the cells and mechanisms that defend the host from infection by other organisms in a non-specific manner. This means that the cells of the innate system recognize and respond to pathogens in a generic way, but unlike the adaptive immune system, it does not confer long-lasting or protective immunity to the host. The innate immune system may be e.g. activated by ligands of pathogen-associated molecular patterns (PAMP) receptors, e.g. Toll-like receptors (TLRs) or other auxiliary substances such as lipopolysaccharides, TNF-alpha, CD40 ligand, or cytokines, monokines, lymphokines, interleukins or chemokines, immunostimulatory nucleic acids, immunostimulatory RNA (isRNA), CpG-DNA, antibacterial agents, or anti-viral agents. Typically a response of the innate immune system includes recruiting immune cells to sites of infection, through the production of chemical factors, including specialized chemical mediators, called cytokines; activation of the complement cascade; identification and removal of foreign substances present in organs, tissues, the blood and lymph, by specialized white blood cells; activation of the adaptive immune system through a process known as antigen presentation; and/or acting as a physical and chemical barrier to infectious agents.

Adjuvant/adjuvant component: An adjuvant or an adjuvant component in the broadest sense is typically a (e.g. pharmacological or immunological) agent or composition that may modify, e.g. enhance, the efficacy of other agents, such as a drug or vaccine. Conventionally the term refers in the context of the invention to a compound or composition that serves as a carrier or auxiliary substance for immunogens and/or other pharmaceutically active compounds. It is to be interpreted in a broad sense and refers to a broad spectrum of substances that are able to increase the immunogenicity of antigens incorporated into or co-administered with an adjuvant in question. In the context of the present invention an adjuvant will preferably enhance the specific immunogenic effect of the active agents of the present invention. Typically, “adjuvant” or “adjuvant component” has the same meaning and can be used mutually. Adjuvants may be divided, e.g., into immuno potentiators, antigenic delivery systems or even combinations thereof. The term “adjuvant” is typically understood not to comprise agents which confer immunity by themselves. An adjuvant assists the immune system unspecifically to enhance the antigen-specific immune response by e.g. promoting presentation of an antigen to the immune system or induction of an unspecific innate immune response. Furthermore, an adjuvant may preferably e.g. modulate the antigen-specific immune response by e.g. shifting the dominating Th2-based antigen specific response to a more Th1-based antigen specific response or vice versa. Accordingly, an adjuvant may favourably modulate cytokine expression/secretion, antigen presentation, type of immune response etc.

Immunostimulatory/immunostimulating RNA: An immunostimulatory/immunostimulating RNA (isRNA) in the context of the invention may typically be an RNA that is capable of inducing an innate immune response by itself. It usually does not comprise an open reading frame and thus does not provide a peptide-antigen or immunogen but elicits an innate immune response e.g. by binding to a specific kind of Toll-like-receptor (TLR) or other suitable receptors. Therefore immunostimulatory/immunostimulating RNAs are preferably non-coding RNAs. However, of course also mRNAs having an open reading frame and encoding a peptide/protein (e.g. an antigenic function) may induce an innate immune response.

Antigen: The term “antigen” refers typically to a substance which may be recognized by the immune system and may be capable of triggering an antigen-specific immune response, e.g. by formation of antibodies or antigen-specific T-cells as part of an adaptive immune response. An antigen may be a protein or peptide. In this context, the first step of an adaptive immune response is the activation of naïve antigen-specific T cells by antigen-presenting cells. This occurs in the lymphoid tissues and organs through which naïve T cells are constantly passing. The three cell types that can serve as antigen-presenting cells are dendritic cells, macrophages, and B cells. Each of these cells has a distinct function in eliciting immune responses. Tissue dendritic cells take up antigens by phagocytosis and macropinocytosis and are stimulated by infection to migrate to the local lymphoid tissue, where they differentiate into mature dendritic cells. Macrophages ingest particulate antigens such as bacteria and are induced by infectious agents to express MHC class II molecules. The unique ability of B cells to bind and internalize soluble protein antigens via their receptors may be important to induce T cells. By presenting the antigen on MHC molecules leads to activation of T cells which induces their proliferation and differentiation into armed effector T cells. The most important function of effector T cells is the killing of infected cells by CD8+ cytotoxic T cells and the activation of macrophages by Th1 cells which together make up cell-mediated immunity, and the activation of B cells by both Th2 and Th1 cells to produce different classes of antibody, thus driving the humoral immune response. T cells recognize an antigen by their T cell receptors which does not recognize and bind antigen directly, but instead recognize short peptide fragments e.g. of pathogens' protein antigens, which are bound to MHC molecules on the surfaces of other cells.

T cells fall into two major classes that have different effector functions. The two classes are distinguished by the expression of the cell-surface proteins CD4 and CD8. These two types of T cells differ in the class of MHC molecule that they recognize. There are two classes of MHC molecules—MHC class I and MHC class II molecules—which differ in their structure and expression pattern on tissues of the body. CD4+ T cells bind to a MHC class II molecule and CD8+ T cells to a MHC class I molecule. MHC class I and MHC class II molecules have distinct distributions among cells that reflect the different effector functions of the T cells that recognize them. MHC class I molecules present peptides of cytosolic and nuclear origin e.g. from pathogens, commonly viruses, to CD8+ T cells, which differentiate into cytotoxic T cells that are specialized to kill any cell that they specifically recognize. Almost all cells express MHC class I molecules, although the level of constitutive expression varies from one cell type to the next. But not only pathogenic peptides from viruses are presented by MHC class I molecules, also self-antigens like tumor antigens are presented by them. MHC class I molecules bind peptides from proteins degraded in the cytosol and transported in the endoplasmic reticulum. The CD8+ T cells that recognize MHC class I: peptide complexes at the surface of infected cells are specialized to kill any cells displaying foreign peptides and so rid the body of cells infected with viruses and other cytosolic pathogens. The main function of CD4+ T cells (CD4+ helper T cells) that recognize MHC class II molecules is to activate other effector cells of the immune system. Thus MHC class II molecules are normally found on B lymphocytes, dendritic cells, and macrophages, cells that participate in immune responses, but not on other tissue cells. Macrophages, for example, are activated to kill the intravesicular pathogens they harbour, and B cells to secrete immunoglobulins against foreign molecules. MHC class II molecules are prevented from binding to peptides in the endoplasmic reticulum and thus MHC class II molecules bind peptides from proteins which are degraded in endosomes. They can capture peptides from pathogens that have entered the vesicular system of macrophages, or from antigens internalized by immature dendritic cells or the immunoglobulin receptors of B cells. Pathogens that accumulate in large numbers inside macrophage and dendritic cell vesicles tend to stimulate the differentiation of Th1 cells, whereas extracellular antigens tend to stimulate the production of Th2 cells. Th1 cells activate the microbicidal properties of macrophages and induce B cells to make IgG antibodies that are very effective of opsonising extracellular pathogens for ingestion by phagocytic cells, whereas Th2 cells initiate the humoral response by activating naïve B cells to secrete IgM, and induce the production of weakly opsonising antibodies such as IgG1 and IgG3 (mouse) and IgG2 and IgG4 (human) as well as IgA and IgE (mouse and human).

Epitope (also called “antigen determinant”): T cell epitopes may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T cells in form of a complex consisting of the peptide fragment and an MHC molecule. B cell epitopes are typically fragments located on the outer surface of (native) protein or peptide antigens.

Vaccine: A vaccine is typically understood to be a composition comprising a compound derived from an antigenic substance, which is preferably derived from the causative agent of a disease, wherein the compound is used to provide immunity against one or several diseases. In the context of the present invention, the term “vaccine” may preferably also refer to a (synthetic/artificial) compound, which is not derived from an antigenic substance from the causative agent of a disease, and wherein the compound is used to provide immunity against one or several diseases. For example, the term “vaccine” as used herein may refer to a composition comprising as an active ingredient a (synthetic/artificial) compound, such as an (artificial) nucleic acid molecule, peptide or protein (which are preferably not derived from an antigenic substance derived from the causative agent of a disease), wherein said compound induces an immune response, preferably an innate immune response. In this context, a vaccine may comprise as an active ingredient a (synthetic/artificial) compound that preferably acts as an adjuvant and/or an immune modulator. In this context, the term “immune modulator” refers to a compound, which enhances or inhibits an immune reaction, for instance by specific interference with a signalling pathway in certain immune cells. In the meaning of the present invention, a vaccine may thus stimulate the body's adaptive and/or innate immune system in order to provide an immune response, for example an immune response against a tumor (cell).

Antigen-providing mRNA: An antigen-providing mRNA may typically be an mRNA, having at least one open reading frame that can be translated by a cell or an organism provided with that mRNA. The product of this translation is a peptide or protein that may act as an antigen, preferably as an immunogen. The product may also be a fusion protein composed of more than one immunogen, e.g. a fusion protein that consist of two or more epitopes, peptides or proteins, wherein the epitopes, peptides or proteins may be linked by linker sequences.

Bi-/multicistronic mRNA: An bi-/multicistronic mRNA typically may have two (bicistronic) or more (multicistronic) coding sequences (cds) (also often referred to as open reading frames (ORF)). A coding sequence/an open reading frame in this context is a sequence of several nucleotide triplets (codons) that can be translated into a peptide or protein. Translation of such an mRNA yields two (bicistronic) or more (multicistronic) distinct translation products (provided the coding sequences/ORFs are not identical). For expression in eukaryotes such mRNAs may for example comprise an internal ribosomal entry site (IRES) sequence.

5′-CAP-Structure: A 5′-CAP is typically a modified nucleotide (CAP analogue), particularly a guanine nucleotide, added to the 5′ end of an mRNA molecule. Preferably, the 5′-CAP is added using a 5′-5′-triphosphate linkage (also named m7GpppN). Further examples of 5′-CAP structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. These modified 5′-CAP structures may be used in the context of the present invention to modify the mRNA sequence of the inventive composition. Further modified 5′-CAP structures which may be used in the context of the present invention are CAP1 (additional methylation of the ribose of the adjacent nucleotide of m7GpppN), CAP2 (additional methylation of the ribose of the 2nd nucleotide downstream of the m7GpppN), CAP3 (additional methylation of the ribose of the 3rd nucleotide downstream of the m7GpppN), CAP4 (additional methylation of the ribose of the 4th nucleotide downstream of the m7GpppN), ARCA (anti-reverse CAP analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In the context of the present invention, a 5′ cap structure may also be formed in chemical RNA synthesis or RNA in vitro transcription (co-transcriptional capping) using cap analogues, or a cap structure may be formed in vitro using capping enzymes (e.g., commercially available capping kits)

Cap analogue: A cap analogue refers to a non-polymerizable di-nucleotide that has cap functionality in that it facilitates translation or localization, and/or prevents degradation of the RNA molecule when incorporated at the 5′ end of the RNA molecule. Non-polymerizable means that the cap analogue will be incorporated only at the 5′terminus because it does not have a 5′ triphosphate and therefore cannot be extended in the 3′ direction by a template-dependent RNA polymerase.

Cap analogues include, but are not limited to, a chemical structure selected from the group consisting of m7GpppG, m7GpppA, m7GpppC; unmethylated cap analogues (e.g., GpppG); dimethylated cap analogue (e.g., m2,7GpppG), trimethylated cap analogue (e.g., m2,2,7GpppG), dimethylated symmetrical cap analogues (e.g., m7Gpppm7G), or anti reverse cap analogues (e.g., ARCA; m7,2′OmeGpppG, m7,2′dGpppG, m7,3′OmeGpppG, m7,3′dGpppG and their tetraphosphate derivatives) (Stepinski et al., 2001. RNA 7(10):1486-95).

Further cap analogues have been described previously (U.S. Pat. No. 7,074,596, WO 2008/016473, WO 2008/157688, WO 2009/149253, WO 2011/015347, and WO 2013/059475). The synthesis of N⁷-(4-chlorophenoxyethyl) substituted dinucleotide cap analogues has been described recently (Kore et al. (2013) Bioorg. Med. Chem. 21(15): 4570-4).

Fragments of proteins: “Fragments” of proteins or peptides in the context of the present invention may, typically, comprise a sequence of a protein or peptide as defined herein, which is, with regard to its amino acid sequence (or its encoded nucleic acid molecule), N-terminally and/or C-terminally truncated compared to the amino acid sequence of the original (native) protein (or its encoded nucleic acid molecule). Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide. In the context of antigens such fragment may have a length of about 6 to about 20 or even more amino acids, e.g. fragments as processed and presented by MHC class I molecules, preferably having a length of about 8 to about 10 amino acids, e.g. 8, 9, or 10, (or even 6, 7, 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T-cells in form of a complex consisting of the peptide fragment and an MHC molecule, i.e. the fragments are typically not recognized in their native form. Fragments of proteins or peptides (e.g. in the context of antigens) may comprise at least one epitope of those proteins or peptides. Furthermore also domains of a protein, like the extracellular domain, the intracellular domain or the transmembrane domain and shortened or truncated versions of a protein may be understood to comprise a fragment of a protein. Preferably, a fragment of a protein comprises a functional fragment of the protein, which means that the fragment exerts the same effect or functionality as the whole protein it is derived from. More preferably, a “fragment” as used herein is at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the peptide or protein, from which it is derived.

Variants of proteins: “Variants” of proteins or peptides as defined in the context of the present invention may be generated, having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific antigenic property. “Variants” of proteins or peptides as defined in the context of the present invention may comprise conservative amino acid substitution(s) compared to their native, i.e. non-mutated physiological, sequence. Those amino acid sequences as well as their encoding nucleotide sequences in particular fall under the term variants as defined herein. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, for example, an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam).

A “variant” of a protein or peptide may have at least 70%, 75%, 80%, 85%, 90%, 95%, 98% or 99% amino acid identity over a stretch of 10, 20, 30, 50, 75 or 100 amino acids of such protein or peptide. Alternatively, More preferably, a “variant” as used herein is at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the peptide or protein, from which it is derived.

Furthermore, variants of proteins or peptides as defined herein, which may be encoded by a nucleic acid molecule, may also comprise those sequences, wherein nucleotides of the encoding nucleic acid sequence are exchanged according to the degeneration of the genetic code, without leading to an alteration of the respective amino acid sequence of the protein or peptide, i.e. the amino acid sequence or at least part thereof may not differ from the original sequence within the above meaning.

Preferably, a variant of a protein comprises a functional variant of the protein, which means that the variant exerts the same effect or functionality as the protein it is derived from.

Identity of a sequence: In order to determine the percentage to which two sequences are identical, e.g. nucleic acid sequences or amino acid sequences as defined herein, preferably the amino acid sequences encoded by a nucleic acid sequence of the polymeric carrier as defined herein or the amino acid sequences themselves, the sequences can be aligned in order to be subsequently compared to one another. Therefore, e.g. a position of a first sequence may be compared with the corresponding position of the second sequence. If a position in the first sequence is occupied by the same component (residue) as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using a mathematical algorithm. A preferred, but not limiting, example of a mathematical algorithm which can be used is the algorithm of Karlin et al. (1993), PNAS USA, 90:5873-5877 or Altschul et al. (1997), Nucleic Acids Res., 25:3389-3402. Such an algorithm is integrated in the BLAST program. Sequences which are identical to the sequences of the present invention to a certain extent can be identified by this program.

Monocistronic mRNA: A monocistronic mRNA may typically be an mRNA, that comprises only one coding sequence (open reading frame). A coding sequence/open reading frame in this context is a sequence of several nucleotide triplets (codons) that can be translated into a peptide or protein.

Nucleic acid: The term nucleic acid means any DNA or RNA molecule and is used synonymous with polynucleotide. Wherever herein reference is made to a nucleic acid or nucleic acid sequence encoding a particular protein and/or peptide, said nucleic acid or nucleic acid sequence, respectively, preferably also comprises regulatory sequences allowing in a suitable host, e.g. a human being, its expression, i.e. transcription and/or translation of the nucleic acid sequence encoding the particular protein or peptide.

Peptide: A peptide is a polymer of amino acid monomers. Usually the monomers are linked by peptide bonds. The term “peptide” does not limit the length of the polymer chain of amino acids. In some embodiments of the present invention a peptide may for example contain less than 50 monomer units. Longer peptides are also called polypeptides, typically having 50 to 600 monomeric units, more specifically 50 to 300 monomeric units.

Pharmaceutically effective amount: A pharmaceutically effective amount in the context of the invention is typically understood to be an amount that is sufficient to induce an immune response or to trigger the desired therapeutical effect.

Protein: A protein typically consists of one or more peptides and/or polypeptides folded into 3-dimensional form, facilitating a biological function.

Poly(C) sequence: A poly(C) sequence is typically a long sequence of cytosine nucleotides, typically about 10 to about 200 cytosine nucleotides, preferably about 10 to about 100 cytosine nucleotides, more preferably about 10 to about 70 cytosine nucleotides or even more, preferably about 20 to about 50, or even about 20 to about 30 cytosine nucleotides. A poly(C) sequence may preferably be located 3′ of the coding region comprised by a nucleic acid.

Poly(A) tail: A poly(A) tail also called “3′-poly(A) tail” or “poly(A) sequence” is typically a long homopolymeric sequence of adenosine nucleotides of up to about 400 adenosine nucleotides, e.g. from about 25 to about 400, preferably from about 50 to about 400, more preferably from about 50 to about 300, even more preferably from about 50 to about 250, most preferably from about 60 to about 250 adenosine nucleotides, added to the 3′ end of an mRNA. In the context of the present invention, the poly(A) tail of an mRNA is preferably derived from a DNA template by RNA in vitro transcription. Alternatively, the poly(A) sequence may also be obtained in vitro by common methods of chemical synthesis without being necessarily transcribed from a DNA-progenitor. Moreover, poly(A) sequences, or poly(A) tails may be generated by enzymatic polyadenylation of the RNA.

Stabilized nucleic acid: A stabilized nucleic acid, typically, exhibits a modification increasing resistance to in vivo degradation (e.g. degradation by an exo- or endo-nuclease) and/or ex vivo degradation (e.g. by the manufacturing process prior to vaccine administration, e.g. in the course of the preparation of the vaccine solution to be administered). Stabilization of RNA can, e.g., be achieved by providing a 5′-CAP-Structure, a poly(A) tail, or any other UTR-modification. It can also be achieved by backbone-modification or modification of the G/C-content or the C-content of the nucleic acid. Various other methods are known in the art and conceivable in the context of the invention.

Carrier/polymeric carrier: A carrier in the context of the invention may typically be a compound that facilitates transport and/or complexation of another compound. Said carrier may form a complex with said other compound. A polymeric carrier is a carrier that is formed of a polymer.

Cationic component: The term “cationic component” typically refers to a charged molecule, which is positively charged (cation) at a pH value of typically about 1 to 9, preferably of a pH value of or below 9 (e.g. 5 to 9), of or below 8 (e.g. 5 to 8), of or below 7 (e.g. 5 to 7), most preferably at physiological pH values, e.g. about 7.3 to 7.4. Accordingly, a cationic peptide, protein or polymer according to the present invention is positively charged under physiological conditions, particularly under physiological salt conditions of the cell in vivo. A cationic peptide or protein preferably contains a larger number of cationic amino acids, e.g. a larger number of Arg, His, Lys or Orn than other amino acid residues (in particular more cationic amino acids than anionic amino acid residues like Asp or Glu) or contains blocks predominantly formed by cationic amino acid residues. The definition “cationic” may also refer to “polycationic” components.

Vehicle: A vehicle is an agent, e.g. a carrier, that may typically be used within a pharmaceutical composition or vaccine for facilitating administering of the components of the pharmaceutical composition or vaccine to an individual.

3′-untranslated region (3′-UTR): A 3′-UTR is typically the part of an mRNA which is located between the protein coding region (i.e. the open reading frame) and the poly(A) sequence of the mRNA. A 3′-UTR of the mRNA is not translated into an amino acid sequence. The 3′-UTR sequence is generally encoded by the gene which is transcribed into the respective mRNA during the gene expression process. The genomic sequence is first transcribed into pre-mature mRNA, which comprises optional introns. The pre-mature mRNA is then further processed into mature mRNA in a maturation process. This maturation process comprises the steps of 5′-capping, splicing the pre-mature mRNA to excise optional introns and modifications of the 3′-end, such as polyadenylation of the 3′-end of the pre-mature mRNA and optional endo- or exonuclease cleavages etc. In the context of the present invention, a 3′-UTR corresponds to the sequence of a mature mRNA which is located 3′ to the stop codon of the protein coding region, preferably immediately 3′ to the stop codon of the protein coding region, and which extends to the 5′-side of the poly(A) sequence, preferably to the nucleotide immediately 5′ to the poly(A) sequence. The term “corresponds to” means that the 3′-UTR sequence may be an RNA sequence, such as in the mRNA sequence used for defining the 3′-UTR sequence, or a DNA sequence which corresponds to such RNA sequence. In the context of the present invention, the term “a 3′-UTR of a gene”, such as “a 3′-UTR of an albumin gene”, is the sequence which corresponds to the 3′-UTR of the mature mRNA derived from this gene, i.e. the mRNA obtained by transcription of the gene and maturation of the pre-mature mRNA. The term “3′-UTR of a gene” encompasses the DNA sequence and the RNA sequence of the 3′-UTR.

5′-untranslated region (5′-UTR): A 5′-UTR is typically understood to be a particular section of messenger RNA (mRNA). It is located 5′ of the open reading frame of the mRNA. Typically, the 5′-UTR starts with the transcriptional start site and ends one nucleotide before the start codon of the open reading frame. The 5′-UTR may comprise elements for controlling gene expression, also called regulatory elements. Such regulatory elements may be, for example, ribosomal binding sites or a 5′-Terminal Oligopyrimidine Tract. The 5′-UTR may be posttranscriptionally modified, for example by addition of a 5′-CAP. In the context of the present invention, a 5′UTR corresponds to the sequence of a mature mRNA which is located between the 5′-CAP and the start codon. Preferably, the 5′-UTR corresponds to the sequence which extends from a nucleotide located 3′ to the 5′-CAP, preferably from the nucleotide located immediately 3′ to the 5′-CAP, to a nucleotide located 5′ to the start codon of the protein coding region, preferably to the nucleotide located immediately 5′ to the start codon of the protein coding region. The nucleotide located immediately 3′ to the 5′-CAP of a mature mRNA typically corresponds to the transcriptional start site. The term “corresponds to” means that the 5′-UTR sequence may be an RNA sequence, such as in the mRNA sequence used for defining the 5′-UTR sequence, or a DNA sequence which corresponds to such RNA sequence. In the context of the present invention, the term “a 5′-UTR of a gene”, such as “a 5′-UTR of a TOP gene”, is the sequence which corresponds to the 5′-UTR of the mature mRNA derived from this gene, i.e. the mRNA obtained by transcription of the gene and maturation of the pre-mature mRNA. The term “5′-UTR of a gene” encompasses the DNA sequence and the RNA sequence of the 5′-UTR.

5′ Terminal Oligopyrimidine Tract (TOP): The 5′ terminal oligopyrimidine tract (TOP) is typically a stretch of pyrimidine nucleotides located at the 5′ terminal region of a nucleic acid molecule, such as the 5′ terminal region of certain mRNA molecules or the 5′ terminal region of a functional entity, e.g. the transcribed region, of certain genes. The sequence starts with a cytidine, which usually corresponds to the transcriptional start site, and is followed by a stretch of usually about 3 to 30 pyrimidine nucleotides. For example, the TOP may comprise 3, 4, 5, 6, 7, 8, 9, 10, 11, 12, 13, 14, 15, 16, 17, 18, 19, 20, 21, 22, 23, 24, 25, 26, 27, 28, 29, 30 or even more nucleotides. The pyrimidine stretch and thus the 5′ TOP ends one nucleotide 5′ to the first purine nucleotide located downstream of the TOP. mRNA that contains a 5′ terminal oligopyrimidine tract is often referred to as TOP mRNA. Accordingly, genes that provide such messenger RNAs are referred to as TOP genes. TOP sequences have, for example, been found in genes and mRNAs encoding peptide elongation factors and ribosomal proteins.

TOP motif: In the context of the present invention, a TOP motif is a nucleic acid sequence which corresponds to a 5′ TOP as defined above. Thus, a TOP motif in the context of the present invention is preferably a stretch of pyrimidine nucleotides having a length of 3-30 nucleotides. Preferably, the TOP-motif consists of at least 3 pyrimidine nucleotides, preferably at least 4 pyrimidine nucleotides, preferably at least 5 pyrimidine nucleotides, more preferably at least 6 nucleotides, more preferably at least 7 nucleotides, most preferably at least 8 pyrimidine nucleotides, wherein the stretch of pyrimidine nucleotides preferably starts at its 5′ end with a cytosine nucleotide. In TOP genes and TOP mRNAs, the TOP-motif preferably starts at its 5′ end with the transcriptional start site and ends one nucleotide 5′ to the first purine residue in said gene or mRNA. A TOP motif in the sense of the present invention is preferably located at the 5′end of a sequence which represents a 5′-UTR or at the 5′ end of a sequence which codes for a 5′-UTR. Thus, preferably, a stretch of 3 or more pyrimidine nucleotides is called “TOP motif” in the sense of the present invention if this stretch is located at the 5′ end of a respective sequence, such as the inventive mRNA, the 5′-UTR element of the inventive mRNA, or the nucleic acid sequence which is derived from the 5′-UTR of a TOP gene as described herein. In other words, a stretch of 3 or more pyrimidine nucleotides which is not located at the 5′-end of a 5′-UTR or a 5′-UTR element but anywhere within a 5′-UTR or a 5′-UTR element is preferably not referred to as “TOP motif”.

TOP gene: TOP genes are typically characterised by the presence of a 5′ terminal oligopyrimidine tract. Furthermore, most TOP genes are characterized by a growth-associated translational regulation. However, also TOP genes with a tissue specific translational regulation are known. As defined above, the 5′-UTR of a TOP gene corresponds to the sequence of a 5′-UTR of a mature mRNA derived from a TOP gene, which preferably extends from the nucleotide located 3′ to the 5′-CAP to the nucleotide located 5′ to the start codon. A 5′-UTR of a TOP gene typically does not comprise any start codons, preferably no upstream AUGs (uAUGs) or upstream open reading frames (uORFs). Therein, upstream AUGs and upstream open reading frames are typically understood to be AUGs and open reading frames that occur 5′ of the start codon (AUG) of the open reading frame that should be translated. The 5′-UTRs of TOP genes are generally rather short. The lengths of 5′-UTRs of TOP genes may vary between 20 nucleotides up to 500 nucleotides, and are typically less than about 200 nucleotides, preferably less than about 150 nucleotides, more preferably less than about 100 nucleotides. Exemplary 5′-UTRs of TOP genes in the sense of the present invention are the nucleic acid sequences extending from the nucleotide at position 5 to the nucleotide located immediately 5′ to the start codon (e.g. the ATG) in the sequences according to SEQ ID NOs. 1-1363, SEQ ID NO: 1395, SEQ ID NO: 1421 and SEQ ID NO: 1422 of the international patent application WO2013/143700 or homologs or variants thereof, whose disclosure is incorporated herewith by reference. In this context a particularly preferred fragment of a 5′UTR of a TOP gene is a 5′-UTR of a TOP gene lacking the 5′ TOP motif. The term ‘5′UTR of a TOP gene’ preferably refers to the 5′-UTR of a naturally occurring TOP gene.

Chemical synthesis of RNA: Chemical synthesis of relatively short fragments of oligonucleotides with defined chemical structure provides a rapid and inexpensive access to custom-made oligonucleotides of any desired sequence. Whereas enzymes synthesize DNA and RNA only in the 5′ to 3′ direction, chemical oligonucleotide synthesis does not have this limitation, although it is most often carried out in the opposite, i.e. the 3′ to 5′ direction. Currently, the process is implemented as solid-phase synthesis using the phosphoramidite method and phosphoramidite building blocks derived from protected nucleosides (A, C, G, and U), or chemically modified nucleosides.

To obtain the desired oligonucleotide, the building blocks are sequentially coupled to the growing oligonucleotide chain on a solid phase in the order required by the sequence of the product in a fully automated process. Upon the completion of the chain assembly, the product is released from the solid phase to the solution, deprotected, and collected. The occurrence of side reactions sets practical limits for the length of synthetic oligonucleotides (up to about 200 nucleotide residues), because the number of errors increases with the length of the oligonucleotide being synthesized. Products are often isolated by HPLC to obtain the desired oligonucleotides in high purity.

Chemically synthesized oligonucleotides find a variety of applications in molecular biology and medicine. They are most commonly used as antisense oligonucleotides, small interfering RNA, primers for DNA sequencing and amplification, probes for detecting complementary DNA or RNA via molecular hybridization, tools for the targeted introduction of mutations and restriction sites, and for the synthesis of artificial genes.

RNA in vitro transcription: The terms “RNA in vitro transcription” or “in vitro transcription” relate to a process wherein RNA is synthesized in a cell-free system (in vitro). DNA, particularly plasmid DNA, is used as template for the generation of RNA transcripts. RNA may be obtained by DNA-dependent in vitro transcription of an appropriate DNA template, which according to the present invention is preferably a linearized plasmid DNA template. The promoter for controlling in vitro transcription can be any promoter for any DNA-dependent RNA polymerase. Particular examples of DNA-dependent RNA polymerases are the T7, T3, and SP6 RNA polymerases. A DNA template for in vitro RNA transcription may be obtained by cloning of a nucleic acid, in particular cDNA corresponding to the respective RNA to be in vitro transcribed, and introducing it into an appropriate vector for in vitro transcription, for example into plasmid DNA. In a preferred embodiment of the present invention the DNA template is linearized with a suitable restriction enzyme, before it is transcribed in vitro. The cDNA may be obtained by reverse transcription of mRNA or chemical synthesis. Moreover, the DNA template for in vitro RNA synthesis may also be obtained by gene synthesis.

Methods for in vitro transcription are known in the art (see, e.g., Geall et al. (2013) Semin. Immunol. 25(2): 152-159; Brunelle et al. (2013) Methods Enzymol. 530:101-14). Reagents used in said method typically include:
1) a linearized DNA template with a promoter sequence that has a high binding affinity for its respective RNA polymerase such as bacteriophage-encoded RNA polymerases;
2) ribonucleoside triphosphates (NTPs) for the four bases (adenine, cytosine, guanine and uracil);
3) optionally a cap analogue as defined above (e.g. m7G(5′)ppp(5′)G (m7G));
4) a DNA-dependent RNA polymerase capable of binding to the promoter sequence within the linearized DNA template (e.g. T7, T3 or SP6 RNA polymerase);
5) optionally a ribonuclease (RNase) inhibitor to inactivate any contaminating RNase;
6) optionally a pyrophosphatase to degrade pyrophosphate, which may inhibit transcription;
7) MgCl₂, which supplies Mg²⁺ ions as a co-factor for the polymerase;
8) a buffer to maintain a suitable pH value, which can also contain antioxidants (e.g. DTT), and/or polyamines such as spermidine at optimal concentrations.

RNA, mRNA: RNA is the usual abbreviation for ribonucleic acid. It is a nucleic acid molecule, i.e. a polymer consisting of nucleotide monomers. These nucleotides are usually adenosine monophosphate (AMP), uridine monophosphate (UMP), guanosine monophosphate (GMP) and cytidine monophosphate (CMP) monomers or analogues thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. Usually RNA may be obtainable by transcription of a DNA sequence, e.g., inside a cell. In eukaryotic cells, transcription is typically performed inside the nucleus or the mitochondria. In vivo, transcription of DNA usually results in the so-called premature RNA (also called pre-mRNA, precursor mRNA or heterogeneous nuclear RNA) which has to be processed into so-called messenger RNA, usually abbreviated as mRNA. Processing of the premature RNA, e.g. in eukaryotic organisms, comprises a variety of different posttranscriptional modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, an open reading frame, optionally a 3′UTR and a poly(A) tail.

In addition to messenger RNA, several non-coding types of RNA exist which may be involved in regulation of transcription and/or translation, and immunostimulation. Within the present invention the term “RNA” further encompasses any type of single stranded (ssRNA) or double stranded RNA (dsRNA) molecule known in the art, such as viral RNA, retroviral RNA and replicon RNA, small interfering RNA (siRNA), antisense RNA (asRNA), circular RNA (circRNA), ribozymes, aptamers, riboswitches, immunostimulating/immunostimulatory RNA, transfer RNA (tRNA), ribosomal RNA (rRNA), small nuclear RNA (snRNA), small nucleolar RNA (snoRNA), microRNA (miRNA), and Piwi-interacting RNA (piRNA).

Fragment of a nucleic acid sequence, particularly an RNA: A fragment of a nucleic acid sequence consists of a continuous stretch of nucleotides corresponding to a continuous stretch of nucleotides in the full-length nucleic acid sequence which is the basis for the nucleic acid sequence of the fragment, which represents at least 20%, preferably at least 30%, more preferably at least 40%, more preferably at least 50%, even more preferably at least 60%, even more preferably at least 70%, even more preferably at least 80%, even more preferably at least 80%, even more preferably at least 85%, even more preferably at least 90%, even more preferably at least 95%, and most preferably at least 98% or 99% of the full-length nucleic acid sequence. Such a fragment, in the sense of the present invention, is preferably a functional fragment of the full-length nucleic acid sequence.

Variant of a nucleic acid sequence, particularly an RNA: A variant of a nucleic acid sequence refers to a variant of nucleic acid sequences which forms the basis of a nucleic acid sequence. For example, a variant nucleic acid sequence may exhibit one or more nucleotide deletions, insertions, additions and/or substitutions compared to the nucleic acid sequence from which the variant is derived. Preferably, a variant of a nucleic acid sequence is at least 40%, preferably at least 50%, more preferably at least 60%, more preferably at least 70%, even more preferably at least 80%, even more preferably at least 90%, most preferably at least 95% identical to the nucleic acid sequence the variant is derived from. Preferably, the variant is a functional variant. A “variant” of a nucleic acid sequence may have at least 70%, 75%, 80%, 85%, 90%, 95%, 98% or 99% k nucleotide identity over a stretch of 10, 20, 30, 50, 75 or 100 nucleotide of such nucleic acid sequence.

Intratumoral administration/application: The term, intratumoral administration/application” refers to the direct delivery of a pharmaceutical composition into or adjacent to a tumor or cancer and/or immediate vicinity of a tumor or cancer. In the context of the present invention the term “intratumoral administration/application” thus typically also refers to locoregional or peritumoral application/administration. Multiple injections into separate regions of the tumor or cancer are also included. Furthermore, intratumoral administration/application includes delivery of a pharmaceutical composition into one or more metastases. Methods for intratumoral delivery of drugs are known in the art (Brincker, 1993. Crit. Rev. Oncol. Hematol. 15(2):91-8; Celikoglu et al., 2008. Cancer Therapy 6, 545-552). For example, the pharmaceutical composition can be administered by conventional needle injection, needle-free jet injection or electroporation or combinations thereof into the tumor or cancer tissue. The pharmaceutical composition can be injected directly into the tumor or cancer (tissue) with great precision by imaging-guided injection, preferably using an imaging technique, such as computer tomograpy, ultrasound, gamma camera imaging, positron emission tomography, or magnetic resonance tumor imaging. Further procedures are selected from the group including, but not limited to, direct intratumoral injection by endoscopy, bronchoscopy, cystoscopy, colonoscopy, laparoscope and catheterization. In addition the pharmaceutical composition can be injected locoregionally or peritumorally by the same methods. Tumor or cancer tissue includes metastases of the primary tumor, e.g. to lymph nodes, skin, soft tissues, bone, visceral organs or other organs of the body.

Decoy receptors: Decoy receptors recognize certain growth factors or cytokines with high affinity and specificity, but are structurally incapable of signaling or presenting the agonist to signaling receptor complexes. They act as a molecular trap for the agonist and for signaling receptor components. A decoy receptor, or sink receptor, is a receptor that binds a ligand, inhibiting it from binding to its normal receptor. For instance, the receptor VEGFR-1 can prevent vascular endothelial growth factor (VEGF) from binding to the VEGFR-2.

Dominant negative receptors: Dominant negative receptors are variants of the particular receptor comprising dominant-negative (DN) mutations as leading to mutant polypeptides that disrupt the activity of the wild type receptor when overexpressed. In a first aspect, the invention relates to an immunostimulatory RNA (isRNA) for use in the treatment or prophylaxis of tumor and/or cancer diseases. In particular, an isRNA is provided for use in the treatment or prophylaxis of tumor and/or cancer diseases, wherein the isRNA is administered intratumorally.

### SUMMARY OF THE INVENTION

The inventors found that the administration, in particular the intratumoral administration, of an isRNA as described herein may be employed for treatment or prophylaxis of tumor or cancer diseases and related disorders. It has been shown that treatment of tumor or cancer diseases with isRNA is surprisingly effective, in particular if administered intratumorally, in decreasing tumor size. Moreover, the application of isRNA according to the invention was able to increase survival in animal models and to protect surviving animals from rechallenge with the same tumor although treatment with the pharmaceutical composition had been stopped upon rechallenge. This finding is in line with generation of long lasting immunologic memory to the tumor. This could not have been expected as the isRNA does not induce an adaptive immune response directly.

As used herein, the terms “tumor”, “cancer” or “cancer disease” refer to a malignant disease, which is preferably selected from the group consisting of Adenocystic carcinoma (Adenoid cystic carcinoma), Adrenocortical carcinoma, AIDS-related cancers, AIDS-related lymphoma, Anal cancer, Appendix cancer, Astrocytoma, Basal cell carcinoma, Bile duct cancer, Bladder cancer, Bone cancer, Osteosarcoma/Malignant fibrous histiocytoma, Brainstem glioma, Brain tumor, cerebellar astrocytoma, cerebral astrocytoma/malignant glioma, ependymoma, medulloblastoma, supratentorial primitive neuroectodermal tumors, visual pathway and hypothalamic glioma, Breast cancer, Bronchial adenomas/carcinoids, Burkitt lymphoma, childhood Carcinoid tumor, gastrointestinal Carcinoid tumor, Carcinoma of unknown primary, primary Central nervous system lymphoma, childhood Cerebellar astrocytoma, childhood Cerebral astrocytoma/Malignant glioma, Cervical cancer, Childhood cancers, Chronic lymphocytic leukemia, Colon Cancer, Cutaneous T-cell lymphoma including Mycosis Fungoides and Sezary Syndrome, Desmoplastic small round cell tumor, Endometrial cancer, Ependymoma, Esophageal cancer, Ewing's sarcoma in the Ewing family of tumors, Childhood Extracranial germ cell tumor, Extragonadal Germ cell tumor, Extrahepatic bile duct cancer, Intraocular melanoma, Retinoblastoma, Gallbladder cancer, Gastric (Stomach) cancer, Gastrointestinal Carcinoid Tumor, Gastrointestinal stromal tumor (GIST), extracranial, extragonadal, or ovarian Germ cell tumor, Gestational trophoblastic tumor, Glioma of the brain stem, Childhood Cerebral Astrocytoma, Childhood Visual Pathway and Hypothalamic Glioma, Gastric carcinoid, Hairy cell leukemia, Head and neck cancer, Heart cancer, Hepatocellular (liver) cancer, Hodgkin lymphoma, Human Papilloma Virus (HPV)-related cancer, Hypopharyngeal cancer, childhood Hypothalamic and visual pathway glioma, Intraocular Melanoma, Islet Cell Carcinoma (Endocrine Pancreas), Kaposi sarcoma, Kidney cancer (renal cell cancer), Laryngeal Cancer, Lip and Oral Cavity Cancer, Liposarcoma, Liver Cancer, Non-Small Cell Lung Cancer, Small Cell Lung Cancer, Lymphomas, AIDS-related Lymphoma, Burkitt Lymphoma, Hodgkin Lymphoma, Non-Hodgkin Lymphomas, Primary Central Nervous System Lymphoma, Malignant Fibrous Histiocytoma of Bone/Osteosarcoma, Childhood Medulloblastoma, Melanoma, Intraocular (Eye) Melanoma, Merkel Cell Carcinoma, Adult Malignant Mesothelioma, Childhood Mesothelioma, Head or Neck Cancer, Mouth Cancer, Childhood Multiple Endocrine Neoplasia Syndrome, Multiple Myeloma/Plasma Cell Neoplasm, Multiple Myeloma (Cancer of the Bone-Marrow), Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, islet cell Pancreatic cancer, Paranasal sinus and nasal cavity cancer, Parathyroid cancer, Penile cancer, Pharyngeal cancer, Pheochromocytoma, Pineal astrocytoma, Pineal germinoma, childhood Pineoblastoma and supratentorial primitive neuroectodermal tumors, Pituitary adenoma, Plasma cell neoplasia/plasmocytoma/Multiple myeloma, Pleuropulmonary blastoma, Primary central nervous system lymphoma, Prostate cancer, Rectal cancer, Renal cell carcinoma (kidney cancer), Cancer of the Renal pelvis and ureter, Retinoblastoma, childhood Rhabdomyosarcoma, Salivary gland cancer, Sarcoma of the Ewing family of tumors, Kaposi Sarcoma, soft tissue Sarcoma, uterine Sarcoma, Skin cancer (nonmelanoma), Skin cancer (melanoma), Merkel cell Skin carcinoma, Small intestine cancer, Squamous cell carcinoma, metastatic Squamous neck cancer with occult primary, soft tissue sarcoma (STS), childhood Supratentorial primitive neuroectodermal tumor, Testicular cancer (seminoma and non-seminoma), Throat cancer, childhood Thymoma, Thymoma and Thymic carcinoma, Thyroid cancer, childhood Thyroid cancer, Transitional cell cancer of the renal pelvis and ureter, gestational Trophoblastic tumor, Urethral cancer, endometrial Uterine cancer, Uterine sarcoma, Vaginal cancer, childhood Visual pathway and hypothalamic glioma, Vulvar cancer, and childhood Wilms tumor (kidney cancer).

Especially preferred examples of tumors or cancers that are suitable for intratumoral, including peritumoral or locoregional administration, preferably imaging guided loco-regional administration, are prostate cancer, lung cancer, breast cancer, brain cancer, head and neck cancer including cancer of the lips, mouth, or tongue, nasopharyngal cancers or lymphoma, thyroid cancer, thymic cancer, colon cancer, stomach cancer, esophageal cancer, liver cancer, biliary cancer, pancreas cancer, ovary cancer, skin cancer, (melanoma and non-melanoma skin cancer), urinary bladder and urothel, uterus and cervix, anal cancer, bone cancers, kidney cancer, adrenal cancer, testicular cancer, cutaneous T cell lymphoma, cutaneous B cell lymphoma, plasmocytoma, other Hodgkin and non-hodgkin lymphomas with injectable, solitary lesions, adenocystic carcinoma, other salivary gland cancers, neuroendocrine tumors, vulvar cancer, sarcoma (incl. pediatric sarcoma), penile cancer lymphomas.

Preferably, a tumor or cancer disease is selected from the group consisting of breast cancer (hormone receptor positive or negative forms); melanoma, preferably advanced and/or metastatic melanoma, most preferably advanced cutaneous melanoma (cMEL); squamous cell cancer of the skin (SCC), preferably unresectable and/or advanced SCC, most preferably cutaneous squamous cell carcinoma (cSCC), or other forms of malignant skin cancer; adenocystic carcinoma (ACC), preferably advanced ACC; cutaneous T-cell lymphoma, preferably advanced cutaneous T-cell lymphoma or cutaneous T-cell lymphoma of mycosis fungoides subtype (CTCL-MF); squamous cell carcinoma of the head and neck (HNSCC), preferably advanced or locally advanced HNSCC; follicular lymphoma (FL); marginal zone lymphoma, preferably nodal marginal zone lymphoma (nMZL); mantle cell lymphoma; primary cutaneous anaplastic large cell lymphoma (PC-ALCL); vulvar carcinoma, preferably vulvar squamous cell carcinoma (VSCC); soft tissue sarcoma; breast cancer; hepatocellular carcinoma; colorectal cancer (including MSI-High CRC); neuroendocrine tumors; pancreas cancer; gastroesophageal cancer; uveal melanoma; penile cancer lymphoma; salivary gland cancer; nasopharynx cancers; lung cancer, preferably locally advanced or advanced non-small cell lung cancer or small cell lung cancer of limited or extensive disease, lung metastases of other malignancies; mesothelioma; urothelial or bladder cancer; thyroid cancer; esophageal and gastric cancer; gastric cancer, esophageal cancer, liver cancer; malignancies with liver metastases; ovarian cancer; cervix cancer; renal cancer; adrenal malignancies, soft-tissue sarcoma, hematological malignancies with injectable lesions like cutaneous T-cell lymphoma; solitary or multiple myeloma; Hodgkin's disease; Non-Hodgkin Lymphoma, preferably indolent cutaneously accesible Non-Hodgkin Lymphoma (CTCL) or non-Hodgkin lymphoma with injectable lesions; sarcoma including its various subtypes; glioma grade I-IV; colorectal, rectal or anal cancer. In particular, the terms “tumor”, “cancer” or “cancer disease” as used herein refer to basal cell carcinoma; or melanoma, preferably advanced and/or metastatic melanoma; squamous cell cancer (SCC), preferably SCC of the skin, more preferably unresectable and/or advanced SCC of the skin; or squamous cell carcinoma of the head and neck (HNSCC), preferably advanced and/or platinum-refractory and/or immunotherapy-refractory HNSCC; or vulvar carcinoma or vulvar squamous cell carcinoma (VSCC), preferably unresectable and/or advanced VSCC, more preferably advanced and/or platinum-refractory and/or immunotherapy-refractory VSCC; or adenocystic carcinoma (ACC), preferably advanced ACC; or cutaneous T-cell lymphoma, preferably advanced cutaneous T-cell lymphoma or cutaneous T-cell lymphoma of mycosis fungoides subtype (CTCL-MF), preferably advanced CTCL-MF, preferably cutaneous T-cell lymphoma refractory to local treatment or to chemotherapy; or follicular lymphoma; or marginal zone lymphoma, preferably nodal marginal zone lymphoma (nMZL); or mantle cell lymphoma; or primary cutaneous anaplastic large cell lymphoma (PC-ALCL); or soft tissue sarcoma (STS); or Human Papilloma Virus (HPV)-related cancer; or breast cancer; or hepatocellular carcinoma; or colorectal cancer (including MSI-High CRC); or neuroendocrine tumors; or pancreas cancer; or gastroesophageal cancer; or uveal melanoma; or penile cancer lymphoma. In certain embodiments of the invention, the terms “tumor”, “cancer” or “cancer disease” as used herein refer to basal cell carcinoma; or melanoma, preferably advanced and/or metastatic melanoma; squamous cell cancer (SCC), preferably SCC of the skin, more preferably unresectable and/or advanced SCC of the skin; or squamous cell carcinoma of the head and neck (HNSCC), preferably advanced and/or platinum-refractory and/or immunotherapy-refractory HNSCC; or vulvar carcinoma or vulvar squamous cell carcinoma (VSCC), preferably unresectable and/or advanced VSCC, more preferably advanced and/or platinum-refractory and/or immunotherapy-refractory VSCC; or adenocystic carcinoma (ACC), preferably advanced ACC; or cutaneous T-cell lymphoma, preferably advanced cutaneous T-cell lymphoma or cutaneous T-cell lymphoma of mycosis fungoides subtype (CTCL-MF), more preferably advanced CTCL-MF, more preferably advanced CTCL-MF refractory to local treatment or to chemotherapy; or follicular lymphoma; or marginal zone lymphoma, preferably nodal marginal zone lymphoma (nMZL); or primary cutaneous anaplastic large cell lymphoma (PC-ALCL); or soft tissue sarcoma (STS); or Human Papilloma Virus (HPV)-related cancer

According to a preferred embodiment, the invention thus concerns an isRNA for use in the treatment or prophylaxis of tumor and/or cancer diseases, wherein the tumor or the cancer disease is selected from the group consisting of melanoma, preferably advanced and/or metastatic melanoma, preferably advanced cutaneous melanoma (cMEL), squamous cell cancer of the skin (SCC), preferably unresectable and/or advanced SCC or cutaneous squamous cell carcinoma (cSCC), and/or squamous cell carcinoma of the head and neck (HNSCC), preferably advanced and/or immunotherapy-refractory platinum-refractory HNSCC, and/or adenocystic carcinoma (ACC), preferably advanced ACC, and/or cutaneous T-cell lymphoma, preferably advanced cutaneous T-cell lymphoma refractory to local treatment or to chemotherapy.

In a further preferred embodiment, the invention concerns an isRNA, preferably as described herein, for use in the treatment and/or prophylaxis of a tumor and/or cancer disease, wherein the tumor or cancer disease is selected from the group consisting of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy.

According to a particularly preferred embodiment the invention concerns an isRNA, preferably as described herein, for use in the treatment and/or prophylaxis of a tumor and/or cancer disease, wherein the tumor or the cancer disease is selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy.

According to a further particularly preferred embodiment, the invention concerns an isRNA for use in the treatment or prophylaxis of tumor and/or cancer diseases, wherein the tumor or the cancer disease is selected from the group consisting of advanced melanoma, preferably advanced cutaneous melanoma (cMEL), squamous cell carcinoma of the skin (SCC), preferably cutaneous squamous cell carcinoma (cSCC), squamous cell carcinoma of the head and neck (HNSCC), and adenoid cystic carcinoma (adenocystic carcinoma (ACC)).

In an alternative preferred embodiment the invention concerns an isRNA for use in the treatment or prophylaxis of tumor and/or cancer diseases, wherein the tumor or the cancer disease is selected from the group consisting of advanced cutaneous melanoma (cMEL), cutaneous squamous cell carcinoma (cSCC), head and neck squamous cell carcinoma (hnSCC), and adenoid cystic carcinoma (ACC).

According to a preferred embodiment, the isRNA as described herein is for use in the treatment of advanced melanoma preferably advanced cutaneous melanoma (cMEL), which was confirmed histologically, preferably an unresectable and/or metastatic (cutaneous) melanoma. More preferably, the melanoma is refractory to standard therapy, in particular to immunotherapy with a checkpoint inhibitor or immunostimulatory agent or with targeted chemotherapy or a combination thereof.

In another embodiment, the isRNA as described herein is for use in the treatment of advanced SCC, preferably cutaneous squamous cell carcinoma (cSCC), which was confirmed histologically, preferably an unresectable and/or metastatic SCC. More preferably, the SCC is refractory to standard therapy or no therapy exists for the SCC.

According to a preferred embodiment, the isRNA as described herein is for use in the treatment of locally advanced or advanced HNSCC, which was confirmed histologically, preferably an unresectable and/or recurrent and/or metastatic HNSCC. More preferably, the HNSCC is refractory to standard therapy, in particular to platinum-based therapy or radiation therapy or to immunotherapy with a checkpoint inhibitor or immunostimulatory agent, or a combination of any of the above.

According to a further preferred embodiment, the isRNA as described herein is for use in the treatment of adenocystic carcinoma (ACC), which was confirmed histologically, preferably an unresectable and/or recurrent and/or metastatic adenocystic carcinoma (ACC). More preferably, the ACC is refractory to standard therapy, in particular to platinum-based therapy or radiation therapy or to immunotherapy with a checkpoint inhibitor or immunostimulatory agent, or a combination of any of the above. According to another embodiment, the isRNA as described herein is for use in the treatment of cutaneous T-cell lymphoma, which was confirmed histologically, preferably an unresectable and/or recurrent and/or metastatic cutaneous T-cell lymphoma. More preferably, the cutaneous T-cell lymphoma is refractory to standard therapy, in particular to platinum-based therapy or radiation therapy or to immunotherapy with a checkpoint inhibitor or immunostimulatory agent, or a combination of any of the above.

According to a further embodiment, the isRNA as described herein is for use in the treatment of vulvar squamous cell cancer (VSCC), which was confirmed histologically, preferably an unresectable and/or recurrent and/or metastatic VSCC. More preferably, the VSCC is refractory to standard therapy, in particular to platinum-based therapy or radiation therapy or to immunotherapy with a checkpoint inhibitor or immunostimulatory agent, or a combination of any of the above.

In a preferred embodiment, the isRNA for use in the treatment or prophylaxis of a tumor and/or cancer disease, preferably as described herein, elicits an innate immune response, which may support an adaptive immune response.

The isRNA for use in the treatment or prophylaxis of a tumor and/or cancer disease as described herein, may preferably be any (double-stranded or single-stranded) RNA, e.g. a coding RNA, as defined herein. In a preferred embodiment, the isRNA for use in the treatment or prophylaxis of a tumor and/or cancer disease as described herein is a non-coding RNA. In this context, the term “non-coding” refers to the fact that the isRNA does preferably not encode a peptide or protein or that the isRNA does preferably not comprise a coding sequence, preferably as described herein.

In some embodiments, the isRNA for use in the treatment or prophylaxis of a tumor and/or cancer disease as described herein may be a single-stranded, a double-stranded or a partially double-stranded RNA, more preferably a single-stranded RNA, and/or a circular or linear RNA, more preferably a linear RNA. More preferably, the isRNA may be a (linear) single-stranded RNA. Even more preferably, the isRNA may be a (long) (linear) single-stranded) non-coding RNA. In this context, it is particular preferred that the isRNA carries a triphosphate at its 5′-end, which is typically the case for in vitro transcribed RNA. An isRNA may also occur as a short RNA oligonucleotide as defined herein.

The isRNA for use in the treatment or prophylaxis of a tumor and/or cancer disease as described herein may furthermore be selected from any class of RNA molecules, naturally occurding or prepared synthetically, and which can induce an innate immune response and which preferably supports an adaptive immune response induced by an antigen. In this context, an immune response may occur in various ways. A substantial factor for a suitable (adaptive) immune response is the stimulation of different T cell sub-populations. T-lymphocytes are typically divided into two sub-populations, the T-helper 1 (Th1) cells and the T-helper 2 (Th2) cells, with which the immune system is capable of destroying intracellular (Th1) and extracellular (Th2) pathogens (e.g. antigens). The two Th cell populations differ in the pattern of the effector proteins (cytokines) produced by them. Thus, Th1 cells assist the cellular immune response by activation of macrophages and cytotoxic T cells. Th2 cells, on the other hand, promote the humoral immune response by stimulation of B-cells for conversion into plasma cells and by formation of antibodies (e.g. against antigens). The Th1/Th2 ratio is therefore of great importance in the induction and maintenance of an adaptive immune response. In connection with the present invention, the Th1/Th2 ratio of the (adaptive) immune response is preferably shifted in the direction towards the cellular response (Th1 response) and a cellular immune response is thereby induced.

According to one example, the innate immune system, which may support an adaptive immune response, may be activated by ligands of Toll-like receptors (TLRs). TLRs are a family of highly conserved pattern recognition receptor (PRR) polypeptides that recognize pathogen-associated molecular patterns (PAMPs) and play a critical role in innate immunity in mammals. Currently at least thirteen family members, designated TLR1-TLR13 (Toll-like receptors: TLR1, TLR2, TLR3, TLR4, TLR5, TLR6, TLR7, TLR8, TLR9, TLR10, TLR11, TLR12 or TLR13), have been identified. Furthermore, a number of specific TLR ligands have been identified. It was found, for example, that un methylated bacterial DNA and synthetic analogs thereof (CpG DNA) are ligands for TLR9 (Hemmi H et al. (2000) Nature 408:740-5; Bauer S et al. (2001) Proc NatlAcadSci USA 98, 9237-42). Furthermore, it has been reported that ligands for certain TLRs include certain nucleic acid molecules and that certain types of RNA are immunostimulatory in a sequence-independent or sequence-dependent manner, wherein these various immunostimulatory RNAs may e.g. stimulate TLR3, TLR7, or TLR8, or intracellular receptors such as RIG-I, MDA-5, etc. For example, Lipford et al. determined certain G,U-containing oligoribonucleotides as immunostimulatory by acting via TLR7 and TLR8 (see WO 03/086280). The immunostimulatory G,U-containing oligoribonucleotides described by Lipford et al. were believed to be derivable from RNA sources including ribosomal RNA, transfer RNA, messenger RNA, and viral RNA.

Preferably, the isRNA for use in the treatment or prophylaxis of a tumor and/or cancer disease as described herein may comprise any RNA sequence known to be immunostimulatory, including, without being limited thereto, RNA sequences representing and/or encoding ligands of TLRs, preferably selected from human family members TLR1-TLR10 or murine family members TLR1-TLR13, more preferably selected from (human) family members TLR1-TLR10, even more preferably from TLR7 and TLR8, ligands for intracellular receptors for RNA (such as RIG-I or MDA-5, etc.) (see e.g. Meylan, E., Tschopp, J. (2006). Toll-like receptors and RNA helicases: two parallel ways to trigger antiviral responses. Mol. Cell 22, 561-569), or any other immunostimulatory RNA sequence. Furthermore, (classes of) immunostimulatory RNA molecules for use in the treatment or prophylaxis of a tumor and/or cancer disease as described herein, may include any other RNA capable of eliciting an immune response. Without being limited thereto, the isRNA for use in the treatment or prophylaxis of a tumor and/or cancer disease as described herein may include ribosomal RNA (rRNA), transfer RNA (tRNA), messenger RNA (mRNA), and viral RNA (vRNA). In preferred embodiments, the isRNA may have a length of 1000 to 5000, of 500 to 5000, of 5 to 5000, or of 5 to 1000, 5 to 500, 5 to 250, of 5 to 100, of 5 to 50 or of 5 to 30 nucleotides.

According to a particularly preferred embodiment, the isRNA for use in the treatment or prophylaxis of a tumor and/or cancer disease as described herein consists of or comprises a nucleic acid of the following formula (I) or (II):

GₗXₘGₙ, (formula (I))

wherein:
G is guanosine (guanine), uridine (uracil) or an analogue of guanosine (guanine) or uridine (uracil);
X is guanosine (guanine), (uridine) uracil, adenosine (adenine), thymidine (thymine), cytidine (cytosine) or an analogue of the above-mentioned nucleotides (nucleosides);
l is an integer from 1 to 40,
   wherein
   when I=1 G is guanosine (guanine) or an analogue thereof,
   when I>1 at least 50% of the nucleotides (nucleosides) are guanosine (guanine) or an analogue thereof;
m is an integer and is at least 3;
   wherein
   when m=3 X is uridine (uracil) or an analogue thereof,
   when m>3 at least 3 successive uridines (uracils) or analogues of uridine (uracil) occur;
n is an integer from 1 to 40,
   wherein
   when n=1 G is guanosine (guanine) or an analogue thereof,
   when n>1 at least 50% of the nucleotides (nucleosides) are guanosine (guanine) or an analogue thereof;

CₗXₘCₙ, (formula (II))

wherein:
C is cytidine (cytosine), uridine (uracil) or an analogue of cytidine (cytosine) or uridine (uracil);
X is guanosine (guanine), uridine (uracil), adenosine (adenine), thymidine (thymine), cytidine (cytosine) or an analogue of the above-mentioned nucleotides (nucleosides);
l is an integer from 1 to 40,
   wherein
   when I=1 C is cytidine (cytosine) or an analogue thereof,
   when I>1 at least 50% of the nucleotides (nucleosides) are cytidine (cytosine) or an analogue thereof;
m is an integer and is at least 3;
   wherein
   when m=3 X is uridine (uracil) or an analogue thereof,
   when m>3 at least 3 successive uridines (uracils) or analogues of uridine (uracil) occur;
n is an integer from 1 to 40,
   wherein
   when n=1 C is cytidine (cytosine) or an analogue thereof,
   when n>1 at least 50% of the nucleotides (nucleosides) are cytidine (cytosine) or an analogue thereof.

The nucleic acids of formula (I) or (II), which may be used as isRNA may be relatively short nucleic acid molecules with a typical length of approximately from 5 to 100 (but may also be longer than 100 nucleotides for specific embodiments, e.g. up to 200 nucleotides), from 5 to 90 or from 5 to 80 nucleotides, preferably a length of approximately from 5 to 70, more preferably a length of approximately from 8 to 60 and, more preferably a length of approximately from 15 to 60 nucleotides, more preferably from 20 to 60, most preferably from 30 to 60 nucleotides. If the nucleic acid of the inventive nucleic acid cargo complex has a maximum length of, for example, 100 nucleotides, m will typically be 98. The number of nucleotides G in the nucleic acid of formula (I) is determined by l or n. l and n, independently of one another, are each an integer from 1 to 40, wherein when l or n=1 G is guanosine or an analogue thereof, and when l or n>1 at least 50% of the nucleotides are guanosine or an analogue thereof. For example, without implying any limitation, when l or n=4 Gl or Gn can be, for example, a GUGU, GGUU, UGUG, UUGG, GUUG, GGGU, GGUG, GUGG, UGGG or GGGG, etc.; when l or n=5 Gl or Gn can be, for example, a GGGUU, GGUGU, GUGGU, UGGGU, UGGUG, UGUGG, UUGGG, GUGUG, GGGGU, GGGUG, GGUGG, GUGGG, UGGGG, or GGGGG, etc.; etc. A nucleotide adjacent to Xₘ in the nucleic acid of formula (I) according to the invention is preferably not a uracil. Similarly, the number of nucleotides C in the nucleic acid of formula (II) according to the invention is determined by l or n. l and n, independently of one another, are each an integer from 1 to 40, wherein when l or n=1 C is cytidine or an analogue thereof, and when l or n>1 at least 50% of the nucleotides are cytidine or an analogue thereof. For example, without implying any limitation, when l or n=4, Cl or Cn can be, for example, a CUCU, CCUU, UCUC, UUCC, CUUC, CCCU, CCUC, CUCC, UCCC or CCCC, etc.; when l or n=5 Cl or Cn can be, for example, a CCCUU, CCUCU, CUCCU, UCCCU, UCCUC, UCUCC, UUCCC, CUCUC, CCCCU, CCCUC, CCUCC, CUCCC, UCCCC, or CCCCC, etc.; etc. A nucleotide adjacent to Xₘ in the nucleic acid of formula (II) according to the invention is preferably not a uracil. Preferably, for formula (I), when l or n>1, at least 60%, 70%, 80%, 90% or even 100% of the nucleotides are guanosine or an analogue thereof, as defined above. The remaining nucleotides to 100% (when guanosine constitutes less than 100% of the nucleotides) in the flanking sequences G1 and/or Gn are uridine or an analogue thereof, as defined hereinbefore. Also preferably, l and n, independently of one another, are each an integer from 2 to 30, more preferably an integer from 2 to 20 and yet more preferably an integer from 2 to 15. The lower limit of l or n can be varied if necessary and is at least 1, preferably at least 2, more preferably at least 3, 4, 5, 6, 7, 8, 9 or 10. This definition applies correspondingly to formula (II).

According to a particularly preferred embodiment, a nucleic acid according to any of formulas (I) or (II) above, which may be used as isRNA in the context of the present invention, may be selected from a nucleic acid sequence consisting or comprising any of the following sequences SEQ ID NOs: 471 to 554, or from a sequence having at least 60%, 70%, 80%, 90%, or even 95% sequence identity with any of these sequences. In some embodiments, the isRNA is selected from a nucleic acid sequence consisting or comprising any one of the nucleic acid sequences SEQ ID NOs: 471 to 554, or a fragment or variant of any one of these sequences.

According to a further preferred embodiment, the isRNA for use in the treatment or prophylaxis of a tumor and/or cancer disease as described herein consists of or comprises a nucleic acid of formula (III) or (IV):

(NᵤGₗXₘGₙNᵥ)ₐ, (formula (III))

wherein:
G is guanosine (guanine), uridine (uracil) or an analogue of guanosine (guanine) or uridine (uracil), preferably guanosine (guanine) or an analogue thereof;
X is guanosine (guanine), uridine (uracil), adenosine (adenine), thymidine (thymine), cytidine (cytosine), or an analogue of these nucleotides (nucleosides), preferably uridine (uracil) or an analogue thereof;
N is a nucleic acid sequence having a length of about 4 to 50, preferably of about 4 to 40, more preferably of about 4 to 30 or 4 to 20 nucleic acids, each N independently being selected from guanosine (guanine), uridine (uracil), adenosine (adenine), thymidine (thymine), cytidine (cytosine) or an analogue of these nucleotides (nucleosides);
a is an integer from 1 to 20, preferably from 1 to 15, most preferably from 1 to 10;
l is an integer from 1 to 40,
   wherein when I=1, G is guanosine (guanine) or an analogue thereof,
   when l>1, at least 50% of these nucleotides (nucleosides) are guanosine (guanine) or an analogue thereof;
m is an integer and is at least 3;
   wherein when m=3, X is uridine (uracil) or an analogue thereof, and
   when m>3, at least 3 successive uridines (uracils) or analogues of uridine (uracil) occur;
n is an integer from 1 to 40,
   wherein when n=1, G is guanosine (guanine) or an analogue thereof,
   when n>1, at least 50% of these nucleotides (nucleosides) are guanosine (guanine) or an analogue thereof;
u,v may be independently from each other an integer from 0 to 50, preferably wherein when u=0, v 1, or when v=0, u≥1;
   wherein the nucleic acid molecule of formula (III) has a length of at least 50 nucleotides, preferably of at least 100 nucleotides, more preferably of at least 150 nucleotides, even more preferably of at least 200 nucleotides and most preferably of at least 250 nucleotides.

(NᵤCₗXₘCₙNᵥ)ₐ, (formula (IV))

wherein:
C is cytidine (cytosine), uridine (uracil) or an analogue of cytidine (cytosine) or uridine (uracil), preferably cytidine (cytosine) or an analogue thereof;
X is guanosine (guanine), uridine (uracil), adenosine (adenine), thymidine (thymine), cytidine (cytosine) or an analogue of the above-mentioned nucleotides (nucleosides), preferably uridine (uracil) or an analogue thereof;
N is each a nucleic acid sequence having independent from each other a length of about 4 to 50, preferably of about 4 to 40, more preferably of about 4 to 30 or 4 to 20 nucleic acids, each N independently being selected from guanosine (guanine), uridine (uracil), adenosine (adenine), thymidine (thymine), cytidine (cytosine) or an analogue of these nucleotides (nucleosides);
a is an integer from 1 to 20, preferably from 1 to 15, most preferably from 1 to 10;
l is an integer from 1 to 40,
   wherein when I=1, C is cytidine (cytosine) or an analogue thereof,
   when I>1, at least 50% of these nucleotides (nucleosides) are cytidine (cytosine) or an analogue thereof;
m is an integer and is at least 3;
   wherein when m=3, X is uridine (uracil) or an analogue thereof,
   when m>3, at least 3 successive uridines (uracils) or analogues of uridine (uracil) occur;
n is an integer from 1 to 40,
   wherein when n=1, C is cytidine (cytosine) or an analogue thereof,
   when n>1, at least 50% of these nucleotides (nucleosides) are cytidine (cytosine) or an analogue thereof.
u, v may be independently from each other an integer from 0 to 50,
   preferably wherein when u=0, v 1, or when v=0, u≥1;
      wherein the nucleic acid molecule of formula (V) according to the invention has a length of at least 50 nucleotides, preferably of at least 100 nucleotides, more preferably of at least 150 nucleotides, even more preferably of at least 200 nucleotides and most preferably of at least 250 nucleotides.

For formula (IV), any of the definitions given above for elements N (i.e. Nᵤ and Nᵥ) and X (Xₘ), particularly the core structure as defined above, as well as for integers a, l, m, n, u and v, similarly apply to elements of formula (IV) correspondingly, wherein in formula (IV) the core structure is defined by CₗXₘCₙ. The definition of bordering elements Nᵤ and Nᵥ is identical to the definitions given above for Nᵤ and Nv.

According to a very particularly preferred embodiment, the nucleic acid molecule, preferably immunostimulating RNA according to formula (III) may be selected from e.g. any of the sequences according to SEQ ID NOs: 555 to 563 or from a sequence having at least 60%, 70%, 80%, 90%, or even 95% sequence identity with any of these sequences. Preferably, the isRNA as used herein comprises or consists of a nucleic acid sequence according to any one of SEQ ID NO: 555 to 563, or a fragment or variant of any one of these sequences.

In this context particularly preferred are immunostimulating RNAs according to any one of SEQ ID NOs: 433 to 437, 1014 to 1016, 1055 or 1056. More preferably, an immunostimulating RNA as used herein, comprises or consists of a nucleic acid sequence according to any one of SEQ ID NOs: 433 to 437, 1014 to 1016, 1055 or 1056, or a fragment or variant of any one of these sequences. Even more preferably, the immunostimulating RNA comprises or consists of a nucleic acid sequence having at least 60%, 70%, 80%, 90%, or even 95% sequence identity with any one of the nucleic acid sequences according to SEQ ID NOs: 433 to 437, 1014 to 1016, 1055 or 1056.

According to a particularly preferred embodiment, an immunostimulating RNA as used herein, comprises or consists of a nucleic acid sequence according to any one of SEQ ID NOs: 433; 434, or 1014 to 1016, or a fragment or variant of any one of these sequences. Even more preferably, the immunostimulating RNA comprises or consists of a nucleic acid sequence having at least 60%, 70%, 80%, 90%, or even 95% sequence identity with the nucleic acid sequence according to any one of SEQ ID NOs: 433, 434 or 1014 to 1016.

According to another very particularly preferred embodiment, the nucleic acid molecule according to formula (IV) may be selected from e.g. any of the sequences according to SEQ ID NOs: 433; 434, or 1014 to 1016, or from a sequence having at least 60%, 70%, 80%, 90%, or even 95% sequence identity with any of these sequences. Preferably, the isRNA as used herein comprises or consists of a nucleic acid sequence according to SEQ ID NO: 433; 434 or 1014 to 1016, or a fragment or variant of any one of these sequences.

Further, all modifications disclosed herein in the context of coding RNA may also be applied to non-coding RNA, if applicable.

The isRNA for use as described herein may be administered naked without being associated with any further vehicle, carrier, transfection or complexation agent.

In a preferred embodiment, the isRNA for use in the treatment or prophylaxis of a tumor and/or cancer disease as described herein is formulated together with further compounds for increasing the transfection efficiency and/or the immunostimulatory properties of the isRNA. Such compounds are also termed herein carriers, vehicles, transfection or complexation agents. Preferably, the isRNA for use in the treatment or prophylaxis of a tumor and/or cancer disease as described herein is complexed with a cationic or polycationic compound, preferably with a cationic or polycationic polymer, a cationic or polycationic peptide or protein, e.g. protamine, a cationic or polycationic polysaccharide and/or a cationic or polycationic lipid. According to a particularly preferred embodiment, the isRNA for use in the treatment or prophylaxis of a tumor and/or cancer disease as described herein is complexed with a cationic or polycationic compound, wherein the cationic or polycationic compound is a polymeric carrier.

Such cationic or polycationic polymers, cationic or polycationic peptides or proteins, cationic or polycationic polysaccharides, cationic or polycationic lipids or polymeric carriers are useful as carriers, vehicles, transfection or complexation agents of nucleic acids in the context of the present invention, in particular of the isRNA for use as described herein. Accordingly, in a further embodiment of the invention it is preferred that the isRNA is associated with or complexed with a cationic or polycationic compound or a polymeric carrier, optionally in a weight ratio selected from a range of about 6:1 (w/w) to about 0.25:1 (w/w), more preferably from about 5:1 (w/w) to about 0.5:1 (w/w), even more preferably of about 4:1 (w/w) to about 1:1 (w/w) or of about 3:1 (w/w) to about 1:1 (w/w), and most preferably a ratio of about 3:1 (w/w) to about 2:1 (w/w) of RNA to cationic or polycationic compound and/or with a polymeric carrier; or optionally in a nitrogen/phosphate ratio of RNA to cationic or polycationic compound and/or polymeric carrier in the range of about 0.1 to 10, preferably in a range of about 0.3 to 4 or 0.3 to 1, and most preferably in a range of about 0.5-1 or 0.7 to 1, and even most preferably in a range of about 0.3 to 0.9 or 0.5 to 0.9.

The ratio of the isRNA for use in the treatment or prophylaxis of a tumor and/or cancer disease as described herein, and the cationic or polycationic compound, may be calculated on the basis of the nitrogen/phosphate ratio (N/P-ratio) of all these components. In the context of the present invention, an N/P-ratio is preferably in the range of about 0.01 to 4, 0.01 to 2, 0.1 to 2 or 0.1 to 1.5 regarding the ratio of nucleic acids: cationic or polycationic peptide contained in the inventive vaccine, and most preferably in the range of about 0.1 to 1. Alternatively, the N/P ratio of the isRNA to the cationic or polycationic compound, preferably the cationic or polycationic peptide or protein, is in the range of about 0.1 to 10, including a range of about 0.3 to 4, of about 0.5 to 2, of about 0.7 to 2 and of about 0.7 to 1.5. Such an N/P ratio is preferably designed to provide good transfection properties in vivo and transport into and through cell membranes. Preferably, for this purpose, cationic or polycationic compound and/or polymeric carriers as used herein, are based on peptide sequences.

Cationic or polycationic compounds, being particularly preferred agents in this context include protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA (SEQ ID NO: 1063 ) or protein transduction domains (PTDs), PpT620, proline-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, pIsl, FGF, Lactoferrin, Transportan, Buforin-2, Bac715-24, SynB, SynB(1), pVEC, hCT-derived peptides, SAP, or histones. In this context, protamine is particularly preferred.

Additionally, preferred cationic or polycationic proteins or peptides may be selected from the following proteins or peptides having the following total formula (V):

(Arg)ₗ;(Lys)ₘ;(His)ₙ;(Orn)ₒ;(Xaa)ₓ, (formula (V)

wherein l+m+n+o+x=8-15, and l, m, n or o independently of each other may be any number selected from 0, 1, 2, 3, 4, 5, 6, 7, 8, 9, 10, 11, 12, 13, 14 or 15, provided that the overall content of Arg, Lys, His and Orn represents at least 50% of all amino acids of the oligopeptide; and Xaa may be any amino acid selected from native (=naturally occurring) or non-native amino acids except of Arg, Lys, His or Orn; and x may be any number selected from 0, 1, 2, 3 or 4, provided, that the overall content of Xaa does not exceed 50% of all amino acids of the oligopeptide. Particularly preferred cationic peptides in this context are e.g. Arg₇ (SEQ ID NO: 1064), Arg₈ (SEQ ID NO: 1065), Arg₈ (SEQ ID NO: 1066), H₃R₉ (SEQ ID NO: 1067), R₉H₃ (SEQ ID NO: 1068), H₃R₉H₃ (SEQ ID NO: 1069), YSSR₉SSY (SEQ ID NO: 1070), (RKH)₄ (SEQ ID NO: 1071), Y(RKH)₂R (SEQ ID NO: 1072), etc. In this context the disclosure of WO 2009/030481 is incorporated herewith by reference.

A polymeric carrier used according to the invention might be a polymeric carrier formed by disulfide-crosslinked cationic components.

According to a further particularly preferred embodiment, cationic or polycationic peptides or proteins of the polymeric carrier, having the empirical sum formula (V) as shown above and which comprise or are additionally modified to comprise at least one —SH moeity, may be, without being restricted thereto, selected from the subgroup consisting of generic formulas Arg₇ (also termed as R₇; SEQ ID NO: 1064), Arg₉ (also termed R₉; SEQ ID NO: 1066), Arg₁₂ (also termed as R₁₂; SEQ ID NO: 1073).

According to a one further particularly preferred embodiment, the cationic or polycationic peptide or protein of the polymeric carrier, when defined according to formula {(Arg)ₗ;(Lys)ₘ;(His)ₙ;(Orn)ₒ;(Xaa)ₓ} (formula (V)) as shown above and which comprise or are additionally modified to comprise at least one —SH moeity, may be, without being restricted thereto, selected from subformula (Va):

{(Arg)ₗ;(Lys)ₘ;(His)ₙ;(Orn)ₒ;(xaa′)ₓ(Cys)_{y}} formula (Va)

wherein (Arg)ₗ;(Lys)ₘ;(His)ₙ;(Orn)ₒ; and x are as defined herein, Xaa′ is any amino acid selected from native (=naturally occurring) or non-native amino acids except of Arg, Lys, His, Orn or Cys and y is any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80 and 81-90, provided that the overall content of Arg (Arginine), Lys (Lysine), His (Histidine) and Orn (Ornithine) represents at least 10% of all amino acids of the oligopeptide.

This embodiment may apply to situations, wherein the cationic or polycationic peptide or protein of the polymeric carrier, e.g. when defined according to empirical formula (Arg)ₗ;(Lys)ₘ;(His)ₙ;(Orn)ₒ;(Xaa)ₓ (formula (V)) as shown above, comprises or has been modified with at least one cysteine as —SH moiety in the above meaning such that the cationic or polycationic peptide as cationic component carries at least one cysteine, which is capable to form a disulfide bond with other components of the polymeric carrier. Examples may comprise any of the following sequences:

(SEQ ID NO: 581)
Cys(Arg₇),
(SEQ ID NO: 582)
Cys(Arg₈),
(SEQ ID NO: 583)
Cys(Arg₉),
(SEQ ID NO: 584)
Cys(Arg₁₀),
(SEQ ID NO: 585)
Cys(Arg₁₁),
(SEQ ID NO: 580)(SEQ ID NO: 571)
Cys(Arg₁₂),
(SEQ ID NO: 586)
Cys(Arg₁₃),
(SEQ ID NO: 587)
Cys(Arg₁₄),
(SEQ ID NO: 588)
Cys(Arg₁₅),
(SEQ ID NO: 589)
Cys(Arg₁₆),
(SEQ ID NO: 590)
Cys(Arg₁₇),
(SEQ ID NO: 591)
Cys(Arg₁₈),
(SEQ ID NO: 592)
Cys(Arg₁₉),
(SEQ ID NO: 593)
Cys(Arg₂₀).

According to another particularly preferred embodiment, the cationic or polycationic peptide or protein of the polymeric carrier, when defined according to formula {(Arg)ₗ;(Lys)ₘ;(His)ₙ;(Orn)ₒ;(Xaa)ₓ} (formula (V)) as shown above, may be, without being restricted thereto, selected from subformula (Vb):

Cys1{(Arg)ₗ;(Lys)ₘ;(His)ₙ;(Orn)ₒ;(Xaa)ₓ}Cys2 (formula (Vb))

wherein empirical formula {(Arg)ₗ;(Lys)ₘ;(His)ₙ;(Orn)ₒ;(Xaa)ₓ} (formula (V)) is as defined herein and forms a core of an amino acid sequence according to (semiempirical) formula (V) and wherein Cys1 and Cys2 are cysteines proximal to, or terminal to (Arg)ₗ;(Ls)ₘ;(His)ₙ;(Orn)ₒ;(Xaa)ₓ. Examples may comprise any of the above sequences flanked by two Cys and following sequences:

(SEQ ID NO: 566)
Cys(Arg₇)Cys,
(SEQ ID NO: 567)
Cys(Arg₈)Cys,
(SEQ ID NO: 568)
Cys(Arg₉)Cys,
(SEQ ID NO: 569
Cys(Arg₁₀)Cys,
(SEQ ID NO: 570)
Cys(Arg₁₁)Cys,
(SEQ ID NO: 579)
Cys(Arg₁₂)Cys,
(SEQ ID NO: 571)
Cys(Arg₁₃)Cys,
(SEQ ID NO: 572)
Cys(Arg₁₄)Cys,
(SEQ ID NO: 573)
Cys(Arg₁₅)Cys,
(SEQ ID NO: 574)
Cys(Arg₁₆)Cys,
(SEQ ID NO: 575)
Cys(Arg₁₇)Cys,
(SEQ ID NO: 576)
Cys(Arg₁₈)Cys,
(SEQ ID NO: 577)
Cys(Arg₁₉)Cys,
(SEQ ID NO: 578)
Cys(Arg₂₀)Cys.

This embodiment may apply to situations, wherein the cationic or polycationic peptide or protein of the polymeric carrier, e.g. when defined according to empirical formula (Arg)ₗ;(Lys)ₘ;(His)ₙ;(Orn)ₒ;(Xaa)ₓ (formula (V)) as shown above, has been modified with at least two cysteines as —SH moieties in the above meaning such that the cationic or polycationic peptide of the inventive polymeric carrier cargo complex as cationic component carries at least two (terminal) cysteines, which are capable to form a disulfide bond with other components of the polymeric carrier.

In a preferred embodiment, the polymeric carrier is formed by, comprises or consists of the peptide CysArg₁₂Cys (CRRRRRRRRRRRRC) (SEQ ID NO: 579) or CysArg₁₂ (CRRRRRRRRRRRR) (SEQ ID NO: 580). In some embodiments, the polymeric carrier compound is formed by, comprises or consists of a (R12C)—(R12C) dimer (Arg₁₂Cys-CysArg₁₂ dimer, (SEQ ID NO: 1077)), wherein the individual peptide monomers in the dimer (CR12 (CysArg₁₂; SEQ ID NO: 580)), are connected via —SH groups. In further preferred embodiments, the polymeric carrier compound is formed by, comprises or consists of a (WR12C) (WR12C) dimer (TrpArg₁₂Cys-CysArg₁₂Trp dimer (SEQ ID NO: 1078)), wherein the individual peptide monomers in the dimer (WR12C (TrpArg₁₂Cys; SEQ ID NO: 1017)), are connected via —SH groups. According to certain embodiments, the polymeric carrier compound is formed by, comprises or consists of a (CR12)-(CR12C)—(CR12) trimer (Arg₁₂Cys-CysArg₁₂Cys-CysArg₁₂ trimer (SEQ ID NO: 1079)), wherein the individual peptide monomers in the dimer (CR12C (CysArg₁₂Cys; SEQ ID NO: 579) and CR12 (CysArg₁₂; SEQ ID NO: 580)), are connected via —SH groups. In specific embodiments, the polymeric carrier consists of a (R12C)—(R12C) dimer, a (WR12C)—(WR12C) dimer, or a (CR12)-(CR12C)—(CR12) trimer (SEQ ID NO: 1079), wherein the individual cationic peptide (elements) in the dimer (e.g., (WR12C (SEQ ID NO: 1017))), or the trimer (e.g., (CR12 (SEQ ID NO: 580))) are connected via —SH groups of their cysteine residues.

According to a second alternative, at least one cationic (or polycationic) component of the polymeric carrier may be selected from e.g. any (non-peptidic) cationic or polycationic polymer suitable in this context, provided that this (non-peptidic) cationic or polycationic polymer exhibits or is modified to exhibit at least one —SH-moiety, which provides for a disulfide bond linking the cationic or polycationic polymer with another component of the polymeric carrier as defined herein. Thus, likewise as defined herein, the polymeric carrier may comprise the same or different cationic or polycationic polymers.

In the specific case that the cationic component of the polymeric carrier comprises a (non-peptidic) cationic or polycationic polymer the cationic properties of the (non-peptidic) cationic or polycationic polymer may be determined upon its content of cationic charges when compared to the overall charges of the components of the cationic polymer. Preferably, the content of cationic charges in the cationic polymer at a (physiological) pH as defined herein is at least 10%, 20%, or 30%, preferably at least 40%, more preferably at least 50%, 60% or 70%, but also preferably at least 80%, 90%, or even 95%, 96%, 97%, 98%, 99% or 100%, most preferably at least 30%, 40%, 50%, 60%, 70%, 80%, 90%, 95%, 96%, 97%, 98%, 99% or 100%, or may be in the range of about 10% to 90%, more preferably in the range of about 30% to 100%, even preferably in the range of about 50% to 100%, e.g. 50, 60, 70, 80%, 90% or 100%, or in a range formed by any two of the afore mentioned values, provided, that the content of all charges, e.g. positive and negative charges at a (physiological) pH as defined herein, in the entire cationic polymer is 100%.

Preferably, the (non-peptidic) cationic component of the polymeric carrier represents a cationic or polycationic polymer, typically exhibiting a molecular weight of about 0.1 or 0.5 kDa to about 100 kDa, preferably of about 1 kDa to about 75 kDa, more preferably of about 5 kDa to about 50 kDa, even more preferably of about 5 kDa to about 30 kDa, or a molecular weight of about 10 kDa to about 50 kDa, even more preferably of about 10 kDa to about 30 kDa. Additionally, the (non-peptidic) cationic or polycationic polymer typically exhibits at least one —SH-moiety, which is capable to form a disulfide linkage upon condensation with either other cationic components or other components of the polymeric carrier as defined herein.

In the above context, the (non-peptidic) cationic component of the polymeric carrier may be selected from acrylates, modified acrylates, such as pDMAEMA (poly(dimethylaminoethyl methylacrylate)), chitosanes, aziridines or 2-ethyl-2-oxazoline (forming oligo ethylenimines or modifed oligoethylenimines), polymers obtained by reaction of bisacrylates with amines forming oligo beta aminoesters or poly amido amines, or other polymers like polyesters, polycarbonates, etc. Each molecule of these (non-peptidic) cationic or polycationic polymers typically exhibits at least one —SH-moiety, wherein these at least one —SH-moiety may be introduced into the (non-peptidic) cationic or polycationic polymer by chemical modifications, e.g. using imonothiolan, 3-thio propionic acid or introduction of —SH-moieties containing amino acids, such as cysteine or any further (modified) amino acid. Such —SH-moieties are preferably as already defined above.

The disulfide-crosslinked cationic components may be the same or different from each other. The polymeric carrier can also contain further components. It is also particularly preferred that the polymeric carrier used according to the present invention comprises mixtures of cationic peptides, proteins or polymers and optionally further components as defined herein, which are crosslinked by disulfide bonds as described herein. In this context, the disclosure of WO 2012/013326 is incorporated herewith by reference.

In this context, the cationic components, which form basis for the polymeric carrier by disulfide-crosslinkage, are typically selected from any suitable cationic or polycationic peptide, protein or polymer suitable for this purpose, particular any cationic or polycationic peptide, protein or polymer capable to complex the isRNA for use as described herein, and thereby preferably condensing the isRNA. The cationic or polycationic peptide, protein or polymer, is preferably a linear molecule. However, branched cationic or polycationic peptides, proteins or polymers may also be used.

Every disulfide-crosslinking cationic or polycationic protein, peptide or polymer of the polymeric carrier, which may be used to complex the isRNA for use as described herein contains at least one —SH moiety, most preferably at least one cysteine residue or any further chemical group exhibiting an —SH moiety, capable to form a disulfide linkage upon condensation with at least one further cationic or polycationic protein, peptide or polymer as cationic component of the polymeric carrier as mentioned herein.

As defined above, the polymeric carrier, which may be used to complex the isRNA for use as described herein may be formed by disulfide-crosslinked cationic (or polycationic) components.

A complex of a nucleic acid, such as the isRNA for use as described herein, complexed with such polymeric carriers are also referred to herein as “polymeric carrier cargo complexes”.

In this context, it is particularly preferred that the isRNA for use in the treatment or prophylaxis of a tumor and/or cancer disease as described herein is complexed with a polymeric carrier as defined above. Preferably, the isRNA, (e.g. comprising an RNA sequence according to any of formulae I to IV), more preferably comprising an RNA sequence according to any one of SEQ ID NOs. 433 to 437, 1014 to 1016, or a fragment or variant of any one of these sequences, most preferably comprising an RNA sequence according to any one of SEQ ID NOs: 433, 434 or 1014 to 1016, or a fragment or variant of any one of these sequences, is complexed with a polymeric carrier comprising or formed by disulfide-crosslinked peptides according to formula V, Va or Vb, preferably a polymeric carrier formed by Cys(Arg₁₂)Cys (SEQ ID NO: 579) or Cys(Arg₁₂) (SEQ ID NO: 580). Such a particularly preferred embodiment is also termed herein “RNAdjuvant”.

In a further particular embodiment, the polymeric carrier which may be used to complex the isRNA for use in the treatment or prophylaxis of a tumor and/or cancer disease as described herein may be selected from a polymeric carrier molecule according to generic formula (VI):

L-P¹S—[S—P²—S]ₙ—S—P³-L formula (VI)

wherein,
P¹ and P³ are different or identical to each other and represent a linear or branched hydrophilic polymer chain, each P¹ and P³ exhibiting at least one —SH-moiety, capable to form a disulfide linkage upon condensation with component P², or alternatively with (AA), (AA)ₓ, or [(AA)ₓ]_{z} if such components are used as a linker between P¹ and P² or P³ and P² and/or with further components (e.g. (AA), (AA)ₓ, [(AA)ₓ]_{z} or L), the linear or branched hydrophilic polymer chain selected independent from each other from polyethylene glycol (PEG), poly-N-(2-hydroxypropyl)methacrylamide, poly-2-(methacryloyloxy)ethyl phosphorylcholines, poly(hydroxyalkyl L-asparagine), poly(2-(methacryloyloxy)ethyl phosphorylcholine), hydroxyethylstarch or poly(hydroxyalkyl L-glutamine), wherein the hydrophilic polymer chain exhibits a molecular weight of about 1 kDa to about 100 kDa, preferably of about 2 kDa to about 25 kDa; or more preferably of about 2 kDa to about 10 kDa, e.g. about 5 kDa to about 25 kDa or 5 kDa to about 10 kDa;
P₂ is a cationic or polycationic peptide or protein, e.g. as defined above for the polymeric carrier formed by disulfide-crosslinked cationic components, and preferably having a length of about 3 to about 100 amino acids, more preferably having a length of about 3 to about 50 amino acids, even more preferably having a length of about 3 to about 25 amino acids, e.g. a length of about 3 to 10, 5 to 15, 10 to 20 or 15 to 25 amino acids, more preferably a length of about 5 to about 20 and even more preferably a length of about 10 to about 20; or
   is a cationic or polycationic polymer, e.g. as defined above for the polymeric carrier formed by disulfide-crosslinked cationic components, typically having a molecular weight of about 0.5 kDa to about 30 kDa, including a molecular weight of about 1 kDa to about 20 kDa, even more preferably of about 1.5 kDa to about 10 kDa, or having a molecular weight of about 0.5 kDa to about 100 kDa, including a molecular weight of about 10 kDa to about 50 kDa, even more preferably of about 10 kDa to about 30 kDa;
   each P² exhibiting at least two —SH-moieties, capable to form a disulfide linkage upon condensation with further components P² or component(s) P¹ and/or P³ or alternatively with further components (e.g. (AA), (AA)ₓ, or [(AA)ₓ]_{z});
—S—S— is a (reversible) disulfide bond (the brackets are omitted for better readability), wherein S preferably represents sulphur or a —SH carrying moiety, which has formed a (reversible) disulfide bond. The (reversible) disulfide bond is preferably formed by condensation of —SH-moieties of either components P¹ and P², P² and P², or P² and P³, or optionally of further components as defined herein (e.g. L, (AA), (AA)ₓ, [(AA)ₓ]_{z}, etc); The —SH-moiety may be part of the structure of these components or added by a modification as defined below;
L is an optional ligand, which may be present or not, and may be selected independent from the other from RGD, Transferrin, Folate, a signal peptide or signal sequence, a localization signal or sequence, a nuclear localization signal or sequence (NLS), an antibody, a cell penetrating peptide, (e.g. TAT or KALA (SEQ ID NO: 1063)), a ligand of a receptor (e.g. cytokines, hormones, growth factors etc), small molecules (e.g. carbohydrates like mannose or galactose or synthetic ligands), small molecule agonists, inhibitors or antagonists of receptors (e.g. RGD peptidomimetic analogues), or any further protein as defined herein, etc.;
n is an integer, typically selected from a range of about 1 to 50, preferably from a range of about 1, 2 or 3 to 30, more preferably from a range of about 1, 2, 3, 4, or 5 to 25, or a range of about 1, 2, 3, 4, or 5 to 20, or a range of about 1, 2, 3, 4, or 5 to 15, or a range of about 1, 2, 3, 4, or 5 to 10, including e.g. a range of about 4 to 9, 4 to 10, 3 to 20, 4 to 20, 5 to 20, or 10 to 20, or a range of about 3 to 15, 4 to 15, 5 to 15, or 10 to 15, or a range of about 6 to 11 or 7 to 10. Most preferably, n is in a range of about 1, 2, 3, 4, or 5 to 10, more preferably in a range of about 1, 2, 3, or 4 to 9, in a range of about 1, 2, 3, or 4 to 8, or in a range of about 1, 2, or 3 to 7.

In this context, the disclosure of WO 2011/026641 and WO 2012/116811 is incorporated herewith by reference. Each of hydrophilic polymers P¹ and P³ typically exhibits at least one —SH-moiety, wherein the at least one —SH-moiety is capable of forming a disulfide linkage upon reaction with component P² or with component (AA) or (AA)ₓ, if used as linker between P¹ and P² or P³ and P² as defined below and optionally with a further component, e.g. L and/or (AA) or (AA)ₓ, e.g. if two or more —SH-moieties are contained. The following subformulae “P¹—S—S—P²” and “P²—S—S—P³” within the generic formula above, wherein any of S, P¹ and P³ are as defined herein, typically represent a situation, wherein one —SH-moiety of hydrophilic polymers P¹ and P³ was condensed with one —SH-moiety of component P² of the generic formula above, wherein both sulphurs of these —SH-moieties form a disulfide bond —S—S—. These —SH-moieties are typically provided by each of the hydrophilic polymers P¹ and P³, e.g. via an internal cysteine or any further (modified) amino acid or compound which carries a —SH moiety. Accordingly, the subformulae “P¹—S—S—P²” and “P²—S—S—P³” may also be written as “P¹—Cys-Cys-P²” and “P²—Cys-Cys-P³”, if the —SH-moiety is provided by a cysteine, wherein the term Cys-Cys represents two cysteines coupled via a disulfide bond, not via a peptide bond. In this case, the term “—S—S—” in these formulae may also be written as “—S-Cys”, as “—Cys-S” or as “—Cys-Cys-”. In this context, the term “—Cys-Cys-” does not represent a peptide bond but a linkage of two cysteines via their —SH-moieties to form a disulfide bond. Accordingly, the term “—Cys-Cys-” also may be understood generally as “—(Cys-S)—(S-Cys)-”, wherein in this specific case S indicates the sulphur of the —SH-moiety of cysteine. Likewise, the terms “—S-Cys” and “—Cys-S” indicate a disulfide bond between a —SH containing moiety and a cysteine, which may also be written as “—S—(S-Cys)” and “—(Cys-S)—S”. Alternatively, the hydrophilic polymers P¹ and P³ may be modified with a —SH moiety, preferably via a chemical reaction with a compound carrying a —SH moiety, such that each of the hydrophilic polymers P¹ and P³ carries at least one such —SH moiety. Such a compound carrying a —SH moiety may be e.g. an (additional) cysteine or any further (modified) amino acid, which carries a —SH moiety. Such a compound may also be any non-amino compound or moiety, which contains or allows to introduce a —SH moiety into hydrophilic polymers P¹ and P³ as defined herein. Such non-amino compounds may be attached to the hydrophilic polymers P¹ and P³ of the polymeric carrier via chemical reactions or binding of compounds, e.g. by binding of a 3-thio propionic acid or thioimolane, by amide formation (e.g. carboxylic acids, sulphonic acids, amines, etc), by Michael addition (e.g maleinimide moieties, unsatured carbonyls, etc), by click chemistry (e.g. azides or alkines), by alkene/alkine methatesis (e.g. alkenes or alkines), imine or hydrozone formation (aldehydes or ketones, hydrazins, hydroxylamins, amines), complexation reactions (avidin, biotin, protein G) or components which allow Sn-type substitution reactions (e.g halogenalkans, thiols, alcohols, amines, hydrazines, hydrazides, sulphonic acid esters, oxyphosphonium salts) or other chemical moieties which can be utilized in the attachment of further components. A particularly preferred PEG derivate in this context is alpha-methoxy-omega-mercapto poly(ethylene glycol). In each case, the SH-moiety, e.g. of a cysteine or of any further (modified) amino acid or compound, may be present at the terminal ends or internally at any position of hydrophilic polymers P¹ and P³. As defined herein, each of hydrophilic polymers P¹ and P³ typically exhibits at least one —SH-moiety preferably at one terminal end, but may also contain two or even more —SH-moieties, which may be used to additionally attach further components as defined herein, preferably further functional peptides or proteins e.g. a ligand, an amino acid component (AA) or (AA)ₓ, antibodies, cell penetrating peptides or enhancer peptides (e.g. TAT, KALA (SEQ ID NO: 1063)), etc.

As defined above, ligands (L), may be optionally used in the polymeric carrier molecule according to generic formula (VI), e.g. for direction of the inventive carrier polymer and its entire “cargo” (e.g. the isRNA for use in the treatment or prophylaxis of a tumor and/or cancer disease as described herein) into specific cells. They may be selected independent from the other from RGD, Transferrin, Folate, a signal peptide or signal sequence, a localization signal or sequence, a nuclear localization signal or sequence (NLS), an antibody, a cell penetrating peptide (CPP), (e.g. TAT, KALA (SEQ ID NO: 1063)), a ligand of a receptor (e.g. cytokines, hormones, growth factors etc), small molecules (e.g. carbohydrates like mannose or galactose or synthetic ligands), small molecule agonists, inhibitors or antagonists of receptors (e.g. RGD peptidomimetic analogues) or any such molecule as further defined below, etc. Particularly preferred are cell penetrating peptides (CPPs), which induce a pH-mediated conformational change in the endosome and lead to an improved release of the inventive polymeric carrier (in complex with a nucleic acid) from the endosome by insertion into the lipid layer of the liposome. Such called CPPs or cationic peptides for transportation, may include, without being limited thereto protamine, nucleoline, spermine or spermidine, poly-L-lysine (PLL), basic polypeptides, poly-arginine, chimeric CPPs, such as Transportan, or MPG peptides, HIV-binding peptides, Tat, HIV-1 Tat (HIV), Tat-derived peptides, oligoarginines, members of the penetratin family, e.g. Penetratin, Antennapedia-derived peptides (particularly from Drosophila antennapedia), pAntp, pIsl, etc., antimicrobial-derived CPPs e.g. Buforin-2, Bac715-24, SynB, SynB(1), pVEC, hCT-derived peptides, SAP, MAP, PpTG20, proline-rich peptides, Loligomers, arginine-rich peptides, Calcitonin-peptides, FGF, Lactoferrin, poly-L-lysine, poly-arginine, histones, VP22 derived or analog peptides, Pestivirus Erns, HSV, VP22 (Herpes simplex), MAP, KALA (SEQ ID NO: 1063) or protein transduction domains (PTDs, PpT620, proline-rich peptides, arginine-rich peptides, lysine-rich peptides, Pep-1, L-oligomers, Calcitonin peptide(s), etc. Particularly preferred in this context is mannose as ligand to target antigen presenting cells, which typically carry mannose receptors on their cell membrane. In a further preferred embodiment of the present invention, galactose as optional ligand can be used to target hepatocytes. Such ligands may be attached to component P¹ and/or P³ by reversible disulfide bonds as defined below or by any other possible chemical attachement, e.g. by amide formation (e.g. carboxylic acids, sulphonic acids, amines, etc), by Michael addition (e.g. maleinimide moieties, α, β unsatured carbonyls, etc), by click chemistry (e.g. azides or alkines), by alkene/alkine methatesis (e.g. alkenes or alkines), imine or hydrozone formation (aldehydes or ketons, hydrazins, hydroxylamins, amines), complexation reactions (avidin, biotin, protein G) or components which allow Sn-type substitution reactions (e.g halogenalkans, thiols, alcohols, amines, hydrazines, hydrazides, sulphonic acid esters, oxyphosphonium salts) or other chemical moieties which can be utilized in the attachment of further components.

In the context of formula (VI) of the present invention components P¹ and P³ represent a linear or branched hydrophilic polymer chain, containing at least one —SH-moiety, each P¹ and P³ independently selected from each other, e.g. from polyethylene glycol (PEG), poly-N-(2-hydroxypropyl)methacrylamide, poly-2-(methacryloyloxy)ethyl phosphorylcholines, poly(hydroxyalkyl L-asparagine) or poly(hydroxyalkyl L-glutamine). P¹ and P³ may be identical or different to each other. Preferably, each of hydrophilic polymers P¹ and P³ exhibits a molecular weight of about 1 kDa to about 100 kDa, preferably of about 1 kDa to about 75 kDa, more preferably of about 5 kDa to about 50 kDa, even more preferably of about 5 kDa to about 25 kDa. Additionally, each of hydrophilic polymers P¹ and P³ typically exhibits at least one —SH-moiety, wherein the at least one —SH-moiety is capable to form a disulfide linkage upon reaction with component P² or with component (AA) or (AA)ₓ, if used as linker between P¹ and P² or P³ and P² as defined below and optionally with a further component, e.g. L and/or (AA) or (AA)ₓ, e.g. if two or more —SH-moieties are contained. The following subformulae “P¹—S—S—P²” and “P²—S—S—P³” within generic formula (VI) above (the brackets are omitted for better readability), wherein any of S, P¹ and P³ are as defined herein, typically represent a situation, wherein one —SH— moiety of hydrophilic polymers P¹ and P³ was condensed with one —SH-moiety of component P² of generic formula (VI) above, wherein both sulphurs of these —SH-moieties form a disulfide bond —S—S— as defined herein in formula (VI). These —SH-moieties are typically provided by each of the hydrophilic polymers P¹ and P³, e.g. via an internal cysteine or any further (modified) amino acid or compound which carries a —SH moiety. Accordingly, the subformulae “P¹—S—S—P²” and “P²—S—S—P³” may also be written as “P¹—Cys-Cys-P²” and “P²—Cys-Cys-P³”, if the —SH— moiety is provided by a cysteine, wherein the term Cys-Cys represents two cysteines coupled via a disulfide bond, not via a peptide bond. In this case, the term “—S—S—” in these formulae may also be written as “—S-Cys”, as “—Cys-S” or as “—Cys-Cys-”. In this context, the term “—Cys-Cys-” does not represent a peptide bond but a linkage of two cysteines via their —SH-moieties to form a disulfide bond. Accordingly, the term “—Cys-Cys-” also may be understood generally as “—(Cys-S)—(S-Cys)-”, wherein in this specific case S indicates the sulphur of the —SH-moiety of cysteine. Likewise, the terms “—S-Cys” and “—Cys-S” indicate a disulfide bond between a —SH containing moiety and a cysteine, which may also be written as “—S—(S-Cys)” and “—(Cys-S)—S”. Alternatively, the hydrophilic polymers P¹ and P³ may be modified with a —SH moiety, preferably via a chemical reaction with a compound carrying a —SH moiety, such that each of the hydrophilic polymers P¹ and P³ carries at least one such —SH moiety. Such a compound carrying a —SH moiety may be e.g. an (additional) cysteine or any further (modified) amino acid, which carries a —SH moiety. Such a compound may also be any non-amino compound or moiety, which contains or allows to introduce a —SH moiety into hydrophilic polymers P¹ and P³ as defined herein. Such non-amino compounds may be attached to the hydrophilic polymers P¹ and P³ of formula (VI) of the polymeric carrier according to the present invention via chemical reactions or binding of compounds, e.g. by binding of a 3-thio propionic acid or thioimolane, by amide formation (e.g. carboxylic acids, sulphonic acids, amines, etc), by Michael addition (e.g maleinimide moieties, α, β unsatured carbonyls, etc), by click chemistry (e.g. azides or alkines), by alkene/alkine methatesis (e.g. alkenes or alkines), imine or hydrozone formation (aldehydes or ketones, hydrazins, hydroxylamins, amines), complexation reactions (avidin, biotin, protein G) or components which allow Sn-type substitution reactions (e.g halogenalkans, thiols, alcohols, amines, hydrazines, hydrazides, sulphonic acid esters, oxyphosphonium salts) or other chemical moieties which can be utilized in the attachment of further components. A particularly preferred PEG derivate in this context is alpha-methoxy-omega-mercapto poly(ethylene glycol). In each case, the SH-moiety, e.g. of a cysteine or of any further (modified) amino acid or compound, may be present at the terminal ends or internally at any position of hydrophilic polymers P¹ and P³. As defined herein, each of hydrophilic polymers P¹ and P³ typically exhibits at least one —SH-moiety preferably at one terminal end, but may also contain two or even more —SH-moieties, which may be used to additionally attach further components as defined herein, preferably further functional peptides or proteins e.g. a ligand, an amino acid component (AA) or (AA)ₓ, antibodies, cell penetrating peptides or enhancer peptides (e.g. TAT, KALA (SEQ ID NO: 1063)), etc.

According to one preferred alternative, such further functional peptides or proteins may comprise so-called cell penetrating peptides (CPPs) or cationic peptides for transportation. Particularly preferred are CPPs, which induce a pH-mediated conformational change in the endosome and lead to an improved release of the inventive polymeric carrier (in complex with a nucleic acid) from the endosome by insertion into the lipid layer of the liposome. Such cell penetrating peptides (CPPs) or cationic peptides for transportation, may include, without being limited thereto protamine, nucleoline, spermine or spermidine, poly-L-lysine (PLL), basic polypeptides, poly-arginine, chimeric CPPs, such as Transportan, or MPG peptides, HIV-binding peptides, Tat, HIV-1 Tat (HIV), Tat-derived peptides, oligoarginines, members of the penetratin family, e.g. Penetratin, Antennapedia-derived peptides (particularly from Drosophila antennapedia), pAntp, pIsl, etc., antimicrobial-derived CPPs e.g. Buforin-2, Bac715-24, SynB, SynB(1), pVEC, hCT-derived peptides, SAP, MAP, PpTG20, proline-rich peptides, Loligomers, arginine-rich peptides, Calcitonin-peptides, FGF, Lactoferrin, poly-L-lysine, poly-arginine, histones, VP22 derived or analog peptides, Pestivirus Ems, HSV, VP22 (Herpes simplex), MAP, KALA (SEQ ID NO: 1063) or protein transduction domains (PTDs, PpT620, prolin-rich peptides, arginine-rich peptides, lysine-rich peptides, Pep-1, L-oligomers, Calcitonin peptide(s), etc.

According to a further preferred embodiment of the present invention, each of hydrophilic polymers P¹ and P³ of formula (VI) of the polymeric carrier used according to the present invention may also contain at least one further functional moiety, which allows attaching further components as defined herein, e.g. a ligand as defined above, or functionalities which allow the attachment of further components, e.g. by amide formation (e.g. carboxylic acids, sulphonic acids, amines, etc), by Michael addition (e.g maleinimide moieties, unsatured carbonyls, etc), by click chemistry (e.g. azides or alkines), by alkene/alkine methatesis (e.g. alkenes or alkines), imine or hydrozone formation (aldehydes or ketons, hydrazins, hydroxylamins, amines), complexation reactions (avidin, biotin, protein G) or components which allow Sn-type substitution reactions (e.g halogenalkans, thiols, alcohols, amines, hydrazines, hydrazides, sulphonic acid esters, oxyphosphonium salts) or other chemical moieties which can be utilized in the attachment of further components. Further functional moieties may comprise an amino acid component (AA) as defined herein or (AA)ₓ, wherein (AA) is preferably an amino component as defined above. In the above context, x is preferably an integer and may be selected from a range of about 1 to 100, preferably from a range of about 1 to 50, more preferably 1 to 30, and even more preferably selected from a number comprising 1, 2, 3, 4, 5, 6, 7, 8, 9, 10, 11, 12, 13, 14 or 15-30, e.g. from a range of about 1 to 30, from a range of about 1 to 15, or from a number comprising 1, 2, 3, 4, 5, 6, 7, 8, 9, 10, 11, 12, 13, 14 or 15, or may be selected from a range formed by any two of the afore mentioned values. Most preferably, x is 1. Such an amino acid component (AA) or (AA)ₓ may be contained in every part of the inventive polymeric carrier according to formula (VI) above and therefore may be attached to all components of the polymeric carrier according to formula (VI). It is particularly preferred that amino acid component (AA) or (AA)ₓ is present as a ligand or part of the repetitive component [S—P²—S]n within formula (VI) of the polymeric carrier.

In the context of the entire formula (VI) of the polymeric carrier may be preferably defined as follows:

L-P¹—S—[Cys-P²—Cys]ₙ-S—P³-L

wherein L, P¹, P², P³ and n are as defined herein, S is sulphur and each Cys provides for one —SH-moiety for the disulfide bond.

According to a particular embodiment, the polymeric carrier according to formula (VI) as defined above, may comprise at least one amino acid component (AA) or (AA)ₓ, as defined above. Such an amino acid component (AA) or (AA)), may be contained in every part of the inventive polymeric carrier according to formula (VI) above and therefore may be attached to all components of the polymeric carrier according to formula (VI). It is particularly preferred that amino acid component (AA) or (AA)ₓ is present as a ligand or part of the repetitive component [S—P2-S]n within formula (VI) of the polymeric carrier. The amino acid component (AA) or (AA)ₓ preferably contains or is flanked (e.g. terminally) by at least one —SH containing moiety, which allows introducing this component (AA) or (AA)ₓ via a disulfide bond into the polymeric carrier according to formula (VI) as defined herein. Such a —SH-containing moiety may be any —SH containing moiety (or, of course, one sulphur of a disulfide bond), e.g. a cysteine residue. In the specific case that the —SH containing moiety represents a cysteine, the amino acid component (AA)ₓ may also be read as —Cys-(AA)ₓ- or -Cys-(AA)ₓ-Cys- wherein Cys represents cysteine and provides for the necessary —SH-moiety for a disulfide bond. The —SH containing moiety may be also introduced into the amino acid component (AA)ₓ using any of modifications or reactions as shown above for components P¹, P² or P³. In the specific case that the amino acid component (AA)ₓ is linked to two components of the polymeric carrier according to formula (VI) it is preferred that (AA) or (AA)ₓ contains at least two —SH-moieties, e.g. at least two cysteines, preferably at its terminal ends. This is particularly preferred if (AA) or (AA)ₓ is part of the repetitive component [S—P2-S]ₙ. Alternatively, the amino acid component (AA) or (AA)ₓ is introduced into the polymeric carrier according to formula (VI) as defined herein via any chemical possible addition reaction. Therefore the amino acid component (AA) or (AA)ₓ contains at least one further functional moiety, which allows attaching same to a further component as defined herein, e.g. component P¹ or P³, P², L, or a further amino acid component (AA) or (AA)ₓ, etc. Such functional moieties may be selected from functionalities which allow the attachment of further components, e.g. functionalities as defined herein, e.g. by amide formation (e.g. carboxylic acids, sulphonic acids, amines, etc), by Michael addition (e.g maleinimide moieties, α, β unsatured carbonyls, etc), by click chemistry (e.g. azides or alkines), by alkene/alkine methatesis (e.g. alkenes or alkines), imine or hydrozone formation (aldehydes or ketons, hydrazins, hydroxylamins, amines), complexation reactions (avidin, biotin, protein G) or components which allow Sn-type substitution reactions (e.g halogenalkans, thiols, alcohols, amines, hydrazines, hydrazides, sulphonic acid esters, oxyphosphonium salts) or other chemical moieties which can be utilized in the attachment of further components.

The amino acid component (AA) or (AA)ₓ in the polymeric carrier of formula (VI) may also occur as a mixed repetitive amino acid component [(AA)ₓ]_{z}, wherein the number of amino acid components (AA) or (AA)ₓ is further defined by integer z. In this context, z may be selected from a range of about 1 to 30, preferably from a range of about 1 to 15, more preferably 1 to 10 or 1 to 5 and even more preferably selected from a number selected from 1, 2, 3, 4, 5, 6, 7, 8, 9, 10, 11, 12, 13, 14 or 15, or may be selected from a range formed by any two of the afore mentioned values.

According to a specific and particularly preferred alternative, the amino acid component (AA) or (AA)ₓ, preferably written as S-(AA)ₓ-S or [S-(AA)ₓ-S] may be used to modify component P², particularly the content of component S—P²—S in repetitive component [S—P²—S]n of the polymeric carrier of formula (VI) above. This may be represented in the context of the entire polymeric carrier according to formula (VI) e.g. by following formula (VIa):

L-P¹—S—{[S—P²—S]ₐ[S-(AA)ₓ-S]_{b}}—S—P³-L,

wherein x, S, L, AA, P¹, P² and P³ are preferably as defined herein. In formula (VIa) above, any of the single components [S—P²—S] and [S-(AA)ₓ-S] may occur in any order in the subformula {[S—P²—S]ₐ[S-(AA)ₓ-S]_{b}}. The numbers of single components [S—P²—S] and [S-(AA)ₓ-S] in the subformula {[S—P²—S]ₐ[S-(AA)ₓ-S]_{b}} are determined by integers a and b, wherein a+b=n. n is an integer and is defined as above for formula (VI).

a is an integer, typically selected independent from integer b from a range of about 1 to 50, preferably from a range of about 1, 2 or 3 to 30, more preferably from a range of about 1, 2, 3, 4, or 5 to 25, or a range of about 1, 2, 3, 4, or 5 to 20, or a range of about 1, 2, 3, 4, or 5 to 15, or a range of about 1, 2, 3, 4, or 5 to 10, including e.g. a range of about 3 to 20, 4 to 20, 5 to 20, or 10 to 20, or a range of about 3 to 15, 4 to 15, 5 to 15, or 10 to 15, or a range of about 6 to 11 or 7 to 10. Most preferably, a is in a range of about 1, 2, 3, 4, or 5 to 10, more preferably in a range of about 1, 2, 3, or 4 to 9, in a range of about 1, 2, 3, or 4 to 8, or in a range of about 1, 2, or 3 to 7.

b is an integer, typically selected independent from integer a from a range of about 0 to 50 or 1 to 50, preferably from a range of about 0, 1, 2 or 3 to 30, more preferably from a range of about 0, 1, 2, 3, 4, or 5 to 25, or a range of about 0, 1, 2, 3, 4, or 5 to 20, or a range of about 0, 1, 2, 3, 4, or 5 to 15, or a range of about 0, 1, 2, 3, 4, or 5 to 10, including e.g. a range of about 3 to 20, 4 to 20, 5 to 20, or 10 to 20, or a range of about 3 to 15, 4 to 15, 5 to 15, or 10 to 15, or a range of about 6 to 11 or 7 to 10. Most preferably, b is in a range of about 1, 2, 3, 4, or 5 to 10, more preferably in a range of about 1, 2, 3, or 4 to 9, in a range of about 1, 2, 3, or 4 to 8, or in a range of about 1, 2, or 3 to 7.

In this context, it is particularly preferred that the isRNA for use in the treatment or prophylaxis of a tumor and/or cancer disease as described herein is complexed at least partially with a cationic or polycationic compound and/or a polymeric carrier, preferably cationic proteins or peptides. In this context, the disclosure of WO 2010/037539 and WO 2012/113513 is incorporated herewith by reference. “Partially” in that context means that only a part of the RNA is complexed with a cationic compound and that the rest of the RNA is (preferably comprised in the same formulation) in uncomplexed form (“free”). Preferably the ratio of complexed RNA to free RNA (in the inventive composition) is selected from a range of about 5:1 (w/w) to about 1:10 (w/w), more preferably from a range of about 4:1 (w/w) to about 1:8 (w/w), even more preferably from a range of about 3:1 (w/w) to about 1:5 (w/w) or 1:3 (w/w), and most preferably the ratio of complexed RNA to free RNA in the inventive composition is selected from a ratio of about 1:1 (w/w).

According to a preferred embodiment, the isRNA for use in the treatment or prophylaxis of a tumor and/or cancer disease as described herein may be complexed with lipids to form one or more liposomes, lipid nanoparticles and/or lipoplexes.

Lipid-based formulations have been increasingly recognized as one of the most promising delivery systems for RNA due to their biocompatibility and their ease of large-scale production. Cationic lipids have been widely studied as synthetic materials for delivery of RNA. After mixing together, nucleic acids are condensed by cationic lipids to form lipid/nucleic acid complexes known as lipoplexes. These lipid complexes are able to protect genetic material from the action of nucleases and to deliver it into cells by interacting with the negatively charged cell membrane. Lipoplexes can be prepared by directly mixing positively charged lipids at physiological pH with negatively charged nucleic acids.

Conventional liposomes consist of a lipid bilayer that can be composed of cationic, anionic, or neutral (phospho)lipids and cholesterol, which encloses an aqueous core. Both the lipid bilayer and the aqueous space can incorporate hydrophobic or hydrophilic compounds, respectively. Liposome characteristics and behaviour in vivo can be modified by addition of a hydrophilic polymer coating, e.g. polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains (Front Pharmacol. 2015 Dec. 1; 6:286).

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm. Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9: 1833-1843).

Cationic liposomes have been traditionally the most commonly used non-viral delivery systems for oligonucleotides, including plasmid DNA, antisense oligos, and siRNA/small hairpin RNA-shRNA). Cationic lipids, such as DOTAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids to form nanoparticles by electrostatic interaction, providing high in vitro transfection efficiency. Furthermore, neutral lipid-based nanoliposomes for RNA delivery as e.g. neutral 1,2-dioleoyl-sn-glycero-3-phosphatidylcholine (DOPC)-based nanoliposomes were developed. (Adv Drug Deliv Rev. 2014 February; 66: 110-116.).

Therefore, in one embodiment, the isRNA for use in the treatment or prophylaxis of a tumor and/or cancer disease as described herein is complexed with cationic lipids and/or neutral lipids and thereby forms liposomes, lipid nanoparticles, lipoplexes or neutral lipid-based nanoliposomes.

According to some embodiments, the isRNA for use as described herein is formulated as a lipid formulation. The lipid formulation is preferably selected from, but not limited to, liposomes, lipoplexes, copolymers, such as PLGA, and lipid nanoparticles.

In one preferred embodiment, a lipid nanoparticle (LNP) comprises:
(a) a nucleic acid,
(b) a cationic lipid,
(c) an aggregation reducing agent (such as polyethylene glycol (PEG) lipid or PEG-modified lipid),
(d) optionally a non-cationic lipid (such as a neutral lipid), and
(e) optionally, a sterol.

In one embodiment, the lipid nanoparticle formulation consists of (i) at least one cationic lipid; (ii) a neutral lipid; (iii) a sterol, e.g., cholesterol; and (iv) a PEG-lipid, in a molar ratio of about 20-60% cationic lipid: 5-25% neutral lipid: 25-55% sterol; 0.5-15% PEG-lipid.

Cationic Lipids

The lipid nanoparticle preferably includes a cationic lipid suitable for forming a lipid nanoparticle. Preferably, the cationic lipid carries a net positive charge at about physiological pH.

The cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)—N,N-dimethyl-2,2-di((9412Z)-octadeca-9,12-dienyl)tetrahydro-3aH-cyclopenta[d][1,3]dioxol-5-amine, (649428431Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (649428431Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,3 1-tetraen-19-yloxy)-N,N-dimethylpropan-1-amine (MC3 Ether), 4-((649428431 Z)-heptatriaconta-6,9,28,31-tetraen-19-yloxy)-N,N-dimethylbutan-1-amine (MC4 Ether), or any combination of any of the foregoing. Other cationic lipids include, but are not limited to, N,N-distearyl-N,N-dimethylammonium bromide (DDAB), 3P—(N—(N′,N′-dimethylaminoethane)-carbamoyl)cholesterol (DC-Chol), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al, PNAS, 107(5), 1864-69, 2010. Other suitable amino lipids include those having alternative fatty acid groups and other dialkylamino groups, including those, in which the alkyl substituents are different (e.g., N-ethyl-N-methylamino-, and N-propyl-N-ethylamino-). In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid.

In a further preferred embodiment, the LNP comprises the cationic lipid with formula (III) according to the patent application PCT/EP2017/064066. In this context, the disclosure of PCT/EP2017/064066 is also incorporated herein by reference.

In certain embodiments, amino or cationic lipids of the invention have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the invention. In certain embodiments, the protonatable lipids have a pKa of the protonatable group in the range of about 4 to about 11, e.g., a pKa of about 5 to about 7.

The cationic lipid can comprise from about 20 mol % to about 70 or 75 mol % or from about 45 to about 65 mol % or about 20, 25, 30, 35, 40, 45, 50, 55, 60, 65, or about 70 mol % of the total lipid present in the particle. In another embodiment, the lipid nanoparticles include from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In one embodiment, the ratio of cationic lipid to nucleic acid is from about 3 to about 15, such as from about 5 to about 13 or from about 7 to about 11.

Non-Cationic Lipids

The non-cationic lipid can be a neutral lipid, an anionic lipid, or an amphipathic lipid. Neutral lipids, when present, can be any of a number of lipid species which exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., lipid particle size and stability of the lipid particle in the bloodstream. Preferably, the neutral lipid is a lipid having two acyl groups (e.g. diacylphosphatidylcholine and diacylphosphatidylethanolamine). In one embodiment, the neutral lipids contain saturated fatty acids with carbon chain lengths in the range of C10 to C20. In another embodiment, neutral lipids with mono or diunsaturated fatty acids with carbon chain lengths in the range of C10 to C20 are used. Additionally, neutral lipids having mixtures of saturated and unsaturated fatty acid chains can be used.

Suitable neutral lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), dimyristoyl phosphatidylcholine (DMPC), distearoyl-phosphatidyl-ethanolamine (DSPE), SM, 16-0-monomethyl PE, 16-O-dimethyl PE, 18-1-trans PE, 1-stearoyl-2-oleoyl-phosphatidyethanolamine (SOPE), cholesterol, or a mixture thereof. Anionic lipids suitable for use in lipid particles of the invention include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

The non-cationic lipid can be from about 5 mol % to about 90 mol %, about 5 mol % to about 10 mol %, about 5, 10, 15, 20, 25, 30, 35, 40, 45, 50, 55, 60, 65, 70, 75, 80, 85, or about 90 mol % of the total lipid present in the particle. In one embodiment, the lipid nanoparticles include from about 0% to about 15 or 45% on a molar basis of neutral lipid, e.g., from about 3 to about 12% or from about 5 to about 10%. For instance, the lipid nanoparticles may include about 15%, about 10%, about 7.5%, or about 7.1% of neutral lipid on a molar basis (based upon 100% total moles of lipid in the lipid nanoparticle).

Sterols

A preferred sterol is cholesterol. The sterol can be about 10 mol % to about 60 mol % or about 25 mol % to about 40 mol % of the lipid particle. In one embodiment, the sterol is about 10, 15, 20, 25, 30, 35, 40, 45, 50, 55, or about 60 mol % of the total lipid present in the lipid particle. In another embodiment, the lipid nanoparticles include from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the lipid nanoparticle).

Aggregation Reducing Agent

The aggregation reducing agent can be a lipid capable of reducing aggregation. Examples of such lipids include, but are not limited to, polyethylene glycol (PEG)-modified lipids, monosialoganglioside Gml, and polyamide oligomers (PAO) such as those described in U.S. Pat. No. 6,320,017, which is incorporated by reference in its entirety. Other compounds with uncharged, hydrophilic, steric-barrier moieties, which prevent aggregation during formulation, like PEG, Gml or ATTA, can also be coupled to lipids. ATTA-lipids are described, e.g., in U.S. Pat. No. 6,320,017, and PEG-lipid conjugates are described, e.g., in U.S. Pat. Nos. 5,820,873, 5,534,499 and 5,885,613, each of which is incorporated by reference in its entirety.

The aggregation reducing agent may be, for example, a polyethyleneglycol (PEG)-lipid including, without limitation, a PEG-diacylglycerol (DAG), a PEG-dialkylglycerol, a PEG-dialkyloxypropyl (DAA), a PEG-phospholipid, a PEG-ceramide (Cer), or a mixture thereof (such as PEG-Cerl4 or PEG-Cer20). The PEG-DAA conjugate may be, for example, a PEG-dilauryloxypropyl (C12), a PEG-dimyristyloxypropyl (C14), a PEG-dipalmityloxypropyl (C16), or a PEG-distearyloxypropyl (C18). Other pegylated-lipids include, but are not limited to, polyethylene glycol-didimyristoyl glycerol (C14-PEG or PEG-C14, where PEG has an average molecular weight of 2000 Da) (PEG-DMG); (R)-2,3-bis(octadecyloxy)propyl-1-(methoxy poly(ethylene glycol)2000)propylcarbamate) (PEG-DSG); PEG-carbamoyl-1,2-di myristyloxypropyla mine, in which PEG has an average molecular weight of 2000 Da (PEG-cDMA); N-Acetylgalactosamine-aR)-2,3-bis(octadecyloxy)propyl-1-(methoxy poly(ethylene glycol)2000)propylcarba mate)) (GaINAc-PEG-DSG); mPEG (mw2000)-diastearoylphosphatidyl-ethanolamine (PEG-DSPE); and polyethylene glycol-dipalmitoylglycerol (PEG-DPG). In one embodiment, the aggregation reducing agent is PEG-DMG. In another embodiment, the aggregation reducing agent is PEG-c-DMA.

The average molecular weight of the PEG moiety in the PEG-modified lipids can range from about 500 to about 8,000 Daltons (e.g., from about 1,000 to about 4,000 Daltons). In one preferred embodiment, the average molecular weight of the PEG moiety is about 2,000 Daltons.

The concentration of the aggregation reducing agent may range from about 0.1 to about 15 mol %, based upon the 100% total moles of lipid in the lipid particle. In one embodiment, the formulation includes less than about 3, 2, or 1 mole percent of PEG or PEG-modified lipid, based upon the total moles of lipid in the lipid particle. In another embodiment, the lipid nanoparticles include from about 0.1% to about 20% on a molar basis of the PEG-modified lipid, e.g., about 0.5 to about 10%, about 0.5 to about 5%, about 10%, about 5%, about 3.5%, about 1.5%, about 0.5%, or about 0.3% on a molar basis (based on 100% total moles of lipids in the lipid nanoparticle).

According to a preferred embodiment, the isRNA for use in the treatment or prophylaxis of a tumor and/or cancer disease as described herein is formulated by using the isRNA as described herein and one or more liposomes, lipoplexes, or lipid nanoparticles. In one embodiment, the inventive composition comprises liposomes. Liposomes typically are artificially-prepared vesicles, which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the isRNA for use in the treatment or prophylaxis of a tumor and/or cancer disease as described herein, in particular when applied as a pharmaceutical composition as described herein.

Lipid Nanoparticles (LNPs)

Preferably, lipid nanoparticles may have the structure of a liposome. A liposome is typically a structure having lipid-containing membranes enclosing an aqueous interior. Liposomes preferably have one or more lipid membranes. In preferred embodiments, liposomes can be single-layered, referred to as unilamellar, or multi-layered, referred to as multilamellar. When complexed with nucleic acids (e.g. RNA), lipid particles may also be lipoplexes, which are preferably composed of cationic lipid bilayers sandwiched between nucleic acid layers. Liposomes can further be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. In certain embodiments, liposome design may include, but is not limited to, opsonins or ligands in order to improve the attachment of liposomes to unhealthy tissue or to activate events such as, but not limited to, endocytosis. Liposomes may contain a low (e.g. an acidic) or a high (e.g. a basic) pH in order to improve the delivery of the pharmaceutical formulations.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are herein incorporated by reference in their entirety. In preferred embodiments, the nucleic acid (e.g. an RNA as described herein) may be encapsulated by the liposome, and/or it may be contained in an aqueous core, which may then be encapsulated by the liposome (see International Pub. Nos. WO2012031046, WO2012031043, WO2012030901 and WO2012006378 and US Patent Publication No. US20130189351, US20130195969 and US20130202684; the contents of each of which are herein incorporated by reference in their entirety).

In another embodiment, the lipid nanoparticles have a median diameter size of from about 50 nm to about 300 nm, such as from about 50 nm to about 250 nm, for example, from about 50 nm to about 200 nm. In another embodiment, nucleic acids may be delivered using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm, from about 70 to about 80 nm, and/or from about 70 to about 90 nm.

In one embodiment, the weight ratio of lipid to RNA is at least about 0.5:1, at least about 1:1, at least about 2:1, at least about 3:1, at least about 4:1, at least about 5:1, at least about 6:1, at least about 7:1, at least about 11:1, at least about 20:1, at least about 25:1, at least about 27:1, at least about 30:1, or at least about 33:1. In one embodiment, the weight ratio of lipid to RNA is from about 1:1 to about 35:1, about 3:1 to about 15:1, about 4:1 to about 15:1, or about 5:1 to about 13:1 or about 25:1 to about 33:1. In one embodiment, the weight ratio of lipid to RNA is from about 0.5:1 to about 12:1.

According to a preferred embodiment, the isRNA for use in the treatment or prophylaxis of a tumor and/or cancer disease as described herein is administered/applied intratumorally (i.t.), locoregionally or peritumorally. As used herein, the term, intratumoral administration/application” refers to the direct delivery of a pharmaceutically active ingredient, such as the isRNA as described herein, e.g. in the form of a composition/formulation comprising the isRNA as described herein, into a tumor, adjacent to a tumor, and/or to the immediate vicinity of a tumor (peritumorally). Said delivery may be achieved by several methods known in the art, comprising but not limited to injection (such as conventional needle injection or needle-free injection, e.g. jet injection) or electroporation or combinations thereof. Methods for intratumoral delivery of drugs are described in the art (see, for instance, Brincker, 1993. Crit. Rev. Oncol. Hematol. 15(2):91-8; Celikoglu et al., 2008. Cancer Therapy 6, 545-552).

As used herein, the term ‘intratumoral’ may also refer to the administration of an active pharmaceutical ingredient to an organ bearing a tumor or cancer, or to a tissue bearing a tumor or cancer. Accordingly, the term ‘intratumoral’ as used herein may also comprise peritumoral or locoregional administration, wherein an active pharmaceutical ingredient is preferably administered to an organ or tissue proximal to the tumor or cancer, preferably to an organ or tissue, which is in direct physical contact with the tumor or cancer. In the context of the present invention, intratumoral, locoregional or peritumoral administration preferably comprises delivery (i.e. by injection) of an active pharmaceutical ingredient to a superficial tumor or cancer, or to a tumor or cancer, which is located inside of a tissue.

In some embodiments, locoregional administration of an active pharmaceutical ingredient (such as the RNA described herein) comprises delivering the active pharmaceutical ingredient to a tumor or cancer or to a tissue or organ bearing a tumor or cancer by administering the active pharmaceutical ingredient to a blood vessel (e.g. an artery, such as the liver artery, or a vein, such as the pulmonary vein) that carries the blood to the tumor or cancer or to the tissue or organ bearing the tumor or cancer.

According to a preferred embodiment, a pharmaceutically active ingredient, such as the isRNA for use in the treatment or prophylaxis of a tumor and/or cancer disease as described herein is administered intratumorally (i.t.), including locoregionally or peritumorally, wherein the administration comprises an injection technique. Therein, a pharmaceutically active ingredient, such as the isRNA for use as described herein, may preferably be injected in a single dose per treatment. Alternatively, multiple injections into the same or separate regions of the tumor or cancer or the tumor bearing organ or tissue are also envisaged. Furthermore, intratumoral administration/application includes delivery of a pharmaceutically active ingredient into one or more metastases, preferably via injection. Administration of the pharmaceutically active ingredient may be performed as single dose or repeat dose treatment with various treatment intervals, preferably as described herein.

In a preferred embodiment, a pharmaceutically active ingredient, such as the isRNA for use in the treatment or prophylaxis of a tumor and/or cancer disease as described herein is administered intratumorally, including locoregionally or peritumorally, by injection. Preferably, the intratumoral administration involves an imaging technique, which preferably enhances the precision of the administration. More preferably, such imaging technique is selected from the group consisting of computer tomograpy, ultrasound, gamma camera imaging, positron emission tomography and magnetic resonance tumor imaging. Furthermore, the intratumoral administration may preferably comprise direct intratumoral injection, which preferably involves at least one of the procedures selected from the group consisting of endoscopy, bronchoscopy, cystoscopy, colonoscopy, laparoscope and catheterization.

In a preferred embodiment, a pharmaceutically active ingredient, such as the isRNA for use in the treatment or prophylaxis of a tumor and/or cancer disease as described herein is administered locoregionally by injection. Preferably, the locoregional administration involves an imaging technique, which preferably enhances the precision of the administration, which is preferably an intratumoral or peritumoral administration as described herein. More preferably, such imaging technique is selected from the group consisting of computer tomograpy, ultrasound, gamma camera imaging, positron emission tomography and magnetic resonance tumor imaging. Furthermore, the locoregional administration may preferably comprise direct locoregional injection, which preferably involves at least one of the procedures selected from the group consisting of endoscopy, bronchoscopy, cystoscopy, colonoscopy, laparoscope and catheterization. As used herein, the term locoregional administration′ may thus also refer to intratumoral or peritumoral administration, preferably injection, of a pharmaceutically active ingredient (e.g. an RNA as described herein), wherein the administration preferably involves an imaging technique, wherein the imaging technique preferably comprises at least one of the procedures selected from the group consisting of endoscopy, bronchoscopy, cystoscopy, colonoscopy, laparoscope and catheterization.

According to a preferred embodiment, the invention provides an isRNA for use in the treatment of a tumor or cancer disease,

wherein the isRNA comprises a nucleic acid sequence according to formula (I) (GₗXₘGₙ), formula (II) (CₗXₘCₙ), formula (III) (NᵤGₗXₘGₙNᵥ)ₐ or formula (IV) (NᵤCₗXₘCₙNᵥ)ₐ, preferably at least one nucleic acid sequence according to any one of SEQ ID NOs: 433 to 437 or 1014 to 1016, or a fragment or variant of any one of these sequences, more preferably according to any one of SEQ ID NOs: 433, 434, or 1014 to 1016, or a fragment or a variant of any one of these nucleic acid sequences,

wherein the isRNA is complexed with a cationic or polycationic compound, preferably with a polymeric carrier, more preferably with a polymeric carrier that is formed by a disulfide-crosslinked cationic component, which preferably comprises a peptide according to formula (V), (Va) and/or (Vb) and/or a compound according to formula (VI) (L-P1-S—[S—P2-S]ₙ—S—P3-L), more preferably at least one of the disulfide-crosslinked cationic peptides Cys-Arg₁₂ (SEQ ID NO: 580), Cys-Arg₁₂-Cys (SEQ ID NO: 579), or Trp-Arg₁₂-Cys (SEQ ID NO: 1074) and wherein the isRNA is preferably administered intratumorally.

According to a particularly preferred embodiment, the invention provides an isRNA for use in the treatment of a tumor or cancer disease,

wherein the isRNA comprises a nucleic acid sequence according to formula (I) (GₗXₘGₙ), formula (II) (CₗXₘCₙ), formula (III) (NᵤGₗXₘGₙNᵥ)ₐ or formula (IV) (NᵤCₗXₘCₙNᵥ)ₐ, preferably at least one nucleic acid sequence according to any one of SEQ ID NOs: 433 to 437, 1014 to 1016, or a fragment or variant of any one of these sequences, more preferably according to any one of SEQ ID NOs: 433, 434, or 1014 to 1016, or a fragment or a variant of any one of these nucleic acid sequences,

wherein the isRNA is complexed with a cationic or polycationic compound, preferably with a polymeric carrier, more preferably with a polymeric carrier that is formed by a disulfide-crosslinked cationic component, which preferably comprises a peptide according to formula (V), (Va) and/or (Vb) and/or a compound according to formula (VI) (L-P¹—S—[S—P2-S]ₙ—S—P3-L), more preferably at least one of the disulfide-crosslinked cationic peptides Cys-Arg₁₂ (SEQ ID NO: 580), Cys-Arg₁₂-Cys (SEQ ID NO: 579), or Trp-Arg₁₂-Cys (SEQ ID NO: 1074), wherein the isRNA is preferably administered intratumorally, and

wherein the tumor or cancer disease is preferably selected from the group consisting of melanoma, preferably advanced and/or metastatic melanoma; squamous cell cancer of the skin (SCC), preferably unresectable and/or advanced SCC; adenocystic carcinoma (ACC), preferably advanced ACC; cutaneous T-cell lymphoma, preferably advanced cutaneous T-cell lymphoma refractory to local treatment or to chemotherapy; and head and neck cancer (HNSCC), preferably advanced HNSCC. In this context, the tumor or cancer disease is preferably selected from the group consisting of advanced melanoma, preferably advanced cutaneous melanoma (cMEL), squamous cell carcinoma of the skin (SCC), preferably cutaneous squamous cell carcinoma (cSCC), squamous cell carcinoma of the head and neck (HNSCC), and adenoid cystic carcinoma (adenocystic carcinoma (ACC)).

In an alternative preferred embodiment, the tumor or the cancer disease is selected from the group consisting of advanced cutaneous melanoma (cMEL), cutaneous squamous cell carcinoma (cSCC), head and neck squamous cell carcinoma (hnSCC), and adenoid cystic carcinoma (ACC).

According to a particularly preferred embodiment, the invention provides an isRNA for use in the treatment of a tumor or cancer disease,

wherein the isRNA comprises a nucleic acid sequence according to formula (I) (GₗXₘGₙ), formula (II) (CₗXₘCₙ), formula (III) (NᵤGₗXₘGₙNᵥ)ₐ or formula (IV) (NᵤCₗXₘCₙNᵥ)ₐ, preferably at least one nucleic acid sequence according to any one of SEQ ID NOs: 433 to 437, 1014 to 1016, or a fragment or variant of any one of these sequences, more preferably according to any one of SEQ ID NOs: 433, 434; 1014 to 1016, or a fragment or a variant of any one of these nucleic acid sequences,

wherein the isRNA is complexed with a cationic or polycationic compound, preferably with a polymeric carrier, more preferably with a polymeric carrier that is formed by a disulfide-crosslinked cationic component, which preferably comprises a peptide according to formula (V), (Va) and/or (Vb) and/or a compound according to formula (VI) (L-P¹—S—[S—P2-S]ₙ—S—P3-L), more preferably at least one of the disulfide-crosslinked cationic peptides Cys-Arg₁₂ (SEQ ID NO: 580), Cys-Arg₁₂-Cys (SEQ ID NO: 579), or Trp-Arg₁₂-Cys (SEQ ID NO: 1074),

wherein the isRNA is preferably administered intratumorally, and

wherein the tumor or cancer disease is preferably selected from the group consisting of melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

wherein the tumor or the cancer disease is preferably selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC);

wherein the tumor or the cancer disease is preferably at an advanced stage and/or refractory to standard therapy.

In certain embodiments, the isRNA as described herein is provided for use in the treatment of a tumor or cancer disease, preferably as defined herein, wherein the treatment comprises administration of at least one additional pharmaceutically active ingredient and wherein the isRNA is preferably administered intratumorally, including peritumorally or locoregionally, preferably as described herein. In particular, the present invention provides the isRNA for use in the treatment or prophylaxis of a tumor or cancer disease as described herein, wherein the treatment comprises administration of at least one additional pharmaceutically active ingredient that is conventionally used in the treatment and/or prophylaxis of a tumor or cancer disease, preferably as described herein and wherein the isRNA is preferably administered intratumorally, including peritumorally or locoregionally. In the context of the present invention, the phrase ‘pharmaceutically active ingredient that is conventionally used in the treatment and/or prophylaxis of a [tumor or cancer disease]’ preferably refers to a pharmaceutically active ingredient that is used—preferably according to standard therapy—in the treatment and/or prophylaxis of a tumor or cancer disease. More preferably, the phrase comprises a pharmaceutically active ingredient that is known in the art to be suitable for treatment and/or prophylaxis of a tumor or cancer disease.

According to a preferred embodiment, the isRNA is provided for use in the treatment of a tumor or cancer disease as described herein, wherein the treatment comprises administration of at least one additional pharmaceutically active ingredient that is conventionally used in the treatment and/or prophylaxis of the respective disease and wherein the isRNA is preferably administered intratumorally.

Even more preferably, the isRNA is provided for use in the treatment of a tumor or cancer disease selected from the group consisting of melanoma, preferably advanced and/or metastatic melanoma, most preferably advanced cutaneous melanoma (cMEL), squamous cell cancer of the skin (SCC), preferably unresectable and/or advanced SCC; most preferably preferably cutaneous squamous cell carcinoma (cSCC); adenocystic carcinoma (ACC), preferably advanced ACC; cutaneous T-cell lymphoma, preferably advanced cutaneous T-cell lymphoma refractory to local treatment or to chemotherapy; and squamous cell carcinoma of the head and neck (HNSCC), preferably advanced HNSCC, wherein the treatment comprises concomitant localized or systemic administration of at least one additional pharmaceutically active ingredient that is conventionally used in the treatment and/or prophylaxis of any of these diseases and wherein the isRNA is preferably administered intratumorally.

According to a preferred embodiment, the isRNA is provided for use in the treatment of a tumor or cancer disease selected from the group consisting of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

for use in the treatment of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC);

wherein the tumor or the cancer disease is preferably at an advanced stage and/or refractory to standard therapy, wherein the treatment comprises concomitant localized or systemic administration of at least one additional pharmaceutically active ingredient that is conventionally used in the treatment and/or prophylaxis of any of these diseases and wherein the isRNA is preferably administered intratumorally.

The at least one additional pharmaceutically active ingredient may be administered by any suitable administration route or technique, preferably as described herein, or by intravenous, oral or local/topical application. In a preferred embodiment, the isRNA as well as the at least one additional pharmaceutically active ingredient are administered intratumorally. In a more preferred embodiment, the isRNA is administered intratumorally and at least one additional pharmaceutically active ingredient is administered systemically (oral or intravenous or subcutaneous or intramuscular or intraperitoneal or intradermal).

In some embodiments, the at least one additional pharmaceutically active ingredient is formulated together with the isRNA for use as described herein. In a particularly preferred embodiment, the at least one additional pharmaceutically active ingredient is formulated separately from the isRNA for use as described herein.

The at least one additional pharmaceutically active ingredient is not limited to a particular class of compounds. For example, the at least one additional pharmaceutically active ingredient may preferably be a compound, which is conventionally used in chemotherapy of the tumor or cancer disease, for which the isRNA as described herein is used. Preferably, the at least one additional pharmaceutically active ingredient is a compound as described herein for use in combination with the isRNA as described herein. Alternatively, the at least one additional pharmaceutically active ingredient may preferably be a therapeutic peptide or protein (e.g. an antibody or a decoy receptor) or a fragment or variant thereof.

In some embodiments, the at least one additional pharmaceutically active ingredient is a compound, which is conventionally used in the treatment and/or prophylaxis of melanoma, preferably advanced and/or metastatic melanoma and most preferably advanced cMEL, wherein the compound is preferably selected from the group consisting of Nivolumab (Opdivo), Ipilimumab (Winglore, Yervoy), Pembrolizumab (Keytruda), dabrafenib mesylate+trametinib dimethyl sulfoxide (Tafinlar+Mekinist), temozolomide (Astromide, Temodal, Temozolomide), vemurafenib (Zelboraf), peginterferon alfa-2b (Pegintron), aldesleukin (Proleukin), bleomycin sulfate (Bleo, Bleoprim), carboplatin (Paraplatin), carmustine (Becenun, Bicnu), cisplatin (Platinol, Cisplatin, Displanor, Nuoxin), cobimetinib fumarate (Cotellic), cyclophosphamide (Endoxan), dabrafenib mesylate (Tafinlar), dacarbazine (Dacarbazine, DBL Dacarbazine, DTIC, DTIC-Dome, Dacarbazina Mayne, Dacarbazine, Dacarbazina, Detimedac, Dacarb, Celdaz, Dabaz, Dacarzine, Dacarin, Dacmed, Oncodac, Dti, Dacarbazine Sandoz, Fauldacar, Dacarex, Daczin, Arzi, Tiferomed, Acocarb, DTI, Evodazin), dactinomycin (Cosmegen, Kosmegen, Lyovac Cosmegen), diphenidol hydrochloride (Bleocina, Raamfen, Dimensal), epirubicin hydrochloride (Pharmorubicin), fotemustine (Muphoran, Mustoforan, Mustophoran, Fotemustine), hydroxyurea (Hydrine, Durea, Myelostat, Riborea, Unidrea, Hydran, Leukocel, Hydroxyurea, Hydrea, hydroxyurea), interferon alfa (Alfanative, Immunex), interferon alfa-2a (Intermax Alpha, Inron-A, Intefen, Viteron-A, Roferon A, Alferon), interferon alfa-2b (Realfa-2B, Shanferon, Intron A, Viraferon, Alfaron, Lemoron, Liveral, Recombinant Human Interferon alfa-2b, ReliFeron, Fni 2B, Infostat, Altevir, Lanstion, interferon beta (Feron), interleukin-2 (Inleusin, Recombinant Human Interleukin-2), irinotecan hydrochloride (Camptolem), lomustine (Belustine, Lomustine, Moostin), melanoma lysate vaccine (Melacine), melphalan (Alkacel, Alkeran, Melphalan), methotrexate (Methotrexate), nivolumab+ipilimumab (Opdivo+Yervoy), Sylatron (Pegintron, Sylatron), talimogene laherparepvec (Imlygic), tamoxifen citrate (Tamoxifen), temozolomide (Emzolam, Temozolomide, Temozolomide LW, Tiloshil, Temolida, Temozod, Temozolomide, Temizole), trametinib dimethyl sulfoxide (Mekinist), vincristine sulfate (Oncovin, Vincristine Sulfate, Vincristine, Fauldvincri, Eldisine), (mequinol+tretinoin) (Solage), Dacarbazine (Dacarbazine), interferon alfa (Biaferone, Isiferone), cisplatin; dacarbazine; vindesine sulfate, fibrin, sealant, tremelimumab, interferon alfa-2b, recombinant interferon alfa, imiquimod, etanercept, sorafenib tosylate; sun itinib malate, aldesleukin, imatinib mesylate; nilotinib, sargramostim, aldesleukin, ipilimumab; sargramostim, ipilimumab, pasireotide, aldesleukin; ipilimumab, vemurafenib, melphalan, cetuximab; cisplatin; cyclophosphamide; dacarbazine; docetaxel; doxorubicin; etoposide; fluorouracil; gemcitabine hydrochloride; irinotecan hydrochloride; oxaliplatin; paclitaxel; pemetrexed disodium; temozolomide, interferon alfa-2b; Sylatron, bleomycin, talimogene laherparepvec, dabrafenib mesylate; dabrafenib mesylate+trametinib dimethyl sulfoxide; trametinib dimethyl sulfoxide; vemurafenib, trametinib dimethyl sulfoxide, dabrafenib mesylate; trametinib dimethyl sulfoxide, dabrafenib mesylate, granulocyte macrophage colony stimulating factor; ipilimumab, bleomycin; undisclosed chemotherapy, ipilimumab; vemurafenib, nivolumab, bortezomib; cabozantinib s-malate; ceritinib; crizotinib; dasatinib; erlotinib hydrochloride; everolimus; gefitinib; imatinib; lapatinib ditosylate; nilotinib; olaparib; palbociclib; pazopanib hydrochloride; ramucirumab; regorafenib; sorafenib tosylate; sunitinib malate; trametinib dimethyl sulfoxide; vorinostat, bortezomib; cabozantinib s-malate; ceritinib; crizotinib; dasatinib; erlotinib hydrochloride; everolimus; gefitinib; imatinib; lapatinib ditosylate; nilotinib; olaparib; palbociclib; pazopanib hydrochloride; ramucirumab; regorafenib; sorafenib tosylate; sunitinib malate; trametinib dimethyl sulfoxide; vorinostat, aminolevulinic acid hydrochloride, dabrafenib mesylate; vemurafenib, pembrolizumab, GSK-2132231A, fotemustine, M-Vax, melphalan; tumor necrosis factor alfa, Canvaxin, aldesleukin; tumor infiltrating lymphocytes, interferon alfa-2a, talimogene laherparepvec, aldesleukin; cisplatin; dacarbazine; filgrastim; interferon alfa; vinblastine, velimogene aliplasmid, oblimersen sodium, Vaccine to Target gp100 Antigen for Metastatic Melanoma, temozolomide, velimogene aliplasmid, sorafenib tosylate, DHA-Paclitaxel, melphalan, vitespen, cisplatin; dacarbazine, peginterferon alfa-2b, lenalidomide, melanoma lysate vaccine, celecoxib, tasisulam sodium, paclitaxel albumin bound, sorafenib tosylate, aldesleukin; histamine dihydrochloride, Canvaxin, sorafenib tosylate, GM2-KLH Vaccine+Q521, elesclomol; paclitaxel, peginterferon alfa-2b, BMS-734019; ipilimumab, aldesleukin; cisplatin; dacarbazine; filgrastim; vinblastine, aldesleukin; cisplatin; dacarbazine; interferon alfa-2b; vinblastine sulfate, dacarbazine; oblimersen sodium, bevacizumab, oblimersen sodium, cisplatin; cytarabine; paclitaxel; treosulfan, Vaccine for Melanoma, melphalan; recombinant tumor necrosis factor, GM2-KLH vaccine; QS-21, Polyvalent Melanoma Vaccine+BCG Vaccine, aldesleukin; cisplatin; dacarbazine; interferon alfa; vinblastine, temozolomide, interferon gamma; melphalan; tumor necrosis factor alfa, Cellular Immunotherapy for Melanoma, retinol, interferon alfa-2a, Corynebacterium granulosum P40, melphalan, cisplatin, bacillus calmette-guerin vaccine; cyclophosphamide, aldesleukin; cisplatin; dacarbazine; interferon alfa; vinblastine, ranibizumab, interferon alfa, bacillus calmette-guerin vaccine, bacillus calmette-guerin vaccine, melphalan, interferon alfa, megestrol, interferon alpha-2, cisplatin; dacarbazine, bacillus calmette-guerin vaccine; dacarbazine, aldesleukin; lisofylline, dabrafenib mesylate, cyclophosphamide; dactinomycin; vincristine, bacillus calmette-guerin vaccine, bacillus calmette-guerin vaccine; dacarbazine, trametinib dimethyl sulfoxide, PV-10, cholecalciferol, timolol maleate, masitinib, fotemustine; interferon alfa-2b, talimogene laherparepvec, sodium biselenite, cisplatin; dacarbazine; vindesine, isotretinoin, Sylatron, fotemustine, seviprotimut-L, dabrafenib mesylate; trametinib dimethyl sulfoxide, peginterferon alfa-2a, aldesleukin; isotretinoin; peginterferon alfa-2b, dabrafenib mesylate; trametinib dimethyl sulfoxide, cobimetinib fumarate; vemurafenib, interferon alfa-2b; M-Vax, nivolumab, dacarbazine; paclitaxel, M-200, cholecalciferol, binimetinib, melphalan, sargramostim, pembrolizumab, gene therapy for malignant melanoma, eltrapuldencel-T, binimetinib; encorafenib, sargramostim; tyrosinase peptide vaccine, pembrolizumab; talimogene laherparepvec, autologous tumor-infiltrating lymphocytes, talimogene laherparepvec, dabrafenib mesylate; ipilimumab; nivolumab; trametinib dimethyl sulfoxide, aldesleukin; cyclophosphamide; fludarabine; tumor infiltrating lymphocytes, PV-10, talimogene laherparepvec, HM-95573, dabrafenib mesylate; trametinib dimethyl sulfoxide, interferon beta, ipilimumab; nivolumab, Darleukin; Fibromun, LN-144, binimetinib, epacadostat; pembrolizumab, oblimersen sodium, agatolimod sodium, lenalidomide, interferon alfa-2a; peginterferon alfa-2a, CSF-470, Melanoma vaccine modified to express HLA A2/4-1BB ligand (Cellular Immunotherapy for Metastatic Melanoma), ipilimumab; nivolumab; sargramostim, ImmuniCell (Cellular Immunotherapy for Cancer and Viral Infections), trabedersen, BTH-1677, trametinib dimethyl sulfoxide, melphalan (Melblez), AS15+recMAGE-A3, binimetinib, binimetinib+encorafenib, Cellular Immunotherapy for Cancer and Viral Infections, CSF-470, dabrafenib mesylate (Tafinlar), dabrafenib mesylate+trametinib dimethyl sulfoxide, Darleukin, encorafenib, epacadostat, masitinib, PV-10, Sargramostim (Leukine), seviprotimut-L, trabedersen, Vaccine for Breast Cancer, Melanoma and Soft Tissue Sarcoma and Vemurafenib (Zelboraf).

In particularly preferred embodiments, the at least one additional pharmaceutically active ingredient is a compound, which is conventionally used in the treatment and/or prophylaxis of melanoma, preferably advanced and/or metastatic melanoma and most preferably advanced cMEL, wherein the compound is a PD-1 inhibitor, preferably an antagonistic PD-1 antibody, preferably selected from the group consisting of Nivolumab (Opdivo), and Pembrolizumab (Keytruda).

In certain embodiments, the at least one additional pharmaceutically active ingredient is a compound, which is conventionally used in the treatment and/or prophylaxis of squamous cell cancer of the skin (SCC), preferably unresectable and/or advanced SCC, wherein the compound is preferably selected from the group consisting of Cetuximab (Erbitux), paclitaxel albumin bound (Abraxane), (gimeracil+oteracil+tegafur) (TS-1), Docetaxel (Docetaxel, Doxel, Taxotere, Docetaxel An, Docel, Nanoxel M, Tautax, Docetaxel −AS, Docetaxel-M, Qvidadotax, Relidoce, Taxelo, Oncodocel, Doxotel, Pacancer, Docetrust, Dodetax, Dodabur, Soulaxcin, Taxedol, Docefim, Docetaxel, Ribodocel, Critidoc, Asodoc, Chemodoc, Docelibbs, Docenat, Dincilezan, Dostradixinol, Docefrez, Ca mitotic, Oncotaxel, Somatixel, Belotaxel, Qvidadotax, Taxceus, Cetadocure, Docetaxel CT, Tevaxter, Docirena, Eurotere, Axtere, Celotax, Taxanit, Drobanos, Cetado, Doxocad, Taxceus, Egidox, Tedocad, Docecad, Docelex, Docetax, Docetaxel, Docetere, Dotax, Taxuba, Monotaxel, Taceedo, Detaxl, Docet, Docetaxel, Ferdotax, Wintaxel), (tegafur+uracil) (Uft, Uft Tefudex, Unitoral, Luporal, Tagracil), Fluorouracil (5-FU), (gimeracil+oteracil+tegafur) ODT (TS-1 Combination OD), bleomycin sulfate (Tecnomicina, Cinaleo, Bleomycin, Bloicin-S, Bonar, Bleocin, Bleomycin Sulfate, Bleo, Bleocel, Bleotex, Oncobleo, Bleonco, Bleosol, Lyoble, Bleomycin Sulfate, Blenamax, Bleomycin, Blenoxane, Bleomicina, Bleomycine Bellon, Bleoprim), carboplatin (Carboplatin, Platamine CS, Carbaccord, Carboplatina, Carboplatino, Paraplatin, Carbosin, Tecnocarb, Carbomerck, Paract, Carboplatine CTRS, Carboplatine Intsel Chimos, Carboplatin, Carbokem, Carbotinol, Fauldcarbo, Evocarb, Citoplatina, Platin), ciprofloxacin (Hypoflox, Ufexil), ciprofloxacin hydrochloride (Ciprofloxacin Pharma, Prodin, Ciproxin), cisplatin (Cisplatin, Stritin, Ifapla, Accocit, Unistin, Cancertin, Cisplan, Citoplax, Nuoxin, Placis, Cisplatino, Displanor, Randa, Cispla, Fauldcispla, Briplatin, Platinex, Platinol, Platinex, Riboplatin, Cisplatine, Platistine CS, Platosin, Accocit, Cisplatino) cyclophosphamide (Endoxan, Cyclophosphamide), doxifluridine (Doxifluridine, May Vladimir), doxorubicin (Doxorubicin Hydrochloride, Adriamycin RDF, Doxorubicin, Doxorubicin PFS), epirubicin, hydrochloride (Brecila, Cloridrato De Epirrubicina, Epirubicin, Farmorubicina, Nuovodox, Adnexa, 4-Eppedo, Favicin), fluorouracil (Agicil, Fluorouracil, Fauldfluor, Oncourcil, Flocil, 5 Flucel), folic acid+methotrexate (Truxofol), human adenovirus type 5 (recombinant) (Oncorine), hydroxyurea (Oxyrea, Durea, Myelostat, Riborea, Unidrea, Ondrea, Hydran, Leukocel, Hydroxyurea, Hydrea), ifosfamide (Holoxan, Ifosfamide EG), levamisole (Zirsol), methotrexate Methotrexate (Tratoben, Methotrexate, Fresexate, Neometho, Fauldmetro, Methotrexate Sodium, Methocel, Hytas, Methaccord, Methofill, Metotrexato, Traxacord, Plastomet, Tevatrex, Metrex, Caditrex, Carditrex, Vibzi, Imutrex, Biotrexate, Methorex, Mexate, Neotrexate, Oncotrex, Remtrex, Trixilem, Hi-Trex, Metorex, Trex, Unitrexate, Ebetrexac, Fauldexato, Lantarel, Maxtrex, Miantrex CS, Rheumatrex, Folex, Folex PFS, Abitrexate, Tevametho, Trexall, Emthexate, Abitrexate, Meadow), mitomycin (Mitomycin C, Mitomycin, Mitonco, Lyomit), nedaplatin (Jiebaishu, Aoxianda, Aqupla), nimesulide (Nimulid), nimotuzumab (Biomab EGFR, Laedemab), nitrofurantoin (Furatsilin), ofloxacin (Entof), paclitaxel (Paclitaxel, Taxol), peplomycin sulfate (Pepleo), picibanil (Picibanil), pirarubicin (Pirarubicin Hydrochloride, Therarubicin, Pinorubin), sodium glycididazole (CMNa), tegafur (Utefos, Icarus, Futraful, Tegafur Gimeracil Oteracil Potassium), temoporfin (Foscan), topotecan hydrochloride (Topotecan), ubenimex (Ubenimex), vinblastine sulfate (Vinblastine, Vblastin), vincristine sulfate (Vincristine, Vincristine Sulfate, Vincristin, Sutivin, vindesine sulfate (Eldisine), carboplatin (Carboplatine Qualimed, Carboplatine, Carboplatino, Carboplatin), cisplatin (Cisplatin), docetaxel (Kamdocon, Naltoxater, Docetaxel), fluorouracil (Fluorouracil, Fluorouracile, Fluorouracil), methotrexate (Methotrexate Sodium, Mexate, Mexate Aq, Biometrox, Medsatrexate, Otaxem), vincristine sulfate (Oncovin), fluorouracil, sunitinib malate, acitretin, fibrin sealant, cetuximab, cetuximab, erlotinib, cisplatin; docetaxel; fluorouracil, undisclosed anti-cancer drug, gefitinib, pravastatin sodium, sirolimus, undisclosed chemotherapy, cisplatin; docetaxel; fluorouracil, sirolimus, fluorouracil; undisclosed taxane, methyl aminolevulinate hydrochloride, cisplatin; docetaxel; fluorouracil, erlotinib hydrochloride, cetuximab, imiquimod, undisclosed Chinese herbal medicine, aspirin; enalapril maleate, undisclosed chemotherapy, cetuximab, (gimeracil+oteracil+tegafur); carboplatin; cisplatin, cisplatin; fluorouracil; nimotuzumab, carboplatin; paclitaxel albumin bound, cisplatin; nedaplatin, bleomycin, nedaplatin, cisplatin; paclitaxel, paclitaxel albumin bound, (gimeracil+oteracil+tegafur), bleomycin; undisclosed chemotherapy, apatinib; docetaxel, undisclosed immunomodulatory supplement, BCM-95, aminolevulinic acid hydrochloride, nedaplatin, cisplatin; palifermin, cetuximab, gefitinib, bevacizumab, belagenpumatucel-L, cisplatin; tirapazamine, cisplatin; tirapazamine, cisplatin; gemcitabine; paclitaxel; topotecan; vinorelbine, cisplatin; fluorouracil, panitumumab, carboplatin; docetaxel; gemcitabine hydrochloride; vinorelbine tartrate, amifostine; fluorouracil, cisplatin; fluorouracil, carboplatin; paclitaxel, tirapazamine, cisplatin; epoetin alfa, figitumumab, melphalan; tumor necrosis factor alf, cisplatin, cisplatin; fluorouracil, cisplatin; undisclosed chemotherapy, docetaxel, contusugene ladenovec, cisplatin; fluorouracil; paclitaxel, docetaxel, human papillomavirus [serotypes 16, 18](bivalent) vaccine, isotretinoin, cisplatin; fluorouracil, misonidazole, paclitaxel, palifermin, endostatin, pilocarpine, cisplatin; docetaxel; filgrastim; fluorouracil; paclitaxel, cisplatin; docetaxel; filgrastim; fluorouracil; paclitaxel, cisplatin; irinotecan hydrochloride, cisplatin; gemcitabine, cisplatin; epirubicin; fluorouracil; undisclosed chemotherapy, methyl aminolevulinate hydrochloride, carboplatin; paclitaxel, carbogen; carbon dioxide; niacinamide, cisplatin; fluorouracil, talimogene laherparepvec, epoetin alfa, cisplatin; fluorouracil; panitumumab, cisplatin; fluorouracil, cisplatin; fluorouracil, aldesleukin, cisplatin; fluorouracil, cisplatin; paclitaxel, cisplatin; fluorouracil, fluorouracil; leucovorin; lobaplatin, cisplatin, cisplatin; ethyl mercaptan; ifosfamide; mesna; mitolactol, doxorubicin; levamisole, (tegafur+uracil), cisplatin; fluorouracil, cisplatin; vinorelbine, carboplatin; cisplatin; gemcitabine hydrochloride, Corynebacterium parvum; doxorubicin, capecitabine; cisplatin; fluorouracil; paclitaxel, fluorouracil; leucovorin; methotrexate, rAd-p53, cetuximab; cisplatin; docetaxel, PV-10, methyl aminolevulinate hydrochloride, cisplatin; fluorouracil, paclitaxel; topotecan hydrochloride, carboplatin; cisplatin; paclitaxel, cisplatin; topoteca n hydrochloride, cisplatin; etoposide, docetaxel; fluorouracil, aspirin, cisplatin; gemcitabine, Lactobacillus brevis CD2, cisplatin; docetaxel, fosbretabulin tromethamine, panitumumab, fluorouracil, paclitaxel, carboplatin; cisplatin; docetaxel; fluorouracil, fluorouracil, erlotinib hydrochloride, cisplatin; undisclosed chemotherapy; vinorelbine, (gimeracil+oteracil+tegafur); carboplatin, cetuximab, contusugene ladenovec, cetuximab, methyl aminolevulinate hydrochloride, cyclophosphamide, (gimeracil+oteracil+tegafur); cisplatin, paclitaxel albumin bound, carboplatin; paclitaxel, cisplatin; gemcitabine, capecitabine; cisplatin, docetaxel, Z-100, cisplatin; ifosfamide; paclitaxel, nimotuzumab, irinotecan hydrochloride, celecoxib; methotrexate, Nutrison, carboplatin; cisplatin; fluorouracil; paclitaxel, cisplatin; paclitaxel, cisplatin; docetaxel; vinorelbine, paclitaxel, (gimeracil+oteracil+tegafur); cisplatin, carboplatin; paclitaxel, methyl aminolevulinate hydrochloride, Aibin, cisplatin; fluorouracil, porfimer sodium, carboplatin; cisplatin; tocotrienol; vinorelbine, (gimeracil+oteracil+tegafur); cisplatin; paclitaxel, docetaxel, ipilimumab, cisplatin, VB-4847, celecoxib; thalidomide, cisplatin; epirubicin; fluorouracil, cisplatin; fluorouracil, fluorouracil, carboplatin; paclitaxel, cetuximab; cisplatin; docetaxel, autologous cytokine induced killer cells, cisplatin; docetaxel; fluorouracil, cisplatin; epirubicin; fluorouracil, tergenpumatucel-L, cetuximab; cisplatin; docetaxel, Elental, cisplatin; nimotuzumab; paclitaxel, eicosapentaenoic acid; undisclosed nutritional supplement, palbociclib, pembrolizumab (Keytruda), nimotuzumab, apatorsen and dacomitinib.

In particularly preferred embodiments, the at least one additional pharmaceutically active ingredient is a compound, which is conventionally used in the treatment and/or prophylaxis of squamous cell cancer of the skin (SCC), preferably unresectable and/or advanced SCC, wherein the compound is a PD-1 inhibitor, preferably an antagonistic PD-1 antibody, preferably selected from the group consisting of Nivolumab (Opdivo), and Pembrolizumab (Keytruda).

In some embodiments, the at least one additional pharmaceutically active ingredient is a compound, which is conventionally used in the treatment and/or prophylaxis of squamous cell carcinoma of the head and neck (HNSCC), preferably advanced HNSCC, wherein the compound is preferably selected from the group consisting of Nivolumab, Cetuximab (Erbitux), paclitaxel albumin bound (Abraxane), gimeracil+oteracil+tegafur (TS-1), docetaxel (Docetaxel, Doxel, Taxotere, Docetaxel An, Nanoxel M, Tautax, Docetaxel −AS, Docetaxel-M, Qvidadotax, Relidoce, Taxelo, Oncodocel, Doxotel, Pacancer, Docetrust, Dodetax, Dodabur, Soulaxcin, Taxedol, Docefim, Ribodocel, Critidoc, Asodoc, Chemodoc, Docelibbs, Docenat, Dincilezan, Dostradixinol, Docefrez, Camitotic, Oncotaxel, Somatixel, Belotaxel, Qvidadotax, Taxceus, Cetadocure, Docetaxel CT, Tevaxter, Docirena, Eurotere, Axtere, Celotax, Taxanit, Drobanos, Cetado, Doxocad, Taxceus, Egidox, Tedocad, Docecad, Docelex, Docetax, Docetere, Dotax, Taxuba, Monotaxel, Taceedo, Detaxl, Docet, Ferdotax, Wintaxel, Kamdocon, Naltoxater), tegafur+uracil (Uft, Uft E, tefudex, Unitoral, Luporal Tagracil), fluorouracil (bn: 5-FU, Fluorouracil, Fluorouracile), gimeracil+oteracil+tegafur ODT (bn TS-1 Combination OD), bleomycin sulfate (Tecnomicina, Cinaleo, Bleomycin, Bloicin-S, Bonar, Bleocin, Bleo Bleomycin Sulfate, Bleocel, Bleotex, Oncobleo, Bleonco, Bleosol, Lyoble, Bleomycin Sulfate, Blenamax, Blenoxane, Bleomicina Bleomycine Belton Bleoprim), carboplatin (Carboplatin Plata mine CS Carbaccord Carboplatina Carboplatino Paraplatin Carbosin Tecnocarb Carbomerck, Paract, Carboplatine CTRS, Carboplatine Intsel Chimos, Carbokem Carbotinol Fauldcarbo, Evocarb, Citoplatina, Platin, Carboplatine Qua limed, Carboplatine, Carboplatino, Carboplatin), ciprofloxacin (Hypoflox, Ufexil), ciprofloxacin hydrochloride (Ciprofloxacin Pharma, Prodin, Ciproxin), cisplatin (Cisplatin, Stritin, Ifapla, Accocit, Unistin, Cancertin, Cisplan, Citoplax, Nuoxin, Placis, Cisplatino, Displanor, Cisplatine, Randa, Cisplatin, Cispla, Fauldcispla, Briplatin, Platinex, Platinol, Riboplatin, Platistine CS, Platosin, Accocit), cyclophosphamide (Endoxan, Cyclophosphamide), doxifluridine (Doxifluridine, May Vladimir, Doxifluridine), doxorubicin (Doxorubicin Hydrochloride, Adriamycin RDF, Doxorubici, Doxorubicin Hydrochloride, Doxorubicin PFS), epirubicin hydrochloride (Brecila, Cloridrato De Epirrubicina, Epirubicin, Farmorubicina, Nuovodox, Adnexa, 4-Eppedo, Favicin), fluorouracil (Agicil, Fluorouracil, Fauldfluor, Oncourcil Flocil, 5 Flucel), folic acid+methotrexate (Truxofol), human adenovirus type 5 (recombinant) (Oncorine), hydroxyurea (Oxyrea, Durea, Myelostat, Riborea, Unidrea, Ondrea, Hydra n, Leukocel, Hydroxyurea, Hydrea), ifosfamide (Holoxan, Ifosfamide EG), levamisole (Zirsol), methotrexate (Tratoben, Methotrexate, Fresexate, Neometho, Fauldmetro, Methotrexate Sodium, Methocel, Hytas, Methaccord, Methofill, Metotrexato, Traxacord, Plastomet, Tevatrex, Metrex, Caditrex, Carditrex, Vibzi, Imutrex, Biotrexate, Methorex, Mexate, Neotrexate, Oncotrex, Remtrex, Trixilem, Hi-Trex, Metorex, Trex, Unitrexate, Ebetrexac, Fauldexato, Lantarel, Maxtrex, Miantrex CS, Rheumatrex, Folex, Folex PFS, Abitrexate, Trexall, Emthexate, Abitrexate, Meadow, Mexate Aq, Biometrox, Otaxem), Mitomycin (Mitomycin C, Mitomycin, Mitonco, Lyomit), Nedaplatin (Jiebaishu, Aoxianda, Aqupla), Nimesulide (Nimulid), Nimotuzumab (Biomab EGFR, Laedemab), Nitrofurantoin (Furatsilin), Ofloxacin (Entof), Paclitaxel (Paclitaxel, Taxol), peplomycin sulfate (Pepleo), picibanil (Picibanil), pirarubicin (Pirarubicin Hydrochloride, Therarubicin, Pinorubin), sodium glycididazole (CMNa), tegafur (Utefos, Icarus, Futraful, Tegafur Gimeracil Oteracil Potassium), temoporfin (Foscan), topotecan hydrochloride (Topotecan), ubenimex (Ubenimex), vinblastine sulfate (Vinblastine, Vblastin, Vincristine, Vincristine Sulfate, Vincristin, Sutivin, Vincrisan, Eldisine), vincristine sulfate (Oncovin), gefitinib, escitalopram oxalate, Gene Therapy to Activate p53 for Oncology, cevimeline hydrochloride, docetaxel; lenograstim, bortezomib; docetaxel; irinotecan, carboplatin; paclitaxel, cisplatin; docetaxel; fluorouracil, human papillomavirus vaccine (Vaccination Against Human Papillomavirus Types 16 and 18 and Unvaccinated Controls), carboplatin; cisplatin; paclitaxel, undisclosed epidermal growth factor receptor inhibitor, cetuximab, sorafenib tosylate; sunitinib malate, amifostine, cisplatin; dexamethasone; docetaxel; metoclopramide, platinum based chemotherapy, cetuximab; nimotuzumab, nimotuzumab, dobutamine hydrochloride; dopexamine hydrochloride; epinephrine; norepinephrine bitartrate, cetuximab; cisplatin, cetuximab; cisplatin; docetaxel; fluorouracil, cisplatin; paclitaxel, Gendicine, Doce onkovis (Docetaxel), Picibanil, docetaxel; lobaplatin, oxytetracycline hydrochloride, cisplatin; mitomycin, bleomycin, nimotuzumab, cisplatin; fluorouracil; nimotuzumab, cetuximab; cisplatin; cyclophosphamide; dacarbazine; docetaxel; doxorubicin; etoposide; fluorouracil; gemcitabine hydrochloride; irinotecan hydrochloride; oxaliplatin; paclitaxel; pemetrexed disodium; temozolomide, varenicline, acetylcysteine, dopamine; norepinephrine, (gimeracil+oteracil+tegafur), nimotuzumab, icotinib hydrochloride, Kushen (kuh-seng), Carboxymethylpachymaran, BCM-95, Lactofos, Pegfilgrastim, methadone, Varanadi ghrita, cisplatin; palifermin, cetuximab, cisplatin; lapatinib ditosylate, gefitinib, zalutumumab, erlotinib hydrochloride, beta carotene; E-Tabs, bevacizumab, levothyroxine; liothyronine sodium; recombinant human thryroid stimulating hormone, cisplatin; docetaxel; fluorouracil; hydroxyurea, fluorouracil, docetaxel; St. John's Wort, carboplatin; cisplatin; fluorouracil; paclitaxel, Biafine, methotrexate, cisplatin; fluorouracil, alpha tocopherol; isotretinoin; recombinant interferon alfa, panitumumab, cisplatin, bleomycin; fluorouracil; leucovorin; methotrexate; vincristine, nimotuzumab, panitumumab, amifostine; fluorouracil, cisplatin; docetaxel; fluorouracil, capecitabine; cisplatin; fluorouracil, pemetrexed disodium, cisplatin; gemcitabine, tranexamic acid, carboplatin; fluorouracil, cefazolin, epoetin alfa, tirapazamine, erlotinib hydrochloride, cetuximab; docetaxel; fluorouracil, megestrol, contusugene ladenovec, cevimeline hydrochloride, cisplatin; fluorouracil, cisplatin; fluorouracil; paclitaxel, isotretinoin, porfiromycin, escitalopram oxalate, gimeracil+oteracil+tegafur, IntraDose (Cisplatin Plus Epinephrine), misonidazole, tirapazamine, carboplatin; cisplatin; docetaxel; fluorouracil, gefitinib, epoetin alfa, paclitaxel, gefitinib, palifermin, pilocarpine, celecoxib, cisplatin; docetaxel; filgrastim; fluorouracil; paclitaxel, cisplatin; fluorouracil; leucovorin, cisplatin; gemcitabine; mannitol, Lactobacillus brevis CD2, Glutamine, carbogen; niacinamidem, cetuximab; cisplatin; docetaxel; fluorouracil, carbogen; carbon dioxide; niacinamide, cisplatin; docetaxel; fluorouracil, Soluble Beta Glucan, contusugene ladenovec, zalutumumab, talimogene laherparepvec, sucralfate, doxepin, methotrexate, amifostine, capsaicin, cisplatin; fluorouracil; panitumumab, aldesleukin, tranexamic acid, zinc sulfate, bacillus calmette-guerin [connaught] vaccine; bleomycin; cyclophosphamide; fluorouracil; methotrexate, amifostine, fluorouracil; hydroxyurea, bacitracin; clotrimazole; gentamicin, cyclophospha mide; doxorubicin, pelareorep, filgrastim, enteral nutrition, pentoxifylline, epirubicin; fluorouracil; leucovorin; mitomycin, porfiromycin, bleomycin; cisplatin; methotrexate; vincristine, bacillus calmette-guerin vaccine; isoniazid; methotrexate, fluorouracil; leucovorin; methotrexate, rAd-p53, PV-10, Leukocyte Interleukin, nimotuzumab, afatinib dimaleate, afatinib dimaleate; cisplatin, carboplatin; cetuximab; fluorouracil, cetuximab; cisplatin, nedaplatin, docetaxel; fluorouracil, nimorazole, gemcitabine hydrochloride, afatinib dimaleate, nedaplatin, contusugene ladenovec, Lactobacillus brevis CD2, cetuximab, nimotuzumab, E-10A, cisplatin; gemcitabine, cisplatin; lobaplatin, endostatin (recombinant)_dapsone; doxycycline; minocycline, carboplatin; cyclophosphamide; docetaxel; doxorubicin; paclitaxel, nivolumab, pelareorep, autologous stem cells; carboplatin; etoposide; ifosfamide, vinflunine ditartrate, gemcitabine, cisplatin; fluorouracil; mitomycin, porfiromycin, Olimel, pembrolizumab, AminoPure, nedaplatin, Liang Ge San, eicosapentaenoic acid, celecoxib; methotrexate, cisplatin; pembrolizumab, (tegafur+uracil); cisplatin; epirubicin; mitomycin, durvalumab; tremelimumab, sodium hypochlorite, cetuximab; cisplatin, cisplatin; paclitaxel, amcasertib, capecitabine, R-TPR-033, VB-4847, gabapentin, raltitrexed, cisplatin; fluorouracil; leucovorin, cisplatin; docetaxel, celecoxib, durvalumab; durvalumab+tremelimumab, TT-10, (sodium alginate+sodium carbonate+propolis+aloe vera+calendula+honey+chamomile); cisplatin, pembrolizumab; talimogene laherparepvec, avelumab, nimorazole, eicosapentaenoic acid, lovastatin, dexamethasone; etoposide; gemcitabine; pegaspargase, ipilimumab; nivolumab, cetuximab; cisplatin; nivolumab, capecitabine; cisplatin; docetaxel, isotretinoin, carboplatin; cisplatin; gemcitabine; paclitaxel, erlotinib hydrochloride, VB-4847 (Proxinium Plus Best Supportive Care), celecoxib; thalidomide, cisplatin; fluorouracil, curcumin, celecoxib, imiquimod, cisplatin; cyclophosphamide; etoposide, sotatercept, cisplatin; docetaxel; fluorouracil, cetuximab; cisplatin; docetaxel, human endostatin, melatonin, epigallocatechin gallate, cisplatin; gemcitabine, gemcitabine; paclitaxel, (gimeracil+oteracil+tegafur), eicosapentaenoic acid; undisclosed nutritional supplement, celecoxib; methotrexate, pembrolizumab (Keytruda), afatinib dimaleate, cetuximab (Erbitux), durvalumab, durvalumab+tremelimumab, E-10A, entrectinib, Leukocyte Interleukin (Multikine), nimotuzumab, nivolumab (Opdivo), pelareorep (Reolysin), TT-10, vinflunine ditartrate (Javlor), acalabrutinib, AlloVax and alpelisib.

In particularly preferred embodiments, the at least one additional pharmaceutically active ingredient is a compound, which is conventionally used in the treatment and/or prophylaxis of squamous cell carcinoma of the head and neck (HNSCC), preferably advanced HNSCC, wherein the compound is a PD-1 inhibitor, preferably an antagonistic PD-1 antibody, preferably selected from the group consisting of Nivolumab, and Pembrolizumab (Keytruda).

In some embodiments, the at least one additional pharmaceutically active ingredient is a compound, which is conventionally used in the treatment and/or prophylaxis of adenocystic carcinoma (ACC), preferably advanced ACC, wherein the compound is preferably selected from the group consisting of Nivolumab, Cetuximab (Erbitux), paclitaxel albumin bound (Abraxane), gimeracil+oteracil+tegafur (TS-1), docetaxel (Docetaxel, Doxel, Taxotere, Docetaxel An, Nanoxel M, Tautax, Docetaxel −AS, Docetaxel-M, Qvidadotax, Relidoce, Taxelo, Oncodocel, Doxotel, Pacancer, Docetrust, Dodetax, Dodabur, Soulaxcin, Taxedol, Docefim, Ribodocel, Critidoc, Asodoc, Chemodoc, Docelibbs, Docenat, Dincilezan, Dostradixinol, Docefrez, Camitotic, Oncotaxel, Somatixel, Belotaxel, Qvidadotax, Taxceus, Cetadocure, Docetaxel CT, Tevaxter, Docirena, Eurotere, Axtere, Celotax, Taxanit, Drobanos, Cetado, Doxocad, Taxceus, Egidox, Tedocad, Docecad, Docelex, Docetax, Docetere, Dotax, Taxuba, Monotaxel, Taceedo, Detaxl, Docet, Ferdotax, Wintaxel, Kamdocon, Naltoxater), tegafur+uracil (Uft, Uft E, tefudex, Unitoral, Luporal Tagracil), fluorouracil (bn: 5-FU, Fluorouracil, Fluorouracile), gimeracil+oteracil+tegafur ODT (bn TS-1 Combination OD), bleomycin sulfate (Tecnomicina, Cinaleo, Bleomycin, Bloicin-S, Bonar, Bleocin, Bleo Bleomycin Sulfate, Bleocel, Bleotex, Oncobleo, Bleonco, Bleosol, Lyoble, Bleomycin Sulfate, Blenamax, Blenoxane, Bleomicina, Bleomycine Belton Bleoprim), carboplatin (Carboplatin Platamine CS Carbaccord Carboplatina Carboplatino Paraplatin Carbosin Tecnocarb Carbomerck, Paract, Carboplatine CTRS, Carboplatine Intsel Chimos, Carbokem Carbotinol Fauldcarbo, Evocarb, Citoplatina, Platin, Carboplatine Qua limed, Carboplatine, Carboplatino, Carboplatin), ciprofloxacin (Hypoflox, Ufexil), ciprofloxacin hydrochloride (Ciprofloxacin Pharma, Prodin, Ciproxin), cisplatin (Cisplatin, Stritin, Ifapla, Accocit, Unistin, Cancertin, Cisplan, Citoplax, Nuoxin, Placis, Cisplatino, Displanor, Cisplatine, Randa, Cisplatin, Cispla, Fauldcispla, Briplatin, Platinex, Platinol, Riboplatin, Platistine CS, Platosin, Accocit), cyclophosphamide (Endoxan, Cyclophosphamide), doxifluridine (Doxifluridine, May Vladimir, Doxifluridine), doxorubicin (Doxorubicin Hydrochloride, Adriamycin RDF, Doxorubici, Doxorubicin Hydrochloride, Doxorubicin PFS), epirubicin hydrochloride (Brecila, Cloridrato De Epirrubicina, Epirubicin, Farmorubicina, Nuovodox, Adnexa, 4-Eppedo, Favicin), fluorouracil (Agicil, Fluorouracil, Fauldfluor, Oncourcil Flocil, 5 Flucel), folic acid+methotrexate (Truxofol), human adenovirus type 5 (recombinant) (Oncorine), hydroxyurea (Oxyrea, Durea, Myelostat, Riborea, Unidrea, Ondrea, Hydran, Leukocel, Hydroxyurea, Hydrea), ifosfamide (Holoxan, Ifosfamide EG), levamisole (Zirsol), methotrexate (Tratoben, Methotrexate, Fresexate, Neometho, Fauldmetro, Methotrexate Sodium, Methocel, Hytas, Methaccord, Methofill, Metotrexato, Traxacord, Plastomet, Tevatrex, Metrex, Caditrex, Carditrex, Vibzi, Imutrex, Biotrexate, Methorex, Mexate, Neotrexate, Oncotrex, Remtrex, Trixilem, Hi-Trex, Metorex, Trex, Unitrexate, Ebetrexac, Fauldexato, Lantarel, Maxtrex, Miantrex CS, Rheumatrex, Folex, Folex PFS, Abitrexate, Trexall, Emthexate, Abitrexate, Meadow, Mexate Aq, Biometrox, Otaxem), Mitomycin (Mitomycin C, Mitomycin, Mitonco, Lyomit), Nedaplatin (Jiebaishu, Aoxianda, Aqupla), Nimesulide (Nimulid), Nimotuzumab (Biomab EGFR, Laedemab), Nitrofurantoin (Furatsilin), Ofloxacin (Entof), Paclitaxel (Paclitaxel, Taxol), peplomycin sulfate (Pepleo), picibanil (Picibanil), pirarubicin (Pirarubicin Hydrochloride, Therarubicin, Pinorubin), sodium glycididazole (CMNa), tegafur (Utefos, Icarus, Futraful, Tegafur Gimeracil Oteracil Potassium), temoporfin (Foscan), topotecan hydrochloride (Topotecan), ubenimex (Ubenimex), vinblastine sulfate (Vinblastine, Vblastin, Vincristine, Vincristine Sulfate, Vincristin, Sutivin, Vincrisan, Eldisine), vincristine sulfate (Oncovin), gefitinib, escitalopram oxalate, Gene Therapy to Activate p53 for Oncology, cevimeline hydrochloride, docetaxel; lenograstim, bortezomib; docetaxel; irinotecan, carboplatin; paclitaxel, cisplatin; docetaxel; fluorouracil, human papillomavirus vaccine (Vaccination Against Human Papillomavirus Types 16 and 18 and Unvaccinated Controls), carboplatin; cisplatin; paclitaxel, undisclosed epidermal growth factor receptor inhibitor, cetuximab, sorafenib tosylate; suntinib malate, amifostine, cisplatin; dexa methasone; docetaxel; metoclopra mide, platinum based chemotherapy, cetuximab; nimotuzumab, nimotuzumab, dobutamine hydrochloride; dopexamine hydrochloride; epinephrine; norepinephrine bitartrate, cetuximab; cisplatin, cetuximab; cisplatin; docetaxel; fluorouracil, cisplatin; paclitaxel, Gendicine, Doce onkovis (Docetaxel), Picibanil, docetaxel; lobaplatin, oxytetracycline hydrochloride, cisplatin; mitomycin, bleomycin, nimotuzumab, cisplatin; fluorouracil; nimotuzumab, cetuximab; cisplatin; cyclophosphamide; dacarbazine; docetaxel; doxorubicin; etoposide; fluorouracil; gemcitabine hydrochloride; irinotecan hydrochloride; oxaliplatin; paclitaxel; pemetrexed disodium; temozolomide, varenicline, acetylcysteine, dopamine; norepinephrine, (gimeracil+oteracil+tegafur), nimotuzumab, icotinib hydrochloride, Kushen (kuh-seng), Carboxymethylpachymaran, BCM-95, Lactofos, Pegfilgrastim, methadone, Varanadi ghrita, cisplatin; palifermin, cetuximab, cisplatin; lapatinib ditosylate, gefitinib, zalutumumab, erlotinib hydrochloride, beta carotene; E-Tabs, bevacizumab, levothyroxine; liothyronine sodium; recombinant human thryroid stimulating hormone, cisplatin; docetaxel; fluorouracil; hydroxyurea, fluorouracil, docetaxel; St. John's Wort, carboplatin; cisplatin; fluorouracil; paclitaxel, Biafine, methotrexate, cisplatin; fluorouracil, alpha tocopherol; isotretinoin; recombinant interferon alfa, panitumumab, cisplatin, bleomycin; fluorouracil; leucovorin; methotrexate; vincristine, nimotuzumab, panitumumab, amifostine; fluorouracil, cisplatin; docetaxel; fluorouracil, capecitabine; cisplatin; fluorouracil, pemetrexed disodium, cisplatin; gemcitabine, tranexamic acid, carboplatin; fluorouracil, cefazolin, epoetin alfa, tirapaza mine, erlotinib hydrochloride, cetuximab; docetaxel; fluorouracil, megestrol, contusugene ladenovec, cevimeline hydrochloride, cisplatin; fluorouracil, cisplatin; fluorouracil; paclitaxel, isotretinoin, porfiromycin, escitalopram oxalate, gimeracil+oteracil+tegafur, IntraDose (Cisplatin Plus Epinephrine), misonidazole, tirapazamine, carboplatin; cisplatin; docetaxel; fluorouracil, gefitinib, epoetin alfa, paclitaxel, gefitinib, palifermin, pilocarpine, celecoxib, cisplatin; docetaxel; filgrastim; fluorouracil; paclitaxel, cisplatin; fluorouracil; leucovorin, cisplatin; gemcitabine; mannitol, Lactobacillus brevis CD2, Glutamine, carbogen; niacinamidem, cetuximab; cisplatin; docetaxel; fluorouracil, carbogen; carbon dioxide; niacinamide, cisplatin; docetaxel; fluorouracil, Soluble Beta Glucan, contusugene ladenovec, zalutumumab, talimogene laherparepvec, sucralfate, doxepin, methotrexate, amifostine, capsaicin, cisplatin; fluorouracil; panitumumab, aldesleukin, tranexamic acid, zinc sulfate, bacillus calmette-guerin [connaught] vaccine; bleomycin; cyclophosphamide; fluorouracil; methotrexate, amifostine, fluorouracil; hydroxyurea, bacitracin; clotrimazole; gentamicin, cyclophosphamide; doxorubicin, pelareorep, filgrastim, enteral nutrition, pentoxifylline, epirubicin; fluorouracil; leucovorin; mitomycin, porfiromycin, bleomycin; cisplatin; methotrexate; vincristine, bacillus calmette-guerin vaccine; isoniazid; methotrexate, fluorouracil; leucovorin; methotrexate, rAd-p53, PV-10, Leukocyte Interleukin, nimotuzumab, afatinib dimaleate, afatinib dimaleate; cisplatin, carboplatin; cetuximab; fluorouracil, cetuximab; cisplatin, nedaplatin, docetaxel; fluorouracil, nimorazole, gemcitabine hydrochloride, afatinib dimaleate, nedaplatin, contusugene ladenovec, Lactobacillus brevis CD2, cetuximab, nimotuzumab, E-10A, cisplatin; gemcitabine, cisplatin; lobaplatin, endostatin (recombinant)_dapsone; doxycycline; minocycline, carboplatin; cyclophosphamide; docetaxel; doxorubicin; paclitaxel, nivolumab, pelareorep, autologous stem cells; carboplatin; etoposide; ifosfamide, vinflunine ditartrate, gemcitabine, cisplatin; fluorouracil; mitomycin, porfiromycin, Olimel, pembrolizumab, AminoPure, nedaplatin, Liang Ge San, eicosapentaenoic acid, celecoxib; methotrexate, cisplatin; pembrolizumab, (tegafur+uracil); cisplatin; epirubicin; mitomycin, durvalumab; tremelimumab, sodium hypochlorite, cetuximab; cisplatin, cisplatin; paclitaxel, amcasertib, capecitabine, R-TPR-033, VB-4847, gabapentin, raltitrexed, cisplatin; fluorouracil; leucovorin, cisplatin; docetaxel, celecoxib, durvalumab; durvalumab +tremelimumab, TT-10, (sodium alginate+sodium carbonate+propolis+aloe vera+calendula+honey+chamomile); cisplatin, pembrolizumab; talimogene laherparepvec, avelumab, nimorazole, eicosapentaenoic acid, lovastatin, dexamethasone; etoposide; gemcitabine; pegaspargase, ipilimumab; nivolumab, cetuximab; cisplatin; nivolumab, capecitabine; cisplatin; docetaxel, isotretinoin, carboplatin; cisplatin; gemcitabine; paclitaxel, erlotinib hydrochloride, VB-4847 (Proxinium Plus Best Supportive Care), celecoxib; thalidomide, cisplatin; fluorouracil, curcumin, celecoxib, imiquimod, cisplatin; cyclophosphamide; etoposide, sotatercept, cisplatin; docetaxel; fluorouracil, cetuximab; cisplatin; docetaxel, human endostatin, melatonin, epigallocatechin gallate, cisplatin; gemcitabine, gemcitabine; paclitaxel, (gimeracil+oteracil+tegafur), eicosapentaenoic acid; undisclosed nutritional supplement, celecoxib; methotrexate, pembrolizumab (Keytruda), afatinib dimaleate, cetuximab (Erbitux), durvalumab, durvalumab+tremelimumab, E-10A, entrectinib, Leukocyte Interleukin (Multikine), nimotuzumab, nivolumab (Opdivo), pelareorep (Reolysin), TT-10, vinflunine ditartrate (Javlor), acalabrutinib, AlloVax and alpelisib.

In some embodiments, the at least one additional pharmaceutically active ingredient is a compound, which is conventionally used in the treatment and/or prophylaxis of cutaneous T-cell lymphoma, preferably advanced cutaneous T-cell lymphoma, wherein the compound is preferably selected from the group consisting of Nivolumab, Cetuximab (Erbitux), paclitaxel albumin bound (Abraxane), gimeracil+oteracil+tegafur (TS-1), docetaxel (Docetaxel, Doxel, Taxotere, Docetaxel An, Nanoxel M, Tautax, Docetaxel −AS, Docetaxel-M, Qvidadotax, Relidoce, Taxelo, Oncodocel, Doxotel, Pacancer, Docetrust, Dodetax, Dodabur, Soulaxcin, Taxedol, Docefim, Ribodocel, Critidoc, Asodoc, Chemodoc, Docelibbs, Docenat, Dincilezan, Dostradixinol, Docefrez, Camitotic, Oncotaxel, Somatixel, Belotaxel, Qvidadotax, Taxceus, Cetadocure, Docetaxel CT, Tevaxter, Docirena, Eurotere, Axtere, Celotax, Taxanit, Drobanos, Cetado, Doxocad, Taxceus, Egidox, Tedocad, Docecad, Docelex, Docetax, Docetere, Dotax, Taxuba, Monotaxel, Taceedo, Detaxl, Docet, Ferdotax, Wintaxel, Kamdocon, Naltoxater), tegafur+uracil (Uft, Uft E, tefudex, Unitoral, Luporal Tagracil), fluorouracil (bn: 5-FU, Fluorouracil, Fluorouracile), gimeracil+oteracil+tegafur ODT (bn TS-1 Combination OD), bleomycin sulfate (Tecnomicina, Cinaleo, Bleomycin, Bloicin-S, Bonar, Bleocin, Bleo Bleomycin Sulfate, Bleocel, Bleotex, Oncobleo, Bleonco, Bleosol, Lyoble, Bleomycin Sulfate, Blenamax, Blenoxane, Bleomicina, Bleomycine Bellon Bleoprim), carboplatin (Carboplatin Platamine CS Carbaccord Carboplatina Carboplatino Paraplatin Carbosin Tecnocarb Carbomerck, Paract, Carboplatine CTRS, Carboplatine Intsel Chimos, Carbokem Carbotinol Fauldcarbo, Evocarb, Citoplatina, Platin, Carboplatine Qualimed, Carboplatine, Carboplatino, Carboplatin), ciprofloxacin (Hypoflox, Ufexil), ciprofloxacin hydrochloride (Ciprofloxacin Pharma, Prodin, Ciproxin), cisplatin (Cisplatin, Stritin, Ifapla, Accocit, Unistin, Cancertin, Cisplan, Citoplax, Nuoxin, Placis, Cisplatino, Displanor, Cisplatine, Randa, Cisplatin, Cispla, Fauldcispla, Briplatin, Platinex, Platinol, Riboplatin, Platistine CS, Platosin, Accocit), cyclophosphamide (Endoxan, Cyclophosphamide), doxifluridine (Doxifluridine, May Vladimir, Doxifluridine), doxorubicin (Doxorubicin Hydrochloride, Adriamycin RDF, Doxorubici, Doxorubicin Hydrochloride, Doxorubicin PFS), epirubicin hydrochloride (Brecila, Cloridrato De Epirrubicina, Epirubicin, Farmorubicina, Nuovodox, Adnexa, 4-Eppedo, Favicin), fluorouracil (Agicil, Fluorouracil, Fauldfluor, Oncourcil Flocil, 5 Flucel), folic acid+methotrexate (Truxofol), human adenovirus type 5 (recombinant) (Oncorine), hydroxyurea (Oxyrea, Durea, Myelostat, Riborea, Unidrea, Ondrea, Hydra n, Leukocel, Hydroxyurea, Hydrea), ifosfamide (Holoxan, Ifosfamide EG), levamisole (Zirsol), methotrexate (Tratoben, Methotrexate, Fresexate, Neometho, Fauldmetro, Methotrexate Sodium, Methocel, Hytas, Methaccord, Methofill, Metotrexato, Traxacord, Plastomet, Tevatrex, Metrex, Caditrex, Carditrex, Vibzi, Imutrex, Biotrexate, Methorex, Mexate, Neotrexate, Oncotrex, Remtrex, Trixilem, Hi-Trex, Metorex, Trex, Unitrexate, Ebetrexac, Fauldexato, Lantarel, Maxtrex, Miantrex CS, Rheumatrex, Folex, Folex PFS, Abitrexate, Trexall, Emthexate, Abitrexate, Meadow, Mexate Aq, Biometrox, Otaxem), Mitomycin (Mitomycin C, Mitomycin, Mitonco, Lyomit), Nedaplatin (Jiebaishu, Aoxianda, Aqupla), Nimesulide (Nimulid), Nimotuzumab (Biomab EGFR, Laedemab), Nitrofurantoin (Furatsilin), Ofloxacin (Entof), Paclitaxel (Paclitaxel, Taxol), peplomycin sulfate (Pepleo), picibanil (Picibanil), pirarubicin (Pirarubicin Hydrochloride, Therarubicin, Pinorubin), sodium glycididazole (CMNa), tegafur (Utefos, Icarus, Futraful, Tegafur Gimeracil Oteracil Potassium), temoporfin (Foscan), topotecan hydrochloride (Topotecan), ubenimex (Ubenimex), vinblastine sulfate (Vinblastine, Vblastin, Vincristine, Vincristine Sulfate, Vincristin, Sutivin, Vincrisan, Eldisine), vincristine sulfate (Oncovin), gefitinib, escitalopram oxalate, Gene Therapy to Activate p53 for Oncology, cevimeline hydrochloride, docetaxel; lenograstim, bortezomib; docetaxel; irinotecan, carboplatin; paclitaxel, cisplatin; docetaxel; fluorouracil, human papillomavirus vaccine (Vaccination Against Human Papillomavirus Types 16 and 18 and Unvaccinated Controls), carboplatin; cisplatin; paclitaxel, undisclosed epidermal growth factor receptor inhibitor, cetuximab, sorafenib tosylate; sunitinib malate, amifostine, cisplatin; dexamethasone; docetaxel; metoclopramide, platinum based chemotherapy, cetuximab; nimotuzumab, nimotuzumab, dobutamine hydrochloride; dopexamine hydrochloride; epinephrine; norepinephrine bitartrate, cetuximab; cisplatin, cetuximab; cisplatin; docetaxel; fluorouracil, cisplatin; paclitaxel, Gendicine, Doce onkovis (Docetaxel), Picibanil, docetaxel; lobaplatin, oxytetracycline hydrochloride, cisplatin; mitomycin, bleomycin, nimotuzumab, cisplatin; fluorouracil; nimotuzumab, cetuximab; cisplatin; cyclophosphamide; dacarbazine; docetaxel; doxorubicin; etoposide; fluorouracil; gemcitabine hydrochloride; irinotecan hydrochloride; oxaliplatin; paclitaxel; pemetrexed disodium; temozolomide, varenicline, acetylcysteine, dopamine; norepinephrine, (gimeracil+oteracil+tegafur), nimotuzumab, icotinib hydrochloride, Kushen (kuh-seng), Carboxymethylpachymaran, BCM-95, Lactofos, Pegfilgrastim, methadone, Varanadi ghrita, cisplatin; palifermin, cetuximab, cisplatin; lapatinib ditosylate, gefitinib, zalutumumab, erlotinib hydrochloride, beta carotene; E-Tabs, bevacizumab, levothyroxine; liothyronine sodium; recombinant human thryroid stimulating hormone, cisplatin; docetaxel; fluorouracil; hydroxyurea, fluorouracil, docetaxel; St. John's Wort, carboplatin; cisplatin; fluorouracil; paclitaxel, Biafine, methotrexate, cisplatin; fluorou racil, alpha tocopherol; isotretinoin; recombinant interferon alfa, panitumumab, cisplatin, bleomycin; fluorouracil; leucovorin; methotrexate; vincristine, nimotuzumab, panitumumab, amifostine; fluorouracil, cisplatin; docetaxel; fluorouracil, capecitabine; cisplatin; fluorouracil, pemetrexed disodium, cisplatin; gemcitabine, tranexamic acid, carboplatin; fluorouracil, cefazolin, epoetin alfa, tirapazamine, erlotinib hydrochloride, cetuximab; docetaxel; fluorouracil, megestrol, contusugene ladenovec, cevimeline hydrochloride, cisplatin; fluorouracil, cisplatin; fluorouracil; paclitaxel, isotretinoin, porfiromycin, escitalopram oxalate, gimeracil+oteracil+tegafur, IntraDose (Cisplatin Plus Epinephrine), misonidazole, tirapazamine, carboplatin; cisplatin; docetaxel; fluorouracil, gefitinib, epoetin alfa, paclitaxel, gefitinib, palifermin, pilocarpine, celecoxib, cisplatin; docetaxel; filgrastim; fluorouracil; paclitaxel, cisplatin; fluorouracil; leucovorin, cisplatin; gemcitabine; mannitol, Lactobacillus brevis CD2, Glutamine, carbogen; niacinamidem, cetuximab; cisplatin; docetaxel; fluorouracil, carbogen; carbon dioxide; niacinamide, cisplatin; docetaxel; fluorouracil, Soluble Beta Glucan, contusugene ladenovec, zalutumumab, talimogene laherparepvec, sucralfate, doxepin, methotrexate, amifostine, capsaicin, cisplatin; fluorouracil; panitumumab, aldesleukin, tranexamic acid, zinc sulfate, bacillus calmette-guerin [connaught] vaccine; bleomycin; cyclophosphamide; fluorouracil; methotrexate, amifostine, fluorouracil; hydroxyurea, bacitracin; clotrimazole; gentamicin, cyclophosphamide; doxorubicin, pelareorep, filgrastim, enteral nutrition, pentoxifylline, epirubicin; fluorouracil; leucovorin; mitomycin, porfiromycin, bleomycin; cisplatin; methotrexate; vincristine, bacillus calmette-guerin vaccine; isoniazid; methotrexate, fluorouracil; leucovorin; methotrexate, rAd-p53, PV-10, Leukocyte Interleukin, nimotuzumab, afatinib dimaleate, afatinib dimaleate; cisplatin, carboplatin; cetuximab; fluorouracil, cetuximab; cisplatin, nedaplatin, docetaxel; fluorouracil, nimorazole, gemcitabine hydrochloride, afatinib dimaleate, nedaplatin, contusugene ladenovec, Lactobacillus brevis CD2, cetuximab, nimotuzumab, E-10A, cisplatin; gemcitabine, cisplatin; lobaplatin, endostatin (recombinant)_dapsone; doxycycline; minocycline, carboplatin; cyclophosphamide; docetaxel; doxorubicin; paclitaxel, nivolumab, pelareorep, autologous stem cells; carboplatin; etoposide; ifosfamide, vinflunine ditartrate, gemcitabine, cisplatin; fluorouracil; mitomycin, porfiromycin, Olimel, pembrolizumab, AminoPure, nedaplatin, Liang Ge San, eicosapentaenoic acid, celecoxib; methotrexate, cisplatin; pembrolizumab, (tegafur uracil); cisplatin; epirubicin; mitomycin, durvalumab; tremelimumab, sodium hypochlorite, cetuximab; cisplatin, cisplatin; paclitaxel, amcasertib, capecitabine, R-TPR-033, VB-4847, gabapentin, raltitrexed, cisplatin; fluorouracil; leucovorin, cisplatin; docetaxel, celecoxib, durvalumab; durvalumab+tremelimumab, TT-10, (sodium alginate+sodium carbonate+propolis+aloe vera+calendula+honey+chamomile); cisplatin, pembrolizumab; talimogene laherparepvec, avelumab, nimorazole, eicosapentaenoic acid, lovastatin, dexamethasone; etoposide; gemcitabine; pegaspargase, ipilimumab; nivolumab, cetuximab; cisplatin; nivolumab, capecitabine; cisplatin; docetaxel, isotretinoin, carboplatin; cisplatin; gemcitabine; paclitaxel, erlotinib hydrochloride, VB-4847 (Proxinium Plus Best Supportive Care), celecoxib; thalidomide, cisplatin; fluorouracil, curcumin, celecoxib, imiquimod, cisplatin; cyclophosphamide; etoposide, sotatercept, cisplatin; docetaxel; fluorouracil, cetuximab; cisplatin; docetaxel, human endostatin, melatonin, epigallocatechin gallate, cisplatin; gemcitabine, gemcitabine; paclitaxel, (gimeracil+oteracil+tegafur), eicosapentaenoic acid; undisclosed nutritional supplement, celecoxib; methotrexate, pembrolizumab (Keytruda), afatinib dimaleate, cetuximab (Erbitux), durvalumab, durvalumab+tremelimumab, E-10A, entrectinib, Leukocyte Interleukin (Multikine), nimotuzumab, nivolumab (Opdivo), pelareorep (Reolysin), TT-10, vinflunine ditartrate (Javlor), acalabrutinib, AlloVax and alpelisib.

In some embodiments, the at least one additional pharmaceutically active ingredient is a compound, which is conventionally used in the treatment and/or prophylaxis of vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy, wherein the compound is preferably selected from the group consisting of mitomycin-C2, cisplatin, carboplatin, vinorelbine, paclitaxel, a tyrosine kinase inhibitor (e.g. erlotinib), nivolumab, bleomycin sulfate (e.g. bleomycin, bleomycin sulfate, blenamax, tevableo, oncobleo, bleo, bloicin-5), 5-fluorouracil (5-FU), Gardasil 9 (human papillomavirus (9-valent) vaccine), omiganan pentahydrochloride, alisertib, ISA-101 (13 synthetic long peptides (25-35 amino acids long) derived from the E6 and E7 oncogenic proteins of the HPV 16 virus), PDS-0101, Vicoryx (P16_37-63 vaccine), TA-CIN (fusion protein vaccine comprising capsid protein L2, E6 and E7 from HPV16) and human papillomavirus 16 E6 peptide vaccine.

According to some embodiments, the at least one additional pharmaceutically active ingredient is a therapeutic peptide or protein (e.g. an antibody, a decoy receptor or a cytokine) or a fragment or variant thereof. Said therapeutic peptide or protein, or a fragment or variant thereof, may be provided as such or in the form of a nucleic acid (e.g. an RNA as described herein) encoding the therapeutic peptide or protein, or a fragment or variant thereof. It is further preferred that the therapeutic peptide or protein (e.g. an antibody, a decoy receptor or a cytokine) comprises a signal peptide, e.g. a secretory signal peptide. Such signal peptides are sequences, which typically exhibit a length of about 15 to 30 amino acids and are preferably located at the N-terminus of the encoded peptide, without being limited thereto. Signal peptides as defined herein preferably allow the localization of the therapeutic peptide or protein, or a fragment or variant thereof, to a certain cellular membrane or a certain cellular compartment, preferably the cell surface, the cytoplasmic membrane, the endoplasmic reticulum (ER) or the endosomal-lysosomal compartment.

In certain embodiments, the signal peptide may be selected from the list of amino acid sequences according to SEQ ID NOs: 1-1115 and SEQ ID NO: 1728 of the international patent application WO2017/081082, or fragments or variants of any of these sequences. On the nucleic acid level, any nucleic acid sequence (e.g. RNA sequence) may be selected, which encodes such amino acid sequences. In this context, the disclosure of WO2017/081082 is herewith incorporated by reference.

Examples of signal peptide sequences as defined herein include, without being limited thereto, signal sequences (or fragments or variants thereof) of classical or non-classical MHC-molecules (e.g. signal sequences of MHC I and II molecules, e.g. of the MHC class I molecule HLA-A*0201), cytokines or immunoglobulines as defined herein, the invariant chain of immunoglobulines or antibodies as defined herein, Lamp1, Tapasin, Erp57, Calretikulin, Calnexin, further membrane associated proteins, proteins associated with the endoplasmic reticulum (ER) or proteins associated with the endosomal-lysosomal compartment. Particularly preferred in the context of the present invention are the signal sequences of MHC class I molecule HLA-A*0201, or a fragment or variant thereof.

According to a preferred embodiment, the at least one additional pharmaceutically active ingredient is a therapeutic peptide or protein, or a fragment or variant thereof, comprising a signal sequence (or a fragment or variant thereof) derived from HLA-A2, HsPLAT, HsEPO, HsALB, IgE, HsCD5, HsIL2, HsCTRB2, human immunoglobulin heavy chain, human immunoglobulin light chain, GpLuc, Mice immunoglobulin kappa, NrChitl, CILp1.1, NgNep1, HsAzu1, HsCD33, VcCtxB, HsCST4, HsIns-iso1, HsSPARC, H₁N1(Netherlands2009), FV, MHCII or JEV.

It is further preferred that the at least one additional pharmaceutically active ingredient is a therapeutic peptide or protein (e.g. an antibody, a decoy receptor or a cytokine), or a fragment or variant thereof, comprising or consisting of an amino acid sequence according to any one of SEQ ID NO: 739-769, or a fragment or variant of any one of these amino acid sequences. Preferably, the at least one additional pharmaceutically active ingredient is a therapeutic peptide or protein (e.g. an antibody, a decoy receptor or a cytokine), or a fragment or variant thereof, comprising or consisting of an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to any one of SEQ ID NO: 739-769.

According to a particularly preferred embodiment, the at least one additional pharmaceutically active ingredient is a checkpoint modulator or a cytokine.

Negative regulatory T cell surface molecules were discovered, which are upregulated in activated T cells in order to dampen their activity, thus reducing the effectiveness of said activated T cells in the killing of tumor cells. These inhibitory molecules were termed negative co-stimulatory molecules due to their homology to the T cell co-stimulatory molecule CD28. These proteins, also referred to as immune checkpoint proteins, function in multiple pathways including the attenuation of early activation signals, competition for positive co-stimulation and direct inhibition of antigen presenting cells (Bour-Jordan et al., 2011. Immunol Rev. 241(1):180-205).

In the context of the present invention, a checkpoint modulator is typically a molecule, such as a protein (e.g. an antibody), a dominant negative receptor, a decoy receptor, or a ligand or a fragment or variant thereof, which modulates the function of an immune checkpoint protein, e.g. it inhibits or reduces the activity of checkpoint inhibitors (or inhibitory checkpoint molecules) or it stimulates or enhances the activity of checkpoint stimulators (or stimulatory checkpoint molecules). Therefore, checkpoint modulators as defined herein, influence the activity of checkpoint molecules.

In this context, inhibitory checkpoint molecules are defined as checkpoint inhibitors and can be used synonymously. In addition, stimulatory checkpoint molecules are defined as checkpoint stimulators and can be used synonymously.

Preferably, the checkpoint modulator is selected from agonistic antibodies, antagonistic antibodies, ligands, dominant negative receptors, and decoy receptors or combinations thereof.

Methods for generating and using mRNA-encoded antibodies are known in the art (e.g. WO2008/083949 or PCT/EP2017/060226).

Preferred inhibitory checkpoint molecules that may be inhibited by a checkpoint modulator in the context of the invention are PD-1, PD-L1, CTLA-4, PD-L2, LAG3, TIM3/HAVCR2, 2B4, A2aR, B7H3, B7H4, BTLA, CD30, CD160, CD155, GAL9, HVEM, IDO1, IDO2, KIR, LAIR1 and VISTA.

Preferred stimulatory checkpoint molecules that may be stimulated by a checkpoint modulator in the context of the invention are CD2, CD27, CD28, CD40, CD137, CD226, CD276, GITR, ICOS, OX40 and CD70.

According to a preferred embodiment, the isRNA is for use as described herein, wherein the use comprises—as an additional pharmaceutically active ingredient—a checkpoint modulator selected from the group consisting of the checkpoint modulator is selected from the group consisting of a PD-1 inhibitor, a PD-L1 inhibitor, a PD-L2 inhibitor, a CTLA-4 inhibitor, a LAG3 inhibitor, a TIM3 inhibitor, a TIGIT-inhibitor, an OX40 stimulator, a 4-1BB stimulator, a CD40L stimulator, a CD28 stimulator and a GITR stimulator.

According to a preferred embodiment, the checkpoint modulator as used herein targets a member of the PD-1 pathway. Members of the PD-1 pathway are typically proteins, which are associated with PD-1 signaling. On the one hand, this group comprises proteins, which induce PD-1 signaling upstream of PD-1 as e.g. the ligands of PD-1, PD-L1 and PD-L2, and the signal transduction receptor PD-1. On the other hand, this group comprises signal transduction proteins downstream of PD-1 receptor. Particularly preferred as members of the PD-1 pathway in the context of the present invention are PD-1, PD-L1 and PD-L2.

In the context of the present invention, a PD-1 pathway antagonist (or PD-1 inhibitor) is preferably defined herein as a compound capable to impair the PD-1 pathway signaling, preferably signaling mediated by the PD-1 receptor. Therefore, the PD-1 pathway antagonist may be any antagonist directed against any member of the PD-1 pathway capable of antagonizing PD-1 pathway signaling.

In a preferred embodiment, the checkpoint modulator used herein is a PD-1 inhibitor or a PD-L1 inhibitor, wherein the PD-1 inhibitor is preferably an antagonistic antibody directed against PD-1 and the PD-L1 inhibitor is preferably an antagonistic antibody directed against PD-L1.

In this context, the antagonist may be an antagonistic antibody as defined herein, targeting any member of the PD-1 pathway, preferably an antagonistic antibody directed against PD-1 receptor, PD-L1 or PD-L2. Such an antagonistic antibody may also be encoded by a nucleic acid. Also, the PD-1 pathway antagonist may be a fragment of the PD-1 receptor blocking the activity of PD1 ligands. B7-1 or fragments thereof may act as PD1-antagonizing ligands as well. Additionally, a PD-1 pathway antagonist may be a protein comprising (or a nucleic acid coding for) an amino acid sequence capable of binding to PD-1 but preventing PD-1 signaling, e.g. by inhibiting PD-1 and B7-H1 or B7-DL interaction (WO 2014/127917; WO2012062218).

Particularly preferred are the anti-PD1 antibodies Nivolumab (MDX-1106/BMS-936558/0N0-4538), (Brahmer et al., 2010. J Clin Oncol. 28(19):3167-75; PMID: 20516446); Pidilizumab (CT-011), (Berger et al., 2008. Clin Cancer Res. 14(10):3044-51; PMID: 18483370); Pembrolizumab (MK-3475, SCH 900475); AMP-224, and MEDI0680 (AMP-514).

Particularly preferred are also the anti-PD-L1 antibodies MDX-1105/BMS-936559 (Brahmer et al. 2012. N Engl J Med. 366(26):2455-65; PMID: 22658128); atezolizumab (MPDL3280A/RG7446); durvalumab (MEDI4736); and avelumab (MSB0010718).

In a further preferred embodiment the checkpoint modulator is a decoy receptor (e.g. a soluble receptor). Preferably, the decoy receptor is a soluble PD1 receptor. More preferably, the decoy receptor is a soluble variant of a PD-1 receptor or a fragment or variant thereof, wherein the PD-1 receptor is derived from a mammal, preferably selected from the group comprising, without being limited thereto, e.g. goat, cattle, swine, dog, cat, donkey, horse, monkey, ape, a rodent (such as a mouse, hamster, rabbit or rat), and, most preferably, human. Even more preferably, the decoy receptor is a soluble variant of a PD-1 receptor or a fragment or variant thereof, wherein the PD-1 receptor comprises an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the amino acid sequence according to SEQ ID NO: 1, or a fragment or variant thereof. In a particularly preferred embodiment, the decoy receptor used herein as a checkpoint modulator is a soluble PD-1 receptor comprising an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the amino acid sequence according to SEQ ID NO: 2, or a fragment or variant thereof.

According to another embodiment, the checkpoint modulator used herein is an OX40 stimulator. OX40 is a member of the TNFR-superfamily of receptors, and is expressed on the surface of antigen-activated mammalian CD4+ and CD8+ T lymphocytes. OX40 ligand (OX40L, also known as gp34, ACT-4-L, and CD252) is a protein that specifically interacts with the OX40 receptor. The term OX40L includes the entire OX40 ligand, soluble OX40 ligand, and fusion proteins comprising a functionally active portion of OX40 ligand covalently linked to a second moiety, e.g., a protein domain. Also included within the definition of OX40L are variants which vary in amino acid sequence from naturally occurring OX40L, but which retain the ability to specifically bind to the OX40 receptor. Further included within the definition of OX40L are variants thereof, which enhance the biological activity of OX40. An OX40 agonist is a molecule which induces or enhances the biological activity of OX40, e.g. signal transduction mediated by OX40. An OX40 agonist is preferably defined herein as a binding molecule capable of specific binding to OX40. Therefore, the OX40 agonist may be any agonist binding to OX40 and capable of stimulating OX40 signaling. In this context, the OX40 agonist may be an agonistic antibody binding to OX40.

OX40 agonists and anti-OX40 monoclonal antibodies are described in WO1995/021251, WO1995/012673 and WO1995/21915. Particularly preferred is the anti-OX40 antibody 9612, a murine anti-OX40 monoclonal antibody directed against the extracellular domain of human OX40 (Weinberg et al., 2006. J. Immunother. 29(6):575-585).

In another embodiment, the checkpoint modulator as used herein is an antagonistic antibody is selected from the group consisting of anti-CTLA4, anti-PD1, anti-PD-L1, anti-Vista, anti-Tim-3, anti-TIGIT, anti-LAG-3, and anti-BTLA. Preferably, an anti-CTLA4 antibody that may be used as a checkpoint modulator is directed against Cytotoxic T lymphocyte antigen-4 (CTLA-4). CTLA-4 is mainly expressed within the intracellular compartment of T cells. After a potent or long-lasting stimulus to a naive T cell via the T cell receptor (TCR), CTLA-4 is transported to the cell surface and concentrated at the immunological synapse. CTLA-4 then competes with CD28 for CD80/CD86 and down-modulates TCR signaling via effects on Akt signaling. Thus CTLA-4 functions physiologically as a signal dampener (Weber, J. 2010. Semin. Oncol. 37(5):430-9).

In preferred embodiments, the isRNA is for use as described herein, wherein the use comprises—as an additional pharmaceutically active ingredient—a CTLA4 antagonist, which is preferably an antagonistic antibody directed against CTLA4 (anti-CTLA4 antibody). The term ‘CTLA4 antagonist’ as used herein comprises any compound, such as an antibody, that antagonizes the physiological function of CTLA4. In the context of the present invention, the term ‘anti-CTLA4 antibody’ may refer to an antagonistic antibody directed against CTLA4 (or a functional fragment or variant of said antibody) or to a nucleic acid, preferably an RNA, encoding said antagonistic antibody (or a functional fragment thereof). A functional fragment or variant of an anti-CTLA4 antibody preferably acts as a CTLA4 antagonist. More preferably, the term ‘anti-CTLA4 antibody’ refers to a monoclonal antibody directed against CTLA4 (or a functional fragment or variant of such an antibody) or to a nucleic acid encoding a monoclonal antibody directed against CTLA4 (or a functional fragment or variant of such an antibody). The term ‘anti-CTLA4 antibody’ as used herein may refer to the heavy or light antibody chain, respectively, or also refer to both antibody chains (heavy and light chain), or to a fragment or variant of any one of these chains. Preferably, the fragment or variant of an anti-CTLA4 antibody as used herein is a functional fragment or variant, preferably as described herein.

Particularly preferred are the anti-CTLA-4 antibodies ipilimumab (Yervoy®), tremelimumab, and AGEN-1884. Further preferred anti-CTLA4 antibodies as used herein comprise BMS 734016; BMS-734016; BMS734016; MDX 010; MDX 101; MDX-010; MDX-101; MDX-CTLA-4; MDX-CTLA4; MDX010; Winglore; and Yervoy, or a functional fragment or variant of any one of these antibodies.

According to some embodiments, the checkpoint modulator as used herein is a CTLA4 antagonist, preferably an anti-CTLA4 antibody. Therein, the anti-CTLA4 antibody preferably comprises two polypeptide chains, typically referred to as ‘heavy chain’ and ‘light chain’, respectively. In a preferred embodiment, the heavy chain comprises or consists of an amino acid sequence according to any one of SEQ ID NO: 645, 832, 661 or 833, or a fragment or variant of any one of these amino acid sequences, preferably according to SEQ ID NO: 645, or a fragment or variant thereof. The heavy chain preferably comprises or consists of an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to any one of SEQ ID NO: 645, 832, 661 or 833, preferably to SEQ ID NO: 645. The light chain preferably comprises or consists of an amino acid sequence according to any one of SEQ ID NO: 677, 834, 693 or 706, or a fragment or variant of any one of these amino acid sequences, preferably according to SEQ ID NO: 677, or a fragment or variant thereof. More preferably, the light chain comprises or consists of an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to any one of SEQ ID NO: 677, 834, 693 or 706, preferably to SEQ ID NO: 677. In preferred embodiments, the anti-CTLA4 antibody thus comprises a heavy chain and a light chain as described above, or a fragment or variant of each of these chains. Most preferably, the anti-CTLA4 antibody comprises a heavy chain and a light chain, or a fragment or variant thereof, wherein the heavy chain, or the fragment or variant thereof, comprises or consists of an amino acid sequence according to SEQ ID NO: 645, or a fragment or variant thereof, and wherein the light chain, or the fragment or variant thereof, comprises or consists of an amino acid sequence according to SEQ ID NO: 677, or a fragment or variant thereof.

According to a further embodiment, the checkpoint modulator as used herein is at least one antibody described in Table 1 or a fragment or variant thereof

TABLE 1
Antibodies directed against checkpoint molecules

| Name | Target |
|---|---|
| Urelumab | 4-1BB/CD137 |
| PF-05082566 | 4-1BB/CD137 |
| 8H9 | B7-H3 |
| Enoblituzumab | B7-H3 |
| Ipilimumab | CD152/CTLA-4 |
| Ticilimumab (=tremelimumab) | CD152/CTLA-4 |
| Tremelimumab | CD152/CTLA-4 |
| Varlilumab | CD27 |
| Teneliximab | CD40 |
| Vorsetuzumab mafodotin | CD70 |
| Lirilumab | KIR2D |
| GSK-3174998 | OX40 |
| MEDI-6469 | OX40 |
| MEDI-6383 | OX40 |
| MEDI-0562 | OX40 |
| PF-04518600 | OX40 |
| RG-7888 | OX40 |
| PF-06801591 | PD-1 |
| BGBA-317 | PD-1 |
| MEDI-0680 | PD-1 |
| MK-3475 | PD-1 |
| Nivolumab | PD-1 |
| PDR-001 | PD-1 |
| Pembrolizumab | PD-1 |
| Pidilizumab | PD-1 |
| REGN-2810 | PD-1 |
| SHR-1210 | PD-1 |
| TSR-042 | PD-1 |
| MDX-1106 | PD-1 |
| Merck 3745 | PD-1 |
| CT-011 | PD-1 |
| MEDI-0680 | PD-1 |
| PDR001 | PD-1 |
| REGN2810 | PD-1 |
| BGB-108 | PD-1 |
| BGB-A317 | PD-1 |
| AMP-224 | PD-1 |
| Atezolizumab | PD-L1 (CD274) |
| Avelumab | PD-L1 (CD274) |
| BMS-936559 | PD-L1 (CD274) |
| Durvalumab | PD-L1 (CD274) |
| MEDI-4736 | PD-L1 (CD274) |
| MPDL33280A | PD-L1 (CD274) |
| YW243.55.S70 | PD-L1 (CD274) |
| MDX-1105 | PD-L1 (CD274) |
| MSB0010718C | PD-L1 (CD274) |

According to a preferred embodiment, the at least one additional pharmaceutically active ingredient is a cytokine.

More preferably, the at least one additional pharmaceutically active ingredient is IL-12 or a fragment or variant thereof. Even more preferably, the at least one additional pharmaceutically active ingredient is an IL-12 analog or a fragment or variant thereof. Most preferably, the at least one additional pharmaceutically active ingredient is a compound, such as a peptide or a protein, a mutated peptide or a mutated protein, a coupled heterodimer, an antibody, preferably an antibody encoded by RNA, or artificial binding domains, which binds to IL-12 receptor and preferably leads to activation of the JAK-STAT signaling pathway. In a preferred embodiment, the at least one additional pharmaceutically active ingredient is an IL-12 receptor agonist.

Naturally occurring IL-12 is typically a heterodimeric cytokine encoded by two separate genes, IL-12A (p35) and IL-12B (p40). The naturally occurring heterodimer is also referred to as p70. In the context of the present invention, the term “IL-12” refers to a protein consisting of or comprising a naturally occuring form of heterodimeric IL-12, a monomeric IL-12A, a monomeric IL-12B as well as fragments or variants of any of these, such as fusions of IL-12A, or a fragment or variant thereof, with IL-12B, or a fragment or variant thereof, wherein said protein may also comprise an amino acid sequence that is not related to IL-12A or IL-12B. For example, the term “IL-12” as used herein also comprises a protein comprising IL-12A, or a fragment or variant thereof, IL-12B, or a fragment or variant thereof, wherein the IL-12A, or a fragment or variant thereof, is covalently linked to the IL-12B, or a fragment or variant thereof, by a linker, wherein the linker is preferably an amino acid sequence not related to IL-12A or IL-12B. More preferably, the linker is a peptide or protein linker, which preferably comprises an amino acid sequence according to SEQ ID NO: 9 or a fragment or variant thereof. Further, the term “IL-12” also comprises a protein, wherein IL-12A, or a fragment or variant thereof, is directly linked to the IL-12B, or a fragment or variant thereof, preferably via covalent linkage, more preferably via a peptide bond. In addition, the terms “IL-12” or “IL-12 analog” as used herein also comprise any compound, such as a peptide or a protein, a mutated peptide or a mutated protein, a coupled heterodimer, an antibody, preferably an antibody encoded by RNA, or artificial binding domains, which binds to an IL-12 receptor and which preferably leads to activation of the JAK-STAT signaling pathway. Hence, the terms “IL-12” or “IL-12 analog” as used herein also comprise a compound, such as a peptide or protein, that functions as an IL-12 receptor agonist.

As used herein, a fragment or variant of IL-12 as defined herein is preferably able to specifically bind to an IL-12 receptor and, more preferably, to function as an IL-12 receptor agonist.

According to a preferred embodiment, the IL-12 as used in the context of the present invention is derived from a mammal, preferably selected from the group comprising, without being limited thereto, e.g. goat, cattle, swine, dog, cat, donkey, horse, monkey, ape, a rodent (such as a mouse, hamster, rabbit or rat), and, most preferably, human. Even more preferably, the IL-12 as used herein comprises an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to any one of the amino acid sequences according to SEQ ID NO: 3 to 8, or a fragment or variant of any of these sequences. Alternatively or in addition, the IL-12 as used herein comprises an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the amino acid sequence according to SEQ ID NO: 9, or a fragment or variant thereof. In a particularly preferred embodiment, the IL-12 as used herein comprises an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the amino acid sequence according to SEQ ID NO: 10, or a fragment or variant thereof.

In an alternative embodiment, the at least one additional pharmaceutically active ingredient is CD40L or a fragment or variant thereof. CD40L (also known as “CD40 ligand”, as “CD40LG” or as “CD154”) is primarily expressed on activated T and B cells. CD40L binds to CD40, which is typically expressed on antigen-presenting cells (APC). The interaction of CD40 and CD40L plays a pivotal role in the development of humoral and cellular immune responses. As used herein, the term “CD40L” refers to a naturally occurring CD40L protein or to a fragment or variant thereof, wherein the fragment or variant is preferably able to specifically bind to a CD40.

According to a preferred embodiment, the CD40L as used in the context of the present invention is derived from a mammal, preferably selected from the group comprising, without being limited thereto, e.g. goat, cattle, swine, dog, cat, donkey, horse, monkey, ape, a rodent (such as a mouse, hamster, rabbit or rat), and, most preferably, human. Even more preferably, the CD40L as used herein comprises an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the amino acid sequence according to SEQ ID NO: 11, or a fragment or variant thereof.

According to a particularly preferred embodiment the at least one additional pharmaceutically active ingredient is a tumour antigen preferably selected from any tumor antigen comprising an amino acid sequence selected from the group consisting of SEQ ID NOs: 1-504; 4558-4560 of PCT/EP2017/059525, or a fragment or variant of any one of said amino acid sequences.

In a preferred embodiment, the present invention thus provides the isRNA for use in the treatment of a tumor or cancer disease as described herein, wherein the treatment comprises administration of at least one coding RNA (as additional pharmaceutically active ingredient) encoding at least one tumor antigen, preferably at least one mRNA, wherein the at least one coding sequence comprises a nucleic acid sequence selected from the group consisting of SEQ ID Nos. 505-4033; 4561-4591 of PCT/EP2017/059525, or a fragment or variant of any one of said sequences.

More preferably, the tumor antigen is selected from the group consisting of 1A01_HLA-A/m; 1A02; 5T4; ACRBP; AFP; AKAP4; alpha-actinin-_4/m; alpha-methylacyl-coenzyme_A_racemase; ANDR; ART-4; ARTC1/m; AURKB; B2MG; B3GN5; B4GN1; B7H4; BAGE-1; BASI; BCL-2; bcr/abl; beta-catenin/m; BING-4; BIRC7; BRCA1/m; BY55; calreticulin; CAMEL; CASP-8/m; CASPA; cathepsin_B; cathepsin_L; CD1A; CD1B; CD1C; CD1D; CD1E; CD20; CD22; CD276; CD33; CD3E; CD3Z; CD44_Isoform_1; CD44_Isoform_6; CD4; CD52; CD55; CD56; CD80; CD86; CD8A; CDC27/m; CDE30; CDK4/m; CDKN2A/m; CEA; CEAM6; CH3L2; CLCA2; CML28; CML66; COA-1/m; coactosin-like_protein; collagen_XXIII; COX-2; CP1B1; CSAG2; CT45A1; CT55; CT-_9/BRD6; CTAG2_Isoform_LAGE-1A; CTAG2_Isoform_LAGE-1B; CTCFL; Cten; cyclin_B1; cyclin_D1; cyp-B; DAM-10; DEP1A; E7; EF1A2; EFTUD2/m; EGFR; EGLN3; ELF2/m; EMMPRIN; EpCam; EphA2; EphA3; ErbB3; ERBB4; ERG; ETV6; EWS; EZH2; FABP7; FCGR3A_Version_1; FCGR3A_Version_2; FGF5; FGFR2; fibronectin; FOS; FOXP3; FUT1; G250; GAGE-1; GAGE-2; GAGE-3; GAGE-4; GAGE-5; GAGE-6; GAGE7b; GAGE-8_(GAGE-2D); GASR; GnT-V; GPC3; GPNMB/m; GRM3; HAGE; hepsin; Her2/neu; HLA-A2/m; homeobox_NKX3.1; HOM-TES-85; HPG1; HS71A; HS71B; HST-2; hTERT; iCE; IF2B3; IL10; IL-13Ra2; IL2-RA; IL2-RB; IL2-RG; IL-5; IMP3; ITA5; ITB1; ITB6; kallikrein-2; kallikrein-3; kallikrein-4; KI20A; KIAA0205; KIF2C; KK-LC-1; LDLR; LGMN; LIRB2; LY6K; MAGA5; MAGA8; MAGAB; MAGE-A10; MAGE-A12; MAGE-A1; MAGE-A2; MAGE-A3; MAGE-A4; MAGE-A6; MAGE-A9; MAGE-B10; MAGE-B16; MAGE-B17; MAGE-_131; MAGE-B2; MAGE-B3; MAGE-B4; MAGE-B5; MAGE-B6; MAGE-C1; MAGE-C2; MAGE-C3; MAGE-D1; MAGE-D2; MAGE-D4; MAGE-_E1; MAGE-E1_(MAGE1); MAGE-E2; MAGE-F1; MAGE-H₁; MAGEL2; mammaglobin_A; MART-1/melan-A; MART-2; MC1_R; M-CSF; mesothelin; MITF; MMP1_1; MMP7; MUC-1; MUM-1/m; MUM-2/m; MYCN; MYO1A; MYO1B; MYO1C; MYO1D; MYO1E; MYO1F; MYO1G; MYO1H; NA17; NA88-A; Neo-PAP; NFYC/m; NGEP; NPM; NRCAM; NSE; NUF2; NY-ESO-1; OA1; OGT; OS-9; osteocalcin; osteopontin; p53; PAGE-4; PAI-1; PAI-2; PAP; PATE; PAX3; PAXS; PD1L1; PDCD1; PDEF; PECA1; PGCB; PGFRB; Pim-1_-Kinase; Pin-1; PLAC1; PMEL; PML; POTEF; POTE; PRAME; PRDX5/m; PRM2; prostein; proteinase-3; PSA; PSB9; PSCA; PSGR; PSM; PTPRC; RAB8A; RAGE-1; RARA; RASH; RASK; RASN; RGSS; RHAMM/CD168; RHOC; RSSA; RU1; RU2; RUNX1; S-100; SAGE; SART-_1; SART-2; SART-3; SEPR; SERPINBS; SIA7F; SIA8A; SIAT9; SIRT2/m; SOX10; SP17; SPNXA; SPXN3; SSX-1; SSX-2; SSX3; SSX-4; ST1A1; STAG2; STAMP-1; STEAP-1; Survivin-2B; survivin; SYCP1; SYT-SSX-1; SYT-SSX-2; TARP; TCRg; TF2AA; TGFB1; TGFR2; TGM-4; TIE2; TKTL1; TPI/m; TRGV11; TRGV9; TRPC1; TRP-p8; TSG10; TSPY1; TVC_(TRGV3); TX101; tyrosinase; TYRP1; TYRP2; UPA; VEGFR1; WT1; and XAGE1.

In preferred embodiments, a tumor antigen, preferably as defined herein, is provided in the form of at least one coding RNA, preferably as defined herein, which comprises at least one coding sequence encoding a peptide or protein comprising a tumor antigen, or a fragment or variant thereof. Said at least one coding RNA comprising at least one coding sequence encoding a peptide or protein comprising a tumor antigen, or a fragment or variant thereof, may preferably be administered intratumorally. Alternatively, said at least one coding RNA may be administered intradermally, intramuscularly or subcutaneously.

In a preferred embodiment, the at least one additional pharmaceutically active ingredient used herein is a coding RNA, preferably an mRNA. Hence, according to certain embodiments, the invention provides an isRNA for use in the treatment of a tumor or cancer disease as described herein, wherein the treatment further comprises administration of at least one coding RNA, preferably at least one mRNA, wherein the isRNA is preferably administered intratumorally (e.g. locoregionally). In a particularly preferred embodiment, the coding RNA, preferably an mRNA, is administered intratumorally (e.g. locoregionally). More preferably, the isRNA as well as the at least one coding RNA are administered intratumorally (e.g. locoregionally).

In a preferred embodiment, the at least one additional pharmaceutically active ingredient used herein is a coding RNA, preferably an mRNA. Hence, according to certain embodiments, the invention provides an isRNA for use in the treatment of a tumor or cancer disease as described herein, wherein the treatment further comprises administration of at least one coding RNA, preferably at least one mRNA, wherein the isRNA is preferably administered intratu morally. In a particularly preferred embodiment, the coding RNA, preferably an mRNA, is administered intratumorally. More preferably, the isRNA as well as the at least one coding RNA are administered intratumorally.

In some embodiments, the at least one coding RNA is formulated together with the isRNA for use as described herein. In a particularly preferred embodiment, the at least one coding RNA is formulated separately from the isRNA, wherein the at least one coding RNA as well as the isRNA are preferably administered intratumorally (e.g. locoregionally).

According to a preferred embodiment, the isRNA is formulated together with the at least one coding RNA, wherein the co-formulation is preferably administered intratumorally (e.g. locoregionally).

In some embodiments, the at least one coding RNA is formulated together with the isRNA for use as described herein. In a particularly preferred embodiment, the at least one coding RNA is formulated separately from the isRNA, wherein the at least one coding RNA as well as the isRNA are preferably administered intratumorally.

According to a preferred embodiment, the isRNA is formulated together with the at least one coding RNA, wherein the co-formulation is preferably administered intratumorally.

According to a preferred embodiment, the at least one coding RNA encodes at least one peptide or protein comprising at least one of the peptides or proteins described herein as an additional pharmaceutically active ingredient. More preferably, the at least one coding RNA encodes at least one peptide or protein comprising at least one peptide or protein selected from the group consisting of
IL-12,
CD40L and
a decoy PD-1 receptor, preferably a soluble PD-1 receptor,

or a fragment or variant of any of these,

wherein the IL-12, the CD40L or the decoy PD-1 receptor, or a fragment or variant of any of these, is preferably as described herein. Even more preferably, the at least one coding RNA encodes at least one peptide or protein comprising at least one peptide or protein selected from the group consisting of
IL-12,
CD40L,
a decoy PD-1 receptor, preferably a soluble PD-1 receptor, and
an anti-CTLA4 antibody,

or a fragment or variant of any of these,

wherein the IL-12, the CD40L, the decoy PD-1 receptor, or the anti-CTLA4 antibody, or a fragment or variant of any of these, is preferably as described herein.

Alternatively, the at least one coding RNA encodes at least one peptide or protein comprising at least one peptide or protein selected from the group consisting of
IL-12,
CD40L, and
an anti-CTLA4 antibody,

or a fragment or variant of any of these,

wherein the IL-12, the CD40L, or the anti-CTLA4 antibody, or a fragment or variant of any of these, is preferably as described herein.

According to a preferred embodiment, the invention provides an isRNA for use in the treatment of a tumor or cancer disease,

wherein the isRNA comprises a nucleic acid sequence according to formula (I) (GₗXₘGₙ), formula (II) (CₗXₘCₙ), formula (III) (NᵤGₗXₘGₙNᵥ)ₐ or formula (IV) (NᵤCₗXₘCₙNᵥ)ₐ, preferably at least one nucleic acid sequence according to any one of SEQ ID NOs: 433 to 434, 1014 to 1016, or a fragment or variant of any of these sequences, wherein the isRNA is complexed with a cationic or polycationic compound, preferably with a polymeric carrier, more preferably with a polymeric carrier that is formed by a disulfide-crosslinked cationic component, which preferably comprises a peptide according to formula (V), (Va) and/or (Vb) and/or a compound according to formula (VI), more preferably at least one of the disulfide-crosslinked cationic peptides Cys-Arg₁₂ (SEQ ID NO: 580), Cys-Arg₁₂-Cys (SEQ ID NO: 579), or Trp-Arg₁₂-Cys (SEQ ID NO: 1074),

wherein the isRNA is preferably administered intratumorally,

wherein the treatment of a tumor or cancer disease comprises administration of at least one coding RNA, preferably at least one mRNA, encoding at least one peptide or protein comprising at least one peptide or protein selected from the group consisting of
IL-12,
CD40L and
a decoy PD-1 receptor, preferably a soluble PD-1 receptor,

or a fragment or variant of any of these,

wherein the IL-12, the CD40L or the decoy PD-1 receptor, or a fragment or variant of any of these, is preferably as described herein.

According to a particularly preferred embodiment, the invention provides an isRNA for use in the treatment of a tumor or cancer disease,

wherein the isRNA comprises a nucleic acid sequence according to formula (I) (GₗXₘGₙ), formula (II) (CₗXₘCₙ), formula (III) (NᵤGₗXₘGₙNᵥ)ₐ or formula (IV) (NᵤCₗXₘCₙNᵥ)ₐ, preferably at least one nucleic acid sequence according to any one of SEQ ID NOs: 433 to 437, 1014 to 1016, 1055 or 1056, or a fragment or variant of any of these sequences, wherein the isRNA is complexed with a cationic or polycationic compound, preferably with a polymeric carrier, more preferably with a polymeric carrier that is formed by a disulfide-crosslinked cationic component, which preferably comprises a peptide according to formula (V), (Va) and/or (Vb) and/or a compound according to formula (VI), more preferably at least one of the disulfide-crosslinked cationic peptides Cys-Arg₁₂ (SEQ ID NO: 580), Cys-Arg₁₂-Cys (SEQ ID NO: 579), or Trp-Arg₁₂-Cys (SEQ ID NO: 1074),

wherein the isRNA is preferably administered intratumorally,

wherein the tumor or cancer disease is preferably selected from the group consisting of melanoma, preferably advanced and/or metastatic melanoma, most preferably advanced cutaneous melanoma (cMEL), squamous cell cancer of the skin (SCC), preferably unresectable and/or advanced SCC, most preferably cutaneous squamous cell carcinoma (cSCC), or other forms of malignant skin cancer, adenocystic carcinoma (ACC), preferably advanced ACC, cutaneous T-cell lymphoma, preferably advanced cutaneous T-cell lymphoma, and squamous cell carcinoma of the head and neck (HNSCC), preferably advanced HNSCC;

wherein the treatment of a tumor or cancer disease comprises administration of at least one coding RNA, preferably at least one mRNA, encoding at least one peptide or protein comprising at least one peptide or protein selected from the group consisting of
IL-12,
CD40L and
a decoy PD-1 receptor, preferably a soluble PD-1 receptor,

or a fragment or variant of any of these, and

wherein the IL-12, the CD40L or the decoy PD-1 receptor, or a fragment or variant of any of these, is preferably as described herein.

According to a further preferred embodiment, the invention provides an isRNA for use in the treatment of a tumor or cancer disease,

wherein the isRNA comprises a nucleic acid sequence according to formula (I) (GₗXₘGₙ), formula (II) (CₗXₘCₙ), formula (III) (NᵤGₗXₘGₙNᵥ)ₐ or formula (IV) (NᵤCₗXₘCₙNᵥ)ₐ, preferably at least one nucleic acid sequence according to any one of SEQ ID NOs: 433 to 437, 1014 to 1016 or 1055 or 1056, more preferably according to any one of SEQ ID NO: 433,434 or 1014 to 1016, or a fragment or variant of any of these sequences,

wherein the isRNA is complexed with a cationic or polycationic compound, preferably with a polymeric carrier, more preferably with a polymeric carrier that is formed by a disulfide-crosslinked cationic component, which preferably comprises a peptide according to formula (V), (Va) and/or (Vb) and/or a compound according to formula (VI), more preferably at least one of the disulfide-crosslinked cationic peptides Cys-Arg₁₂ (SEQ ID NO: 580), Cys-Arg₁₂-Cys (SEQ ID NO: 579), or Trp-Arg₁₂-Cys (SEQ ID NO: 1074),

wherein the isRNA is preferably administered intratumorally, including peritumorally or locoregionally, wherein the treatment of a tumor or cancer disease comprises administration of at least one coding RNA, preferably at least one mRNA, encoding at least one peptide or protein comprising at least one peptide or protein selected from the group consisting of
IL-12,
CD40L,
an anti-CTLA4 antibody, and
optionally, a decoy PD-1 receptor, preferably a soluble PD-1 receptor,

or a fragment or variant of any of these,

wherein the IL-12, the CD40L, the decoy PD-1 receptor, or the anti-CTLA4 antibody, or a fragment or variant of any of these, is preferably as described herein.

According to a particularly preferred embodiment, the invention provides an isRNA for use in the treatment of a tumor or cancer disease,

wherein the isRNA comprises a nucleic acid sequence according to formula (I) (GₗXₘGₙ), formula (II) (CₗXₘCₙ), formula (III) (NᵤGₗXₘGₙNᵥ)ₐ or formula (IV) (NᵤCₗXₘCₙNᵥ)ₐ, preferably at least one nucleic acid sequence according to any one of SEQ ID NOs: 433 to 437, 1014 to 1016, preferably according to any one of SEQ ID NOs: 433, 434 or 1014 to 1016, or a fragment or variant of any of these sequences,

wherein the isRNA is complexed with a cationic or polycationic compound, preferably with a polymeric carrier, more preferably with a polymeric carrier that is formed by a disulfide-crosslinked cationic component, which preferably comprises a peptide according to formula (V), (Va) and/or (Vb) and/or a compound according to formula (VI), more preferably at least one of the disulfide-crosslinked cationic peptides Cys-Arg₁₂ (SEQ ID NO: 580), Cys-Arg₁₂-Cys (SEQ ID NO: 579), or Trp-Arg₁₂-Cys (SEQ ID NO: 1074),

wherein the isRNA is preferably administered intratumorally, including peritumorally or locoregionally,

wherein the tumor or cancer disease is preferably selected from the group consisting of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC);

wherein the tumor or the cancer disease is preferably at an advanced stage and/or refractory to standard therapy;

wherein the treatment of a tumor or cancer disease comprises administration of at least one coding RNA, preferably at least one mRNA, encoding at least one peptide or protein comprising at least one peptide or protein selected from the group consisting of
IL-12,
CD40L,
an anti-CTLA4 antibody, and
optionally, a decoy PD-1 receptor, preferably a soluble PD-1 receptor,

or a fragment or variant of any of these,

wherein the IL-12, the CD40L, the decoy PD-1 receptor, or the anti-CTLA4 antibody, or a fragment or variant of any of these, is preferably as described herein.

According to preferred embodiments of the invention, the treatment or prophylaxis of a cancer or tumor disease as described herein comprises administration of a decoy PD-1 receptor as described herein in cases, where the subject does not receive or has not received a treatment with a PD-1 antagonist and/or a PD-L1 antagonist. In cases, where the patient does not receive or has not received anti PD-1 and/or anti PD-L1 treatment (e.g. an anti PD-1 antibody or an anti PD-L1 antibody) it is particularly preferred that the at least one mRNA encodes a decoy PD-1 receptor.

In other cases, where the subject receives or has received a treatment with a PD-1 or PD-L1 antagonist, the treatment or prophylaxis of a cancer or tumor disease as envisaged herein does preferably not comprise the administration of a decoy PD-1 receptor as described herein or a nucleic acid encoding a decoy PD-1 receptor or a fragment or variant thereof.

According to a preferred embodiment, the treatment of a tumor or cancer disease in a subject that receives or has received a treatment with a PD-1 or a PD-L1 antagonist comprises administration of at least one coding RNA, preferably at least one mRNA, encoding at least one peptide or protein comprising at least one peptide or protein selected from the group consisting of
IL-12,
CD40L, and
an anti-CTLA4 antibody,

or a fragment or variant of any of these,

wherein the IL-12, the CD40L, or the anti-CTLA4 antibody, or a fragment or variant of any of these, is preferably as described herein.

In further preferred embodiments, the treatment of a tumor or cancer disease in a subject that does not receive or has not received a treatment with a PD-1 or a PD-L1 antagonist comprises administration of at least one coding RNA, preferably at least one mRNA, encoding at least one peptide or protein comprising at least one peptide or protein selected from the group consisting of
IL-12,
CD40L, and
an anti-CTLA4 antibody, and
a decoy PD-1 receptor, preferably a soluble PD-1 receptor,

or a fragment or variant of any of these,

wherein the IL-12, the CD40L, the anti-CTLA4 antibody, or the decoy PD-1 receptor, or a fragment or variant of any of these, is preferably as described herein.

According to a preferred embodiment, the at least one coding RNA encodes a peptide or protein comprising IL-12 or a fragment or variant thereof as defined herein. In some embodiments, the at least one coding RNA encodes a peptide or protein, a mutated peptide or a mutated protein, a coupled heterodimer, an antibody, preferably an antibody encoded by RNA, or artificial binding domains comprising an IL-12 analog or a fragment or variant thereof as defined herein. Preferably, the encoded peptide or protein comprises an amino acid according to any one of SEQ ID NO: SEQ ID NO: 3 to 8, or a fragment or variant of any of these sequences. The encoded peptide or protein may preferably also comprise an amino acid sequence according to SEQ ID NO: 9, or a fragment or variant thereof. More preferably, the encoded peptide or protein comprises an amino acid according to SEQ ID NO: 10, or a fragment or variant thereof.

Most preferably, the encoded peptide or protein comprises an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to any one of the amino acid sequences according to SEQ ID NO: 3 to 8, or a fragment or variant of any of these sequences. In a particularly preferred embodiment, the encoded peptide or protein comprises an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the amino acid sequence according to SEQ ID NO: 10, or a fragment or variant thereof.

In a further preferred embodiment, the at least one coding RNA encodes a peptide or protein comprising IL-12 or a fragment or variant thereof as defined herein, wherein the at least one coding RNA comprises a nucleic acid sequence according to any one of SEQ ID NO: 14 to 19 or 21 or SEQ ID NOs: 440 to 445 or 447, preferably any one of SEQ ID NOs: 440 to 445 or 447, or a fragment or variant of any of these sequences. More preferably, the at least one coding RNA encodes a peptide or protein comprising IL-12 or a fragment or variant thereof as defined herein, wherein the at least one coding RNA comprises a nucleic acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to any one of the nucleic acid sequences according to SEQ ID NO: 14 to 19 or 21 or SEQ ID NOs: 440 to 445 or 447, preferably any one of SEQ ID NOs: 440 to 445 or 447, or a fragment or variant of any of these sequences.

Alternatively or in addition, the at least one coding RNA encodes a peptide or protein comprising IL-12 or a fragment or variant thereof as defined herein, wherein the at least one coding RNA comprises a nucleic acid sequence according to SEQ ID NO: 20 or 446, preferably according to SEQ ID NO: 446, or a fragment or variant of any of these sequences. More preferably, the at least one coding RNA encodes a peptide or protein comprising IL-12 or a fragment or variant thereof as defined herein, wherein the at least one coding RNA comprises a nucleic acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to a nucleic acid sequence according to SEQ ID NO: 20 or 446, preferably according to SEQ ID NO: 446, or a fragment or variant of any of these sequences.

In a particularly preferred embodiment, the at least one coding RNA encodes a peptide or protein comprising IL-12 or a fragment or variant thereof as defined herein, wherein the at least one coding RNA comprises a nucleic acid sequence according to SEQ ID NO: 21 or SEQ ID NO: 447, preferably SEQ ID NO: 447, or a fragment or variant thereof. More preferably, the at least one coding RNA encodes a peptide or protein comprising IL-12 or a fragment or variant thereof as defined herein, wherein the at least one coding RNA comprises a nucleic acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the nucleic acid sequence according to SEQ ID NO: 21 or SEQ ID NO: 447, preferably SEQ ID NO: 447, or a fragment or variant thereof.

According to another embodiment, the at least one coding RNA encodes a peptide or protein comprising CD40L or a fragment or variant thereof as defined herein. Preferably, the encoded peptide or protein comprises an amino acid according to SEQ ID NO: 11, or a fragment or variant thereof. More preferably, the encoded peptide or protein comprises an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the amino acid sequence according to SEQ ID NO: 11, or a fragment or variant thereof.

In a further preferred embodiment, the at least one coding RNA encodes a peptide or protein comprising CD40L or a fragment or variant thereof as defined herein, wherein the at least one coding RNA comprises a nucleic acid sequence according to SEQ ID NO: 22 or SEQ ID NO: 448, preferably SEQ ID NO: 448, or a fragment or variant thereof. More preferably, the at least one coding RNA encodes a peptide or protein comprising CD40L or a fragment or variant thereof as defined herein, wherein the at least one coding RNA comprises a nucleic acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the nucleic acid sequence according to SEQ ID NO: 22 or SEQ ID NO: 448, preferably SEQ ID NO: 448, or a fragment or variant of any of these sequences.

According to an alternative embodiment, the at least one coding RNA encodes a peptide or protein comprising a decoy PD-1 receptor or a fragment or variant thereof as defined herein, more preferably a soluble PD-1 receptor or a fragment or variant thereof as defined herein. Preferably, the encoded peptide or protein comprises an amino acid according to any one of SEQ ID NO: 2 or SEQ ID NO: 1042, or a fragment or variant thereof. More preferably, the encoded peptide or protein comprises an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the amino acid sequence according to SEQ ID NO: 2 or SEQ ID NO: 1042, or a fragment or variant thereof.

In a further preferred embodiment, the at least one coding RNA encodes a peptide or protein comprising a decoy PD-1 receptor, more preferably a soluble PD-1 receptor, or a fragment or variant thereof as defined herein, wherein the at least one coding RNA comprises a nucleic acid sequence according to SEQ ID NO: 13 or SEQ ID NO: 439, preferably SEQ ID NO: 439, or a fragment or variant thereof. More preferably, the at least one coding RNA encodes a peptide or protein comprising a decoy PD-1 receptor, more preferably a soluble PD-1 receptor, or a fragment or variant thereof as defined herein, wherein the at least one coding RNA comprises a nucleic acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the nucleic acid sequence according to SEQ ID NO: 13 or SEQ ID NO: 439, preferably SEQ ID NO: 439, or a fragment or variant of any of these sequences.

According to an alternative embodiment, the at least one coding RNA encodes a peptide or protein comprising a decoy PD-1 receptor or a fragment or variant thereof as defined herein, more preferably a soluble PD-1 receptor or a fragment or variant thereof as defined herein. Preferably, the encoded peptide or protein comprises an amino acid according to any one of SEQ ID NO: 1, or a fragment or variant thereof. More preferably, the encoded peptide or protein comprises an amino acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the amino acid sequence according to SEQ ID NO: 1, or a fragment or variant thereof.

In a further preferred embodiment, the at least one coding RNA encodes a peptide or protein comprising a decoy PD-1 receptor, more preferably a soluble PD-1 receptor, or a fragment or variant thereof as defined herein, wherein the at least one coding RNA comprises a nucleic acid sequence according to SEQ ID NO: 12 or SEQ ID NO: 438, preferably SEQ ID NO: 438, or a fragment or variant thereof. More preferably, the at least one coding RNA encodes a peptide or protein comprising a decoy PD-1 receptor, more preferably a soluble PD-1 receptor, or a fragment or variant thereof as defined herein, wherein the at least one coding RNA comprises a nucleic acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the nucleic acid sequence according to SEQ ID NO: 12 or SEQ ID NO: 438, preferably SEQ ID NO: 438, or a fragment or variant of any of these sequences.

According to a preferred embodiment, the at least one coding RNA encodes a peptide or protein comprising a CTLA4 antagonist as described herein. Preferably, the at least one coding RNA encodes a peptide or protein comprising an anti-CTLA4 antibody as described herein, or a fragment or variant thereof as defined herein, wherein the fragment or variant is preferably a functional fragment or a functional variant. More preferably, the at least one coding RNA encodes a peptide or protein comprising an anti-CTLA4 antibody as described herein, or a fragment or variant thereof as defined herein, wherein the at least one coding RNA comprises a nucleic acid sequence according to any one of SEQ ID NO: 646-660, 662-676, 678-692, 694-705, or 707-715, preferably according to any one of SEQ ID NO: 646-660, 679-692, or 710-715, or a fragment or variant of any one of these nucleic acid sequences. Even more preferably, the at least one coding RNA encodes a peptide or protein comprising an anti-CTLA4 antibody, or a fragment or variant thereof as defined herein, wherein the at least one coding RNA comprises a nucleic acid sequence identical or at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical to the nucleic acid sequence according to SEQ ID NO: 646-660, 662-676, 678-692, 694-705, or 707-715, preferably according to any one of SEQ ID NO: 646-660, 679-692, or 710-715.

It is further preferred that the at least one coding RNA encodes a peptide or protein comprising a heavy chain of an anti-CTLA4 antibody, or a fragment or variant thereof, and a light chain of an anti-CTLA4 antibody, or a fragment or variant thereof, wherein the heavy chain and the light chain, or a fragment or variant thereof, is preferably as described herein.

Preferably, the at least one coding RNA encodes a peptide or protein comprising a heavy chain of an anti-CTLA4 antibody, or a fragment or variant thereof, and a light chain of an anti-CTLA4 antibody, or a fragment or variant thereof,

wherein the heavy chain, or the fragment or variant thereof, is encoded by a nucleic acid sequence selected from any one of SEQ ID NO: 646-660, 662-676, or 710-715, preferably from any one of SEQ ID NO: 646-660, or a fragment or variant of any one of these nucleic acid sequences, and

wherein the light chain, or the fragment or variant thereof, is encoded by a nucleic acid sequence selected from any one of SEQ ID NO: 678-692, 694-705, 707-709, or 710-715, preferably from any one of SEQ ID NO: 678-692, or a fragment or variant of any one of these nucleic acid sequences.

Therein, the heavy chain and the light chain, or the fragment or variant of any of these, respectively, is preferably encoded either by one and the same coding RNA or by different coding RNAs.

According to a preferred embodiment, the invention relates to the isRNA for use as described herein, wherein the use comprises—as an additional pharmaceutically active ingredient—an anti-CTLA4 antibody or a fragment or variant thereof as described herein, wherein the anti-CTLA4 antibody is provided in the form of two separate RNAs (formulated separately or together), wherein

the heavy chain of the anti-CTLA4 antibody, or a fragment or variant thereof, is encoded by an RNA comprising or consisting of a nucleic acid sequence selected from any one of SEQ ID NO: 646-660, 662-676, or 710-715, preferably from any one of SEQ ID NO: 646-660, or a fragment or variant of any one of these nucleic acid sequences, and

wherein the light chain of the anti-CTLA4 antibody, or a fragment or variant thereof, is encoded by another RNA comprising or consisting of a nucleic acid sequence selected from any one of SEQ ID NO: 678-692, 694-705, 707-709, or 710-715, preferably from any one of SEQ ID NO: 678-692, or a fragment or variant of any one of these nucleic acid sequences.

In an alternative embodiment, the invention relates to the isRNA for use as described herein, wherein the use comprises—as an additional pharmaceutically active ingredient—an anti-CTLA4 antibody or a fragment or variant thereof as described herein, wherein the anti-CTLA4 antibody, or the fragment or variant thereof, is provided in the form of an RNA, wherein one RNA encodes both, the heavy chain of the anti-CTLA4 antibody, or a fragment or variant thereof, and the light chain of the anti-CTLA4 antibody, or a fragment or variant thereof, and wherein the RNA preferably comprises or consists of a nucleic acid sequence according to any one of SEQ ID NO: 710-712, 713-715, or a fragment or variant of any one of these nucleic acid sequences.

The present invention further provides the isRNA for use in the treatment of a tumor or cancer disease as described herein, wherein the treatment comprises administration of at least one coding RNA (as additional pharmaceutically active ingredient), preferably at least one mRNA, wherein the at least one coding RNA encodes at least two peptides or proteins selected from the group consisting of
IL-12,
CD40L,
an anti-CTLA4 antibody, and
optionally, a decoy PD-1 receptor, preferably a soluble PD-1 receptor,

or a fragment or variant of any of these, and

wherein the IL-12, the CD40L, the decoy PD-1 receptor, or the anti-CTLA4 antibody, or a fragment or variant of any of these, is preferably as described herein.

In the context of the present invention, an anti-CTLA4 antibody as used herein may be referred to as ‘(one) peptide or protein’ (in the singular) even though the (mature) anti-CTLA4 antibody, or the fragment or variant thereof, preferably comprises at least two peptides or proteins, namely a heavy chain, or a fragment or variant thereof, and a light chain, or a fragment or variant thereof.

In certain embodiments, the treatment of a tumor or cancer disease thus comprises the administration of at least one coding RNA (as additional pharmaceutically active ingredient), preferably at least two, three, four or five coding RNAs, encoding at least two, preferably two, three or four, of the peptides or proteins, or a fragment or variant of any of these, preferably resulting in the expression of said at least two peptides or proteins, or a fragment or variant of any of these, upon administration of the at least one coding RNA to a subject.

According to a particularly preferred embodiment, the treatment of a tumor or cancer disease comprises administration of at least two or three coding RNAs (as additional pharmaceutically active ingredients), wherein each of the coding RNAs encodes a different one of a peptide or protein selected from the group consisting of
IL-12,
CD40L,
an anti-CTLA4 antibody, and
optionally, a decoy PD-1 receptor, preferably a soluble PD-1 receptor,

or a fragment or variant of any of these, and

wherein the IL-12, the CD40L, the decoy PD-1 receptor, or the anti-CTLA4 antibody, or a fragment or variant of any of these, is preferably as described herein.

In another embodiment, the treatment of a tumor or cancer disease comprises administration at least one coding RNA (as additional pharmaceutically active ingredient(s)), wherein the at least one coding RNA is a bi- or multicistronic RNA encoding at least one peptide or protein, preferably two, three or four peptides or proteins, selected from the group consisting of
IL-12,
CD40L,
an anti-CTLA4 antibody, and
optionally, a decoy PD-1 receptor, preferably a soluble PD-1 receptor,

or a fragment or variant of any of these, and

wherein the IL-12, the CD40L, the decoy PD-1 receptor, or the anti-CTLA4 antibody, or a fragment or variant of any of these, is preferably as described herein.

In this context, it was surprisingly found by the inventors that the expression of the combinations of two, preferably three or four, of the peptides or proteins defined herein is particularly beneficial in the treatment of a tumor or cancer disease, in particular when combined with the isRNA as described herein.

According to a preferred embodiment, the present invention provides the isRNA for use in the treatment of a tumor or cancer disease as described herein, wherein the treatment comprises administration of at least one coding RNA (as additional pharmaceutically active ingredient), preferably at least one mRNA, wherein

the at least one coding RNA comprises at least one coding sequence encoding a peptide or protein comprising CD40L or a fragment or variant thereof, and

the same or a different coding RNA comprises at least one coding sequence encoding a peptide or protein comprising IL 12 or a fragment or variant thereof.

In a further preferred embodiment, the present invention provides the isRNA for use in the treatment of a tumor or cancer disease as described herein, wherein the treatment comprises administration of at least one coding RNA (as additional pharmaceutically active ingredient), preferably at least one mRNA, wherein

the at least one coding RNA comprises at least one coding sequence encoding a peptide or protein comprising CD40L or a fragment or variant thereof, and

the same or a different coding RNA comprises at least one coding sequence encoding a peptide or protein comprising a decoy PD-1 receptor, preferably a soluble PD-1 receptor, or a fragment or variant thereof.

According to another embodiment, the present invention provides the isRNA for use in the treatment of a tumor or cancer disease as described herein, wherein the treatment comprises administration of at least one coding RNA (as additional pharmaceutically active ingredient), preferably at least one mRNA, wherein

the at least one coding RNA comprises at least one coding sequence encoding a peptide or protein comprising IL 12 or a fragment or variant thereof, and

the same or a different coding RNA comprises at least one coding sequence encoding a peptide or protein comprising a decoy PD 1 receptor, preferably a soluble PD-1 receptor, or a fragment or variant thereof.

According to a preferred embodiment, the present invention provides the isRNA for use in the treatment of a tumor or cancer disease as described herein, wherein the treatment comprises administration of at least one coding RNA (as additional pharmaceutically active ingredient), preferably at least one mRNA, wherein

the at least one coding RNA comprises at least one coding sequence encoding a peptide or protein comprising IL12 or a fragment or variant thereof, and

the same or a different coding RNA comprises at least one coding sequence encoding a peptide or protein comprising an anti-CTLA4 antibody or a fragment or variant thereof.

According to a preferred embodiment, the present invention provides the isRNA for use in the treatment of a tumor or cancer disease as described herein, wherein the treatment comprises administration of at least one coding RNA (as additional pharmaceutically active ingredient), preferably at least one mRNA, wherein

the at least one coding RNA comprises at least one coding sequence encoding a peptide or protein comprising CD40L or a fragment or variant thereof, and

the same or a different coding RNA comprises at least one coding sequence encoding a peptide or protein comprising an anti-CTLA4 antibody or a fragment or variant thereof.

According to a preferred embodiment, the present invention provides the isRNA for use in the treatment of a tumor or cancer disease as described herein, wherein the treatment comprises administration of at least one coding RNA (as additional pharmaceutically active ingredient), preferably at least one mRNA, wherein

the at least one coding RNA comprises at least one coding sequence encoding a peptide or protein comprising a decoy PD 1 receptor, preferably a soluble PD-1 receptor, or a fragment or variant thereof, and

the same or a different coding RNA comprises at least one coding sequence encoding a peptide or protein comprising an anti-CTLA4 antibody or a fragment or variant thereof.

In a particularly preferred embodiment, the present invention provides the isRNA for use in the treatment of a tumor or cancer disease as described herein, wherein the treatment comprises administration of at least one coding RNA (as additional pharmaceutically active ingredient), preferably at least one mRNA, wherein

the at least one coding RNA comprises at least one coding sequence encoding a peptide or protein comprising IL-12 or a fragment or variant thereof,

the same or a different coding RNA comprises at least one coding sequence encoding a peptide or protein comprising a decoy PD 1 receptor, preferably a soluble PD-1 receptor, or a fragment or variant thereof, and the same or a different coding RNA comprises at least one coding sequence encoding a peptide or protein comprising CD40L or a fragment or variant thereof.

In that embodiment, the coding sequence encoding a peptide or protein comprising IL-12 or a fragment or variant thereof, the coding sequence encoding a peptide or protein comprising a decoy PD 1 receptor, preferably a soluble PD-1 receptor, or a fragment or variant thereof, and the coding sequence encoding a peptide or protein comprising CD40L or a fragment or variant thereof may preferably be located on a separate coding RNA, preferably a separate mRNA. Alternatively, at least two of the coding sequences encoding a peptide or protein comprising IL-12 or a fragment or variant thereof, encoding a peptide or protein comprising a decoy PD 1 receptor, preferably a soluble PD-1 receptor, or a fragment or variant thereof, and encoding a peptide or protein comprising CD40L or a fragment or variant thereof, are located on the same coding RNA, which is preferably a bi- or multicistronic RNA.

According to certain preferred embodiments, the present invention provides the isRNA for use in the treatment of a tumor or cancer disease as described herein, wherein the treatment comprises administration of at least one coding RNA (as additional pharmaceutically active ingredient), preferably at least one mRNA, wherein

the at least one coding RNA comprises at least one coding sequence encoding a peptide or protein comprising or consisting of IL-12 or a fragment or variant thereof,

the same or a different coding RNA comprises at least one coding sequence encoding a peptide or protein comprising or consisting of CD40L or a fragment or variant thereof, and

the same or a different coding RNA comprises at least one coding sequence encoding a peptide or protein comprising or consisting of an anti-CTLA4 antibody, preferably as described herein, or a fragment or variant thereof.

This embodiment is particularly preferred if the patient receives or has received a treatment with a PD-1 antagonist or a PD-L1 antagonist, such as an anti-PD-1 or anti-PD-L1 antibody.

According to certain preferred embodiments, the present invention provides the isRNA for use in the treatment of a tumor or cancer disease as described herein, wherein the treatment comprises administration of at least one coding RNA (as additional pharmaceutically active ingredient), preferably at least one mRNA, wherein

the at least one coding RNA comprises at least one coding sequence encoding a peptide or protein comprising or consisting of IL-12 or a fragment or variant thereof,

the same or a different coding RNA comprises at least one coding sequence encoding a peptide or protein comprising or consisting of a decoy PD 1 receptor, preferably a soluble PD-1 receptor, or a fragment or variant thereof,

the same or a different coding RNA comprises at least one coding sequence encoding a peptide or protein comprising or consisting of CD40L or a fragment or variant thereof, and

the same or a different coding RNA comprises at least one coding sequence encoding a peptide or protein comprising or consisting of an anti-CTLA4 antibody, preferably as described herein, or a fragment or variant thereof.

This embodiment is particularly preferred if the patient does not receive or has not received a treatment with a PD-1 antagonist or a PD-L1 antagonist, such as an anti PD-1 or anti-PD-L1 antibody.

In these embodiments, the coding sequence encoding a peptide or protein comprising or consisting of IL-12 or a fragment or variant thereof, optionally the coding sequence encoding a peptide or protein comprising or consisting of a decoy PD 1 receptor, preferably a soluble PD-1 receptor, or a fragment or variant thereof, the coding sequence encoding a peptide or protein comprising or consisting of CD40L or a fragment or variant thereof, and the coding sequence encoding a peptide or protein comprising or consisting of an anti-CTLA4 antibody or a fragment or variant thereof, may preferably be located on a separate coding RNA, preferably a separate mRNA. Alternatively, at least two of the coding sequences encoding a peptide or protein comprising or consisting of IL-12 or a fragment or variant thereof, the coding sequence encoding a peptide or protein comprising or consisting of a decoy PD 1 receptor, preferably a soluble PD-1 receptor, or a fragment or variant thereof, the coding sequence encoding a peptide or protein comprising or consisting of CD40L or a fragment or variant thereof, and the coding sequence encoding a peptide or protein comprising or consisting of an anti-CTLA4 antibody or a fragment or variant thereof, are located on the same coding RNA, which is preferably a bi- or multicistronic RNA.

In some embodiments, the present invention provides the isRNA for use in the treatment of a tumor or cancer disease as described herein, wherein the treatment comprises administration of at least one coding RNA (as additional pharmaceutically active ingredient), preferably at least one mRNA, wherein

the at least one coding RNA comprises at least one coding sequence encoding a peptide or protein comprising or consisting of IL-12 or a fragment or variant thereof,

the same or a different coding RNA comprises at least one coding sequence encoding a peptide or protein comprising or consisting of CD40L or a fragment or variant thereof, and

the same or a different coding RNA comprises at least one coding sequence encoding a peptide or protein comprising or consisting of an anti-CTLA4 antibody, preferably as described herein, or a fragment or variant thereof.

In these embodiments, the coding sequence encoding a peptide or protein comprising or consisting of IL-12 or a fragment or variant thereof, the coding sequence encoding a peptide or protein comprising or consisting of CD40L or a fragment or variant thereof, and the coding sequence encoding a peptide or protein comprising or consisting of an anti-CTLA4 antibody or a fragment or variant thereof, may preferably be located on a separate coding RNA, preferably a separate mRNA. Alternatively, at least two of the coding sequences encoding a peptide or protein comprising or consisting of IL-12 or a fragment or variant thereof, the coding sequence encoding a peptide or protein comprising or consisting of CD40L or a fragment or variant thereof, and the coding sequence encoding a peptide or protein comprising or consisting of an anti-CTLA4 antibody or a fragment or variant thereof, are located on the same coding RNA, which is preferably a bi- or multicistronic RNA.

In a preferred embodiment, the present invention provides the isRNA for use in the treatment of a tumor or cancer disease as described herein, wherein the treatment comprises administration of at least one coding RNA (as additional pharmaceutically active ingredient), preferably at least one mRNA, wherein the at least one coding sequence encodes at least one tumor antigen or a fragment or variant thereof. Preferably the coding sequence comprises at least one nucleic acid sequence preferably selected from the group consisting of SEQ ID Nos. 505-4033; 4561-4591 of PCT/EP2017/059525, or a fragment or variant of any one of said sequences

In a preferred embodiment, the present invention provides the isRNA for use in the treatment of a tumor or cancer disease as described herein, wherein the treatment comprises administration of at least one coding RNA (as additional pharmaceutically active ingredient), preferably at least one mRNA, wherein the at least one coding sequence comprises
a) a nucleic acid sequence selected from the group consisting of SEQ ID NO: 14 to 19 or SEQ ID NOs: 440 to 445 or 447, preferably from the group consisting of SEQ ID NOs: 440 to 445 or 447, or a fragment or variant of any of these sequences, preferably a nucleic acid sequence according to SEQ ID NO: 21 or SEQ ID NO: 447, preferably SEQ ID NO: 447, or a fragment or variant of any of these sequences;
b) a nucleic acid sequence according to SEQ ID NO: 22 or SEQ ID NO: 448, preferably SEQ ID NO: 448, or a fragment or variant thereof,
c) a nucleic acid sequence according to any one of SEQ ID NO: 646-660 or 662-676, preferably according to any one of SEQ ID NO: 646-660, or a fragment or variant of any one of these nucleic acid sequences, and a nucleic acid sequence according to any one of SEQ ID NO: 678-692, 694-705 or 707-709, preferably according to any one of SEQ ID NO: 678-692, or a fragment or variant of any of these nucleic acid sequences; or
   a nucleic acid sequence according to any one of SEQ ID NO: 710-712 or 713-715, or a fragment or variant of any of these nucleic acid sequences, and/or
d) optionally, a nucleic acid sequence according to SEQ ID NO: 13 or SEQ ID NO: 439, preferably SEQ ID NO: 439, or a fragment or variant thereof.

According to one embodiment, the at least one coding RNA or the isRNA as described herein may be in the form of a modified RNA, wherein any modification as defined herein, may be introduced into the at least one coding RNA or into the isRNA as described herein. According to a preferred embodiment, the at least one coding RNA as described herein comprises at least one coding sequence comprising a nucleic acid sequence that is modified compared to the nucleic acid sequence of the coding sequence of the corresponding wild type RNA, and wherein the amino acid sequence encoded by said coding sequence is preferably not modified compared to the amino acid sequence encoded by the coding sequence of the corresponding wild type RNA. Modifications as defined herein preferably lead to a stabilization of the at least one coding RNA as used herein.

In a further preferred embodiment, the present invention thus provides the isRNA for use in the treatment of a tumor or cancer disease as described herein, wherein the treatment comprises administration of at least one coding RNA (as additional pharmaceutically active ingredient), preferably at least one mRNA, wherein the at least one coding sequence comprises
a) a nucleic acid sequence selected from the group consisting of SEQ ID NOs: 14-19; 440-445; 447; 25-30; 36-41; 47-52; 58-63; 69-74; 80-85; 91-96; 102-107; 113-118; 124-129; 135-140; 601-606; 612-617; 623-628; 716-725; 727; 1018-1021 and 1059-1062, or a fragment or variant of any of these sequences, preferably from the group consisting of SEQ ID NOs: 21; 447; 32; 43; 54; 65; 76; 87; 98; 109; 120; 131; 142; 608; 619; 630; 632-644; 726 and 1058, or a fragment or variant of any of these sequences;
b) a nucleic acid sequence selected from the group consisting of SEQ ID NOs: 22; 448; 33; 44; 55; 66; 77; 88; 99; 110; 121; 132; 143; 609; 620; 631; 728-738 and 1025-1028, or a fragment or variant of any of these sequences;
c) a nucleic acid sequence according to any one of SEQ ID NO: 646-660; 662-676 or 1029-1036, preferably according to any one of SEQ ID NO: 646-660, or a fragment or variant of any one of these nucleic acid sequences, and a nucleic acid sequence according to any one of SEQ ID NO: 678-692, 694-705; 707-709 or 1037-1041, preferably according to any one of SEQ ID NO: 678-692, or a fragment or variant of any of these nucleic acid sequences;
   or
   a nucleic acid sequence according to any one of SEQ ID NO: 710-712 or 713-715, or a fragment or variant of any of these nucleic acid sequences, and/or
d) optionally, a nucleic acid sequence selected from the group consisting of SEQ ID NOs: 12; 438; 23; 34; 45; 56; 67; 78; 89; 100; 111; 122; 133; 599; 610; 621 and 1022-1024, or a fragment or variant of any of these sequences, preferably from the group consisting of SEQ ID NOs: 13, 439; 24; 439; 35; 46; 57; 68; 79; 90; 101; 112; 123; 134; 600; 611; 622 and 1043-1054, or a fragment or variant of any of these sequences.

In a preferred embodiment, the present invention thus provides the isRNA for use in the treatment of a tumor or cancer disease as described herein, wherein the treatment comprises administration of at least one coding RNA (as additional pharmaceutically active ingredient), preferably at least one mRNA, wherein the at least one coding sequence comprises
a) a nucleic acid sequence selected from the group consisting of SEQ ID NOs: 25-30; 36-41; 47-52; 58-63; 69-74; 80-85; 91-96; 102-107; 113-118; 124-129; 135-140; 601-606; 612-617; 623-628; 716-725; 727; 1018-1021 and 1059-1062, or a fragment or variant of any of these sequences, preferably from the group consisting of SEQ ID NOs: 32; 43; 54; 65; 76; 87; 98; 109; 120; 131; 142; 608; 619; 630; 632-644; 726 and 1058, or a fragment or variant of any of these sequences;
b) a nucleic acid sequence selected from the group consisting of SEQ ID NOs: 33; 44; 55; 66; 77; 88; 99; 110; 121; 132; 143; 609; 620; 631; 728-738; 1025-1028 and 1025-1028, or a fragment or variant of any of these sequences;
c) a nucleic acid sequence according to any one of SEQ ID NO: 646-660; 662-676 or 1029-1036, preferably according to any one of SEQ ID NO: 646-660, or a fragment or variant of any one of these nucleic acid sequences, and a nucleic acid sequence according to any one of SEQ ID NO: 678-692, 694-705; 707-709 or 1037-1041, preferably according to any one of SEQ ID NO: 678-692, or a fragment or variant of any of these nucleic acid sequences;
   or
   a nucleic acid sequence according to any one of SEQ ID NO: 710-712 or 713-715, or a fragment or variant of any of these nucleic acid sequences, and/or
d) optionally, a nucleic acid sequence selected from the group consisting of SEQ ID NOs: 23; 34; 45; 56; 67; 78; 89; 100; 111; 122; 133; 599; 610; 621; 1022-1024, or a fragment or variant of any of these sequences, preferably from the group consisting of SEQ ID NOs: 24; 35; 46; 57; 68; 79; 90; 101; 112; 123; 134; 600; 611; 622 and 1043-1054, or a fragment or variant of any of these sequences.

In this context, a coding RNA as used herein comprising at least one coding sequence that comprises a (wild type or modified) nucleic acid sequence encoding IL-12 or a fragment or variant thereof as described herein, may preferably comprise a nucleic acid sequence according to any of SEQ ID NOs: 20; 31; 42; 53; 64; 75; 86; 97; 108; 119; 130; 141; 446; 607; 618 or 629, or a fragment or variant of any of these sequences.

According to one embodiment, the at least one coding RNA as described herein may thus be provided as a “stabilized RNA”, that is to say as an RNA that is essentially resistant to in vivo degradation (e.g. by an exo- or endo-nuclease). Such stabilization can be effected, for example, by a modified phosphate backbone of the at least one coding RNA as used herein. A backbone modification in connection with the present invention is a modification in which phosphates of the backbone of the nucleotides contained in the RNA are chemically modified. Nucleotides that may be preferably used in this connection contain e.g. a phosphorothioate-modified phosphate backbone, preferably at least one of the phosphate oxygens contained in the phosphate backbone being replaced by a sulfur atom. Stabilized RNAs may further include, for example: non-ionic phosphate analogues, such as, for example, alkyl and aryl phosphonates, in which the charged phosphonate oxygen is replaced by an alkyl or aryl group, or phosphodiesters and alkylphosphotriesters, in which the charged oxygen residue is present in alkylated form. Such backbone modifications typically include, without implying any limitation, modifications from the group consisting of methylphosphonates, phosphoramidates and phosphorothioates (e.g. cytidine-5′-O-(1-thiophosphate)).

In the following, specific modifications are described, which are preferably capable of “stabilizing” the at least one coding RNA as defined herein.

Chemical Modifications:

The term “RNA modification” as used herein may refer to chemical modifications comprising backbone modifications as well as sugar modifications or base modifications.

In this context, a modified RNA as defined herein may contain nucleotide analogues/modifications, e.g. backbone modifications, sugar modifications or base modifications. A backbone modification in connection with the present invention is a modification, in which phosphates of the backbone of the nucleotides contained in an RNA as defined herein are chemically modified. A sugar modification in connection with the present invention is a chemical modification of the sugar of the nucleotides of the RNA as defined herein. Furthermore, a base modification in connection with the present invention is a chemical modification of the base moiety of the nucleotides of the RNA. In this context, nucleotide analogues or modifications are preferably selected from nucleotide analogues, which are applicable for transcription and/or translation.

Sugar Modifications:

The modified nucleosides and nucleotides, which may be incorporated into a modified RNA as described herein, can be modified in the sugar moiety. For example, the 2′ hydroxyl group (OH) can be modified or replaced with a number of different “oxy” or “deoxy” substituents. Examples of “oxy”−2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH₂CH₂O)nCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O.

The sugar group can also contain one or more carbons that possess the opposite stereochemical configuration than that of the corresponding carbon in ribose. Thus, a modified RNA can include nucleotides containing, for instance, arabinose as the sugar.

Backbone Modifications:

The phosphate backbone may further be modified in the modified nucleosides and nucleotides, which may be incorporated into a modified RNA as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

Base Modifications:

The modified nucleosides and nucleotides, which may be incorporated into a modified RNA as described herein can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particularly preferred embodiments of the present invention, the nucleotide analogues/modifications are selected from base modifications, which are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate.

In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methylcytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group.

In specific embodiments, a modified nucleoside is 5′-O-(1-thiophosphate)-adenosine, 5′-O-(1-thiophosphate)-cytidine, 5′-O-(1-thiophosphate)-guanosine, 5′-O-(1-thiophosphate)-uridine or 5′-O-(1-thiophosphate)-pseudouridine.

In further specific embodiments, a modified RNA as described herein may comprise nucleoside modifications selected from 6-aza-cytidine, 2-thio-cytidine, α-thio-cytidine, Pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, Pyrrolo-cytidine, inosine, α-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, α-thio-adenosine, 8-azido-adenosine, 7-deaza-adenosine.

Lipid Modification:

According to a further embodiment, a modified RNA as defined herein can contain a lipid modification. Such a lipid-modified RNA typically comprises an RNA as defined herein, preferably a coding RNA as described herein. Such a lipid-modified RNA as defined herein typically further comprises at least one linker covalently linked with that RNA, and at least one lipid covalently linked with the respective linker. Alternatively, the lipid-modified RNA comprises at least one RNA as defined herein, preferably a coding RNA as described herein, and at least one (bifunctional) lipid covalently linked (without a linker) with that RNA. According to a third alternative, the lipid-modified RNA comprises an RNA molecule as defined herein, preferably a coding RNA as described herein, at least one linker covalently linked with that RNA, and at least one lipid covalently linked with the respective linker, and also at least one (bifunctional) lipid covalently linked (without a linker) with that RNA. In this context, it is particularly preferred that the lipid modification is present at the terminal ends of a linear RNA sequence.

G/C Content Optimization:

According to an especially preferred embodiment of the invention, the at least one coding RNA as described herein is modified. Preferably the RNA is stabilized by modifying and preferably increasing the G (guanosine)/C (cytosine) content of at least one coding region thereof. Therein, the G/C content of the RNA of the coding region is increased compared to the G/C content of the coding region of its particular wild type coding sequence, i.e. the corresponding unmodified RNA. However, the encoded amino acid sequence of the RNA is preferably not modified compared to the encoded amino acid sequence of the particular wild type/unmodified RNA.

The modification of the G/C-content of the at least one coding RNA as described herein is based on the fact that RNA sequences having an increased G (guanosine)/C (cytosine) content are typically more stable than RNA sequences having an increased A (adenosine)/U (uracil) content. The codons of a coding sequence or a whole RNA might therefore be varied compared to the wild type coding sequence or RNA, such that they include an increased amount of G/C nucleotides, while the translated amino acid sequence is preferably retained. In respect to the fact that several codons code for one and the same amino acid (so-called degeneration of the genetic code), the most favourable codons for the stability can be determined (so-called alternative codon usage). Depending on the amino acid to be encoded by the at least one RNA, there are various possibilities for modification of the RNA sequence, compared to its wild type sequence. In the case of amino acids which are encoded by codons, which contain exclusively G or C nucleotides, no modification of the codon is necessary. Thus, the codons for Pro (CCC or CCG), Arg (CGC or CGG), Ala (GCC or GCG) and Gly (GGC or GGG) require no modification, since no A or U is present. In contrast, codons which contain A and/or U nucleotides can be modified by substitution of other codons, which code for the same amino acids but contain no A and/or U. Examples of these are: the codons for Pro can be modified from CCU or CCA to CCC or CCG; the codons for Arg can be modified from CGU or CGA or AGA or AGG to CGC or CGG; the codons for Ala can be modified from GCU or GCA to GCC or GCG; the codons for Gly can be modified from GGU or GGA to GGC or GGG. In other cases, although A or U nucleotides cannot be eliminated from the codons, it is however possible to decrease the A and U content by using codons which contain a lower content of A and/or U nucleotides. Examples of these are: the codons for Phe can be modified from UUU to UUC; the codons for Leu can be modified from UUA, UUG, CUU or CUA to CUC or CUG; the codons for Ser can be modified from UCU or UCA or AGU to UCC, UCG or AGC; the codon for Tyr can be modified from UAU to UAC; the codon for Cys can be modified from UGU to UGC; the codon for His can be modified from CAU to CAC; the codon for Gln can be modified from CAA to CAG; the codons for Ile can be modified from AUU or AUA to AUC; the codons for Thr can be modified from ACU or ACA to ACC or ACG; the codon for Asn can be modified from MU to MC; the codon for Lys can be modified from MA to AAG; the codons for Val can be modified from GUU or GUA to GUC or GUG; the codon for Asp can be modified from GAU to GAC; the codon for Glu can be modified from GM to GAG; the stop codon UAA can be modified to UAG or UGA. In the case of the codons for Met (AUG) and Trp (UGG), on the other hand, there is no possibility of sequence modification. The substitutions listed above can be used either individually or in all possible combinations to increase the G/C content of the at least one mRNA of the composition of the present invention compared to its particular wild type mRNA (i.e. the original sequence). Thus, for example, all codons for Thr occurring in the wild type sequence can be modified to ACC (or ACG). Preferably, however, for example, combinations of the above substitution possibilities are used:
substitution of all codons coding for Thr in the original sequence (wild type mRNA) to ACC (or ACG) and
substitution of all codons originally coding for Ser to UCC (or UCG or AGC); substitution of all codons coding for Ile in the original sequence to AUC and
substitution of all codons originally coding for Lys to AAG and
substitution of all codons originally coding for Tyr to UAC; substitution of all codons coding for Val in the original sequence to GUC (or GUG) and
substitution of all codons originally coding for Glu to GAG and
substitution of all codons originally coding for Ala to GCC (or GCG) and
substitution of all codons originally coding for Arg to CGC (or CGG) and
substitution of all codons originally coding for Gly to GGC (or GGG) and
substitution of all codons originally coding for Asn to AAC and
substitution of all codons originally coding for Phe to UUC and
substitution of all codons originally coding for Cys to UGC and
substitution of all codons originally coding for Leu to CUG (or CUC) and
substitution of all codons originally coding for Gln to CAG and
substitution of all codons originally coding for Pro to CCC (or CCG) and
substitution of all codons originally coding for His to CAC and
substitution of all codons originally coding for Asp to GAC and
substitution of all codons originally coding for the stop codon to UGA (or UAG); etc.

Preferably, the G/C content of the coding region of the at least one coding RNA as described herein is increased by at least 7%, more preferably by at least 15%, particularly preferably by at least 20%, compared to the G/C content of the coding region of the wild type RNA. According to a specific embodiment at least 5%, 10%, 20%, 30%, 40%, 50%, 60%, more preferably at least 70%, even more preferably at least 80% and most preferably at least 90%, 95% or even 100% of the substitutable codons in the region coding for a protein or peptide as defined herein or its fragment or variant thereof or the whole sequence of the wild type RNA sequence or coding sequence are substituted, thereby increasing the G/C content of said sequence. In this context, it is particularly preferable to increase the G/C content of the at least one coding RNA as described herein to the maximum (i.e. 100% of the substitutable codons), in particular in the coding region, compared to the wild type sequence.

According to the invention, a further preferred modification of the coding sequence of the at least one coding RNA is based on the finding that the translation efficiency is also determined by a different frequency in the occurrence of tRNAs in cells. Thus, if so-called “rare codons” are present in the at least one coding region of the at least one coding RNA as described herein to an increased extent, the corresponding modified at least one coding RNA is translated to a significantly poorer degree than in the case where codons encoding relatively “frequent” tRNAs are present. According to the invention, in the modified at least one coding RNA as described herein, the region which encodes one of the above defined peptides or proteins is modified compared to the corresponding region of the wild type RNA such that at least one codon of the wild type sequence, which encodes a tRNA which is relatively rare in the cell, is exchanged for a codon, which enodes a tRNA which is relatively frequent in the cell and carries the same amino acid as the relatively rare tRNA. By this modification, the sequence of the at least one coding region of the at least one coding RNA as described herein is modified such that codons for which frequently occurring tRNAs are available are inserted. In other words, according to the invention, by this modification all codons of the wild type sequence which code for a tRNA which is relatively rare in the cell can in each case be exchanged for a codon which codes for a tRNA which is relatively frequent in the cell and which, in each case, carries the same amino acid as the relatively rare tRNA. Which tRNAs occur relatively frequently in the cell and which, in contrast, occur relatively rarely is known to a person skilled in the art; cf. e.g. Akashi, Curr. Opin. Genet. Dev. 2001, 11(6): 660-666. The codons which use for the particular amino acid the tRNA which occurs the most frequently, e.g. the Gly codon, which uses the tRNA, which occurs the most frequently in the (human) cell, are particularly preferred. According to the invention, it is particularly preferable to link the sequential G/C content which is increased, in particular maximized, in the modified at least one coding RNA as described herein, with the “frequent” codons without modifying the amino acid sequence of the protein encoded by the coding region of the RNA. This preferred embodiment allows provision of a particularly efficiently translated and stabilized (modified) at least one coding RNA as described herein. The determination of a modified at least one coding RNA as described herein (increased G/C content; exchange of tRNAs) can be carried out using the computer program explained in WO 02/098443—the disclosure content of which is included in its full scope in the present invention. Using this computer program, the nucleotide sequence of any desired coding RNA can be modified with the aid of the genetic code or the degenerative nature thereof such that a maximum G/C content results, in combination with the use of codons which code for tRNAs occurring as frequently as possible in the cell, the amino acid sequence coded by the modified at least one coding RNA preferably not being modified compared to the non-modified sequence. Alternatively, it is also possible to modify only the G/C content or only the codon usage compared to the original sequence. The source code in Visual Basic 6.0 (development environment used: Microsoft Visual Studio Enterprise 6.0 with Servicepack 3) is also described in WO 02/098443. In a further preferred embodiment of the present invention, the A/U content in the environment of the ribosome binding site of the at least one coding RNA as described herein is increased compared to the A/U content in the environment of the ribosome binding site of its particular wild type RNA. This modification (an increased A/U content around the ribosome binding site) increases the efficiency of ribosome binding to the at least one RNA. An effective binding of the ribosomes to the ribosome binding site (Kozak sequence: GCCGCCACCAUGG (SEQ ID NO: 429), the AUG forms the start codon) in turn has the effect of an efficient translation of the at least one coding RNA. According to a further embodiment of the present invention the at least one coding RNA as described herein may be modified with respect to potentially destabilizing sequence elements. Particularly, the coding region and/or the 5′ and/or 3′ untranslated region of this RNA may be modified compared to the particular wild type RNA such that it contains no destabilizing sequence elements, the coded amino acid sequence of the modified at least one coding RNA preferably not being modified compared to its particular wild type RNA. It is known that, for example, in sequences of eukaryotic RNAs destabilizing sequence elements (DSE) occur, to which signal proteins bind and regulate enzymatic degradation of RNA in vivo. For further stabilization of the modified at least one coding RNA, optionally in the region which encodes for a protein or peptide as defined herein, one or more such modifications compared to the corresponding region of the wild type RNA can therefore be carried out, so that no or substantially no destabilizing sequence elements are contained there. According to the invention, DSE present in the untranslated regions (3′- and/or 5′-UTR) can also be eliminated from the at least one coding RNA described herein by such modifications. Such destabilizing sequences are e.g. AU-rich sequences (AURES), which occur in 3′-UTR sections of numerous unstable RNAs (Caput et al., Proc. Natl. Acad. Sci. USA 1986, 83: 1670 to 1674). The at least one coding RNA as described herein is therefore preferably modified compared to the wild type RNA such that the at least one coding RNA contains no such destabilizing sequences. This also applies to those sequence motifs which are recognized by possible endonucleases, e.g. the sequence GAACAAG, which is contained in the 3′-UTR segment of the gene which codes for the transferrin receptor (Binder et al., EMBO J. 1994, 13: 1969 to 1980). These sequence motifs are also preferably removed in the at least one coding RNA as described herein.

In a preferred embodiment, the present invention thus provides the isRNA for use in the treatment of a tumor or cancer disease as described herein, wherein the treatment comprises administration of at least one coding RNA (as additional pharmaceutically active ingredient), preferably at least one mRNA, wherein the at least one coding sequence comprises
a) a nucleic acid sequence selected from the group consisting of SEQ ID NOs: 25-30; 80-85; 91-96; 102-107; 113-118; 601-606; 124-129; 135-140; 612-617; 623-628; 716-725; 727; 1018-1021 and 1059-1062, or a fragment or variant of any of these sequences, preferably from the group consisting of SEQ ID NOs: 32; 87; 98; 109; 120; 131; 142; 608; 619; 630; 632; 636-644; 726 and 1058, or a fragment or variant of any of these sequences;
b) a nucleic acid sequence selected from the group consisting of SEQ ID NOs: 33; 88; 99; 110; 121; 132; 143; 609; 620; 631; 728-738 and 1025-1028, or a fragment or variant of any of these sequences;
c) a nucleic acid sequence according to any one of SEQ ID NO: 646, 650-658, 662, 666-674 or 1029-1036, preferably according to any one of SEQ ID NO: 646 or 650-658, or a fragment or variant of any one of these nucleic acid sequences, and a nucleic acid sequence according to any one of SEQ ID NO: 678, 682-690, 694, 698-705, 707 or 1037-1041, preferably according to any one of SEQ ID NO: 678 or 682-690, or a fragment or variant of any of these nucleic acid sequences;
   or
   a nucleic acid sequence according to SEQ ID NO: 710 or 713, or a fragment or variant of any of these nucleic acid sequences, and/or
d) optionally, a nucleic acid sequence selected from the group consisting of SEQ ID NOs: 23; 78; 89; 100; 111; 122; 133; 599; 610; 621 and 1022-1024, or a fragment or variant of any of these sequences, preferably from the group consisting of SEQ ID NOs: 24; 79; 90; 101; 112; 123; 134; 600; 611; 622; 1043 and 1047-1054, or a fragment or variant of any of these sequences.

Adaptation to Human Codon Usage:

According to the invention, a further preferred modification of the at least one coding RNA as described herein is based on the finding that codons coding for the same amino acid occur in different frequencies. According to the invention, in the modified at least one coding RNA as described herein, the region which encodes at least one of the above defined peptides or proteins (coding sequence) is preferably modified compared to the corresponding region of the wild type RNA such that the frequency of the codons encoding the same amino acid corresponds to the naturally occurring frequency of that codon present in the human coding usage as e.g. shown in Table 2.

This means, for example, that for the amino acid alanine (Ala) present in the amino acid sequence of the encoded protein according to the invention, the wild type coding sequence is adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see Table 2).

TABLE 2
Human codon usage table (most frequent codon marked with an asterisk)

| Amino acid | codon | fraction | /1000 |
|---|---|---|---|
| Ala | GCG | 0.10 | 7.4 |
| Ala | GCA | 0.22 | 15.8 |
| Ala | GCT | 0.28 | 18.5 |
| Ala | GCC* | 0.40 | 27.7 |
| Cys | TGT | 0.42 | 10.6 |
| Cys | TGC* | 0.58 | 12.6 |
| Asp | GAT | 0.44 | 21.8 |
| Asp | GAC* | 0.56 | 25.1 |
| Glu | GAG* | 0.59 | 39.6 |
| Glu | GAA | 0.41 | 29.0 |
| Phe | TTT | 0.43 | 17.6 |
| Phe | TTC* | 0.57 | 20.3 |
| Gly | GGG | 0.23 | 16.5 |
| Gly | GGA | 0.26 | 16.5 |
| Gly | GGT | 0.18 | 10.8 |
| Gly | GGC* | 0.33 | 22.2 |
| His | CAT | 0.41 | 10.9 |
| His | CAC* | 0.59 | 15.1 |
| Ile | ATA | 0.14 | 7.5 |
| Ile | ATT | 0.35 | 16.0 |
| Ile | ATC* | 0.52 | 20.8 |
| Lys | AAG* | 0.60 | 31.9 |
| Lys | AAA | 0.40 | 24.4 |
| Leu | TTG | 0.12 | 12.9 |
| Leu | TTA | 0.06 | 7.7 |
| Leu | CTG* | 0.43 | 39.6 |
| Leu | CTA | 0.07 | 7.2 |
| Leu | CTT | 0.12 | 13.2 |
| Leu | CTC | 0.20 | 19.6 |
| Met | ATG* | 1 | 22.0 |
| Asn | AAT | 0.44 | 17.0 |
| Asn | AAC* | 0.56 | 19.1 |
| Pro | CCG | 0.11 | 6.9 |
| Pro | CCA | 0.27 | 16.9 |
| Pro | CCT | 0.29 | 17.5 |
| Pro | CCC* | 0.33 | 19.8 |
| Gin | CAG* | 0.73 | 34.2 |
| Gin | CAA | 0.27 | 12.3 |
| Arg | AGG | 0.22 | 12.0 |
| Arg | AGA* | 0.21 | 12.1 |
| Arg | CGG | 0.19 | 11.4 |
| Arg | CGA | 0.10 | 6.2 |
| Arg | CGT | 0.09 | 4.5 |
| Arg | CGC | 0.19 | 10.4 |
| Ser | AGT | 0.14 | 12.1 |
| Ser | AGC* | 0.25 | 19.5 |
| Ser | TCG | 0.06 | 4.4 |
| Ser | TCA | 0.15 | 12.2 |
| Ser | TCT | 0.18 | 15.2 |
| Ser | TCC | 0.23 | 17.7 |
| Thr | ACG | 0.12 | 6.1 |
| Thr | ACA | 0.27 | 15.1 |
| Thr | ACT | 0.23 | 13.1 |
| Thr | ACC* | 0.38 | 18.9 |
| Val | GTG* | 0.48 | 28.1 |
| Val | GTA | 0.10 | 7.1 |
| Val | GTT | 0.17 | 11.0 |
| Val | GTC | 0.25 | 14.5 |
| Trp | TGG* | 1 | 13.2 |
| Tyr | TAT | 0.42 | 12.2 |
| Tyr | TAC* | 0.58 | 15.3 |
| Stop | TGA* | 0.61 | 1.6 |
| Stop | TAG | 0.17 | 0.8 |
| Stop | TAA | 0.22 | 1.0 |

In a preferred embodiment, the present invention thus provides the isRNA for use in the treatment of a tumor or cancer disease as described herein, wherein the treatment comprises administration of at least one coding RNA (as additional pharmaceutically active ingredient), preferably at least one mRNA, wherein the at least one coding sequence comprises
a) a nucleic acid sequence selected from the group consisting of SEQ ID NOs: 47-52 and 58-63, or a fragment or variant of any of these sequences, preferably from the group consisting of SEQ ID NOs: 54; 65 and 634, or a fragment or variant of any of these sequences;
b) a nucleic acid sequence selected from the group consisting of SEQ ID NOs: 55-66, or a fragment or variant of any of these sequences;
c) a nucleic acid sequence according to any one of SEQ ID NO: 648, 659, 664 or 675, preferably according to SEQ ID NO: 648 or 659, or a fragment or variant of any one of these nucleic acid sequences, and a nucleic acid sequence according to any one of SEQ ID NO: 680, 691, 696 or 708, preferably according to SEQ ID NO: 680 or 691, or a fragment or variant of any of these nucleic acid sequences;
   or
   a nucleic acid sequence according to SEQ ID NO: 711 or 714, or a fragment or variant of any of these nucleic acid sequences, and/or
d) optionally, a nucleic acid sequence selected from the group consisting of SEQ ID NOs: 45 and 56, or a fragment or variant of any of these sequences, preferably from the group consisting of SEQ ID NOs: 46-57 and 1045, or a fragment or variant of any of these sequences.

Codon-Optimization:

According to a particularly preferred embodiment it is preferred, that all codons of the wild type sequence of the coding region of the at least one coding RNA as described herein which code for a tRNA which is relatively rare in the cell is in each case exchanged for a codon which codes for a tRNA which is relatively frequent in the cell and which, in each case, carries the same amino acid as the relatively rare tRNA. Therefore it is particularly preferred that the most frequent codons are used for each encoded amino acid (see Table 2, most frequent codons are marked with asterisks).

This means, for example, that for the amino acid alanine (Ala) present in the amino acid sequence of the encoded peptide or protein according to the invention, the wild type coding sequence is adapted in a way that the most frequent human codon “GCC” is always used for said amino acid, or for the amino acid Cysteine (Cys), the wild type sequence is adapted in a way that the most frequent human codon “TGC” is always used for said amino acid etc.

In a preferred embodiment, the present invention thus provides the isRNA for use in the treatment of a tumor or cancer disease as described herein, wherein the treatment comprises administration of at least one coding RNA (as additional pharmaceutically active ingredient), preferably at least one mRNA, wherein the at least one coding sequence comprises
a) a nucleic acid sequence selected from any one of SEQ ID NOs: 69 to 74, or a fragment or variant of any of these sequences, preferably a nucleic acid sequence according to SEQ ID NOs: 76 and 635, or a fragment or variant thereof;
b) a nucleic acid sequence according to SEQ ID NO: 77, or a fragment or variant thereof;
c) a nucleic acid sequence according to any one of SEQ ID NO: 649, 660, 665 or 676, preferably according to SEQ ID NO: 649 or 660, or a fragment or variant of any of these nucleic acid sequences, and a nucleic acid sequence according to any one of SEQ ID NO: 681, 692, 697 or 709, preferably according to SEQ ID NO: 681 or 692, or a fragment or variant of any of these nucleic acid sequences;
   or
   a nucleic acid sequence according to SEQ ID NO: 712 or 715, or a fragment or variant of any of these nucleic acid sequences, and/or
d) optionally, a nucleic acid sequence according to SEQ ID NO: 67, or a fragment or variant thereof, more preferably a nucleic acid sequence according to SEQ ID NO: 68 or 1046, or a fragment or variant thereof.

C-Enrichment:

According to another embodiment, the at least one coding RNA as described herein may be modified by increasing the C content of the RNA, preferably of the coding region of the at least one coding RNA.

In a particularly preferred embodiment of the present invention, the C content of the coding region of the at least one coding RNA as described herein is modified, particularly increased, compared to the C content of the coding region of its particular wild type RNA, i.e. the unmodified mRNA. The amino acid sequence encoded by the at least one coding RNA is preferably not modified as compared to the amino acid sequence encoded by the particular wild type RNA.

In a preferred embodiment of the present invention, the modified RNA is modified such that at least 10%, 20%, 30%, 40%, 50%, 60%, 70% or 80%, or at least 90% of the theoretically maximal cytosine-content or even a maximal cytosine-content is achieved.

In further preferred embodiments, at least 10%, 20%, 30%, 40%, 50%, 60%, 70%, 80%, 90% or even 100% of the codons of the RNA wild type sequence, which are “cytosine content optimizable” are replaced by codons with a higher cytosine-content as present in the wild type sequence.

In a further preferred embodiment, some of the codons of the wild type coding sequence may additionally be modified such that a codon for a relatively rare tRNA in the cell is exchanged by a codon for a relatively frequent tRNA in the cell, provided that the substituted codon for a relatively frequent tRNA carries the same amino acid as the relatively rare tRNA of the original wild type codon. Preferably, all of the codons for a relatively rare tRNA are replaced by a codon for a relatively frequent tRNA in the cell, except codons encoding amino acids, which are exclusively encoded by codons not containing any cytosine, or except for glutamine (Gln), which is encoded by two codons each containing the same number of cytosines.

In a further preferred embodiment of the present invention, the modified RNA is modified such that at least 80%, or at least 90% of the theoretically maximal cytosine-content or even a maximal cytosine-content is achieved by means of codons, which code for relatively frequent tRNAs in the cell, wherein the amino acid sequence remains unchanged.

Due to the naturally occurring degeneracy of the genetic code, more than one codon may encode a particular amino acid. Accordingly, 18 out of 20 naturally occurring amino acids are encoded by more than 1 codon (with Tryp and Met being an exception), e.g. by 2 codons (e.g. Cys, Asp, Glu), by three codons (e.g. Ile), by 4 codons (e.g. Al, Gly, Pro) or by 6 codons (e.g. Leu, Arg, Ser). However, not all codons encoding the same amino acid are utilized equally frequent under in vivo conditions. Depending on each single organism, a typical codon usage profile is established.

The term “cytosine content-optimizable codon” as used within the context of the present invention refers to codons, which exhibit a lower amount of cytosines than other codons coding for the same amino acid. Accordingly, any wild type codon, which may be replaced by another codon coding for the same amino acid and exhibiting a higher number of cytosines within that codon, is considered to be cytosine-optimizable (C-optimizable). Any such substitution of a C-optimizable wild type codon by the specific C-optimized codon within a wild type coding region increases its overall C-content and reflects a C-enriched modified RNA sequence. A C-maximized RNA sequence contains C-optimized codons for all potentially C-optimizable codons. Accordingly, 100% or all of the theoretically replaceable C-optimizable codons are under such conditions actually replaced by C-optimized codons over the entire length of the coding region.

In this context, cytosine-content optimizable codons are codons, which contain a lower number of cytosines than other codons coding for the same amino acid.

Any of the codons GCG, GCA, GCU codes for the amino acid Ala, which may be exchanged by the codon GCC encoding the same amino acid, and/or
the codon UGU that codes for Cys may be exchanged by the codon UGC encoding the same amino acid, and/or
the codon GAU which codes for Asp may be exchanged by the codon GAC encoding the same amino acid, and/or
the codon that UUU that codes for Phe may be exchanged for the codon UUC encoding the same amino acid, and/or
any of the codons GGG, GGA, GGU that code Gly may be exchanged by the codon GGC encoding the same amino acid, and/or
the codon CAU that codes for His may be exchanged by the codon CAC encoding the same amino acid, and/or
any of the codons AUA, AUU that code for Ile may be exchanged by the codon AUC, and/or
any of the codons UUG, UUA, CUG, CUA, CUU coding for Leu may be exchanged by the codon CUC encoding the same amino acid, and/or
the codon AAU that codes for Asn may be exchanged by the codon MC encoding the same amino acid, and/or
any of the codons CCG, CCA, CCU coding for Pro may be exchanged by the codon CCC encoding the same amino acid, and/or
any of the codons AGG, AGA, CGG, CGA, CGU coding for Arg may be exchanged by the codon CGC encoding the same amino acid, and/or
any of the codons AGU, AGC, UCG, UCA, UCU coding for Ser may be exchanged by the codon UCC encoding the same amino acid, and/or
any of the codons ACG, ACA, ACU coding for Thr may be exchanged by the codon ACC encoding the same amino acid, and/or
any of the codons GUG, GUA, GUU coding for Val may be exchanged by the codon GUC encoding the same amino acid, and/or
the codon UAU coding for Tyr may be exchanged by the codon UAC encoding the same amino acid.

In any of the above instances, the number of cytosines is increased by 1 per exchanged codon. Exchange of all non C-optimized codons (corresponding to C-optimizable codons) of the coding region results in a C-maximized coding sequence. In the context of the invention, at least 70% of the non C-optimized codons are replaced by C-optimized codons of the wild type sequence are replaced by C-optimized codons, preferably at least 80%, more preferably at least 90% within the coding region.

It may be preferred that for some amino acids the percentage of C-optimizable codons replaced by C-optimized codons is less than 70%, while for other amino acids the percentage of replaced codons is higher than 70% to meet the overall percentage of C-optimization of at least 70% of all C-optimizable wild type codons of the coding region.

Preferably, in the C-optimized RNAs of the invention, at least 50% of the C-optimizable wild type codons for any given amino acid are replaced by C-optimized codons, e.g. any modified C-enriched RNA preferably contains at least 50% C-optimized codons at C-optimizable wild type codon positions coding for any single of the above mentioned amino acids Ala, Cys, Asp, Phe, Gly, His, Ile, Leu, Asn, Pro, Arg, Ser, Thr, Val and Tyr, preferably at least 60%.

In this context, codons encoding amino acids, which are not cytosine content-optimizable and which are, however, encoded by at least two codons, may be used without any further selection process. However, the codon of the wild type sequence that codes for a relatively rare tRNA in the cell, e.g. a human cell, may be exchanged for a codon that codes for a relatively frequent tRNA in the cell, whereby both code for the same amino acid. Accordingly, the relatively rare codon GM coding for Glu may be exchanged by the relative frequent codon GAG coding for the same amino acid, and/or

the relatively rare codon AAA coding for Lys may be exchanged by the relative frequent codon AAG coding for the same amino acid, and/or

the relatively rare codon CM coding for Gln is exchanged for the relative frequent codon CAG encoding the same amino acid.

In this context, the amino acids Met (AUG) and Trp (UGG), which are encoded by only one codon each, remain unchanged. Stop codons are not cytosine-content optimized, however, the relatively rare stop codons amber, ochre (UAA, UAG) may be exchanged by the relatively frequent stop codon opal (UGA).

The substitutions listed above may obviously be used individually but also in all possible combinations in order to optimize the cytosine-content of the modified RNA compared to the wild type RNA sequence.

Accordingly, the region of the modified RNA encoding a peptide or protein may be changed compared to the coding region of the wild type RNA in such a way that an amino acid encoded by at least two or more codons, of which one comprises one additional cytosine, such a codon may be exchanged by the C-optimized codon comprising one additional cytosine, whereby the amino acid is unaltered compared to the wild type sequence.

In a preferred embodiment, the present invention thus provides the isRNA for use in the treatment of a tumor or cancer disease as described herein, wherein the treatment comprises administration of at least one coding RNA (as additional pharmaceutically active ingredient), preferably at least one mRNA, wherein the at least one coding sequence comprises
a) a nucleic acid sequence selected from any one of SEQ ID NOs: 36 to 41, or a fragment or variant of any of these sequences, preferably a nucleic acid according to SEQ ID NO: 43 and 633 or a fragment or variant thereof;
b) a nucleic acid sequence according to SEQ ID NO: 44, or a fragment or variant thereof,
c) a nucleic acid sequence according to SEQ ID NO: 647 or 663, preferably according to SEQ ID NO: 647, or a fragment or variant of any of these nucleic acid sequences, and a nucleic acid sequence according to SEQ ID NO: 679 or 695, preferably according to SEQ ID NO: 679, or a fragment or variant of any of these nucleic acid sequences, and/or
d) optionally, a nucleic acid sequence according to SEQ ID NO: 34, or a fragment or variant thereof, preferably a nucleic acid sequence according to SEQ ID NO: 35 and 1044, or a fragment or variant thereof.

According to a further embodiment, the at least one coding RNA as described herein preferably comprises at least one of the following structural elements: a 5′- and/or 3′-untranslated region element (UTR element), particularly a 5′-UTR element which comprises or consists of a nucleic acid sequence, which is derived from the 5′-UTR of a TOP gene or from a fragment, homolog or a variant thereof, or a 5′- and/or 3′-UTR element which may be derivable from a gene that provides a stable mRNA or from a homolog, fragment or variant thereof; a histone stem-loop structure, preferably a histone stem-loop in its 3′ untranslated region; a 5′-CAP structure; a poly-A tail (poly(A) sequence); or a poly(C) sequence.

In a preferred embodiment the at least one coding RNA as described herein comprises at least one 5′- or 3′-UTR element. In this context an UTR element comprises or consists of a nucleic acid sequence, which is derived from the 5′- or 3′-UTR of any naturally occurring gene or which is derived from a fragment, a homolog or a variant of the 5′- or 3′-UTR of a gene. Preferably, the 5′- or 3′-UTR element used according to the present invention is heterologous to the coding region of the at least one coding RNA as described herein. Even if 5′- or 3′-UTR elements derived from naturally occurring genes are preferred, also synthetically engineered UTR elements may be used in the context of the present invention.

In a particularly preferred embodiment, the at least one coding RNA comprises at least one 5′-untranslated region element (5′-UTR element), which comprises or consists of a nucleic acid sequence, which is derived from the 5′-UTR of a TOP gene or which is derived from a fragment, homolog or variant of the 5′-UTR of a TOP gene.

It is particularly preferred that the 5′-UTR element does not comprise a TOP-motif or a 5′-TOP, as defined above.

In some embodiments, the nucleic acid sequence of the 5′-UTR element, which is derived from a 5′-UTR of a TOP gene, terminates at its 3′-end with a nucleotide located at position 1, 2, 3, 4, 5, 6, 7, 8, 9 or 10 upstream of the start codon (e.g. A(U/T)G) of the gene or mRNA it is derived from. Thus, the 5′-UTR element does not comprise any part of the protein coding region. Thus, preferably, the only protein coding part of the at least one coding RNA as described herein is provided by the coding region.

The nucleic acid sequence, which is derived from the 5′-UTR of a TOP gene, is preferably derived from a eukaryotic TOP gene, preferably a plant or animal TOP gene, more preferably a chordate TOP gene, even more preferably a vertebrate TOP gene, most preferably a mammalian TOP gene, such as a human TOP gene.

For example, the 5′-UTR element is preferably selected from 5′-UTR elements comprising or consisting of a nucleic acid sequence which is derived from a nucleic acid sequence selected from the group consisting of SEQ ID Nos. 1-1363, SEQ ID NO: 1395, SEQ ID NO: 1421 and SEQ ID NO: 1422 of the patent application WO2013/143700, the disclosure of which is incorporated herein by reference, from the homologs of SEQ ID Nos: 1-1363, SEQ ID NO: 1395, SEQ ID NO: 1421 and SEQ ID NO: 1422 of the patent application WO2013/143700, from a variant thereof, or preferably from a corresponding RNA sequence. The term “homologs of SEQ ID Nos: 1-1363, SEQ ID NO: 1395, SEQ ID NO: 1421 and SEQ ID NO: 1422 of the patent application WO2013/143700” refers to sequences of other species than Homo sapiens, which are homologous to the sequences according to SEQ ID Nos. 1-1363, SEQ ID NO: 1395, SEQ ID NO: 1421 and SEQ ID NO: 1422 of the patent application WO2013/143700.

In a preferred embodiment, the 5′-UTR element comprises or consists of a nucleic acid sequence, which is derived from a nucleic acid sequence extending from nucleotide position 5 (i.e. the nucleotide that is located at position 5 in the sequence) to the nucleotide position immediately 5′ to the start codon (located at the 3′ end of the sequences), e.g. the nucleotide position immediately 5′ to the ATG sequence, of a nucleic acid sequence selected from SEQ ID Nos. 1-1363, SEQ ID NO: 1395, SEQ ID NO: 1421 and SEQ ID NO: 1422 of the patent application WO2013/143700, from the homologs of SEQ ID Nos: 1-1363, SEQ ID NO: 1395, SEQ ID NO: 1421 and SEQ ID NO: 1422 of the patent application WO2013/143700 from a variant thereof, or a corresponding RNA sequence. It is particularly preferred that the 5′-UTR element is derived from a nucleic acid sequence extending from the nucleotide position immediately 3′ to the 5′-TOP to the nucleotide position immediately 5′ to the start codon (located at the 3′ end of the sequences), e.g. the nucleotide position immediately 5′ to the ATG sequence, of a nucleic acid sequence selected from SEQ ID Nos: 1-1363, SEQ ID NO: 1395, SEQ ID NO: 1421 and SEQ ID NO: 1422 of the patent application WO2013/143700, from the homologs of SEQ ID Nos. 1-1363, SEQ ID NO: 1395, SEQ ID NO: 1421 and SEQ ID NO: 1422 of the patent application WO2013/143700, from a variant thereof, or a corresponding RNA sequence.

In some embodiments, the 5′-UTR element may be any 5′-UTR element described in WO2016/107877. In this context, the disclosure of WO2016/107877 relating to 5′-UTR elements/sequences is herewith incorporated by reference. Particularly preferred 5′-UTR elements are nucleic acid sequences according to SEQ ID NOs: 25 to 30 and SEQ ID NOs: 319 to 382 of the patent application WO2016/107877, or fragments or variants of these sequences. In this context, it is particularly preferred that the 5′-UTR element comprises or consists of a corresponding RNA sequence of the nucleic acid sequence according SEQ ID NOs: 25 to 30 and SEQ ID NOs: 319 to 382 of the patent application WO2016/107877.

In certain embodiments, the 5′-UTR element may be any 5′-UTR element as described in WO2017/036580. In this context, the disclosure of WO2017/036580 relating to 5′-UTR elements/sequences is herewith incorporated by reference. Particularly preferred 5′-UTR elements are nucleic acid sequences according to SEQ ID NOs: 1 to 151 of the patent application WO2017/036580, or fragments or variants of these sequences. In this context, it is particularly preferred that the 5′-UTR element comprises or consists of a corresponding RNA sequence of the nucleic acid sequence according to SEQ ID NOs: 1 to 151 of the patent application WO2017/036580.

In a particularly preferred embodiment, the 5′-UTR element comprises or consists of a nucleic acid sequence, which is derived from a 5′-UTR of a TOP gene encoding a ribosomal protein or from a variant of a 5′-UTR of a TOP gene encoding a ribosomal protein. For example, the 5′-UTR element comprises or consists of a nucleic acid sequence which is derived from a 5′-UTR of a nucleic acid sequence according to any of SEQ ID NOs: 67, 170, 193, 244, 259, 554, 650, 675, 700, 721, 913, 1016, 1063, 1120, 1138, and 1284-1360 of the patent application WO2013/143700, a corresponding RNA sequence, a homolog thereof, or a variant thereof as described herein, preferably lacking the 5′-TOP motif. As described above, the sequence extending from position 5 to the nucleotide immediately 5′ to the ATG (which is located at the 3′end of the sequences) corresponds to the 5′-UTR of said sequences.

Preferably, the 5′-UTR element comprises or consists of a nucleic acid sequence, which is derived from a 5′-UTR of a TOP gene encoding a ribosomal large protein (RPL) or from a homolog or variant of a 5′-UTR of a TOP gene encoding a ribosomal large protein (RPL). For example, the 5′-UTR element comprises or consists of a nucleic acid sequence which is derived from a 5′-UTR of a nucleic acid sequence according to any of SEQ ID NOs: 67, 259, 1284-1318, 1344, 1346, 1348-1354, 1357, 1358, 1421 and 1422 of the patent application WO2013/143700, a corresponding RNA sequence, a homolog thereof, or a variant thereof as described herein, preferably lacking the 5′-TOP motif.

In a particularly preferred embodiment, the 5′-UTR element comprises or consists of a nucleic acid sequence which is derived from the 5′-UTR of a ribosomal protein Large 32 gene, preferably from a vertebrate ribosomal protein Large 32 (L32) gene, more preferably from a mammalian ribosomal protein Large 32 (L32) gene, most preferably from a human ribosomal protein Large 32 (L32) gene, or from a variant of the 5′-UTR of a ribosomal protein Large 32 gene, preferably from a vertebrate ribosomal protein Large 32 (L32) gene, more preferably from a mammalian ribosomal protein Large 32 (L32) gene, most preferably from a human ribosomal protein Large 32 (L32) gene, wherein preferably the 5′-UTR element does not comprise the 5′-TOP of said gene.

A preferred sequence for a 5′-UTR element corresponds to SEQ ID NO: 1368 of the patent application WO2013/143700.

Accordingly, in a particularly preferred embodiment, the 5′-UTR element comprises or consists of a nucleic acid sequence, which has an identity of at least about 20%, preferably of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence as mentioned above (according to SEQ ID NO: 408 (5′-UTR of human ribosomal protein Large 32 lacking the 5′ terminal oligopyrimidine tract:

(SEQ ID NO: 1075)
GGCGCTGCCTACGGAGGTGGCAGCCATCTCCTTCTCGGCATC;

corresponding to SEQ ID NO: 1368 of the patent application WO2013/143700)) or preferably to a corresponding RNA sequence, or wherein the at least one 5′UTR element comprises or consists of a fragment of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO: 409 or more preferably to a corresponding RNA sequence, wherein, preferably, the fragment is as described above, i.e. being a continuous stretch of nucleotides representing at least 20% etc. of the full-length 5′-UTR.

Preferably, the fragment exhibits a length of at least about 20 nucleotides or more, preferably of at least about 30 nucleotides or more, more preferably of at least about 40 nucleotides or more. Preferably, the fragment is a functional fragment as described herein.

In some embodiments, the at least one coding RNA as described herein comprises a 5′-UTR element, which comprises or consists of a nucleic acid sequence, which is derived from the 5′-UTR of a vertebrate TOP gene, such as a mammalian, e.g. a human TOP gene, selected from RPSA, RPS2, RPS3, RPS3A, RPS4, RPS5, RPS6, RPS7, RPS8, RPS9, RPS10, RPS11, RPS12, RPS13, RPS14, RPS15, RPS15A, RPS16, RPS17, RPS18, RPS19, RPS20, RPS21, RPS23, RPS24, RPS25, RPS26, RPS27, RPS27A, RPS28, RPS29, RPS30, RPL3, RPL4, RPL5, RPL6, RPL7, RPL7A, RPL8, RPL9, RPL10, RPL10A, RPL11, RPL12, RPL13, RPL13A, RPL14, RPL15, RPL17, RPL18, RPL18A, RPL19, RPL21, RPL22, RPL23, RPL23A, RPL24, RPL26, RPL27, RPL27A, RPL28, RPL29, RPL30, RPL31, RPL32, RPL34, RPL35, RPL35A, RPL36, RPL36A, RPL37, RPL37A, RPL38, RPL39, RPL40, RPL41, RPLP0, RPLP1, RPLP2, RPLP3, RPLP0, RPLP1, RPLP2, EEF1A1, EEF1B2, EEF1D, EEF1G, EEF2, EIF3E, EIF3F, EIF3H, EIF2S3, EIF3C, EIF3K, EIF3EIP, EIF4A2, PABPC1, HNRNPA1, TPT1, TUBB1, UBA52, NPM1, ATP5G2, GNB2L1, NME2, UQCRB, or from a homolog or variant thereof, wherein preferably the 5′-UTR element does not comprise a TOP-motif or the 5′-TOP of said genes, and wherein optionally the 5′-UTR element starts at its 5′-end with a nucleotide located at position 1, 2, 3, 4, 5, 6, 7, 8, 9 or 10 downstream of the 5′ terminal oligopyrimidine tract (TOP) and wherein further optionally the 5′-UTR element which is derived from a 5′-UTR of a TOP gene terminates at its 3′-end with a nucleotide located at position 1, 2, 3, 4, 5, 6, 7, 8, 9 or 10 upstream of the start codon (A(U/T)G) of the gene it is derived from.

In certain embodiments, the at least one coding RNA as described herein comprises a 5′-UTR element, which comprises or consists of a nucleic acid sequence, which is derived from the 5′-UTR of a gene selected from the group consisting of Mp68 (6.8 kDa mitochondrial proteolipid), Nosip (Nitric oxide synthase-interacting protein), HSD17B4 (hydroxysteroid (17-beta) dehydrogenase 4), Rpl31 (60S ribosomal protein L31), TUBB4B (Tubulin beta-4B chain), ATP5A1 (ATP synthase subunit alpha (mitochondrial)) and Ndufa4.1 (Cytochrome c oxidase subunit NDUFA4), or from a variant of any of these genes, wherein the gene or the variant thereof is preferably a vertebrate gene, more preferably a mammalian gene, and even more preferably a human gene.

According to a further preferred embodiment, the at least one coding RNA as described herein comprises a 5′-UTR element, which comprises or consists of a nucleic acid sequence, which is derived from the 5′-UTR of a gene selected from the group consisting of Mp68 (6.8 kDa mitochondrial proteolipid), Nosip (Nitric oxide synthase-interacting protein), HSD17B4 (hydroxysteroid (17-beta) dehydrogenase 4), Rpl31 (60S ribosomal protein L31), TUBB4B (Tubulin beta-4B chain), ATP5A1 (ATP synthase subunit alpha (mitochondrial)), Ndufa4.1 (Cytochrome c oxidase subunit NDUFA4), ribosomal protein Large 32 gene (RPL32), a ribosomal protein Large 35 gene (RPL35), a ribosomal protein Large 21 gene (RPL21), an ATP synthase, H+ transporting, mitochondrial F1 complex, alpha subunit 1, cardiac muscle (ATP5A1) gene, an androgen-induced 1 gene (AIG1), cytochrome c oxidase subunit VIc gene (COX6C) and a N-acylsphingosine amidohydrolase (acid ceramidase) 1 gene (ASAH1), or from a variant of any of these genes, wherein the gene or the variant thereof is preferably a vertebrate gene, more preferably a mammalian gene, and even more preferably a human gene.

In further particularly preferred embodiments, the 5′-UTR element comprises or consists of a nucleic acid sequence, which is derived from the 5′-UTR of a ribosomal protein Large 32 gene (RPL32), a ribosomal protein Large 35 gene (RPL35), a ribosomal protein Large 21 gene (RPL21), an ATP synthase, H+transporting, mitochondrial F1 complex, alpha subunit 1, cardiac muscle (ATP5A1) gene, an hydroxysteroid (17-beta) dehydrogenase 4 gene (HSD17B4), an androgen-induced 1 gene (AIG1), cytochrome c oxidase subunit VIc gene (COX6C), or a N-acylsphingosine amidohydrolase (acid ceramidase) 1 gene (ASAH1) or from a variant thereof, preferably from a vertebrate ribosomal protein Large 32 gene (RPL32), a vertebrate ribosomal protein Large 35 gene (RPL35), a vertebrate ribosomal protein Large 21 gene (RPL21), a vertebrate ATP synthase, H+ transporting, mitochondrial F1 complex, alpha subunit 1, cardiac muscle (ATP5A1) gene, a vertebrate hydroxysteroid (17-beta) dehydrogenase 4 gene (HSD17B4), a vertebrate androgen-induced 1 gene (AIG1), a vertebrate cytochrome c oxidase subunit VIc gene (COX6C), or a vertebrate N-acylsphingosine amidohydrolase (acid ceramidase) 1 gene (ASAH1) or from a variant thereof, more preferably from a mammalian ribosomal protein Large 32 gene (RPL32), a ribosomal protein Large 35 gene (RPL35), a ribosomal protein Large 21 gene (RPL21), a mammalian ATP synthase, H+ transporting, mitochondrial F1 complex, alpha subunit 1, cardiac muscle (ATP5A1) gene, a mammalian hydroxysteroid (17-beta) dehydrogenase 4 gene (HSD17B4), a mammalian androgen-induced 1 gene (AIG1), a mammalian cyto-chrome c oxidase subunit VIc gene (COX6C), or a mammalian N-acylsphingosine ami-dohydrolase (acid ceramidase) 1 gene (ASAH1) or from a variant thereof, most preferably from a human ribosomal protein Large 32 gene (RPL32), a human ribosomal protein Large 35 gene (RPL35), a human ribosomal protein Large 21 gene (RPL21), a human ATP synthase, H+transporting, mitochondrial F1 complex, alpha subunit 1, cardiac muscle (ATP5A1) gene, a human hydroxysteroid (17-beta) dehydrogenase 4 gene (HSD17B4), a human androgen-induced 1 gene (AIG1), a human cytochrome c oxidase subunit VIc gene (COX6C), or a human N-acylsphingosine amidohydrolase (acid ceramidase) 1 gene (ASAH1) or from a variant thereof, wherein preferably the 5′-UTR element does not comprise the 5′-TOP of said gene.

Accordingly, in a particularly preferred embodiment, the 5′-UTR element comprises or consists of a nucleic acid sequence, which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO: 1368, or SEQ ID NOs: 1412-1420 of the patent application WO2013/143700, or a corresponding RNA sequence, or wherein the at least one 5′-UTR element comprises or consists of a fragment of a nucleic acid sequence which has an identity of at least about 20%, preferably of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO: 1368, or SEQ ID NOs: 1412-1420 of the patent application WO2013/143700, wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

According to a particularly preferred embodiment, the 5′-UTR element comprises or consists of a nucleic acid sequence, which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to any one of SEQ ID NO: 838, 840, 842, 844, 846, 848, 850, or 1004-1013, or a corresponding RNA sequence, or wherein the at least one 5′-UTR element comprises or consists of a fragment of a nucleic acid sequence which has an identity of at least about 20%, preferably of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to any one of SEQ ID NO: 838, 840, 842, 844, 846, 848, 850, or 1004-1013, wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

In a further preferred embodiment, the 5′-UTR element comprises or consists of a nucleic acid sequence, which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to any one of SEQ ID NO: 838, 840, 842, 844, 846, 848 or 850, or a corresponding RNA sequence, preferably selected from SEQ ID NO: 839, 841, 843, 845, 847, 849 and 851, or wherein the at least one 5′-UTR element comprises or consists of a fragment of a nucleic acid sequence which has an identity of at least about 20%, preferably of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to any one of SEQ ID NO: 838, 840, 842, 844, 846, 848 or 850, wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

Accordingly, in a particularly preferred embodiment, the 5′-UTR element comprises or consists of a nucleic acid sequence, which has an identity of at least about 20%, preferably of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO: 410 (5′-UTR of ATP5A1 lacking the 5′ terminal oligopyrimidine tract: GCGGCTCGGCCATTTTGTCCCAGTCAGTCCGGAGGCTGCGGCTGCAGAAGTACCGCCTGCGGAGTAACTGCAAAG (SEQ ID NO: 1076); corresponding to SEQ ID NO: 1414 of the patent application WO2013/143700 (5′-UTR of ATP5A1 lacking the 5′ terminal oligopyrimidine tract) or preferably to a corresponding RNA sequence (SEQ ID NO: 411), or wherein the at least one 5′UTR element comprises or consists of a fragment of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO: 26 (of the patent application WO2013/143700) or more preferably to a corresponding RNA sequence, wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

In a further preferred embodiment, the at least one coding RNA as described herein further comprises at least one 3′-UTR element, which comprises or consists of a nucleic acid sequence derived from the 3′-UTR of a chordate gene, preferably a vertebrate gene, more preferably a mammalian gene, most preferably a human gene, or from a variant of the 3′-UTR of a chordate gene, preferably a vertebrate gene, more preferably a mammalian gene, most preferably a human gene.

The term ‘3’-UTR element′ refers to a nucleic acid sequence, which comprises or consists of a nucleic acid sequence that is derived from a 3′-UTR or from a variant of a 3′-UTR. A 3′-UTR element in the sense of the present invention may represent the 3′-UTR of an mRNA. Thus, in the sense of the present invention, preferably, a 3′-UTR element may be the 3′-UTR of an mRNA, preferably of an artificial mRNA, or it may be the transcription template for a 3′-UTR of an mRNA. Thus, a 3′-UTR element preferably is a nucleic acid sequence, which corresponds to the 3′-UTR of an mRNA, preferably to the 3′-UTR of an artificial mRNA, such as an mRNA obtained by transcription of a genetically engineered vector construct. Preferably, the 3′-UTR element fulfils the function of a 3′-UTR or encodes a sequence, which fulfils the function of a 3′-UTR.

Preferably, the inventive mRNA comprises a 3′-UTR element which may be derivable from a gene that relates to an mRNA with an enhanced half-life (that provides a stable mRNA), for example a 3′-UTR element as defined and described below. Preferably, the 3′ UTR element, is a nucleic acid sequence derived from a 3′ UTR of a gene, which preferably encodes a stable mRNA, or from a homolog, a fragment or a variant of said gene

In a particularly preferred embodiment, the 3′-UTR element comprises or consists of a nucleic acid sequence which is derived from a 3′-UTR of a gene selected from the group consisting of an albumin gene, an α-globin gene, a β-globin gene, a tyrosine hydroxylase gene, a lipoxygenase gene, and a collagen alpha gene, such as a collagen alpha 1(I) gene, or from a variant of a 3′-UTR of a gene selected from the group consisting of an albumin gene, an α-globin gene, a β-globin gene, a tyrosine hydroxylase gene, a lipoxygenase gene, and a collagen alpha gene, such as a collagen alpha 1(I) gene according to SEQ ID NO: 1369-1390 of the patent application WO2013/143700 whose disclosure is incorporated herein by reference. In a particularly preferred embodiment, the 3′-UTR element comprises or consists of a nucleic acid sequence, which is derived from a 3′-UTR of an albumin gene, preferably a vertebrate albumin gene, more preferably a mammalian albumin gene, most preferably a human albumin gene, most preferably a human albumin gene according to SEQ ID NO: 420 (according SEQ ID No: 1369 of the patent application WO2013/143700). The mRNA sequence may comprise or consist of a nucleic acid sequence which is derived from the 3′-UTR of the human albumin gene according to GenBank Accession number NM_000477.5, or from a fragment or variant thereof.

In this context it is particularly preferred that the at least one coding RNA as described herein comprises a 3′-UTR element comprising a corresponding RNA sequence derived from the nucleic acids according to SEQ ID NO: 1369-1390 of the patent application WO2013/143700 or a fragment, homolog or variant thereof.

Most preferably, the 3′-UTR element comprises the nucleic acid sequence derived from a fragment of the human albumin gene (albumin7 3′UTR) according to SEQ ID NO: 422 or 424 (according to SEQ ID No: 1376 of the patent application WO2013/143700).

In this context it is particularly preferred that the 3′-UTR element of the at least one RNA of the inventive composition comprises or consists of a corresponding RNA sequence of the nucleic acid sequence according to SEQ ID NO: 423 or 425.

In another particularly preferred embodiment, the 3′-UTR element comprises or consists of a nucleic acid sequence which is derived from a 3′-UTR of an α-globin gene, preferably a vertebrate α- or β-globin gene, more preferably a mammalian α- or β-globin gene, most preferably a human α- or β-globin gene according to SEQ ID NO: 412 (corresponding to SEQ ID NO: 1370 of the patent application WO2013/143700 (3′-UTR of Homo sapiens hemoglobin, alpha 1 (HBA1))), or according to SEQ ID NO: 414 (corresponding to SEQ ID NO: 1371 of the patent application WO2013/143700 (3′-UTR of Homo sapiens hemoglobin, alpha 2 (HBA2))), and/or according to SEQ ID NO: 416 (corresponding to SEQ ID NO: 1372 of the patent application WO2013/143700 (3′-UTR of Homo sapiens hemoglobin, beta (HBB)).

For example, the 3′-UTR element may comprise or consist of the center, α-complex-binding portion of the 3′-UTR of an α-globin gene, according to SEQ ID NO: 418 (corresponding to SEQ ID NO: 1393 of the patent application WO2013/143700).

In this context it is particularly preferred that the 3′-UTR element of the RNA of the inventive composition comprises or consists of a corresponding RNA sequence of the nucleic acid sequence according to SEQ ID NO: 419, according to the above or a homolog, a fragment or variant thereof.

The term ‘a nucleic acid sequence which is derived from the 3’-UTR of a [ . . . ] gene′ preferably refers to a nucleic acid sequence which is based on the 3′-UTR sequence of a [ . . . ] gene or on a part thereof, such as on the 3′-UTR of an albumin gene, an α-globin gene, a β-globin gene, a tyrosine hydroxylase gene, a lipoxygenase gene, or a collagen alpha gene, such as a collagen alpha 1(I) gene, preferably of an albumin gene or on a part thereof. This term includes sequences corresponding to the entire 3′-UTR sequence, i.e. the full length 3′-UTR sequence of a gene, and sequences corresponding to a fragment of the 3′-UTR sequence of a gene, such as an albumin gene, α-globin gene, β-globin gene, tyrosine hydroxylase gene, lipoxygenase gene, or collagen alpha gene, such as a collagen alpha 1(I) gene, preferably of an albumin gene.

The term ‘a nucleic acid sequence which is derived from a variant of the 3’-UTR of a [ . . . ] gene′ preferably refers to a nucleic acid sequence which is based on a variant of the 3′-UTR sequence of a gene, such as on a variant of the 3′-UTR of an albumin gene, an α-globin gene, a β-globin gene, a tyrosine hydroxylase gene, a lipoxygenase gene, or a collagen alpha gene, such as a collagen alpha 1(I) gene, or on a part thereof as described above. This term includes sequences corresponding to the entire sequence of the variant of the 3′-UTR of a gene, i.e. the full length variant 3′-UTR sequence of a gene, and sequences corresponding to a fragment of the variant 3′-UTR sequence of a gene. A fragment in this context preferably consists of a continuous stretch of nucleotides corresponding to a continuous stretch of nucleotides in the full-length variant 3′-UTR, which represents at least 20%, preferably at least 30%, more preferably at least 40%, more preferably at least 50%, even more preferably at least 60%, even more preferably at least 70%, even more preferably at least 80%, and most preferably at least 90% of the full-length variant 3′-UTR. Such a fragment of a variant, in the sense of the present invention, is preferably a functional fragment of a variant as described herein.

In some embodiments, the 3′-UTR element may be any 3′-UTR element described in WO2016/107877. In this context, the disclosure of WO2016/107877 relating to 3′-UTR elements/sequences is herewith incorporated by reference. Particularly preferred 3′-UTR elements are SEQ ID NOs: 1 to 24 and SEQ ID NOs: 49 to 318 of the patent application WO2016/107877, or fragments or variants of these sequences. In this context, it is particularly preferred that the 3′-UTR element comprises or consists of a corresponding RNA sequence of the nucleic acid sequence according SEQ ID NOs: 1 to 24 and SEQ ID NOs: 49 to 318 of the patent application WO2016/107877.

In certain embodiments, the 3′-UTR element may be any 3′-UTR element as described in WO2017/036580. In this context, the disclosure of WO2017/036580 relating to 3′-UTR elements/sequences is herewith incorporated by reference. Particularly preferred 3′-UTR elements are nucleic acid sequences according to SEQ ID NOs: 152 to 204 of the patent application WO2017/036580, or fragments or variants of these sequences. In this context, it is particularly preferred that the 3′-UTR element comprises or consists of a corresponding RNA sequence of the nucleic acid sequence according SEQ ID NOs: 152 to 204 of the patent application WO2017/036580.

According to one embodiment, the at least one coding RNA as described herein further comprises at least one 3′-UTR element, which comprises or consists of a nucleic acid sequence derived from the 3′-UTR of a gene selected from the group consisting of 40S ribosomal protein S9 (RPS9), Proteasome Subunit Beta 3 (PSMB3), Caspase 1 (CASP1), and Cytochrome c oxidase subunit 6B1 (COX6B1), or a variant of any of these genes, wherein the gene or the variant thereof is preferably a vertebrate gene, more preferably a mammalian gene, and even more preferably a human gene.

In a particularly preferred embodiment, the 3′-UTR element comprises or consists of a nucleic acid sequence which is derived from a 3′-UTR of a gene selected from the group consisting of an albumin gene, an α-globin gene, a β-globin gene, a tyrosine hydroxylase gene, a lipoxygenase gene, and a collagen alpha gene, such as a collagen alpha 1(I) gene, preferably as described herein, a 40S ribosomal protein S9 gene (RPS9), a Proteasome Subunit Beta 3 gene (PSMB3), a Caspase 1 gene (CASP1), and a Cytochrome c oxidase subunit 6B1 gene (COX6B1), or a variant of any of these genes, wherein the gene or the variant thereof is preferably a vertebrate gene, more preferably a mammalian gene, and even more preferably a human gene.

In a further preferred embodiment, the 3′-UTR element comprises or consists of a nucleic acid sequence, which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to any one of SEQ ID NO: 852, 854, 856 or 858, or a corresponding RNA sequence, preferably selected from SEQ ID NO: 853, 855, 857 or 859, or wherein the at least one 3′-UTR element comprises or consists of a fragment of a nucleic acid sequence which has an identity of at least about 20%, preferably of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to any one of SEQ ID NO: 852, 854, 856 or 858, wherein, preferably, the fragment is as described above, i.e. being a continuous stretch of nucleotides representing at least 20% etc. of the full-length 3′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

According to a particularly preferred embodiment, the 3′-UTR element comprises or consists of a nucleic acid sequence, which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to any one of SEQ ID NO: 852, 854, 856, 858, 412, 414, 416, 418, 420, 422 or 424, or a corresponding RNA sequence, or wherein the at least one 3′-UTR element comprises or consists of a fragment of a nucleic acid sequence which has an identity of at least about 20%, preferably of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to any one of SEQ ID NO: 852, 854, 856, 858, 412, 414, 416, 418, 420, 422 or 424, or, wherein, preferably, the fragment is as described above, i.e. being a continuous stretch of nucleotides representing at least 20% etc. of the full-length 3′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

Preferably, the at least one 5′-UTR element and the at least one 3′-UTR element act synergistically to increase protein production from the at least one coding RNA as described herein.

In a preferred embodiment, the at least one coding RNA as described herein thus comprises at least one 5′-UTR element, preferably as described herein, and at least one 3′-UTR element, preferably as described herein. Particularly preferred 5′-UTR elements, 3′-UTR elements and the respective combinations thereof are summarized in the following Table A. Therein, the 5′-UTR elements and the 3′-UTR elements are preferably as described herein.

TABLE A
Preferred combinations of 5′-UTR elements and 3′-UTR elements:

| | SEQ ID NO: | SEQ ID NO: | | SEQ ID NO: | SEQ ID NO: |
|---|---|---|---|---|---|
| 5′-UTR element: | RNA | DNA | 3′-UTR element: | RNA | DNA |
| Mp68 | 839 | 838 | RPS9 | 853 | 852 |
| Nosip | 841 | 840 | RPS9 | 853 | 852 |
| HSD17B4 | 843 | 842 | PSMB3 | 855 | 854 |
| Rpl31 | 845 | 844 | CASP1 | 857 | 856 |
| TUBB4B | 847 | 846 | RPS9 | 853 | 852 |
| ATP5A1 | 849 | 848 | PSMB3 | 855 | 854 |
| Ndufa4 | 851 | 850 | RPS9 | 853 | 852 |
| Mp68 | 839 | 838 | COX6B | 859 | 858 |
| Rpl31 | 845 | 844 | PSMB3 | 855 | 854 |
| HSD17B4 | 843 | 842 | COX6B | 859 | 858 |

According to preferred embodiments, a 5′-UTR element as indicated in column 1 (‘5′-UTR element’) of Table A and as described herein is combined in the at least one coding RNA as used herein with the 3′-UTR element indicated in the same row in column 4 (‘C3-UTR element’) of Table A and as described herein. For example, a 5′-UTR element derived from an Mp68 gene or a variant thereof as described herein may preferably be combined with a 3′-UTR element derived from an RPS9 gene or a variant thereof as described herein. More preferably, a 5′-UTR element comprising an RNA sequence according to the SEQ ID NO: indicated in column 2 (‘SEQ ID NO: RNA’) of Table A or a DNA sequence according to the SEQ ID NO: indicated in column 3 (‘SEQ ID NO: DNA’) of Table A, or a fragment or variant of said RNA or DNA sequence, may be combined with the 3′-UTR element in the same row of Table A, i.e. preferably the 3′-UTR element comprising an RNA sequence according to the SEQ ID NO: indicated in column 5 (‘SEQ ID NO: RNA’) in the same row of Table A or a DNA sequence according to the SEQ ID NO: indicated in column 6 (‘SEQ ID NO: DNA’) in the same row of Table A, or a fragment or variant of said RNA or DNA sequence. For example, the at least one coding RNA as used herein may comprise a 5′-UTR element comprising or consisting of the nucleic acid sequence defined by SEQ ID NO: 841, or a fragment or variant thereof as defined herein, and a 3′-UTR element comprising or consisting of the nucleic acid sequence defined by SEQ ID NO: 853, or a fragment or variant thereof as defined herein. According to another preferred embodiment of the invention, an isRNA or the at least one coding RNA as described herein, can be modified by the addition of a so-called “5′ cap” structure, which preferably stabilizes the RNA as described herein. A 5′-cap is an entity, typically a modified nucleotide entity, which generally “caps” the 5′-end of a mature mRNA. A 5′-cap may typically be formed by a modified nucleotide, particularly by a derivative of a guanine nucleotide. Preferably, the 5′-cap is linked to the 5′-terminus via a 5′-5′-triphosphate linkage. A 5′-cap may be methylated, e.g. m7GpppN, wherein N is the terminal 5′ nucleotide of the nucleic acid carrying the 5′-cap, typically the 5′-end of an mRNA. m7GpppN is the 5′-cap structure, which naturally occurs in mRNA transcribed by polymerase II and is therefore preferably not considered as modification comprised in a modified RNA in this context. Accordingly, a modified RNA of the present invention may comprise a m7GpppN as 5′-cap, but additionally the modified RNA typically comprises at least one further modification as defined herein.

Further examples of 5′cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. These modified 5′-cap structures are regarded as at least one modification in this context.

Particularly preferred modified 5′-cap structures are cap1 (methylation of the ribose of the adjacent nucleotide of m7G), cap2 (additional methylation of the ribose of the 2nd nucleotide downstream of the m7G), cap3 (additional methylation of the ribose of the 3rd nucleotide downstream of the m7G), cap4 (methylation of the ribose of the 4th nucleotide downstream of the m7G), ARCA (anti-reverse cap analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In some embodiments, the at least one coding RNA as described herein preferably comprises a poly(A) and/or a poly(C) sequence.

In a particularly preferred embodiment, the at least one coding RNA as described herein comprises additionally to the coding region encoding at least one peptide or protein as described above or a fragment or variant thereof, a poly(A) sequence, also called poly-A tail, preferably at the 3′ terminus of the RNA. If present, such a poly(A) sequence comprises a sequence of about 25 to about 400 adenosine nucleotides, preferably a sequence of about 50 to about 400 adenosine nucleotides, more preferably a sequence of about 50 to about 300 adenosine nucleotides, even more preferably a sequence of about 50 to about 250 adenosine nucleotides, most preferably a sequence of about 60 to about 250 adenosine nucleotides. In this context the term “about” refers to a deviation of +10% of the value(s) it is attached to. This poly(A) sequence is preferably located 3′ of the coding region comprised in the at least one coding RNA as described herein.

Preferably, the poly(A) sequence in at least one coding RNA as described herein is derived from a DNA template by RNA in vitro transcription. Alternatively, the poly(A) sequence may also be obtained in vitro by common methods of chemical-synthesis without being necessarily transcribed from a DNA-progenitor. Moreover, poly(A) sequences, or poly(A) tails may be generated by enzymatic polyadenylation of the at least one RNA using commercially available polyadenylation kits and corresponding protocols known in the art.

Alternatively, the at least one coding RNA as described herein optionally comprises a polyadenylation signal, which is defined herein as a signal, which conveys polyadenylation to a (transcribed) RNA by specific protein factors (e.g. cleavage and polyadenylation specificity factor (CPSF), cleavage stimulation factor (CstF), cleavage factors I and II (CF I and CF II), poly(A) polymerase (PAP)). In this context, a consensus polyadenylation signal is preferred comprising the NN(U/T)ANA consensus sequence. In a particularly preferred aspect, the polyadenylation signal comprises one of the following sequences: AA(U/T)AAA or A(U/T)(U/T)AAA (wherein uridine is usually present in RNA and thymidine is usually present in DNA).

According to a further preferred embodiment, the at leasat one coding RNA as described herein can be modified by a sequence of at least 10 cytosines, preferably at least 20 cytosines, more preferably at least 30 cytosines (so-called “poly(C) sequence”). Particularly, the RNA may contain a poly(C) sequence of typically about 10 to 200 cytosine nucleotides, preferably about 10 to 100 cytosine nucleotides, more preferably about 10 to 70 cytosine nucleotides or even more preferably about 20 to 50 or even 20 to 30 cytosine nucleotides. This poly(C) sequence is preferably located 3′ of the coding region, more preferably 3′ of an optional poly(A) sequence comprised in the at least one coding RNA as described herein.

In a particularly preferred embodiment, the at least one coding RNA as described herein comprises a histone stem-loop sequence/structure. Such histone stem-loop sequences are preferably selected from histone stem-loop sequences as disclosed in WO 2012/019780, whose disclosure is incorporated herewith by reference.

A histone stem-loop sequence, suitable to be used within the present invention, is preferably selected from at least one of the following formulae (VII) or (VIII):

formula (VII) (stem-loop sequence without stem bordering elements):

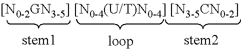

formula (VIII) (stem-loop sequence with stem bordering elements):

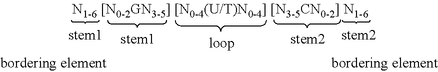

wherein:
stem1 or stem2 bordering elements N1-6 is a consecutive sequence of 1 to 6, preferably of 2 to 6, more preferably of 2 to 5, even more preferably of 3 to 5, most preferably of 4 to 5 or 5 N, wherein each N is independently from another selected from a nucleotide selected from A, U, T, G and C, or a nucleotide analogue thereof;
stem1 [N₀₋₂GN₃₋₅] is reverse complementary or partially reverse complementary with element stem2, and is a consecutive sequence between of 5 to 7 nucleotides;
   wherein N₀₋₂ is a consecutive sequence of 0 to 2, preferably of 0 to 1, more preferably of 1 N, wherein each N is independently from another selected from a nucleotide selected from A, U, T, G and C or a nucleotide analogue thereof;
   wherein N₃₋₅ is a consecutive sequence of 3 to 5, preferably of 4 to 5, more preferably of 4 N, wherein each N is independently from another selected from a nucleotide selected from A, U, T, G and C or a nucleotide analogue thereof, and
   wherein G is guanosine or an analogue thereof, and may be optionally replaced by a cytidine or an analogue thereof, provided that its complementary nucleotide cytidine in stem2 is replaced by guanosine;
loop sequence [N₀₋₄(U/T)N₀₋₄] is located between elements stem1 and stem2, and is a consecutive sequence of 3 to 5 nucleotides, more preferably of 4 nucleotides;
   wherein each N₀₋₄ is independent from another a consecutive sequence of 0 to 4, preferably of 1 to 3, more preferably of 1 to 2 N, wherein each N is independently from another selected from a nucleotide selected from A, U, T, G and C or a nucleotide analogue thereof; and
   wherein U/T represents uridine, or optionally thymidine;
stem2 [N₃₋₅CN₀₋₂] is reverse complementary or partially reverse complementary with element stem1, and is a consecutive sequence between of 5 to 7 nucleotides;
   wherein N₃₋₅ is a consecutive sequence of 3 to 5, preferably of 4 to 5, more preferably of 4 N, wherein each N is independently from another selected from a nucleotide selected from A, U, T, G and C or a nucleotide analogue thereof;
   wherein N₀₋₂ is a consecutive sequence of 0 to 2, preferably of 0 to 1, more preferably of 1 N, wherein each N is independently from another selected from a nucleotide selected from A, U, T, G or C or a nucleotide analogue thereof; and
   wherein C is cytidine or an analogue thereof, and may be optionally replaced by a guanosine or an analogue thereof provided that its complementary nucleoside guanosine in stem1 is replaced by cytidine;

wherein

stem1 and stem2 are capable of base pairing with each other forming a reverse complementary sequence, wherein base pairing may occur between stem1 and stem2, e.g. by Watson-Crick base pairing of nucleotides A and U/T or G and C or by non-Watson-Crick base pairing e.g. wobble base pairing, reverse Watson-Crick base pairing, Hoogsteen base pairing, reverse Hoogsteen base pairing or are capable of base pairing with each other forming a partially reverse complementary sequence, wherein an incomplete base pairing may occur between stem1 and stem2, on the basis that one ore more bases in one stem do not have a complementary base in the reverse complementary sequence of the other stem.

According to a further preferred embodiment of the first inventive aspect, the at least one mRNA of the inventive composition sequence may comprise at least one histone stem-loop sequence according to at least one of the following specific formulae (Vila) or (Villa):

formula (Vila) (stem-loop sequence without stem bordering elements):

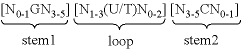

formula (Villa) (stem-loop sequence with stem bordering elements):

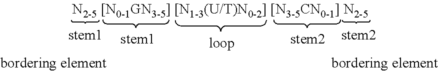

wherein:

N, C, G, T and U are as defined above.

According to a further more particularly preferred embodiment of the first aspect, the at least one mRNA of the inventive composition sequence may comprise at least one histone stem-loop sequence according to at least one of the following specific formulae (VIIb) or (VIIIb):

formula (VIIb) (stem-loop sequence without stem bordering elements):

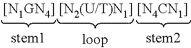

formula (VIIIb) (stem-loop sequence with stem bordering elements):

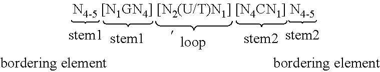

wherein:

N, C, G, T and U are as defined above.

A particular preferred histone stem-loop sequence is the sequence according to SEQ ID No: 426.

More preferably the stem-loop sequence is the corresponding RNA sequence of the nucleic acid sequence according to SEQ ID NO: 427.

According to another particularly preferred embodiment, the at least one coding RNA as described herein may additionally or alternatively encode a secretory signal peptide. Such signal peptides are sequences, which typically exhibit a length of about 15 to 30 amino acids and are preferably located at the N-terminus of the encoded peptide, without being limited thereto. Signal peptides as defined herein preferably allow the transport of the antigen, antigenic protein or antigenic peptide as encoded by the at least one coding RNA as described herein into a defined cellular compartiment, preferably the cell surface, the endoplasmic reticulum (ER) or the endosomal-lysosomal compartiment. Examples of secretory signal peptide sequences as defined herein include, without being limited thereto, signal sequences of classical or non-classical MHC-molecules (e.g. signal sequences of MHC I and II molecules, e.g. of the MHC class I molecule HLA-A*0201), signal sequences of cytokines or immunoglobulines as defined herein, signal sequences of the invariant chain of immunoglobulines or antibodies as defined herein, signal sequences of Lamp1, Tapasin, Erp57, Calretikulin, Calnexin, and further membrane associated proteins or of proteins associated with the endoplasmic reticulum (ER) or the endosomal-lysosomal compartiment. Particularly preferably, signal sequences of MHC class I molecule HLA-A*0201 may be used according to the present invention.

In some embodiments, the at least one coding RNA as described herein, preferably an mRNA, comprises, preferably in 5′ to 3′ direction, the following elements:
a) a 5′-CAP structure, preferably m7GpppN,
b) at least one coding sequence encoding at least one peptide or protein comprising IL-12, a decoy PD-1 receptor, preferably a soluble PD-1 receptor as described herein, CD40L, an anti-CTLA4 antibody, and/or a tumor antigen or a fragment or variant of any of these proteins,
c) a 3′-UTR element comprising a nucleic acid sequence, which is derived from an α-globin gene, preferably comprising the corresponding RNA sequence of the nucleic acid sequence according to SEQ ID NO: 418, or a homolog, a fragment or a variant thereof,
d) a poly(A) tail, preferably consisting of 10 to 200, 10 to 100, 40 to 80 or 50 to 70 adenosine nucleotides,
e) a poly(C) tail, preferably consisting of 10 to 200, 10 to 100, 20 to 70, 20 to 60 or 10 to 40 cytosine nucleotides, and
f) a histone stem-loop, preferably comprising the RNA sequence according to SEQ ID NO: 427.

According to a preferred embodiment, the at least one coding RNA as described herein comprises
a) a nucleic acid sequence selected from the group consisting of SEQ ID NOs: 146-151; 157-162; 168-173; 179-184; 190-195; 201-206; 212-217; 223-228; 234-239; 245-250; 256-261 and 267-272, or a fragment or variant of any of these sequences, preferably from the group consisting of SEQ ID NOs: 153; 164; 175; 186; 197; 208; 219; 230; 241; 252; 263, 274; 992 and 598, or a fragment or variant of any of these sequences,
b) a nucleic acid sequence selected from the group consisting of SEQ ID NOs: 154; 165; 176; 187; 198; 209; 220; 231; 242; 253; 264, 275 and 596, or a fragment or variant of any of these sequences,
c) a nucleic acid sequence according to any one of SEQ ID NO: 860-874, 875-889 or 594, preferably according to SEQ ID NO: 594, or a fragment or variant of any of these nucleic acid sequences, and a nucleic acid sequence according to any one of SEQ ID NO: 890-904, 905-919 or 595, preferably according to SEQ ID NO: 595, or a fragment or variant of any of these nucleic acid sequences;
   or
   a nucleic acid sequence according to SEQ ID NO: 920-922 or 923-925, or a fragment or variant of any of these nucleic acid sequences, and/or
d) optionally, a nucleic acid sequence selected from the group consisting of SEQ ID NOs: 144; 155; 166; 177; 188; 199; 210; 221; 232; 243; 254 and 265 or a fragment or variant of any of these sequences, preferably from the group consisting of SEQ ID NOs: 145; 156; 167; 178; 189; 200; 211; 222; 233; 244; 255; 266 and 597, or a fragment or variant of any of these sequences.

According to a further embodiment, the at least one coding RNA as described herein comprises
a) a nucleic acid sequence selected from the group consisting of SEQ ID NOs: 146-151; 451-456; 157-162; 168-173; 179-184; 190-195; 201-206; 212-217; 223-228; 234-239; 245-250; 256-261 and 267 272, or a fragment or variant of any of these sequences, preferably from the group consisting of SEQ ID NOs: 153; 458; 164; 175; 186; 197; 208; 219; 230; 241; 252; 263, 274; 992 and 598, or a fragment or variant of any of these sequences,
b) a nucleic acid sequence selected from the group consisting of SEQ ID NOs: 154; 459; 165; 176; 187; 198; 209; 220; 231; 242; 253; 264, 275 and 596, or a fragment or variant of any of these sequences,
c) a nucleic acid sequence according to any one of SEQ ID NO: 860-874, 875-889 or 594, preferably according to SEQ ID NO: 594, or a fragment or variant of any of these nucleic acid sequences, and a nucleic acid sequence according to any one of SEQ ID NO: 890-904, 905-919 or 595, preferably according to SEQ ID NO: 595, or a fragment or variant of any of these nucleic acid sequences;
   or
   a nucleic acid sequence according to SEQ ID NO: 920-922 or 923-925, or a fragment or variant of any of these nucleic acid sequences, and/or
d) optionally, a nucleic acid sequence selected from the group consisting of SEQ ID NOs: 144; 449; 155; 166; 177; 188; 199; 210; 221; 232; 243; 254 and 265, or a fragment or variant of any of these sequences, preferably from the group consisting of SEQ ID NOs: 145; 450; 156; 167; 178; 189; 200; 211; 222; 233; 244; 255; 266 and 597, or a fragment or variant of any of these sequences.

In a preferred embodiment, the at least one coding RNA as described herein comprises
a) a nucleic acid sequence selected from the group consisting of SEQ ID NOs: 451-456; 157-162; 168-173; 179-184; 190-195; 201-206; 212-217; 223-228; 234-239; 245-250; 256-261 and 267-272, or a fragment or variant of any of these sequences, preferably from the group consisting of SEQ ID NOs: 164; 175; 186; 197; 208; 219; 230; 241; 252; 263, 274; 992 and 598, or a fragment or variant of any of these sequences,
b) a nucleic acid sequence selected from the group consisting of SEQ ID NOs: 459; 165; 176; 187; 198; 209; 220; 231; 242; 253; 264, 275 and 596, or a fragment or variant of any of these sequences,
c) a nucleic acid sequence according to any one of SEQ ID NO: 860-874, 875-889 or 594, preferably according to SEQ ID NO: 594, or a fragment or variant of any of these nucleic acid sequences, and a nucleic acid sequence according to any one of SEQ ID NO: 890-904, 905-919 or 595, preferably according to SEQ ID NO: 595, or a fragment or variant of any of these nucleic acid sequences;
   or
   a nucleic acid sequence according to SEQ ID NO: 920-922 or 923-925, or a fragment or variant of any of these nucleic acid sequences, and/or
d) optionally, a nucleic acid sequence selected from the group consisting of SEQ ID NOs: 144; 449; 166; 177; 188; 199; 210; 221; 232; 243; 254 and 265, or a fragment or variant of any of these sequences, preferably from the group consisting of SEQ ID NOs: 145; 450; 167; 178; 189; 200; 211; 222; 233; 244; 255; 266 and 597, or a fragment or variant of any of these sequences.

According to a particularly preferred embodiment, the present invention provides an isRNA for use in the treatment of a tumor or cancer disease as described herein, preferably comprising intratumoral administration of the isRNA, wherein the treatment comprises administration of at least three, preferably at least four or five, coding RNAs as described herein (as additional pharmaceutically active ingredients), preferably at least three, more preferably at least four or five, mRNAs, wherein a first coding RNA comprises a nucleic acid sequence selected from the group consisting of SEQ ID NOs: 153; 164;

175; 186; 197; 208; 219; 230; 241; 252; 263; 274; 992 and 598, or a fragment or variant of any of these sequences, a second coding RNA comprises a nucleic acid sequence selected from the group consisting of SEQ ID NOs: 154; 165; 176; 187; 198; 209; 220; 231; 242; 253; 264; 275 and 596, or a fragment or variant of any of these sequences, a third coding RNA comprises a nucleic acid sequence selected from the group consisting of a nucleic acid sequence according to any one of SEQ ID NO: 860-874 or 594, preferably according to SEQ ID NO: 594, or a fragment or variant of any of these nucleic acid sequences, and a nucleic acid sequence according to any one of SEQ ID NO: 890-904 or 595, preferably according to SEQ ID NO: 595, or a fragment or variant of any of these nucleic acid sequences, and/or optionally, a fourth coding RNA comprises a nucleic acid sequence selected from the group consisting of SEQ ID NOs: 145; 156; 167; 178; 189; 200; 211; 222; 233; 244; 255; 266 and 597, or a fragment or variant of any of these sequences,

wherein the first, the second, the third and the fourth coding RNAs are preferably administered intratumorally.

According to a particularly preferred embodiment, the present invention provides an isRNA for use in the treatment of a tumor or cancer disease as described herein, preferably comprising intratumoral administration of the isRNA, wherein the treatment comprises administration of at least three, preferably at least four or five, coding RNAs as described herein (as additional pharmaceutically active ingredients), preferably at least three, more preferably at least four or five, mRNAs, wherein
a) a first coding RNA comprises a nucleic acid sequence selected from the group consisting of SEQ ID NOs: 153; 164; 175; 186; 197; 208; 219; 230; 241; 252; 263; 274; 992 and 598, or a fragment or variant of any of these sequences,
b) a second coding RNA comprises a nucleic acid sequence selected from the group consisting of SEQ ID NOs: 154; 165; 176; 187; 198; 209; 220; 231; 242; 253; 264; 275 and 596, or a fragment or variant of any of these sequences,
c) a third coding RNA comprises a nucleic acid sequence selected from the group consisting of a nucleic acid sequence according to any one of SEQ ID NO: 860-874 or 594, preferably according to SEQ ID NO: 594, or
   a fragment or variant of any of these nucleic acid sequences, and
   a fourth coding RNA comprises a nucleic acid sequence according to any one of SEQ ID NO: 890-904 or 595, preferably according to SEQ ID NO: 595, or a fragment or variant of any of these nucleic acid sequences, and/or
d) optionally, a fifth coding RNA comprises a nucleic acid sequence selected from the group consisting of SEQ ID NOs: 145; 156; 167; 178; 189; 200; 211; 222; 233; 244; 255; 266 and 597, or a fragment or variant of any of these sequences

wherein the first, the second, the third, the fourth and the fifth coding RNAs are preferably administered intratumorally.

In a particularly preferred embodiment, the present invention provides an isRNA for use in the treatment of a tumor or cancer disease as described herein, preferably comprising intratumoral administration of the isRNA, wherein the treatment comprises administration of at least three, preferably at least four of five, coding RNAs as described herein (as additional pharmaceutically active ingredients), preferably at least three, more preferably at least four or five, mRNAs, wherein
a) a first coding RNA comprises a nucleic acid sequence selected from the group consisting SEQ ID NOs: 164; 175; 186; 197; 208; 219; 230; 241; 252; 263; 274; 992 and 598, or a fragment or variant of any of these sequences,
b) a second coding RNA comprises a nucleic acid sequence selected from the group consisting of SEQ ID NOs: 165; 176; 187; 198; 209; 220; 231; 242; 253; 264; 275 and 596, or a fragment or variant of any of these sequences,
c) a third coding RNA comprises a nucleic acid sequence selected from the group consisting of a nucleic acid sequence according to any one of SEQ ID NO: 860-874 or 594, preferably according to SEQ ID NO: 594, or
   a fragment or variant of any of these nucleic acid sequences, and
   a fourth coding RNA comprises a nucleic acid sequence according to any one of SEQ ID NO: 890-904 or 595, preferably according to SEQ ID NO: 595, or a fragment or variant of any of these nucleic acid sequences, and/or
d) optionally, a fifth coding RNA comprises a nucleic acid sequence selected from the group consisting of SEQ ID NOs: 156; 167; 178; 189; 200; 211; 222; 233; 244; 255; 266 and 597, or a fragment or variant of any of these sequences,

wherein the first, the second, the third, the fourth and the fifth coding RNAs are preferably administered intratumorally.

In certain embodiments, the at least one coding RNA as described herein, preferably an mRNA, comprises, preferably in 5′ to 3′ direction, the following elements:
a) a 5′-CAP structure, preferably m7GpppN,
b) a 5′-UTR element, which comprises or consists of a nucleic acid sequence, which is derived from the 5′-UTR of a TOP gene, preferably comprising an RNA sequence corresponding to the nucleic acid sequence according to SEQ ID NO: 408, or a homolog, a fragment or a variant thereof,
c) at least one coding sequence encoding at least one peptide or protein comprising IL-12, a decoy PD-1 receptor, preferably a soluble PD-1 receptor as described herein, CD40L, an anti-CTLA4 antibody, and/or a tumor antigen or a fragment or variant of any of these proteins,
d) a 3′-UTR element comprising a nucleic acid sequence, which is derived from an α-globin gene, preferably comprising the corresponding RNA sequence of the nucleic acid sequence according to SEQ ID NO: 418, or a homolog, a fragment or a variant thereof; and/or
   a 3′-UTR element comprising a nucleic acid sequence, which is derived from an albumin gene, preferably comprising the corresponding RNA sequence of the nucleic acid sequence according to SEQ ID NO: 422 or 424, or a homolog, a fragment or a variant of any of these sequences,
e) a poly(A) tail, preferably consisting of 10 to 200, 10 to 100, 40 to 80 or 50 to 70 adenosine nucleotides,
f) a poly(C) tail, preferably consisting of 10 to 200, 10 to 100, 20 to 70, 20 to 60 or 10 to 40 cytosine nucleotides, and
g) a histone stem-loop, preferably comprising the RNA sequence according to SEQ ID NO: 427.

According to a preferred embodiment, the at least one coding RNA as described herein comprises
a) a nucleic acid sequence selected from the group consisting of SEQ ID NOs: 278-283; 289-294; 300-305; 311-316; 322-327; 333-338; 344-349; 355-360; 366-371; 377-382; 388-393; 399-404 and 462-467, or a fragment or variant of any of these sequences, preferably from the group consisting of SEQ ID NOs: 285; 296; 307; 318; 329; 340; 351; 362; 373; 384; 395; 406; 430; 469 and 992, or a fragment or variant of any of these sequences,
b) a nucleic acid sequence selected from the group consisting of SEQ ID NO: 286; 297; 308; 319; 330; 341; 352; 363; 374; 385; 396; 470 and 407, or a fragment or variant of any of these sequences,
c) a nucleic acid sequence according to any one of SEQ ID NO: 926-940 or 941-955, preferably according to SEQ ID NO: 926-940, or a fragment or variant of any of these nucleic acid sequences, and a nucleic acid sequence according to any one of SEQ ID NO: 956-970 or 971-985, preferably according to SEQ ID NO: 956-970, or a fragment or variant of any of these nucleic acid sequences;
   or
   a nucleic acid sequence according to SEQ ID NO: 986-988 or 989-991, or a fragment or variant of any of these nucleic acid sequences, and/or
d) optionally, a nucleic acid sequence selected from the group consisting of SEQ ID NOs: 276; 287; 298; 309; 320; 331; 342; 353; 364; 375; 386; 460 and 397, or a fragment or variant of any of these sequences, preferably from the group consisting of SEQ ID NOs: 277; 288; 299; 310; 321; 332; 343; 354; 365; 376; 461; 387 and 398, or a fragment or variant of any of these sequences.

According to another embodiment, the at least one coding RNA as described herein comprises
a) a nucleic acid sequence selected from the group consisting of SEQ ID NOs: 289-294; 300-305; 311-316; 322-327; 333-338; 344-349; 355-360; 366-371; 377-382; 388-393 and 399-404, or a fragment or variant of any of these sequences,
b) a nucleic acid sequence selected from the group consisting of SEQ ID NO: 286; 297; 308; 319; 330; 341; 352; 363; 374; 385; 396 and 407, or a fragment or variant of any of these sequences,
c) a nucleic acid sequence according to any one of SEQ ID NO: 926-940 or 941-955, preferably according to SEQ ID NO: 926-940, or a fragment or variant of any of these nucleic acid sequences, and a nucleic acid sequence according to any one of SEQ ID NO: 956-970 or 971-985, preferably according to SEQ ID NO: 956-970, or a fragment or variant of any of these nucleic acid sequences;
   or
   a nucleic acid sequence according to SEQ ID NO: 986-988 or 989-991, or a fragment or variant of any of these nucleic acid sequences, and/or
d) optionally, a nucleic acid sequence selected from the group consisting of SEQ ID NOs: 296; 307; 318; 329; 340; 351; 362; 373; 384; 395; 406; 430 and 992, or a fragment or variant of any of these sequences, preferably from the group consisting of SEQ ID NOs: 277; 288; 299; 310; 321; 332; 343; 354; 365; 376; 387 and 398, or a fragment or variant of any of these sequences.

In a preferred embodiment, the at least one coding RNA as described herein comprises
a) a nucleic acid sequence selected from the group consisting of SEQ ID NOs: 278-283; 289-294; 300-305; 311-316; 322-327; 333-338; 344-349; 355-360; 366-371; 377-382; 388-393; 399-404 and 462-467, or a fragment or variant of any of these sequences, preferably from the group consisting of SEQ ID NOs: 285; 296; 307; 318; 329; 340; 351; 362; 373; 384; 395; 406; 430; 469 and 992, or a fragment or variant of any of these sequences,
b) a nucleic acid sequence selected from the group consisting of SEQ ID NO: 286; 297; 308; 319; 330; 341; 352; 363; 374; 385; 396 and 407, or a fragment or variant of any of these sequences,
c) a nucleic acid sequence according to any one of SEQ ID NO: 926-940 or 941-955, preferably according to SEQ ID NO: 926-940, or a fragment or variant of any of these nucleic acid sequences, and a nucleic acid sequence according to any one of SEQ ID NO: 956-970 or 971-985, preferably according to SEQ ID NO: 956-970, or a fragment or variant of any of these nucleic acid sequences;
   or
   a nucleic acid sequence according to SEQ ID NO: 986-988 or 989-991, or a fragment or variant of any of these nucleic acid sequences, and/or
d) optionally, a nucleic acid sequence selected from the group consisting of SEQ ID NOs: 276; 287; 298; 309; 320; 331; 342; 353; 364; 375; 386; 460 and 397, or a fragment or variant of any of these sequences, preferably from the group consisting of SEQ ID NOs: 277; 288; 299; 310; 321; 332; 343; 354; 365; 376; 461; 387 and 398, or a fragment or variant of any of these sequences.

According to a particularly preferred embodiment, the present invention provides an isRNA for use in the treatment of a tumor or cancer disease as described herein, preferably comprising intratumoral administration of the isRNA, wherein the treatment comprises administration of at least three, preferably at least four or five, coding RNAs as described herein (as additional pharmaceutically active ingredients), preferably at least three, more preferably at least four or five, mRNAs, wherein
a) a first coding RNA comprises a nucleic acid sequence selected from the group consisting of SEQ ID NOs: 285; 296; 307; 318; 329; 340; 351; 362; 373; 384; 395; 406; 430; 469 and 992, or a fragment or variant of any of these sequences,
b) a second coding RNA comprises a nucleic acid sequence selected from the group consisting of SEQ ID NOs: 286; 297; 308; 319; 330; 341; 352; 363; 374; 385; 396; 470 and 407, or a fragment or variant of any of these sequences,
c) a third coding RNA comprises a nucleic acid sequence selected from the group consisting of a nucleic acid sequence according to any one of SEQ ID NO: 926-940, or a fragment or variant of any of these nucleic acid sequences, and
   a fourth coding RNA comprises a nucleic acid sequence according to any one of SEQ ID NO: 956-970, or a fragment or variant of any of these nucleic acid sequences, and/or
d) optionally, a fifth coding RNA comprises a nucleic acid sequence selected from the group consisting of SEQ ID NOs: 277; 288; 299; 310; 321; 332; 343; 354; 365; 376; 387; 461 and 398, or a fragment or variant of any of these sequences;

wherein the first, the second, the third, the fourth and the fifth coding RNAs are preferably administered intratumorally.

In a further preferred embodiment, the present invention provides an isRNA for use in the treatment of a tumor or cancer disease as described herein, preferably comprising intratumoral administration of the isRNA, wherein the treatment comprises administration of at least three, preferably at least four or five, coding RNAs as described herein (as additional pharmaceutically active ingredients), preferably at least three, more preferably at least four or five, mRNAs, wherein
a) a first coding RNA comprises a nucleic acid sequence selected from the group consisting of SEQ ID NOs: 285; 296; 307; 318; 329; 340; 351; 362; 373; 384; 395; 406; 430; 469 and 992, or a fragment or variant of any of these sequences,
b) a second coding RNA comprises a nucleic acid sequence selected from the group consisting of SEQ ID NOs: 286; 297; 308; 319; 330; 341; 352; 363; 374; 385; 396; 470 and 407, or a fragment or variant of any of these sequences,
c) a third coding RNA comprises a nucleic acid sequence selected from the group consisting of a nucleic acid sequence according to any one of SEQ ID NO: 926-940, or a fragment or variant of any of these nucleic acid sequences, and
   a fourth coding RNA comprises a nucleic acid sequence according to any one of SEQ ID NO: 956-970, or a fragment or variant of any of these nucleic acid sequences, and/or
d) optionally a fifth coding RNA comprises a nucleic acid sequence selected from the group consisting of SEQ ID NOs: 277; 288; 299; 310; 321; 332; 343; 354; 365; 376; 387; 461 and 398, or a fragment or variant of any of these sequences,

wherein the first, the second, the third, the fourth and the fifth coding RNAs are preferably administered intratumorally.

According to a particularly preferred embodiment, the present invention provides an isRNA for use in the treatment of a tumor or cancer disease as described herein, preferably comprising intratumoral administration of the isRNA, wherein the treatment comprises administration of at least three, preferably at least four or five, coding RNAs as described herein (as additional pharmaceutically active ingredients), preferably at least three, more preferably at least four or five, mRNAs, wherein
a) a first coding RNA comprises a nucleic acid sequence selected from the group consisting of SEQ ID NOs: 285; 296; 307; 318; 329; 340; 351; 362; 373; 384; 395; 406; 430; 469 and 992, or a fragment or variant of any of these sequences,
b) a second coding RNA comprises a nucleic acid sequence selected from the group consisting of SEQ ID NOs: 286; 297; 308; 319; 330; 341; 352; 363; 374; 385; 396; 470 and 407, or a fragment or variant of any of these sequences,
c) a third coding RNA comprises a nucleic acid sequence selected from the group consisting of a nucleic acid sequence according to any one of SEQ ID NO: 926-940, or a fragment or variant of any of these nucleic acid sequences, and
   a fourth coding RNA comprises a nucleic acid sequence according to any one of SEQ ID NO: 956-970, or a fragment or variant of any of these nucleic acid sequences, and/or
b) optionally, a fifth coding RNA comprises a nucleic acid sequence selected from the group consisting of SEQ ID NOs: 277; 288; 299; 310; 321; 332; 343; 354; 365; 376; 387; 461 and 398, or a fragment or variant of any of these sequences;

wherein the first, the second, the third, the fourth and the fifth coding RNAs are preferably administered intratumorally.

According to a preferred embodiment, the present invention relates to an isRNA for use in the treatment of a tumor or cancer disease as described herein, wherein the treatment comprises administration at least one coding RNA as described herein (as additional pharmaceutically active ingredient), wherein the isRNA is administered as RNA complexed with one or more cationic or polycationic compounds, preferably a polymeric carrier as described herein, and the at least one coding RNA, more preferably an mRNA, is administered as free RNA.

According to a further preferred embodiment, the invention provides an isRNA for use in the treatment of a tumor or cancer disease,

wherein the isRNA comprises a nucleic acid sequence according to formula (I) (GₗXₘGₙ), formula (II) (CₗXₘCₙ), formula (III) (NᵤGₗXₘGₙNᵥ)ₐ or formula (IV) (NᵤCₗXₘCₙNᵥ)ₐ, preferably at least one nucleic acid sequence according to any one of SEQ ID NOs: 433 to 437, 1014 to 1016, 1055 or 1056, or a fragment or variant of any of these sequences, more preferably according to any one of SEQ ID NOs: 433, 434, 1014 to 1016, or a fragment or a variant thereof,

wherein the isRNA is complexed with a cationic or polycationic compound, preferably with a polymeric carrier, more preferably with a polymeric carrier that is formed by a disulfide-crosslinked cationic component, which preferably comprises a peptide according to formula (V), (Va) and/or (Vb) and/or a compound according to formula (VI), more preferably at least one of the disulfide-crosslinked cationic peptides Cys-Arg₁₂ (SEQ ID NO: 580), Cys-Arg₁₂-Cys (SEQ ID NO: 579), or Trp-Arg₁₂-Cys (SEQ ID NO: 1074),

wherein the isRNA is preferably administered intratumorally,

wherein the tumor or cancer disease is preferably selected from the group consisting of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC);

wherein the tumor or the cancer disease is preferably at an advanced stage and/or refractory to standard therapy;

wherein the treatment of a tumor or cancer disease comprises administration of at least three, preferably at least four or five, coding RNAs as described herein (as additional pharmaceutically active ingredients), preferably at least three, more preferably at least four or five, mRNAs, wherein
a) a first coding RNA comprises a nucleic acid sequence selected from the group consisting of of SEQ ID NOs: 285; 296; 307; 318; 329; 340; 351; 362; 373; 384; 395; 406; 430; 469 and 992, or a fragment or variant of any of these sequences,
b) a second coding RNA comprises a nucleic acid sequence selected from the group consisting of SEQ ID NOs: 286; 297; 308; 319; 330; 341; 352; 363; 374; 385; 396; 470 and 407, or a fragment or variant of any of these sequences,
c) a third coding RNA comprises a nucleic acid sequence selected from the group consisting of a nucleic acid sequence according to any one of SEQ ID NO: 926-940, or a fragment or variant of any of these nucleic acid sequences, and
   a fourth coding RNA comprises a nucleic acid sequence according to any one of SEQ ID NO: 956-970, or a fragment or variant of any of these nucleic acid sequences, and/or
d) optionally, a fifth coding RNA comprises a nucleic acid sequence selected from the group consisting of SEQ ID NOs: 277; 288; 299; 310; 321; 332; 343; 354; 365; 376; 387; 461 and 398, or a fragment or variant of any of these sequences,

wherein the first, the second, the third, the fourth and the fifth coding RNAs are preferably administered intratumorally.

According to a further aspect of the present invention, a combination of an isRNA and at least one coding RNA is provided, wherein the at least one coding RNA encodes at least one peptide or protein comprising IL 12, CD40L, a decoy PD 1 receptor, preferably a soluble PD-1 receptor, and an anti-CTLA4 antibody, or a fragment or variant of any of these proteins.

The isRNA in the combination according to the invention is preferably an isRNA as described herein with respect to the is RNA for use in the treatment of a tumor or cancer disease. The at least one coding RNA of the combination according to the invention is preferably a coding RNA as described herein, more preferably a coding RNA as described herein with respect to the at least one coding RNA that is used as an additional pharmaceutically active ingredient. Additionally or alternatively the at least one coding RNA may encode at least one tumor antigen, preferably as defined herein, or a fragment or variant thereof.

In a preferred embodiment, the combination comprises an isRNA and at least one coding RNA, which are formulated together or separately, preferably as described herein.

Independent of their formulation, the isRNA and the at least one coding RNA may preferably be administered concomitantly. Alternatively, the isRNA and the at least one coding RNA of the combination may be administered in a time-staggered manner.

The phrases “administered in combination”, co-administration or “concomitant administration” as used herein refer to a situation, where one pharmaceutically active ingredient, such as the isRNA described herein, is administered to a subject before, concomittantly or after the administration of at least one additional pharmaceutically active ingredient, such as the at least one coding RNA as described herein, to the same subject. The time interval between the administration of the pharmaceutically active ingredients depends on the nature and biological effect of the particular pharmaceutically active compononent and can be determined by a physician. Preferably, the time interval is less than about 48 hours, more preferably less than about 24 hours, 12 hours, 6 hours, 4 hours, 2 hours, 1 hour, most preferably less than about 30 minutes, 15 minutes or 5 minutes. In a particularly preferred embodiment, the phrase “administered in combination” refers to concomitant administration of pharmaceutically active ingredients, i.e. the simultaneous administration of at least two compounds or the administration of at least two compounds within a time frame that typically comprises less than 5 minutes. The phrase “administered in combination” does not only refer to a situation, where the pharmaceutically active ingredients are in physical contact with each other or formulated together. The phrase “administered in combination” as used herein comprises also the separate administration of the pharmaceutically active ingredients (e.g. by two separate injections). Alternatively, one pharmaceutically active ingredient, such as the isRNA described herein, may be administered in combination by mixing the ingredient with at least one additional pharmaceutically active ingredient, such as the at least one coding RNA, prior to administration and administering the mixture to a subject.

The phrases “administered in combination”, co-administration or “concomitant administration” as used herein further comprise a situation, wherein one pharmaceutically active ingredient, such as the isRNA described herein, is administered to a subject before, concomittantly or after, more preferably after, the administration of at least one additional pharmaceutically active ingredient, such as the at least one coding RNA as described herein, to the same subject. In some embodiments, the time interval between the administration of the pharmaceutically active ingredients is at least one, two, three, four, five, six, seven, eight, nine, ten, fifteen, twenty or thirty minutes. In certain embodiments, the isRNA is administered at least one, two, three, four, five, six, seven, eight, nine, ten, fifteen, twenty or thirty minutes after administration of the at least one coding RNA as described herein.

The isRNA and the at least one coding RNA of the combination according to the invention are preferably administered at the same site or at different sites, preferably by injection. Most preferably, at least one of the isRNA and the at least one coding RNA of the combination, preferably both, are administered intratumorally, preferably as described herein.

In another aspect, the invention provides the combination of an isRNA and at least one coding RNA as described herein for use as a medicament. The invention further provides the combination of an isRNA and at least one coding RNA for use in the manufacture of a medicament.

According to one embodiment, the combination as described herein is provided for use in the treatment or prophylaxis of a disease selected from the group consisting of tumor and cancer diseases, infectious diseases, allergies and autoimmune diseases. In a preferred embodiment, the combination as described herein is provided for use in the treatment or prophylaxis of a tumor or cancer disease, preferably as defined herein. According to a particularly preferred embodiment, the combination is for use in the treatment or prophylaxis of melanoma, preferably advanced and/or metastatic melanoma, most preferably advanced cutaneous melanoma (cMEL), squamous cell cancer of the skin (SCC), preferably unresectable and/or advanced SCC, most preferably cutaneous squamous cell carcinoma (cSCC), or other forms of malignant skin cancer, adenocystic carcinoma (ACC), preferably advanced ACC, cutaneous T-cell lymphoma, preferably advanced cutaneous T-cell lymphoma, and squamous cell carcinoma of the head and neck (HNSCC), preferably advanced HNSCC. Alternatively, the combination is for use in the treatment or prophylaxis of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

for treatment or prophylaxis of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy.

Preferably, the combination as described herein is for use in the treatment or prophylaxis of a disease as described herein, wherein the treatment or prophylaxis comprises administration of at least one additional pharmaceutically active ingredient, preferably as described herein. In a preferred embodiment, the combination is for use in the treatment or prophylaxis of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

for treatment or prophylaxis of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy;

wherein the treatment or prophylaxis comprises administration of at least one additional pharmaceutically active ingredient, wherein the at least one additional pharmaceutically active ingredient is a compound conventionally used in the treatment or prophylaxis of any of said diseases, preferably a compound as described herein in that context.

According to a particularly preferred embodiment, the combination as described herein is for use in the treatment or prophylaxis of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

for treatment or prophylaxis of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy;

wherein the treatment or prophylaxis comprises administration of a PD 1 inhibitor or a PD-L1 inhibitor, preferably as described herein, more preferably an antagonistic antibody directed against PD-1 or PD-L1.

In a further preferred embodiment, the combination as described herein is for use in the treatment or prophylaxis of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

for treatment or prophylaxis of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy;

wherein the treatment or prophylaxis comprises administration of an anti-CTLA4 antibody, preferably as described herein.

In a further aspect, the present invention concerns a coding RNA as described herein. The coding RNA according to the invention is typically characterized by any one of the features described herein in the context of the at least one coding RNA that may also be used as an additional pharmaceutically active ingredient as described herein with respect to the isRNA for use according to the invention. In particular, the coding RNA according to the invention encodes a peptide or protein comprising at least one peptide or protein selected from the group consisting of IL 12, CD40L, a decoy PD-1 receptor, preferably a soluble PD-1 receptor, an anti-CTLA4 antibody, and a tumor antigen, or a fragment or variant of any of these proteins.

According to a preferred embodiment, the coding RNA according to the invention encodes a peptide or protein comprising IL 12, CD40L and a decoy PD-1 receptor, preferably a soluble PD-1 receptor, or a fragment or variant of any of these proteins. For example, the coding RNA encoding said peptide or protein may be a multicistronic RNA comprising three open reading frames, wherein each open reading frame encodes a different peptide or protein selected from the group consisting of IL 12, CD40L, a decoy PD-1 receptor, preferably a soluble PD-1 receptor, an anti-CTLA4 antibody, and a tumor antigen, or a fragment or variant of any of these proteins.

In a further aspect, the present invention concerns the coding RNA according to the invention for use in the treatment or prophylaxis of a disease selected from the group consisting of tumor and cancer diseases, infectious diseases, allergies and autoimmune diseases. In a preferred embodiment, the coding RNA as described herein is provided for use in the treatment or prophylaxis of a tumor or cancer disease, preferably as defined herein. According to a particularly preferred embodiment, the coding RNA is for use in the treatment or prophylaxis of melanoma, preferably advanced and/or metastatic melanoma, most preferably advanced cutaneous melanoma (cMEL), squamous cell cancer of the skin (SCC), preferably unresectable and/or advanced SCC, most preferably cutaneous squamous cell carcinoma (cSCC), or other forms of malignant skin cancer, adenocystic carcinoma (ACC), preferably advanced ACC, cutaneous T-cell lymphoma, preferably advanced cutaneous T-cell lymphoma, and squamous cell carcinoma of the head and neck (HNSCC), preferably advanced HNSCC. In a further preferred embodiment, the coding RNA is for use in the treatment or prophylaxis of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

for treatment or prophylaxis of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy.

The coding RNA according to the invention is preferably provided for use in the treatment or prophylaxis of a disease as described herein, preferably in the treatment or prophylaxis of melanoma, preferably advanced and/or metastatic melanoma, most preferably advanced cutaneous melanoma (cMEL), squamous cell cancer of the skin (SCC), preferably unresectable and/or advanced SCC, most preferably cutaneous squamous cell carcinoma (cSCC), or other forms of malignant skin cancer, adenocystic carcinoma (ACC), preferably advanced ACC, cutaneous T-cell lymphoma, preferably advanced cutaneous T-cell lymphoma, and squamous cell carcinoma of the head and neck (HNSCC), preferably advanced HNSCC;

wherein the coding RNA comprises at least one coding sequence encoding a peptide or protein comprising IL-12, CD40L or a decoy PD-1 receptor, preferably a soluble PD-1 receptor, or a fragment or variant thereof, and wherein the treatment or prophylaxis comprises administration of a second coding RNA and/or a third coding RNA, wherein the second or third coding RNA comprises at least one coding sequence encoding a peptide or protein comprising CD40L or a decoy PD 1 receptor, preferably a soluble PD-1 receptor, or a fragment or variant thereof, so that the peptide(s) or protein(s) encoded by the coding RNAs together comprise IL-12, CD40L or a decoy PD-1 receptor, preferably a soluble PD-1 receptor, or a fragment or variant thereof. Therein, the coding RNA as well as the second and/or third coding RNAs are preferably coding RNAs as described herein.

The coding RNA according to the invention is preferably provided for use in the treatment or prophylaxis of a disease as described herein, preferably in the treatment or prophylaxis of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

for treatment or prophylaxis of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy;

wherein the coding RNA comprises at least one coding sequence encoding a peptide or protein comprising IL-12, CD40L, a decoy PD-1 receptor, preferably a soluble PD-1 receptor, or an anti-CTLA4 antibody, or a fragment or variant of any of these peptides or proteins, and wherein the treatment or prophylaxis comprises administration of a second coding RNA, a third coding RNA and/or a fourth coding RNA, wherein the second, third or fourth coding RNA comprises at least one coding sequence encoding a peptide or protein comprising IL12, CD40L, a decoy PD 1 receptor, preferably a soluble PD-1 receptor, or an anti-CTLA4 antibody or a fragment or variant thereof, so that the peptide(s) or protein(s) encoded by the coding RNAs together comprise IL-12, CD40L, a decoy PD-1 receptor, preferably a soluble PD-1 receptor, and an anti-CTLA4 antibody, or a fragment or variant thereof. Therein, the coding RNA as well as the second, third or fourth coding RNAs are preferably coding RNAs as described herein.

In certain embodiments, the coding RNA is provided for use in the treatment or prophylaxis of a disease as described herein, wherein the treatment or prophylaxis further comprises chemotherapy, radiation therapy and/or surgery. According to a preferred embodiment, the treatment or prophylaxis comprises the administration of at least one additional pharmaceutically active ingredient. In a further preferred embodiment, the treatment or prophylaxis comprises the administration of a compound that is conventionally used in the treatment of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

in the treatment or prophylaxis of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy;

preferably a compound as described herein in that context. More preferably, the treatment or prophylaxis comprises the administration of a PD 1 inhibitor or a PD L1 inhibitor, preferably an antagonistic antibody directed against PD-1 or PD-L1. In a particularly preferred embodiment, the treatment or prophylaxis comprises the administration, preferably intratumorally, of an isRNA, preferably an isRNA as described herein.

In some embodiments, the treatment or prophylaxis comprises the administration of a compound that is conventionally used in the treatment of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

in the treatment or prophylaxis of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy; preferably a compound as described herein in that context. More preferably, the treatment or prophylaxis comprises the administration of an anti-CTLA4 antibody. In a particularly preferred embodiment, the treatment or prophylaxis comprises the administration, preferably intratumorally, of an isRNA, preferably an isRNA as described herein.

The coding RNA for use as described herein is preferably administered intratumorally. In a particularly preferred embodiment, the coding RNA for use as described herein is administered intratumorally.

In a further aspect, the present invention provides a pharmaceutical composition comprising an isRNA, preferably as described herein, at least one coding RNA, preferably as described herein, or the combination thereof, wherein the pharmaceutical composition comprises a pharmaceutically acceptable carrier and/or vehicle.

Preferably, the pharmaceutical composition is prepared for intratumoral application, preferably by injection into tumor tissue. Sterile injectable forms of the inventive pharmaceutical composition may be aqueous or oleaginous suspension. These suspensions may be formulated according to techniques known in the art using suitable dispersing or wetting agents and suspending agents.

A pharmaceutically acceptable carrier typically includes the liquid or non-liquid basis of a composition comprising an isRNA, preferably as described herein, at least one coding RNA, preferably as described herein, or the combination thereof as described herein. If the composition is provided in liquid form, the carrier will typically be pyrogen-free water; isotonic saline or buffered (aqueous) solutions, e.g. phosphate, citrate etc. buffered solutions. The injection buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of cells due to osmosis or other concentration effects. Reference media are e.g. liquids occurring in “in vivo” methods, such as blood, lymph, cytosolic liquids, or other body liquids, or e.g. liquids, which may be used as reference media in “in vitro” methods, such as common buffers or liquids. Such common buffers or liquids are known to a skilled person. Ringer-Lactate solution is particularly preferred as a liquid basis.

However, one or more compatible solid or liquid fillers or diluents or encapsulating compounds, which are suitable for administration to a patient to be treated, may be used as well for the pharmaceutical composition according to the invention. The term “compatible” as used herein means that these constituents of the inventive pharmaceutical composition are capable of being mixed with the components of the inventive pharmaceutical composition in such a manner that no interaction occurs which would substantially reduce the pharmaceutical effectiveness of the pharmaceutical composition under typical use conditions.

The inventive pharmaceutical composition may comprise further components for facilitating administration and uptake of components of the pharmaceutical composition. Such further components may be an appropriate carrier or vehicle, additional adjuvants for supporting any immune response, antibacterial and/or antiviral agents.

A further component of the inventive pharmaceutical composition may be an immunotherapeutic agent that can be selected from immunoglobulins, preferably IgGs, monoclonal or polyclonal antibodies, polyclonal serum or sera, etc.

Preferably, such a further immunotherapeutic agent may be provided as a peptide/protein or may be encoded by a nucleic acid, preferably by a DNA or an RNA, more preferably an mRNA.

The inventive pharmaceutical composition typically comprises a “safe and effective amount” of the components of the inventive pharmaceutical composition, particularly of the isRNA, the coding RNA as defined herein or the combination thereof as defined herein. As used herein, a “safe and effective amount” means an amount of the RNA molecule(s) as defined herein as such that is sufficient to significantly induce a positive modification of the disease, preferably of a tumor or cancer disease. At the same time, however, a “safe and effective amount” is small enough to avoid serious side-effects and to permit a sensible relationship between advantage and risk. The determination of these limits typically lies within the scope of sensible medical judgment.

The inventive pharmaceutical composition may be used for human and also for veterinary medical purposes, preferably for human medical purposes, as a pharmaceutical composition in general.

According to another particularly preferred aspect, the pharmaceutical composition as described herein may be provided or used as a vaccine. Typically, such a vaccine is as defined above for pharmaceutical compositions. Preferably, such a vaccine typically contains the isRNA as described herein, the at least one coding RNA as described herein or the combination thereof as described herein. The vaccine may also comprise a pharmaceutically acceptable carrier, adjuvant, and/or vehicle as defined herein for the pharmaceutical composition. In the specific context of the vaccine, the choice of a pharmaceutically acceptable carrier is determined in principle by the manner, in which the inventive vaccine is administered. The vaccine may preferably be administered locally into tumor tissue.

The vaccine can additionally contain one or more auxiliary substances in order to increase its immunogenicity or immunostimulatory capacity, if desired. Particularly preferred are adjuvants as auxiliary substances or additives as defined for the pharmaceutical composition.

In a further aspect, the invention relates to a kit or kit of parts comprising the isRNA as described herein, at least one coding RNA as described herein, the combination as described comprising the isRNA and at least one coding RNA as described herein or comprising the pharmaceutical composition or vaccine as described herein, or the components thereof and optionally technical instructions with information on the administration and dosage of the components.

Besides the isRNA and/or at least one coding RNA, the kit may additionally contain a pharmaceutically acceptable vehicle, an adjuvant and at least one further component e.g. an additional pharmaceutically active component/compound as defined herein, as well as means for administration and technical instructions. The components of the composition, in particular the isRNA or the at least one coding RNA as described herein, and possibly further components may be provided in lyophilized form. In a preferred embodiment, prior to use of the kit, the provided vehicle is then added to the lyophilized components in a predetermined amount as written e.g. in the provided technical instructions.

In a particularly preferred embodiment, the kit may comprise the isRNA, preferably complexed by a polymeric carrier, as described herein in lyophilized form and the at least one coding RNA as described herein in lyophilized form and additionally a pharmaceutically acceptable vehicle, an adjuvant and at least one further component e.g. an additional pharmaceutically active component/compound as defined herein, as well as means for administration and technical instructions. Preferably, the kit comprises the isRNA, preferably complexed by a polymeric carrier, as described herein in lyophilized form and at least three, preferably at least four or five, coding RNAs encoding IL-12, CD40L, a decoy PD-1 receptor, preferably a soluble PD-1 receptor, an ati-CTLA4 antibody, and/or a tumor antigen as described herein, or a fragment or variant of any of these, in lyophilized form and a liquid for reconstitution, e.g. Ringer's Lactate solution or water.

The present invention furthermore several applications and uses of the isRNA as described herein, the at least one coding RNA as described herein, the combination thereof as described herein, or, respectively, the pharmaceutical composition, or the vaccine, or the kit or kit of parts as defined herein. As a main aspect of the invention, the isRNA as described herein, the at least one coding RNA as described herein, the combination thereof as described herein, or, respectively, the pharmaceutical composition or the kit or kit of parts may be used as a medicament, preferably for treatment or prophylaxis of a disease as described herein, more preferably for treatment of tumor or cancer diseases, most preferably for treatment of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

for the treatment of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy.

The present invention thus provides the isRNA as described herein, the at least one coding RNA as described herein, the combination thereof as described herein, or, respectively, the pharmaceutical composition, or the vaccine, or the kit or kit of parts for use in the manufacture of a medicament. In this context, the treatment is preferably carried out by intratumoral application, especially by injection into tumor tissue. According to another aspect, the present invention is directed to the second medical use of the isRNA as described herein, the at least one coding RNA as described herein, the combination thereof as described herein, or, respectively, the pharmaceutical composition, or the vaccine, or the kit or kit of parts as described above, wherein these subject matters are used for preparation of a medicament particularly for intratumoral application (administration) for treatment of tumor or cancer diseases, preferably as described herein.

In one aspect, the present invention provides a method of treating or preventing a disorder, wherein the method comprises administering, preferably intratumorally, to a subject in need thereof an effective amount of a medicament as described herein, preferably of the isRNA as described herein, of the at least one coding RNA as described herein, of the combination thereof as described herein, or, respectively, of the pharmaceutical composition, or of the vaccine. More preferably, the method is for treating or preventing a tumor or cancer disease as described herein, most preferably a disorder selected from the group consisting of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the disorder is preferably at an advanced stage and/or refractory to standard therapy.

The present invention thus provides the isRNA as described herein, the at least one coding RNA as described herein, the combination thereof as described herein, or, respectively, the pharmaceutical composition, or the vaccine, or the kit or kit of parts as defined herein as a medicament. Accordingly, the term “medicament” as used herein typically refers to the isRNA as described herein, the at least one coding RNA as described herein, the combination thereof as described herein (preferably formulated in order to allow administration, e.g. in a liquid formulation), or, respectively, the pharmaceutical composition, or the vaccine, or the kit or kit of parts as defined herein. In this context, it is preferred that the isRNA and/or the at least one coding RNA as described herein are provided in lyophilized form and are re-solubilized prior to administration, for instance by addition of a suitable vehicle as known in the art or as described herein, such as Ringer's Lactate solution or water.

The medicament as described herein may be administered by conventional needle injection or needle-free jet injection into the tumor tissue. In a preferred embodiment the medicament is administered by jet injection. Jet injection refers to a needle-free injection method, wherein a fluid comprising the composition and, optionally, further suitable excipients is forced through an orifice, thus generating an ultra-fine liquid stream of high pressure that is capable of penetrating mammalian skin. In principle, the liquid stream forms a hole in the skin, through which the liquid stream is pushed into the target tissue, namely the tumor tissue. According to the invention, jet injection may be used for intratumoral application of the medicament as described herein.

The medicament may be administered by conventional needle injection or needle-free jet injection adjacent to and/or in close proximity to the tumor tissue. In a preferred embodiment the medicament is administered by jet injection adjacent to and/or in close proximity to the tumor tissue. According to the invention, jet injection may be used for intratumoral application (adjacent to and/or in close proximity to the tumor tissue), particularly for injection of the medicament.

In some embodiments, the medicament may be administered orally, parenterally, by inhalation spray, topically, rectally, nasally, buccally, vaginally or via an implanted reservoir. The term parenteral as used herein includes subcutaneous, intravenous, intramuscular, intraarticular, intranodal, intrasynovial, intrasternal, intrathecal, intrahepatic, intralesional, intracranial, transdermal, intradermal, intrapulmonal, intraperitoneal, intracardial, intraarterial, and sublingual injection or infusion techniques. Further particularly preferred administration routes are intradermal and intramuscular injection. Preferably, the administration comprises an imaging technique, preferably as described herein. More preferably, the medicament is administered locoregionally, preferably as described herein. Even more preferably, the medicament is administered locoregionally, wherein the administration comprises an imaging technique, preferably as described herein.

According to a further embodiment, the treatment or prophylaxis of a disease, preferably of a tumor or cancer disease as described herein, comprises administration of at least one pharmaceutical composition comprising the isRNA as described herein and the administration of at least one further pharmaceutical composition comprising at least one coding RNA as described herein, wherein the pharmaceutical compositions may be administered via the same or via different routes. More preferably, the pharmaceutical compositions are administered via the same route, preferably intratumorally, e.g. by intratumoral or peritumoral injection.

According to a specific embodiment, the medicament may be administered to the patient as a single dose or as several doses. In certain embodiments, the medicament may be administered to a patient as a single dose followed by a second dose later and optionally even a third, fourth (or more) dose subsequent thereto etc.

Preferably, the medicament comprises at least 25 μg of isRNA and/or coding RNA per dose. Alternatively, a single dose of the medicament may comprise at least 15, 20, 25, 30, 35, 40, 45, 50, 55, 60, 65, 70, 75, 80, 85, 90, 95 or 100 μg of isRNA and/or coding RNA as described herein. More specifically, the amount of isRNA and/or coding RNA comprised in a single dose is typically at least 100 μg or 200 μg, preferably from 200 μg to 1.000 μg, more preferably from 300 μg to 850 μg, even more preferably from 300 μg to 700 μg. Where the medicament comprises more than one type of RNA, the values above preferably refer to the amounts of each single type of RNA.

According to a particularly preferred embodiment, the present invention provides an isRNA as described herein for use in the treatment of a tumor or cancer disease, preferably a disease selected from the group consisting of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the disease is preferably at an advanced stage and/or refractory to standard therapy.

Preferably, the treatment comprises administration, preferably intratumorally, of an isRNA as described herein, which is complexed by a cationic or polycationic compound. In a preferred embodiment, the isRNA comprises a nucleic acid sequence according to formula (I) (GₗXₘGₙ), formula (II) (CₗXₘCₙ), formula (III) (NᵤGₗXₘGₙNᵥ)ₐ or formula (IV) (NᵤCₗXₘCₙNᵥ)ₐ, preferably at least one nucleic acid sequence according to any one of SEQ ID NOs: 433 to 437, 1014 to 1016, 1055 or 1056, or a fragment or variant of any of these sequences, preferably according to any one of SEQ ID NOs: 433, 434 or 1014 to 1016, or a fragment or variant of any of these nucleic acid sequences, wherein the isRNA is complexed with a cationic or polycationic compound, preferably with a polymeric carrier, more preferably with a polymeric carrier that is formed by a disulfide-crosslinked cationic component, which preferably comprises a peptide according to formula (V), (Va) and/or (Vb) and/or a compound according to formula (VI), more preferably at least one of the disulfide-crosslinked cationic peptides Cys-Arg₁₂ (SEQ ID NO: 580) or Cys-Arg₁₂-Cys (SEQ ID NO: 579). More preferably, the isRNA comprises a nucleic acid sequence according to SEQ ID NOs: 433 to 437, 1014 to 1016, or a fragment or variant of any of these sequences, preferably according to SEQ ID NO: 433, or a fragment or variant thereof, which is complexed with a cationic or polycationic compound as described herein, preferably with the disulfide-crosslinked peptide Cys-Arg₁₂-Cys (SEQ ID NO: 579). Most preferably, the isRNA in this consists of an RNA sequence according to SEQ ID NO: 433, or a fragment or variant thereof, which is complexed with the disulfide-crosslinked peptide Cys-Arg₁₂-Cys (SEQ ID NO: 579).

The treatment preferably comprises intratumoral administration of the isRNA as described above (or a pharmaceutical composition comprising said isRNA, respectively) to a subject suffering from a disease selected from the group consisting of
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the disease is preferably at an advanced stage and/or refractory to standard therapy.

In a preferred embodiment, the subject receiving the treatment suffers from advanced and/or metastatic melanoma, unresectable and/or advanced SCC, unresectable and/or advanced adenocystic carcinoma (ACC), unresectable and/or advanced or cutaneous T-cell lymphoma or advanced and/or platinum-refractory HNSCC. Preferably, the subject has injectable tumor lesions. More preferably, the subject has no other treatment options.

More preferably, the treatment comprises intratumoral administration of the isRNA as described above (or a pharmaceutical composition comprising said isRNA, respectively) to a subject suffering from advanced melanoma, preferably advanced cutaneous melanoma, who is being treated with a checkpoint inhibitor, preferably as described herein. Most preferably, the subject is treated with a PD-1 or PD-L1 inhibitor, preferably as described herein, more preferably an antagonistic antibody against PD-1 or an antagonistic antibody against PD-L1.

The treatment preferably comprises intratumoral administration of a single dose of the isRNA as described above (or a pharmaceutical composition comprising said isRNA, respectively) to the subject, either once or repeatedly. In this context, a single dose preferably comprises from 20 μg to 500 μg, more preferably from 50 μg to 350 μg, of the isRNA as described above. In preferred embodiments, a single dose comprises at least 25 μg, 50 μg, 75 μg, 100 μg, 125 μg, 150 μg, 175 μg, 200 μg, 225 μg or at least 250 μg of the isRNA as described above. Most preferably, a single dose comprises about 25 μg, about 50 μg, about 100 μg or about 150 μg of the isRNA as described above.

The treatment may comprise repeated intratumoral administration to the subject of a single dose of the isRNA as described above (or a pharmaceutical composition comprising said isRNA, respectively), wherein at least 2, 3, 4, 5, 6, 7, 8, 9, 10, 11, 12 or more single doses are preferably administered to the subject and wherein the interval between the administration of two single doses is at least 1, 2, 3, 4, 5, 6, 7, 8, 9, 10, 11, 12, 13 or 14 days. The interval between the administration of two single doses may be constant or may be varied throughout the treatment. For example, the treatment may comprise intratumoral administration of a single dose once weekly for four weeks, followed by further single doses, preferably 3 to 8 further single doses, which are administered every two weeks.

According to another embodiment, the treatment comprises intratumoral administration of the isRNA as described above (or a pharmaceutical composition comprising said isRNA, respectively) to a subject suffering from a tumor or cancer disease, preferably selected from the group consisting of breast cancer (hormone receptor positive or negative forms); melanoma, preferably advanced and/or metastatic melanoma; squamous cell cancer of the skin (SCC), preferably unresectable and/or advanced SCC, or other forms of malignant skin cancer; adenocystic carcinoma (ACC), preferably advanced ACC; cutaneous T-cell lymphoma, preferably advanced cutaneous T-cell lymphoma; squamous cell carcinoma of the head and neck (HNSCC), preferably advanced HNSCC; salivary gland cancer; nasopharynx cancers; lung cancer or lung metastases of other malignancies; mesothelioma; bladder cancer; thyroid cancer; esophageal and gastric cancer; liver cancer; malignancies with liver metastases; ovarian cancer; cervix cancer; renal cancer; hematological malignancies with injectable lesions like cutaneous T-cell lymphoma; solitary or multiple myeloma; Hodgkin's disease; non-Hodgkin lymphoma with injectable lesions; sarcoma including its various subtypes; glioma grade I-IV; colorectal, rectal and anal cancer, more preferably selected from the group consisting of melanoma, preferably advanced and/or metastatic melanoma, most preferably advanced cutaneous melanoma (cMEL), squamous cell cancer of the skin (SCC), preferably unresectable and/or advanced SCC, most preferably cutaneous squamous cell carcinoma (cSCC), or other forms of malignant skin cancer, adenocystic carcinoma (ACC), preferably advanced ACC, cutaneous T-cell lymphoma, preferably advanced cutaneous T-cell lymphoma, and squamous cell carcinoma of the head and neck (HNSCC), preferably advanced HNSCC, wherein the treatment further comprises administration, preferably intratumorally, of at least one coding RNA as defined herein. In that embodiment, the treatment thus preferably comprises administration of the combination according to the present invention as described herein.

In that embodiment, the isRNA is preferably as described above and the at least one coding RNA is preferably an mRNA as described herein encoding at least one peptide or protein comprising IL-12, a decoy PD-1 receptor, preferably a soluble PD-1 receptor, CD40L, or an anti-CTLA4 antibody or a fragment or variant of any of these proteins as described herein.

More preferably, the treatment comprises intratumoral administration of the isRNA as described above (or a pharmaceutical composition comprising said isRNA, respectively) and further comprises administration, preferably intratumorally, of at least three, preferably at least four or five, coding RNAs as described herein, preferably at least three, more preferably at least four or five, mRNAs, wherein

a first coding RNA comprises a nucleic acid sequence selected from the group consisting of SEQ ID NOs: 285; 296; 307; 318; 329; 340; 351; 362; 373; 384; 395; 406; 430; 469 and 992, or a fragment or variant of any of these sequences,

a second coding RNA comprises a nucleic acid sequence selected from the group consisting of SEQ ID NOs: 286; 297; 308; 319; 330; 341; 352; 363; 374; 385; 396; 470 and 407, or a fragment or variant of any of these sequences,

a third coding RNA comprises a nucleic acid sequence selected from the group consisting of SEQ ID NOs: 926-940, or

a fragment or variant of any of these sequences,

a fourth coding RNA comprises a nucleic acid sequence selected from the group consisting of SEQ ID NOs: 956-970, or a fragment or variant of any of these sequences, and/or

optionally, a fifth coding RNA comprises a nucleic acid sequence selected from the group consisting of SEQ ID NOs: 277; 288; 299; 310; 321; 332; 343; 354; 365; 376; 387; 461 and 398, or a fragment or variant of any of these sequences. Therein, the first, second, third, fourth and fifth coding RNAs are preferably formulated separately and administered concomitantly.

Even more preferably, the treatment comprises intratumoral administration of the isRNA as described above (or a pharmaceutical composition comprising said isRNA, respectively) and further comprises administration, preferably intratumorally, of at least three, preferably of at least four or five, coding RNAs as described herein, preferably at least three, more preferably at least four or five, mRNAs, wherein

a first coding RNA comprises a nucleic acid sequence selected from the group consisting of SEQ ID NOs: 285; 296; 307; 318; 329; 340; 351; 362; 373; 384; 395; 406; 430; 469 and 992, or a fragment or variant of any of these sequences,

a second coding RNA comprises a nucleic acid sequence selected from the group consisting of SEQ ID NOs: 286; 297; 308; 319; 330; 341; 352; 363; 374; 385; 396; 470 and 407, or a fragment or variant of any of these sequences,

a third coding RNA comprises a nucleic acid sequence selected from the group consisting of SEQ ID NOs: 926-940, or

a fragment or variant of any of these sequences,

a fourth coding RNA comprises a nucleic acid sequence selected from the group consisting of SEQ ID NOs: 956-970, or a fragment or variant of any of these sequences, and/or

optionally, a fifth coding RNA comprises a nucleic acid sequence selected from the group consisting of SEQ ID NOs: 277; 288; 299; 310; 321; 332; 343; 354; 365; 376; 387; 461 and 398, or a fragment or variant of any of these sequences. Therein, the first, second, third, fourth and fifth coding RNAs are preferably formulated separately and administered concomitantly.

Most preferably, the treatment comprises intratumoral administration of the isRNA as described above (or a pharmaceutical composition comprising said isRNA, respectively) and further comprises administration, preferably intratumorally, of at least three, preferably at least four or five, coding RNAs as described herein, preferably at least three, more preferably at least four of five, mRNAs, wherein

a first coding RNA comprises a nucleic acid sequence selected from the group consisting of SEQ ID NOs: 285; 296; 307; 318; 329; 340; 351; 362; 373; 384; 395; 406; 430; 469 and 992, or a fragment or variant of any of these sequences,

a second coding RNA comprises a nucleic acid sequence selected from the group consisting of SEQ ID NOs: 286; 297; 308; 319; 330; 341; 352; 363; 374; 385; 396; 470 and 407, or a fragment or variant of any of these sequences,

a third coding RNA comprises a nucleic acid sequence selected from the group consisting of SEQ ID NOs: 926-940, or

a fragment or variant of any of these sequences,

a fourth coding RNA comprises a nucleic acid sequence selected from the group consisting of SEQ ID NOs: 956-970, or a fragment or variant of any of these sequences, and/or

optionally, a fifth coding RNA comprises a nucleic acid sequence selected from the group consisting of SEQ ID NOs: 277; 288; 299; 310; 321; 332; 343; 354; 365; 376; 387; 461 and 398, or a fragment or variant of any of these sequences. Therein, the first, second, third, fourth and fifth coding RNAs are preferably formulated separately and administered concomitantly.

Hence, the treatment preferably comprises

intratumoral administration of an isRNA comprising a nucleic acid sequence according to SEQ ID NOs: 433 to 437, 1014 to 1016 or a fragment or variant of any of these sequences, preferably according to SEQ ID NO: 433 or a fragment or variant thereof, which is complexed with a cationic or polycationic compound as described herein, preferably with the disulfide-crosslinked peptide Cys-Arg₁₂-Cys (SEQ ID NO: 579), and

intratumoral, peritumoral or locoregional administration of at least five coding RNAs, preferably an mRNA, wherein

a first coding RNA comprises a nucleic acid sequence selected from the group consisting of SEQ ID NOs: 296; 307; 318; 329; 340; 351; 362; 373; 384; 395; 406; 430 and 992, or a fragment or variant of any of these sequences,

a second coding RNA comprises a nucleic acid sequence selected from the group consisting of SEQ ID NOs: 297; 308; 319; 330; 341; 352; 363; 374; 385; 396 and 407, or a fragment or variant of any of these sequences,

a third coding RNA comprises a nucleic acid sequence selected from the group consisting of SEQ ID NOs: 926-940, or

a fragment or variant of any of these sequences,

a fourth coding RNA comprises a nucleic acid sequence selected from the group consisting of SEQ ID NOs: 956-970, or a fragment or variant of any of these sequences, and/or

optionally, a fifth coding RNA comprises a nucleic acid sequence selected from the group consisting of SEQ ID NOs: 288; 299; 310; 321; 332; 343; 354; 365; 376; 387 and 398, or a fragment or variant of any of these sequences,

wherein the first, second, third, fourth and fifth coding RNAs are preferably formulated separately and administered concomitantly.

According to an alternative embodiment, the treatment comprises

intratumoral administration of an isRNA comprising a nucleic acid sequence according to SEQ ID NOs: 433 to 437, 1014 to 1016, 10555 or 1056 or a fragment or variant of any of these sequences, preferably according to SEQ ID NO: 433 or a fragment or variant thereof, which is complexed with a cationic or polycationic compound as described herein, preferably with the disulfide-crosslinked peptide Cys-Arg₁₂-Cys (SEQ ID NO: 579), and

intratumoral, peritumoral or locoregional administration of at least five coding RNAs, preferably an mRNA, wherein a first coding RNA comprises a nucleic acid sequence selected from the group consisting of SEQ ID NOs: 285; 296; 307; 318; 329; 340; 351; 362; 373; 384; 395; 406; 430; 469 and 992, or a fragment or variant of any of these sequences,

a second coding RNA comprises a nucleic acid sequence selected from the group consisting of SEQ ID NOs: 286; 297; 308; 319; 330; 341; 352; 363; 374; 385; 396; 470 and 407, or a fragment or variant of any of these sequences,

a third coding RNA comprises a nucleic acid sequence selected from the group consisting of SEQ ID NOs: 926-940, or

a fragment or variant of any of these sequences,

a fourth coding RNA comprises a nucleic acid sequence selected from the group consisting of SEQ ID NOs: 956-970,

or a fragment or variant of any of these sequences, and/or

optionally, a fifth coding RNA comprises a nucleic acid sequence selected from the group consisting of SEQ ID NOs: 277; 288; 299; 310; 321; 332; 343; 354; 365; 376; 387; 461 and 398, or a fragment or variant of any of these sequences,

wherein the first, second, third, fourth and fifth coding RNAs are preferably formulated separately and administered concomitantly.

Most preferably, the treatment preferably comprises

Intratumoral, peritumoral or locoregional administration of an isRNA comprising a nucleic acid sequence according to SEQ ID NO: 433 to 437, 1014 to 1016, 1055 or 1056, or a fragment or variant of any of these sequences, preferably according to SEQ ID NO: 433 or a fragment or variant thereof, which is complexed with a cationic or polycationic compound as described herein, preferably with the disulfide-crosslinked peptide Cys-Arg₁₂-Cys (SEQ ID NO: 579), and intratumoral, peritumoral or locoregional administration of at least five coding RNAs, preferably an mRNA, wherein

a first coding RNA comprises a nucleic acid sequence selected from the group consisting of of SEQ ID NOs: 296; 307; 318; 329; 340; 351; 362; 373; 384; 395; 406; 430 and 992, or a fragment or variant of any of these sequences,

a second coding RNA comprises a nucleic acid sequence selected from the group consisting of SEQ ID NOs: 297; 308; 319; 330; 341; 352; 363; 374; 385; 396 and 407, or a fragment or variant of any of these sequences,

a third coding RNA comprises a nucleic acid sequence selected from the group consisting of SEQ ID NOs: 926-940, or

a fragment or variant of any of these sequences,

a fourth coding RNA comprises a nucleic acid sequence selected from the group consisting of SEQ ID NOs: 956-970, or a fragment or variant of any of these sequences, and/or

optionally, a fifth coding RNA comprises a nucleic acid sequence selected from the group consisting of SEQ ID NOs: 288; 299; 310; 321; 332; 343; 354; 365; 376; 387 and 398, or a fragment or variant of any of these sequences,

wherein the first, second, third, fourth and fifth coding RNAs are preferably formulated separately and administered concomitantly.

In that embodiment, the isRNA, the dosage regimen of the isRNA and the administration of the isRNA are preferably as described above with respect to the isRNA for use in the treatment of a tumor or cancer disease.

The dosage regimen and the administration of the at least one coding RNA, which is administered concomitantly with the isRNA as described above, is preferably identical to the dosage regimen and the administration as described above with respect to the isRNA for use in the treatment of a tumor or cancer disease. The treatment thus preferably comprises intratumoral administration of a single dose of the at least one coding RNA as described herein (or a pharmaceutical composition comprising said RNA, respectively) to the subject, either once or repeatedly. In this context, a single dose preferably comprises from 20 μg to 500 μg, more preferably from 50 μg to 350 μg, of the at least one coding RNA as described above. In preferred embodiments, a single dose comprises at least 25 μg, 50 μg, 75 μg, 100 μg, 125 μg, 150 μg, 175 μg, 200 μg, 225 μg or at least 250 μg of the at least one coding RNA as described above. Most preferably, a single dose comprises about 25 μg, about 50 μg, about 100 μg or about 150 μg of the at least one coding RNA as described herein. More preferably, the treatment comprises administration of at least three coding RNAs as described above, wherein a single dose comprises the above indicated amounts of each of the coding RNAs. The treatment preferably comprises repeated administration of a single dose as described above. Preferably, the doses of the at least one coding RNA are administered concomitantly with the isRNA as described above and preferably following the schedule described in that context.

In a particularly preferred embodiment the subject receiving the isRNA, the at least one coding RNA, the combination thereof or the pharmaceutical composition or vaccine comprising said RNA(s) may be a patient with cancer or tumor who receives or received standard treatments of cancer. Preferably, the patient has achieved partial response or stable disease after having received standard treatments.

The standard treatments of cancer include chemotherapy, radiation, chemoradiation and surgery dependent on the particular cancer or tumor type to be treated, wherein these treatments are applied individually or in combination.

In some embodiments, the subject receiving the isRNA, the at least one coding RNA, the combination thereof or the pharmaceutical composition or vaccine comprising said RNA(s) may be a patient with a tumor or cancer disease, preferably as defined herein, more preferably a disease selected from the group consisting of melanoma, preferably advanced and/or metastatic melanoma, most preferably advanced cutaneous melanoma (cMEL), squamous cell cancer of the skin (SCC), preferably unresectable and/or advanced SCC, most preferably cutaneous squamous cell carcinoma (cSCC), or other forms of malignant skin cancer, adenocystic carcinoma (ACC), preferably advanced ACC, cutaneous T-cell lymphoma, preferably advanced cutaneous T-cell lymphoma, and squamous cell carcinoma of the head and neck (HNSCC), preferably advanced HNSCC, who received or receives chemotherapy (e.g. first-line or second-line chemotherapy), radiotherapy, chemoradiation (combination of chemotherapy and radiotherapy), kinase inhibitors, antibody therapy and/or checkpoint modulators (e.g. CTLA4 inhibitors, PD1 pathway inhibitors), or a patient, who has achieved partial response or stable disease after having received one or more of the treatments specified above.

According to certain embodiments, the subject receiving the isRNA, the at least one coding RNA, the combination thereof or the pharmaceutical composition or vaccine comprising said RNA(s) may be a subject suffering from a disease selected from the group consisting of melanoma, preferably advanced and/or metastatic melanoma, most preferably advanced cutaneous melanoma (cMEL), squamous cell cancer of the skin (SCC), preferably unresectable and/or advanced SCC, most preferably cutaneous squamous cell carcinoma (cSCC), or other forms of malignant skin cancer, adenocystic carcinoma (ACC), preferably advanced ACC, cutaneous T-cell lymphoma, preferably advanced cutaneous T-cell lymphoma, and squamous cell carcinoma of the head and neck (HNSCC), preferably advanced HNSCC, who received or receives, preferably via intratumoral administration, a compound conventionally used in any of these diseases as described herein.

More preferably, the subject receiving the isRNA, the at least one coding RNA, the combination thereof or the pharmaceutical composition or vaccine comprising said RNA(s) suffers from a disease selected from the group consisting of melanoma, preferably advanced and/or metastatic melanoma, most preferably advanced cutaneous melanoma (cMEL), squamous cell cancer of the skin (SCC), preferably unresectable and/or advanced SCC, most preferably cutaneous squamous cell carcinoma (cSCC), or other forms of malignant skin cancer, adenocystic carcinoma (ACC), preferably advanced ACC, cutaneous T-cell lymphoma, preferably advanced cutaneous T-cell lymphoma, and squamous cell carcinoma of the head and neck (HNSCC), preferably advanced HNSCC, and received or received a checkpoint modulator.

According to some embodiments, the subject receiving the isRNA, the at least one coding RNA, the combination thereof or the pharmaceutical composition or vaccine comprising said RNA(s) may be a patient with a tumor or cancer disease selected from
melanoma, preferably advanced and/or metastatic melanoma, more preferably advanced cutaneous melanoma (cMEL);
squamous cell cancer (SCC) of the skin, preferably unresectable and/or advanced SCC of the skin, more preferably cutaneous squamous cell carcinoma (cSCC), even more preferably unresectable and/or advanced cSCC;
squamous cell carcinoma of the head and neck (HNSCC), preferably advanced HNSCC, more preferably immunotherapy-refractory or platinum-refractory HNSCC, even more preferably immunotherapy-refractory advanced HNSCC or platinum-refractory advanced HNSCC;
adenocystic carcinoma (ACC), preferably advanced ACC;
cutaneous T-cell lymphoma, preferably advanced cutaneous T-cell lymphoma or cutaneous T-cell lymphoma of mycosis fungoides subtype, more preferably cutaneous T-cell lymphoma refractory to local treatment or to chemotherapy, even more preferably advanced cutaneous T-cell lymphoma refractory to local treatment or to chemotherapy, or cutaneous T-cell lymphoma of mycosis fungoides subtype refractory to local treatment or to chemotherapy, and
vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy; or

selected from cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC),

wherein the tumor or the cancer disease is preferably at an advanced stage and/or refractory to standard therapy. More preferably, the subject receiving the isRNA, the at least one coding RNA, the combination thereof or the pharmaceutical composition or vaccine comprising said RNA(s) is a patient suffering from a tumor or cancer disease as described herein and who received or receives chemotherapy (e.g. first-line or second-line chemotherapy), radiotherapy, chemoradiation (combination of chemotherapy and radiotherapy), kinase inhibitors, antibody therapy and/or checkpoint modulators (e.g. CTLA4 inhibitors, PD1 pathway inhibitors), or a patient, who has achieved partial response or stable disease after having received one or more of the treatments specified above. More preferably, the subject is a patient suffering from a tumor or cancer disease as described herein and who received or receives, preferably via intratumoral administration, a compound conventionally used in any of these diseases as described herein, more preferably a patient who receives or received, preferably via intratumoral administration, a checkpoint modulator.

The subject receiving the isRNA, the at least one coding RNA, the combination thereof or the pharmaceutical composition or vaccine comprising said RNA(s) preferably suffers from melanoma, preferably advanced and/or metastatic melanoma, and received or received, preferably via intratumoral administration, at least one of the following treatments:
checkpoint modulator monotherapy, such as Pembrolizumab, Nivolumab or Ipilimumab;
checkpoint modulator combination therapy, such as Nivolumab and Ipilimumab;
combination therapy comprising Dabrafenib and Trametinib, or Vemurafenib and cobimetinib or single agent therapy using Vemurafenib or Dabrafenib;
high-dose IL-2 treatment;
Imatinib;
cytotoxic treatments using compounds such as Dacarbazine, Temozolomide, Paclitaxel, Albumin-bound paclitaxel, Carboplatin/paclitaxel;
biochemotherapy using compounds such as Decarbazine and/or temozolomide and/or carboplatin with/without vinblastine and/or nitrosourea and/or IL-2 and/or interferon alfa2b.

The subject receiving the isRNA, the at least one coding RNA, the combination thereof or the pharmaceutical composition or vaccine comprising said RNA(s) preferably suffers from squamous cell carcinoma of the head and neck (HNSCC), preferably advanced HNSCC, and received or received, preferably via intratumoral administration, at least one of the following treatments:
treatments using single agents, e.g. Cisplatin, Carboplatin, Paclitaxel, Docetaxel, 5-FU, Methotrexate, Cetuximab, Gemcitabine, Capecitabine, Vinorelbine, Afatinib;
treatments using a combination of agents; such as
Carboplatin and infusional 5-FU;
5-FU and hydroxyurea;
Cisplatin and paclitaxel;
Cisplatin and infusional 5-FU;
Cisplatin and 5-FU;
Carboplatin and paclitaxel;
Cisplatin followed by cisplatin and 5-FU;
Docetaxel and cisplatin and 5-FU;
Paclitaxel and cisplatin and infusional 5-FU;
Docetaxel and cisplatin;
Cisplatin and epirubicin and paclitaxel;
Cisplatin or carboplatin and 5-FU and cetuximab;
Cisplatin or carboplatin and docetaxel or paclitaxel;
Cisplatin and Cetuximab;
Cisplatin and decetaxel and cetuximab;
Cisplatin and paclitaxel cetuximab;
Carboplatin and cetuximab;
Cisplatin and gemcitabine;
Gemcitabine and vinorelbine.

The subject receiving the isRNA, the at least one coding RNA, the combination thereof or the pharmaceutical composition or vaccine comprising said RNA(s) preferably suffers from squamous cell cancer of the skin (SCC), preferably unresectable and/or advanced SCC, and received or received, preferably via intratumoral administration, at least one of the following treatments:
5-FU;
Cisplatin;
Cisplatin and 5-FU;
Interferon alpha;
Cis-retinoic acid;
Interferon alpha and Cis-retinoic acid and Cisplatin;
Cetuximab;
vismodegib;
Cisplatin and 5-FU and Cetuximab;
Imiquimod;
Photodynamic therapy (amino levulinic acid, porfimer sodium);
Vigorous crytotherapy;
Electrodesiccation;
Diclofenac;
Chemical peel (trichloroacetic acid);
Cryotherapy and 5-FU and Imiquimod;
Retinoids (acitretin, isotretinoin);
Calcineurin inhibitors and/or mTOR inhibitors (rapamycin, temsirolimus, Sirolimus, everolimus, ridaforolimus, Deforolimus);
cisplatin and/or carboplatin and/or 5-FU and/or Paclitaxel and/or Decetaxel.

The subject receiving the isRNA, the at least one coding RNA, the combination thereof or the pharmaceutical composition or vaccine comprising said RNA(s) preferably suffers from adenocystic carcinoma (ACC), preferably advanced ACC, and received or received, preferably via intratumoral administration, at least one of the following treatments: treatments using as single agents or in combination
radiotherapy;
Cisplatin;
Paclitaxel;
Mitoxantron;
Doxo-/Epirubicin;
Metothrexate;
Vinorelbine;
External-beam radiation therapy.

The subject receiving the isRNA, the at least one coding RNA, the combination thereof or the pharmaceutical composition or vaccine comprising said RNA(s) preferably suffers from cutaneous T-cell lymphoma, preferably advanced cutaneous T-cell lymphoma or mycosis fungoides subtype T cell lymphoma, and received or received, preferably via intratumoral administration, at least one of the following treatments, treatments using as single agents or in combination
Corticosteroids;
Carmustine;
Nitrogen mustard (mechlorethamine hydrochloride);
Bexarotene;
Taxarotene gel;
Imiquimod;
phototherapy;
electron beam therapy (TSEBT);
Alemtuzumab;
Retinoids;
Interferon;
Vorinostat;
Romidepsin;
extracorporeak photopheresis (ECP);
Methotrexate;
liposomal doxorubicin;
Gemcitabine;
Pentostatin;
Temozolomide;
Pralatrexate;
allogenic cell transplant.

The subject receiving the isRNA, the at least one coding RNA, the combination thereof or the pharmaceutical composition or vaccine comprising said RNA(s) preferably suffers from vulvar cancer, preferably vulvar squamous cell cancer (VSCC), more preferably advanced VSCC, even more preferably VSCC refractory to surgery or chemotherapy, most preferably advanced VSCC refractory to surgery or chemotherapy, and received or received, preferably via intratumoral administration, at least one of the following treatments, treatments using as single agents or in combination:
mitomycin-C2;
cisplatin;
carboplatin;
vinorelbine;
paclitaxel;
a tyrosine kinase inhibitor (e.g. erlotinib);
nivolumab;
bleomycin sulfate (e.g. bleomycin, bleomycin sulfate, blenamax, tevableo, oncobleo, bleo, bloicin-S);
5-fluorouracil (5-FU);
Gardasil 9 (human papillomavirus (9-valent) vaccine);
omiganan pentahydrochloride;
alisertib;
ISA-101 (13 synthetic long peptides (25-35 amino acids long) derived from the E6 and E7 oncogenic proteins of the HPV 16 virus);
PDS-0101;
Vicoryx (P16_37-63 vaccine);
TA-CIN (fusion protein vaccine comprising capsid protein L2, E6 and E7 from HPV16); and
human papillomavirus 16 E6 peptide vaccine.

### BRIEF DESCRIPTION OF THE FIGURES

The examples and figures shown in the following are merely illustrative and shall describe the present invention in a further way. These figures and examples shall not be construed to limit the present invention thereto.

FIG. 1: Panel (A) shows an analysis of the median tumor growth of Balb/C mice bearing CT26 tumors after intratumoral treatment with RNAdjuvant, mRNA encoding IL-12 and mRNA-encoded soluble PD-1.

Respective combinations of these compounds, including control groups, were tested as indicated in the figure. The experiment was performed as described in Example 1.

Panel (B) shows survival proportions of mice bearing CT26 tumors after intratumoral treatment with RNAdjuvant, mRNA encoding IL-12 and mRNA encoded soluble PD-1. Respective combinations of these compounds, including control groups, were tested as indicated in the figure. The experiment was performed as described in Example 1. Kaplan-Meier survival curves are presented.

FIG. 2: shows survival proportions of Balb/C mice bearing CT26 tumors after intratumoral treatment with RNAdjuvant and intraperitoneal treatment of an anti-PD-1 antibody. Respective combinations of these compounds, including control groups, were tested as indicated in the figure. The experiment was performed as described in Example 2. Kaplan-Meier survival curves are presented.

FIG. 3: shows survival proportions of Balb/C mice bearing CT26 tumors after intratumoral treatment with RNAdjuvant, mRNA encoding IL-12 and mRNA-encoded CD40L compared to intratumoral treatment with mRNA encoding IL-12 alone. Respective combinations of these compounds, including control groups, were tested as indicated in the figure. The experiment was performed as described in Example 3. Kaplan-Meier survival curves are presented.

FIG. 4: shows an analysis of the median tumor growth after re-challenge of Balb/C mice with syngeneic CT26 colon carcinoma cells at day 113 after the first tumor challenge. Mice were previously treated intratumorally with RNAdjuvant alone or in combination with anti-PD1 treatment. Respective combinations of these compounds, including control groups, were tested as indicated in the figure. The experiment was performed as described in Example 4.

FIG. 5: shows an analysis of the median tumor growth after re-challenge of Balb/C mice with syngeneic CT26 colon carcinoma cells at day 113 after the first tumor challenge. Mice were previously treated intratumorally with RNAdjuvant alone or in combination with an mRNA encoding CD40L and an mRNA-encoded IL-12. Respective combinations of these compounds, including control groups, were tested as indicated in the figure. The experiment was performed as described in Example 5.

FIG. 6: shows an analysis of the median tumor growth of Balb/C mice bearing CT26 tumors after intratumoral treatment with immunostimulating RNA (RNAdjuvant), mRNA encoding soluble PD1 (solPD1) and CD40 ligand (CD40L) in combination with a checkpoint inhibitor anti CTLA4 antibody. The experiment was performed as described in Example 7.

FIG. 7: shows an analysis of the median tumor growth of the untreated lesion of Balb/C mice bearing CT26 tumors in both flanks after intratumoral treatment of one lesion with immunostimulating RNA (RNAdjuvant), mRNA encoding soluble PD1 (solPD1) and CD40 ligand (CD40L) in combination with an anti-CTLA4 checkpoint antibody. The experiment was performed as described in Example 8.

FIG. 8: Panel (A) shows an analysis of the median tumor growth of Balb/C mice bearing E.G7-OVA tumors after intratumoral treatment with immunostimulatory RNAdjuvant and vaccinated i.d. with OVA (RNActive) or in combination with an anti-PD1 checkpoint inhibitor (administered i.p.) and PpLuc RNActive or buffer as unspecific control. The experiment was performed as described in Example 10. Panel (B) shows survival proportions of mice bearing E.G7-OVA tumors after intratumoral treatment with immunostimulatory RNAdjuvant and vaccinated i.d. with OVA (RNActive) or in combination with a checkpoint inhibitor anti PD1 (administered i.p.) and PpLuc RNActive or buffer as unspecific control. The experiment was performed as described in Example 10. Kaplan-Meier survival curves are presented.

FIG. 9: Panel (A) shows an analysis of the median tumor growth of Balb/C mice bearing E.G7-OVA tumors after intratumoral treatment with immunostimulatory RNAdjuvant and an mRNA encoding IL12 vaccinated i.d. with OVA (RNActive) and PpLuc RNActive or buffer as unspecific control. The experiment was performed as described in Example 11.

Panel (B) shows survival proportions of mice bearing E.G7-OVA tumors after intratumoral treatment with immunostimulatory RNAdjuvant and an mRNA encoding IL12 vaccinated i.d. with OVA (RNActive) and PpLuc RNActive or buffer as unspecific control. The experiment was performed as described in Example 11. Kaplan-Meier survival curves are presented.

FIG. 10: Panel (A) shows translated mRNA products of IL12 in the supernantant of RNA transfected A375 cells after 5 hours. The experiment was performed as described in Example 13.

Panel (B) shows translated mRNA products of IL12 in the supernantant of RNA transfected A375 cells after 24 hours. The experiment was performed as described in Example 13.

FIG. 11: Panel (A) shows translated mRNA products of solPD1 in the supernantant of RNA transfected A375 cells after 5 hours. The experiment was performed as described in Example 13.

Panel (B) shows translated mRNA products of solPD1 in the supernantant of RNA transfected A375 cells after 24 hours. The experiment was performed as described in Example 13.

FIG. 12: Panel (A) shows translated mRNA products of anti-CTLA4 antibody in the supernantant of RNA transfected A375 cells after 5 hours. The experiment was performed as described in Example 13.

Panel (B) shows translated mRNA products of anti-CTLA4 antibody in the supernantant of RNA transfected A375 cells after 24 hours. The experiment was performed as described in Example 13.

FIG. 13: shows membrane bound translated mRNA product of CD40LG on transfected A375 cells after 24 hours analyzed by FACS analysis. The experiment was performed as described in Example 13.

### DETAILED DESCRIPTION OF THE INVENTION

The present invention and preferred embodiments thereof are further described by the following items:
1. Immunostimulatory RNA (isRNA) for use in the treatment or prophylaxis of a tumor or cancer disease preferably selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy.
2. The isRNA for use according to item 1, wherein the isRNA is administered intratumorally, including peritumorally or locoregionally.
3. The isRNA for use according to item 2, wherein the isRNA is administered by injection.
4. The isRNA for use according to any of the preceding items, wherein the isRNA is a non-coding RNA.
5. The isRNA for use according to any of the preceding items, wherein the isRNA comprises
   a nucleic acid sequence according to

(GₗXₘGₙ), formula (I)
wherein:
G is guanosine (guanine), uridine (uracil) or an analogue of guanosine (guanine) or uridine (uracil);
X is guanosine (guanine), (uridine) uracil, adenosine (adenine), thymidine (thymine), cytidine (cytosine) or an analogue of the above-mentioned nucleotides (nucleosides);
l is an integer from 1 to 40,
wherein
when I=1 G is guanosine (guanine) or an analogue thereof,
when I>1 at least 50% of the nucleotides (nucleosides) are guanosine (guanine) or an analogue thereof;
m is an integer and is at least 3;
wherein
when m=3 X is uridine (uracil) or an analogue thereof,
when m>3 at least 3 successive uridines (uracils) or analogues of uridine (uracil) occur;
n is an integer from 1 to 40,
wherein
when n=1 G is guanosine (guanine) or an analogue thereof,
when n>1 at least 50% of the nucleotides (nucleosides) are guanosine (guanine) or an analogue thereof;
a nucleic acid sequence according to
formula (II) (CₗXₘCₙ)
wherein:
C is cytidine (cytosine), uridine (uracil) or an analogue of cytidine (cytosine) or uridine (uracil);
X is guanosine (guanine), uridine (uracil), adenosine (adenine), thymidine (thymine), cytidine (cytosine) or an analogue of the above-mentioned nucleotides (nucleosides);
l is an integer from 1 to 40,
wherein
when I=1 C is cytidine (cytosine) or an analogue thereof,
when I>1 at least 50% of the nucleotides (nucleosides) are cytidine (cytosine) or an analogue thereof;
m is an integer and is at least 3;
wherein
when m=3 X is uridine (uracil) or an analogue thereof,
when m>3 at least 3 successive uridines (uracils) or analogues of uridine (uracil) occur;
n is an integer from 1 to 40,
wherein
when n=1 C is cytidine (cytosine) or an analogue thereof,
when n>1 at least 50% of the nucleotides (nucleosides) are cytidine (cytosine) or an analogue thereof;
a nucleic acid sequence according to

(NᵤGₗXₘGₙNᵥ)ₐ formula (III)
wherein:
G is guanosine (guanine), uridine (uracil) or an analogue of guanosine (guanine) or uridine (uracil), preferably guanosine (guanine) or an analogue thereof;
X is guanosine (guanine), uridine (uracil), adenosine (adenine), thymidine (thymine), cytidine (cytosine), or an analogue of these nucleotides (nucleosides), preferably uridine (uracil) or an analogue thereof;
N is a nucleic acid sequence having a length of about 4 to 50, preferably of about 4 to 40, more preferably of about 4 to 30 or 4 to 20 nucleic acids, each N independently being selected from guanosine (guanine), uridine (uracil), adenosine (adenine), thymidine (thymine), cytidine (cytosine) or an analogue of these nucleotides (nucleosides);
a is an integer from 1 to 20, preferably from 1 to 15, most preferably from 1 to 10;
l is an integer from 1 to 40,
wherein when I=1, G is guanosine (guanine) or an analogue thereof,
when I>1, at least 50% of these nucleotides (nucleosides) are guanosine (guanine) or an analogue thereof;
m is an integer and is at least 3;
wherein when m=3, X is uridine (uracil) or an analogue thereof, and
when m>3, at least 3 successive uridines (uracils) or analogues of uridine (uracil) occur;
n is an integer from 1 to 40,
wherein when n=1, G is guanosine (guanine) or an analogue thereof,
when n>1, at least 50% of these nucleotides (nucleosides) are guanosine (guanine) or an analogue thereof;
u,v may be independently from each other an integer from 0 to 50,
preferably wherein when u=0, v≥1, or when v=0, u≥1;
wherein the nucleic acid molecule of formula (III) has a length of at least 50 nucleotides, preferably of at least 100 nucleotides, more preferably of at least 150 nucleotides, even more preferably of at least 200 nucleotides and most preferably of at least 250 nucleotides; and/or
a nucleic acid sequence according to

(NᵤCₗXₘCₙNᵥ)ₐ formula (IV)
wherein:
C is cytidine (cytosine), uridine (uracil) or an analogue of cytidine (cytosine) or uridine (uracil), preferably cytidine (cytosine) or an analogue thereof;
X is guanosine (guanine), uridine (uracil), adenosine (adenine), thymidine (thymine), cytidine (cytosine) or an analogue of the above-mentioned nucleotides (nucleosides), preferably uridine (uracil) or an analogue thereof;
N is each a nucleic acid sequence having independent from each other a length of about 4 to 50, preferably of about 4 to 40, more preferably of about 4 to 30 or 4 to 20 nucleic acids, each N independently being selected from guanosine (guanine), uridine (uracil), adenosine (adenine), thymidine (thymine), cytidine (cytosine) or an analogue of these nucleotides (nucleosides);
a is an integer from 1 to 20, preferably from 1 to 15, most preferably from 1 to 10;
l is an integer from 1 to 40,
wherein when I=1, C is cytidine (cytosine) or an analogue thereof,
when I>1, at least 50% of these nucleotides (nucleosides) are cytidine (cytosine) or an analogue thereof;
m is an integer and is at least 3;
wherein when m=3, X is uridine (uracil) or an analogue thereof,
when m>3, at least 3 successive uridines (uracils) or analogues of uridine (uracil) occur;
n is an integer from 1 to 40,
wherein when n=1, C is cytidine (cytosine) or an analogue thereof,
when n>1, at least 50% of these nucleotides (nucleosides) are cytidine (cytosine) or an analogue thereof.
u, v may be independently from each other an integer from 0 to 50,
preferably wherein when u=0, v≥1, or when v=0, u≥1;
wherein the nucleic acid molecule of formula (V) according to the invention has a length of at least 50 nucleotides, preferably of at least 100 nucleotides, more preferably of at least 150 nucleotides, even more preferably of at least 200 nucleotides and most preferably of at least 250 nucleotides.
6. The isRNA for use according to any of the preceding items, wherein the isRNA comprises at least one nucleic acid sequence according to any one of SEQ ID NOs: 433 to 437 or 1014 to 1016, preferably according to any one of SEQ ID NO: 433, 434 or 1014 to 1016, or a fragment or variant of any one of these nucleic acid sequences.
7. The isRNA for use according to any of the preceding items, wherein the isRNA is complexed with a cationic or polycationic compound, preferably with a cationic or polycationic polymer, a cationic or polycationic peptide or protein, e.g. protamine, a cationic or polycationic polysaccharide and/or a cationic or polycationic lipid.
8. The isRNA for use according to item 7, wherein the cationic or polycationic compound is a polymeric carrier.
9. The isRNA for use according to item 8, wherein the polymeric carrier is formed by a disulfide-crosslinked cationic component, preferably a disulfide-crosslinked cationic peptide, wherein the disulfide-crosslinked cationic peptide preferably comprises
   a peptide according to formula V

(Arg)ₗ;(Lys)ₘ;(His)ₙ;(Orn)ₒ;(Xaa)ₓ, (formula (V),
wherein l+m+n+o+x=8-15, and l, m, n or o independently of each other may be any number selected from 0, 1, 2, 3, 4, 5, 6, 7, 8, 9, 10, 11, 12, 13, 14 or 15, provided that the overall content of Arg, Lys, His and Orn represents at least 50% of all amino acids of the oligopeptide; and Xaa may be any amino acid selected from native (=naturally occurring) or non-native amino acids except of Arg, Lys, His or Orn; and x may be any number selected from 0, 1, 2, 3 or 4, provided, that the overall content of Xaa does not exceed 50% of all amino acids of the oligopeptide;
a peptide according to formula Va

{(Arg)ₗ;(Lys)ₘ;(His)ₙ;(Orn)ₒ;(Xaa′)ₓ(Cys)_{y}} formula (Va),
wherein (Arg)ₗ;(Lys)ₘ;(His)ₙ;(Orn)ₒ; and x are as defined for formula V, Xaa′ is any amino acid selected from native (=naturally occurring) or non-native amino acids except of Arg, Lys, His, Orn or Cys and y is any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80 and 81-90, provided that the overall content of Arg (Arginine), Lys (Lysine), His (Histidine) and Orn (Ornithine) represents at least 10% of all amino acids of the oligopeptide;
a peptide according to formula Vb

Cys1{(Arg)ₗ;(Lys)ₘ;(His)ₙ;(Orn)ₒ;(Xaa)ₓ}Cys2 formula (Vb)
wherein empirical formula {(Arg)ₗ;(Lys)ₘ;(His)ₙ;(Orn)ₒ;(Xaa)ₓ} is as defined for formula (V) and forms a core of an amino acid sequence according to (semiempirical) formula (V) and wherein Cys1 and Cys2 are cysteines proximal to, or terminal to (Arg)ₗ;(Lys)ₘ;(His)ₙ;(Orn)ₒ;(Xaa)ₓ; and/or
a compound according to formula VI

L-P¹—S—[S—P²—S]ₙ—S—P³-L formula (VI)
wherein,
   P¹ and P³ are different or identical to each other and represent a linear or branched hydrophilic polymer chain, each P¹ and P³ exhibiting at least one —SH-moiety, capable to form a disulfide linkage upon condensation with component P², or alternatively with (AA), (AA)ₓ, or [(AA)ₓ]_{z} if such components are used as a linker between P¹ and P² or P³ and P² and/or with further components (e.g. (AA), (AA)ₓ, [(AA)ₓ]_{z} or L), the linear or branched hydrophilic polymer chain selected independent from each other from polyethylene glycol (PEG), poly-N-(2-hydroxypropyl)methacrylamide, poly-2-(methacryloyloxy)ethyl phosphorylcholines, poly(hydroxyalkyl L-asparagine), poly(2-(methacryloyloxy)ethyl phosphorylcholine), hydroxyethylstarch or poly(hydroxyalkyl L-glutamine), wherein the hydrophilic polymer chain exhibits a molecular weight of about 1 kDa to about 100 kDa, preferably of about 2 kDa to about 25 kDa; or more preferably of about 2 kDa to about 10 kDa, e.g. about 5 kDa to about 25 kDa or 5 kDa to about 10 kDa;
   P2 is a cationic or polycationic peptide or protein, e.g. as defined above for the polymeric carrier formed by disulfide-crosslinked cationic components, and preferably having a length of about 3 to about 100 amino acids, more preferably having a length of about 3 to about 50 amino acids, even more preferably having a length of about 3 to about 25 amino acids, e.g. a length of about 3 to 10, 5 to 15, 10 to 20 or 15 to 25 amino acids, more preferably a length of about 5 to about 20 and even more preferably a length of about 10 to about 20;
      or
      is a cationic or polycationic polymer, e.g. as defined above for the polymeric carrier formed by disulfide-crosslinked cationic components, typically having a molecular weight of about 0.5 kDa to about 30 kDa, including a molecular weight of about 1 kDa to about 20 kDa, even more preferably of about 1.5 kDa to about 10 kDa, or having a molecular weight of about 0.5 kDa to about 100 kDa, including a molecular weight of about 10 kDa to about 50 kDa, even more preferably of about 10 kDa to about 30 kDa;
      each P² exhibiting at least two —SH-moieties, capable to form a disulfide linkage upon condensation with further components P² or component(s) P′ and/or P³ or alternatively with further components (e.g. (AA), (AA)ₓ, or [(AA)ₓ]_{z});
   —S—S— is a (reversible) disulfide bond (the brackets are omitted for better readability), wherein S preferably represents sulphur or a —SH carrying moiety, which has formed a (reversible) disulfide bond. The (reversible) disulfide bond is preferably formed by condensation of —SH-moieties of either components P¹ and P², P² and P², or P² and P³, or optionally of further components as defined herein (e.g. L, (AA), (AA)ₓ, [(AA)ₓ]_{z}, etc); The —SH-moiety may be part of the structure of these components or added by a modification as defined below;
   L is an optional ligand, which may be present or not, and may be selected independent from the other from RGD, Transferrin, Folate, a signal peptide or signal sequence, a localization signal or sequence, a nuclear localization signal or sequence (NLS), an antibody, a cell penetrating peptide, (e.g. TAT or KALA (SEQ ID NO: 1063)), a ligand of a receptor (e.g. cytokines, hormones, growth factors etc), small molecules (e.g. carbohydrates like mannose or galactose or synthetic ligands), small molecule agonists, inhibitors or antagonists of receptors (e.g. RGD peptidomimetic analogues), or any further protein as defined herein, etc.;
   n is an integer, typically selected from a range of about 1 to 50, preferably from a range of about 1, 2 or 3 to 30, more preferably from a range of about 1, 2, 3, 4, or 5 to 25, or a range of about 1, 2, 3, 4, or 5 to 20, or a range of about 1, 2, 3, 4, or 5 to 15, or a range of about 1, 2, 3, 4, or 5 to 10, including e.g. a range of about 4 to 9, 4 to 10, 3 to 20, 4 to 20, 5 to 20, or 10 to 20, or a range of about 3 to 15, 4 to 15, 5 to 15, or 10 to 15, or a range of about 6 to 11 or 7 to 10. Most preferably, n is in a range of about 1, 2, 3, 4, or 5 to 10, more preferably in a range of about 1, 2, 3, or 4 to 9, in a range of about 1, 2, 3, or 4 to 8, or in a range of about 1, 2, or 3 to 7.
10. The isRNA for use according to item 8 or 9, wherein the polymeric carrier comprises at least one of the disulfide-crosslinked cationic peptides Cys-Arg₁₂ (SEQ ID NO: 580) or Cys-Arg₁₂-Cys (SEQ ID NO: 579).
11. The isRNA for use according to any of items 7 to 10, wherein the N/P ratio of the isRNA to the cationic or polycationic compound, preferably the cationic or polycationic peptide or protein, is in the range of about 0.1 to 10, including a range of about 0.3 to 4, of about 0.5 to 2, of about 0.7 to 2 and of about 0.7 to 1.5.
12. The isRNA for use according to any of the preceding items, wherein the isRNA is complexed with one or more lipids, thereby forming liposomes, lipid nanoparticles and/or lipoplexes.
13. The isRNA for use according to any of the preceding items, wherein the treatment comprises administration of at least one additional pharmaceutically active ingredient.
14. The isRNA for use according to item 13, wherein the at least one additional pharmaceutically active ingredient is a compound that is used in the treatment of a tumor or cancer disease preferably selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy.
15. The isRNA for use according to item 13 or 14, wherein the at least one additional pharmaceutically active ingredient is a checkpoint modulator, or a fragment or variant thereof.
16. The isRNA for use according to item 15, wherein the checkpoint modulator is selected from the group consisting of a PD-1 inhibitor, a PD-L1 inhibitor, a PD-L2 inhibitor a CTLA-4 inhibitor, a LAG3 inhibitor, a TIM3 inhibitor, a TIGIT inhibitor an OX40 stimulator, a 4-1BB stimulator, a CD40L stimulator, a CD28 stimulator and a GITR stimulator, or a fragment or variant of any one of these checkpoint modulators.
17. The isRNA for use according to item 16, wherein the checkpoint modulator is a PD-1 inhibitor or a PD-L1 inhibitor, wherein the PD-1 inhibitor is preferably an antagonistic antibody directed against PD-1 and the PD-L1 inhibitor is an antagonistic antibody directed against PD-L1, or a fragment or variant of said antibody.
18. The isRNA for use according to item 16, wherein the checkpoint modulator is a CTLA-4 inhibitor, preferably an anti-CTLA4 antibody, or a fragment or variant thereof.
19. The isRNA for use according to item 13 or 14, wherein the at least one additional pharmaceutically active ingredient is an interleukin, preferably IL-12, or a fragment or variant thereof.
20. The isRNA for use according to any of items 1 to 19, wherein the treatment comprises administration of at least one coding RNA, preferably at least one mRNA.
21. The isRNA for use according to item 20, wherein the at least one coding RNA comprises at least one coding sequence encoding at least one peptide or protein comprising at least one peptide or protein selected from the group consisting of
   IL-12,
   CD40L,
   a decoy PD-1 receptor, and
   an anti-CTLA4 antibody,
or a fragment or variant of any of these.
22. The isRNA for use according to item 20 or 21, wherein the at least one coding RNA comprises at least one coding sequence encoding a peptide or protein comprising IL-12 or a fragment or variant thereof, or an IL-12 analog, or a fragment or variant thereof.
23. The isRNA for use according to item 20 or 21, wherein the at least one coding RNA comprises at least one coding sequence encoding a peptide or protein comprising CD40L or a fragment or variant thereof.
24. The isRNA for use according to item 20 or 21, wherein the at least one coding RNA comprises at least one coding sequence encoding a peptide or protein comprising a decoy PD-1 receptor or a fragment or variant thereof.
25. The isRNA for use according to item 20 or 21, wherein the at least one coding RNA comprises at least one coding sequence encoding a peptide or protein comprising an anti-CTLA4 antibody, or a fragment or variant thereof.
26. The isRNA for use according to item 20 or 21, wherein
   the at least one coding RNA comprises at least one coding sequence encoding a peptide or protein comprising CD40L or a fragment or variant thereof and
   the same or a different coding RNA comprises at least one coding sequence encoding a peptide or protein comprising IL-12 or a fragment or variant thereof.
27. The isRNA for use according to item 20 or 21, wherein
   the at least one coding RNA comprises at least one coding sequence encoding a peptide or protein comprising CD40L or a fragment or variant thereof, and
   the same or a different coding RNA comprises at least one coding sequence encoding a peptide or protein comprising a decoy PD-1 receptor or a fragment or variant thereof.
28. The isRNA for use according to item 20 or 21, wherein
   the at least one coding RNA comprises at least one coding sequence encoding a peptide or protein comprising IL-12 or a fragment or variant thereof, and
   the same or a different coding RNA comprises at least one coding sequence encoding a peptide or protein comprising a decoy PD-1 receptor or a fragment or variant thereof.
28. The isRNA for use according to item 20 or 21, wherein
   the at least one coding RNA comprises at least one coding sequence encoding a peptide or protein comprising IL-12 or a fragment or variant thereof, and
   the same or a different coding RNA comprises at least one coding sequence encoding a peptide or protein comprising at least one tumor antigen or a fragment or variant thereof.
29. The isRNA for use according to item 20 or 21, wherein
   the at least one coding RNA comprises at least one coding sequence encoding a peptide or protein comprising CD40L or a fragment or variant thereof, and
   the same or a different coding RNA comprises at least one coding sequence encoding a peptide or protein comprising an anti-CTLA4 antibody, or a fragment or variant thereof.
30. The isRNA for use according to item 20 or 21, wherein
   the at least one coding RNA comprises at least one coding sequence encoding a peptide or protein comprising IL-12 or a fragment or variant thereof, and
   the same or a different coding RNA comprises at least one coding sequence encoding a peptide or protein comprising an anti-CTLA4 antibody, or a fragment or variant thereof.
31. The isRNA for use according to item 20 or 21, wherein
   the at least one coding RNA comprises at least one coding sequence encoding a peptide or protein comprising a decoy PD-1 receptor or a fragment or variant thereof, and
   the same or a different coding RNA comprises at least one coding sequence encoding a peptide or protein comprising an anti-CTLA4 antibody, or a fragment or variant thereof.
32. The isRNA for use according to item 20 or 21, wherein
   the at least one coding RNA comprises at least one coding sequence encoding a peptide or protein comprising IL-12 or a fragment or variant thereof,
   the same or a different coding RNA comprises at least one coding sequence encoding a peptide or protein comprising CD40L or a fragment or variant thereof, and
   the same or a different coding RNA comprises at least one coding sequence encoding a peptide or protein comprising an anti-CTLA4 antibody, or a fragment or variant thereof.
33. The isRNA for use according to item 20 or 21, wherein
   the at least one coding RNA comprises at least one coding sequence encoding a peptide or protein comprising IL-12 or a fragment or variant thereof,
   the same or a different coding RNA comprises at least one coding sequence encoding a peptide or protein comprising CD40L or a fragment or variant thereof,
   the same or a different coding RNA comprises at least one coding sequence encoding a peptide or protein comprising an anti-CTLA4 antibody, or a fragment or variant thereof, and
   optionally, the same or a different coding RNA comprises at least one coding sequence encoding a peptide or protein comprising a decoy PD-1 receptor or a fragment or variant thereof.
34. The isRNA for use according to any of items 1 to 33, wherein the subject does not receive or has not received a treatment with a PD-1 or PD-L1 antagonist and wherein the use comprises administration of at least one peptide or protein comprising a decoy PD-1 receptor or a fragment or variant thereof, or administration of a nucleic acid, preferably a coding RNA, more preferably an mRNA, comprising a nucleic acid sequence encoding at least one peptide or protein comprising a decoy PD-1 receptor or a fragment or variant thereof.
35. The isRNA for use according to any of items 1 to 34, wherein the subject receives or has received a treatment with a PD-1 or a PD-L1 antagonist and wherein the use does not comprise administration of at least one peptide or protein comprising a decoy PD-1 receptor or a fragment or variant thereof, or administration of a nucleic acid, preferably a coding RNA, more preferably an mRNA, comprising a nucleic acid sequence encoding at least one peptide or protein comprising a decoy PD-1 receptor or a fragment or variant thereof.
36. The isRNA for use according to item 33, wherein each coding sequence encoding a peptide or protein is located on a separate coding RNA, preferably a separate mRNA.
37. The isRNA for use according to item 33, wherein at least two of the coding sequences encoding a peptide or protein are located on the same coding RNA, which is preferably a bi- or multicistronic RNA.
38. The isRNA for use according to any of items 20 to 37, wherein the at least one coding RNA is administered intratumorally.
39. The isRNA for use according to any of items 20 to 38, wherein the at least one coding RNA is administered intradermally, intramuscularly or subcutaneously.
40. The isRNA for use according to item 36, wherein the separate coding RNAs are formulated together and administered intratumorally.
41. The isRNA for use according to any of items 20 to 40, wherein the isRNA is formulated together with the at least one coding RNA.
42. The isRNA for use according to item 37, wherein the co-formulation is administered intratumorally.
43. The isRNA for use according to any of items 20 to 42, wherein the at least one coding RNA comprises at least one coding sequence encoding a peptide or protein comprising IL-12, preferably at least one of IL-12A or IL-12B, or a fragment or variant of any of these proteins.
44. The isRNA for use according to item 43, wherein the encoded peptide or protein comprises an amino acid sequence selected from the group consisting of SEQ ID NO: 3 to 8, or a fragment or variant of any of these sequences.
45. The isRNA for use according to item 43 or 44, wherein the at least one coding sequence comprises a nucleic acid sequence selected from the group consisting of SEQ ID NOs: 440 to 445 or a fragment or variant of any of these sequences.
46. The isRNA for use according to any of items 43 to 45, wherein the encoded peptide or protein comprises IL-12A and IL-12B or a fragment or variant of each of these proteins.
47. The isRNA for use according to item 46, wherein the encoded peptide or protein comprises an amino acid sequence according to SEQ ID NO: 10 or a fragment or variant thereof.
48. The isRNA for use according to any of items 43 to 47, wherein the at least one coding sequence comprises a nucleic acid sequence according to SEQ ID NO: 447 or a fragment or variant thereof.
49. The isRNA for use according to any of items 20 to 48, wherein the at least one coding RNA comprises at least one coding sequence encoding a peptide or protein comprising CD40L or a fragment or variant thereof, wherein the encoded peptide or protein preferably comprises an amino acid sequence according to SEQ ID NO: 11 or a fragment or variant thereof.
50. The isRNA for use according to item 49, wherein the at least one coding sequence comprises a nucleic acid sequence according to SEQ ID NO: 448 or a fragment or variant thereof.
51. The isRNA for use according to any of items 20 to 50, wherein the at least one coding RNA comprises at least one coding sequence encoding a peptide or protein comprising a decoy PD-1 receptor or a fragment or variant thereof, preferably the extracellular part of a PD-1 receptor or a fragment or variant thereof.
52. The isRNA for use according to item 51, wherein the decoy PD-1 receptor is a peptide or protein comprising soluble PD-1 or a fragment or variant thereof.
53. The isRNA for use according to item 51 or 52, wherein the encoded peptide or protein comprises an amino acid sequence according to SEQ ID NO: 2 or 1042, or a fragment or variant thereof.
54. The isRNA for use according to any of items 51 to 53, wherein the at least one coding sequence comprises a nucleic acid sequence according to SEQ ID NO: 439 or a fragment or variant thereof.
55. The isRNA for use according to any of items 20 to 54, wherein the at least one coding RNA comprises at least one coding sequence encoding a peptide or protein comprising an anti-CTLA4 antibody or a fragment or variant thereof.
56. The isRNA for use according to item 55, wherein the encoded peptide or protein comprises an amino acid sequence according to SEQ ID NO: 645 and/or 677, or a fragment or variant of any of these amino acid sequences.
57. The isRNA for use according to item 55 or 56, wherein the at least one coding sequence comprises a nucleic acid sequence according to SEQ ID NO: 646 and/or 678, or a fragment or variant of any of these nucleic acid sequences.
58. The isRNA for use according to items 20 to 57, wherein the at least one coding RNA comprises at least one coding sequence encoding a peptide or protein comprising at least one tumor antigen, or a fragment or variant of a tumor antigen.
59. The isRNA for use according to item 58, wherein the tumor antigen is preferably selected from the group consisting of 1A01_HLA-A/m; 1A02; 5T4; ACRBP; AFP; AKAP4; alpha-actinin-_4/m; alpha-methylacyl-coenzyme_A_racemase; ANDR; ART-4; ARTC1/m; AURKB; B2MG; B3GN5; B4GN1; B7H4; BAGE-1; BASI; BCL-2; bcr/abl; beta-catenin/m; BING-4; BIRC7; BRCA1/m; BY55; calreticulin; CAMEL; CASP-8/m; CASPA; cathepsin_B; cathepsin_L; CD1A; CD1B; CD1C; CD1D; CD1E; CD20; CD22; CD276; CD33; CD3E; CD3Z; CD44_Isoform_1; CD44_Isoform_6; CD4; CD52; CD55; CD56; CD80; CD86; CD8A; CDC27/m; CDE30; CDK4/m; CDKN2A/m; CEA; CEAM6; CH3L2; CLCA2; CML28; CML66; COA-1/m; coactosin-like_protein; collagen_MII; COX-2; CP1B1; CSAG2; CT45A1; CT55; CT-_9/BRD6; CTAG2_Isoform_LAGE-1A; CTAG2_Isoform_LAGE-1B; CTCFL; Cten; cyclin_B1; cyclin_D1; cyp-B; DAM-10; DEP1A; E7; EF1A2; EFTUD2/m; EGFR; EGLN3; ELF2/m; EMMPRIN; EpCam; EphA2; EphA3; ErbB3; ERBB4; ERG; ETV6; EWS; EZH2; FABP7; FCGR3A_Version_1; FCGR3A_Version_2; FGF5; FGFR2; fibronectin; FOS; FOXP3; FUT1; G250; GAGE-1; GAGE-2; GAGE-3; GAGE-4; GAGE-5; GAGE-6; GAGE7b; GAGE-8_(GAGE-2D); GASR; GnT-V; GPC3; GPNMB/m; GRM3; HAGE; hepsin; Her2/neu; HLA-A2/m; homeobox_NKX3.1; HOM-TES-85; HPG1; HS71A; HS71B; HST-2; hTERT; iCE; IF2B3; IL10; IL-13Ra2; IL2-RA; IL2-RB; IL2-RG; IL-5; IMP3; ITA5; ITB1; ITB6; kallikrein-2; kallikrein-3; kallikrein-4; KI20A; KIAA0205; KIF2C; KK-LC-1; LDLR; LGMN; LIRB2; LY6K; MAGA5; MAGA8; MAGAB; MAGE-A10; MAGE-Al2; MAGE-A1; MAGE-A2; MAGE-A3; MAGE-A4; MAGE-A6; MAGE-A9; MAGE-B10; MAGE-B16; MAGE-B17; MAGE-_B1; MAGE-B2; MAGE-B3; MAGE-B4; MAGE-B5; MAGE-B6; MAGE-C1; MAGE-C2; MAGE-C3; MAGE-D1; MAGE-D2; MAGE-D4; MAGE-_E1; MAGE-E1_(MAGE1); MAGE-E2; MAGE-F1; MAGE-H₁; MAGEL2; mammaglobin_A; MART-1/melan-A; MART-2; MC1_R; M-CSF; mesothelin; MITF; MMP1_1; MMP7; MUC-1; MUM-1/m; MUM-2/m; MYCN; MYO1A; MYO1B; MYO1C; MYO1Da MYO1E; MYO1F; MYO1G; MYO1H; NA17; NA88-A; Neo-PAP; NFYC/m; NGEP; NPM; NRCAM; NSE; NUF2; NY-ESO-1; OA1; OGT; OS-9; osteocalcin; osteopontin; p53; PAGE-4; PAI-1; PAI-2; PAP; PATE; PAX3; PAXS; PD1L1; PDCD1; PDEF; PECA1; PGCB; PGFRB; Pim-1_-Kinase; Pin-1; PLAC1; PMEL; PML; POTEF; POTE; PRAME; PRDX5/m; PRM2; prostein; proteinase-3; PSA; PSB9; PSCA; PSGR; PSM; PTPRC; RAB8A; RAGE-1; RARA; RASH; RASK; RASN; RGSS; RHAMM/CD168; RHOC; RSSA; RU1; RU2; RUNX1; S-100; SAGE; SART-_1; SART-2; SART-3; SEPR; SERPINBS; SIA7F; SIA8A; SIAT9; SIRT2/m; SOX10; SP17; SPNXA; SPXN3; SSX-1; SSX-2; SSX3; SSX-4; ST1A1; STAG2; STAMP-1; STEAP-1; Survivin-2B; survivin; SYCP1; SYT-SSX-1; SYT-SSX-2; TARP; TCRg; TF2AA; TGFB1; TGFR2; TGM-4; TIE2; TKTL1; TPI/m; TRGV11; TRGV9; TRPC1; TRP-p8; TSG10; TSPY1; TVC_(TRGV3); TX101; tyrosinase; TYRP1; TYRP2; UPA; VEGFR1; WT1; and XAGE1.
60. The isRNA for use according to item 58 or 59, wherein the encoded peptide or protein comprises an amino acid sequence selected from the group consisting of SEQ ID NOs: 1-504; 4558-4560 of PCT/EP2017/059525, or a fragment or variant of any of these sequences.
61. The isRNA for use according to any of items 58 to 60, wherein the at least one coding sequence of the at least one coding RNA comprises a nucleic acid sequence selected from the group consisting of SEQ ID NOs: 505-4033; 4561-4591 of PCT/EP2017/059525, or a fragment or variant of any of these sequences.
62. The isRNA for use according to any of items 58 to 61, wherein the at least one coding RNA is not administered intratumorally.
63. The isRNA for use according to any of items 58 to 62, wherein the at least one coding RNA is administered intradermally, intramuscularly or subcutaneously.
64. The isRNA for use according to any of items 20 to 63, wherein the at least one coding RNA comprises at least one coding sequence comprising a nucleic acid sequence that is modified compared to the nucleic acid sequence of the coding sequence of the corresponding wild type RNA, and wherein the amino acid sequence encoded by said coding sequence is preferably not modified compared to the amino acid sequence encoded by the coding sequence of the corresponding wild type RNA.
65. The isRNA for use according to item 64, wherein the at least one coding sequence comprises
   a) a nucleic acid sequence selected from the group consisting of SEQ ID NOs: 25-30; 36-41; 47-52; 58-63; 69-74; 80-85; 91-96; 102-107; 113-118; 124-129; 135-140; 601-606; 612-617; 623-628; 716-725; 727; 1018-1021 and 1059-1062, or a fragment or variant of any of these sequences, preferably from the group consisting of 32; 43; 54; 65; 76; 87; 98; 109; 120; 131; 142; 608; 619; 630; 632-644; 726 and 1058, or a fragment or variant of any of these sequences;
   b) a nucleic acid sequence selected from the group consisting of SEQ ID NOs: 33; 44; 55; 66; 77; 88; 99; 110; 121; 132; 143; 609; 620; 631; 728-738 and 1025-1028, or a fragment or variant of any of these sequences,
   c) a nucleic acid sequence selected from the group consisting of SEQ ID NO: 646-660; 662-676; 678-692; 694-705; 707-715 and 1029-1041, or a fragment or variant of any of these nucleic acid sequences, and/or
   d) optionally, a nucleic acid sequence selected from the group consisting of SEQ ID NOs: 23; 34; 45; 56; 67; 78; 89; 100; 111; 122; 133; 599; 610; 621 and 1022-1024, or a fragment or variant of any of these sequences, preferably from the group consisting of SEQ ID NOs: 24; 35; 46; 57; 68; 79; 90; 101; 112; 123; 134; 600; 611; 622 and 1043-1054, or a fragment or variant of any of these sequences.
66. The isRNA for use according to any of items 20 to 65, wherein the at least one coding RNA comprises at least one coding sequence having a modified, preferably an increased, G/C content compared to the G/C content of the coding sequence of the corresponding wild type RNA, and wherein the amino acid sequence encoded by said coding sequence is preferably not modified compared to the amino acid sequence encoded by the coding sequence of the corresponding wild type RNA.
67. The isRNA for use according to item 66, wherein the at least one coding sequence comprises
   a) a nucleic acid sequence selected from the group consisting of SEQ ID NOs: 25-30; 80-85; 91-96; 102-107; 113-118; 601-606; 124-129; 135-140; 612-617; 623-628; 716-725; 727 and 1018-1021 and 1059-1062, or a fragment or variant of any of these sequences, preferably from the group consisting of SEQ ID NOs: 32; 87; 98; 109; 120; 131; 142; 608; 619; 630; 632; 636-644 and 726 and 1058, or a fragment or variant of any of these sequences;
   b) a nucleic acid sequence selected from the group consisting of SEQ ID NOs: 33; 88; 99; 110; 121; 132; 143; 609; 620; 631; 728-738 and 1025-1028, or a fragment or variant of any of these sequences,
   c) a nucleic acid sequence selected from the group consisting of SEQ ID NOs: 646; 650-658; 662; 666-674; 678; 682-690; 694; 698-705; 707; 710; 713 and 1029-1041, or a fragment or variant of any of these nucleic acid sequences, and/or
   d) optionally, a nucleic acid sequence selected from the group consisting of SEQ ID NOs: 23; 78; 89; 100; 111; 122; 133; 599; 610; 621 and 1022-1024, or a fragment or variant of any of these sequences, preferably from the group consisting of SEQ ID NOs: 24; 79; 90; 101; 112; 123; 134; 600; 611; 622 and 1043-1054, or a fragment or variant of any of these sequences.
68. The isRNA for use according to any of items 20 to 67, wherein the at least one coding RNA comprises a 5′-CAP structure.
69. The isRNA for use according to any of items 20 to 68, wherein the at least one coding RNA comprises a 5′-UTR element and/or a 3′-UTR element.
70. The isRNA for use according to any of items 20 to 69, wherein the at least one coding RNA comprises a poly(A) and/or a poly(C) sequence.
71. The isRNA for use according to any of items 20 to 70, wherein the at least one coding RNA comprises a histone stem-loop sequence.
72. The isRNA for use according to any of items 20 to 71, wherein the at least one coding RNA comprises
   a) a nucleic acid sequence selected from the group consisting of SEQ ID NOs: 146-151; 451-456; 157-162; 168-173; 179-184; 190-195; 201-206; 212-217; 223-228; 234-239; 245-250; 256-261 and 267-272, or a fragment or variant of any of these sequences, preferably from the group consisting of SEQ ID NOs: 153; 458; 164; 175; 186; 197; 208; 219; 230; 241; 252; 263, 274; 992 and 598, or a fragment or variant of any of these sequences,
   b) a nucleic acid sequence selected from the group consisting of SEQ ID NOs: 154; 459; 165; 176; 187; 198; 209; 220; 231; 242; 253; 264, 275 and 596, or a fragment or variant of any of these sequences,
   c) a nucleic acid sequence selected from the group consisting of SEQ ID NOs: 594; 595; 860-925, or a fragment or variant of any of these sequences, and/or
   d) optionally, a nucleic acid sequence selected from the group consisting of SEQ ID NOs: 144; 449; 155; 166; 177; 188; 199; 210; 221; 232; 243; 254 and 265, or a fragment or variant of any of these sequences, preferably from the group consisting of SEQ ID NOs: 145; 450; 156; 167; 178; 189; 200; 211; 222; 233; 244; 255; 266 and 597, or a fragment or variant of any of these sequences.
73. The isRNA for use according to item any of items 20 to 72, wherein the treatment comprises administration, preferably intratumoral administration, of at least three coding RNAs, wherein
   a first coding RNA comprises a nucleic acid sequence selected from the group consisting of SEQ ID NOs: 153; 164; 175; 186; 197; 208; 219; 230; 241; 252; 263; 274; 992; 458; 598; 32; 43; 54; 65; 76; 87; 98; 109; 120; 131; 142; 608; 619; 630; 632-644; 726 and 1058, or a fragment or variant of any of these sequences,
   a second coding RNA comprises a nucleic acid sequence selected from the group consisting of SEQ ID NOs: 33; 44; 55; 66; 77; 88; 99; 110; 121; 132; 143; 609; 620; 631; 728-738; 1025-1028; 154; 165; 176; 187; 198; 209; 220; 231; 242; 253; 264; 275; 459 and 596, or a fragment or variant of any of these sequences,
   a third coding RNA comprises a nucleic acid sequence selected from the group consisting of SEQ ID NOs: 920-922; 923-925; 646-660; 662-676; 678-692; 694-705; 707-715 or 1029-1041, or a fragment or variant of any of these sequences, and
   optionally, a fourth coding RNA comprises a nucleic acid sequence selected from the group consisting of SEQ ID NOs: 145; 156; 167; 178; 189; 200; 211; 222; 233; 244; 255; 266; 450; 597; 24; 35; 46; 57; 68; 79; 90; 101; 112; 123; 134; 600; 611; 622 and 1043-1054, or a fragment or variant of any of these sequences.
74. The isRNA for use according to claim any of items 20 to 73, wherein the treatment comprises administration, preferably intratumoral administration, of at least four coding RNAs, wherein
   a first coding RNA comprises a nucleic acid sequence selected from the group consisting of SEQ ID NOs: 153; 164; 175; 186; 197; 208; 219; 230; 241; 252; 263; 274; 992; 458 and 598, or a fragment or variant of any of these sequences,
   a second coding RNA comprises a nucleic acid sequence selected from the group consisting of SEQ ID NOs: 154; 165; 176; 187; 198; 209; 220; 231; 242; 253; 264; 275; 459 and 596, or a fragment or variant of any of these sequences,
   a third coding RNA comprises a nucleic acid sequence selected from the group consisting of SEQ ID NOs: 594 and 860-874, or a fragment or variant of any of these sequences,
   a fourth coding RNA comprises a nucleic acid sequence selected from the group consisting of SEQ ID NOs: 595 and 890-904, or a fragment or variant of any of these sequences, and
   optionally, a fifth coding RNA comprises a nucleic acid sequence selected from the group consisting of SEQ ID NOs: 145; 156; 167; 178; 189; 200; 211; 222; 233; 244; 255; 266; 450 and 597, or a fragment or variant of any of these sequences.
75. The isRNA for use according to any of items 20 to 74, wherein the at least one coding RNA comprises
   a) a nucleic acid sequence selected from the group consisting of SEQ ID NOs: 278-283; 289-294; 300-305; 311-316; 322-327; 333-338; 344-349; 355-360; 366-371; 377-382; 388-393; 399-404 and 462-467, or a fragment or variant of any of these sequences, preferably from the group consisting of SEQ ID NOs: 285; 296; 307; 318; 329; 340; 351; 362; 373; 384; 395; 406; 430; 469 and 992, or a fragment or variant of any of these sequences,
   b) a nucleic acid sequence selected from the group consisting SEQ ID NO: 286; 297; 308; 319; 330; 341; 352; 363; 374; 385; 396; 470 and 407, or a fragment or variant of any of these sequences, and/or
   c) a nucleic acid sequence selected from the group consisting of SEQ ID NOs: 926-955; or a fragment or variant of any of these nucleic acid sequences, and a nucleic acid sequence selected from the group consisting of SEQ ID NOs: 956-985, or a fragment or variant of any of these nucleic acid sequences; or a nucleic acid sequence selected from the group consisting of SEQ ID NOs: 986-991, or a fragment or variant of any of these nucleic acid sequences, and/or
   d) optionally, a nucleic acid sequence selected from the group consisting of SEQ ID NOs: 276; 287; 298; 309; 320; 331; 342; 353; 364; 375; 386; 460 and 397, or a fragment or variant of any of these sequences, preferably from the group consisting of SEQ ID NOs: 277; 288; 299; 310; 321; 332; 343; 354; 365; 376; 461; 387 and 398 or a fragment or variant of any of these sequences.
76. The isRNA for use according to any of items 20 to 65, wherein the treatment comprises administration, preferably intratumoral administration, of at least four coding RNAs, wherein
   a first coding RNA comprises a nucleic acid sequence selected from the group consisting of SEQ ID NOs: 285; 296; 307; 318; 329; 340; 351; 362; 373; 384; 395; 406; 430; 469 and 992, or a fragment or variant of any of these sequences,
   a second coding RNA comprises a nucleic acid sequence selected from the group consisting of SEQ ID NOs: 286; 297; 308; 319; 330; 341; 352; 363; 374; 385; 396; 470 and 407, or a fragment or variant of any of these sequences,
   a third coding RNA comprises a nucleic acid sequence selected from the group consisting of SEQ ID NOs: 926-940, or a fragment or variant of any of these sequences, and/or
   a fourth coding RNA comprises a nucleic acid sequence selected from the group consisting of SEQ ID NOs: 956-970, or a fragment or variant of any of these sequences and/or
   optionally, a fifth coding RNA comprises a nucleic acid sequence selected from the group consisting of SEQ ID NOs: 277; 288; 299; 310; 321; 332; 343; 354; 365; 376; 387; 461 and 398, or a fragment or variant of any of these sequences.
77. The isRNA for use according to any of items 20 to 76, wherein the isRNA is administered as RNA complexed with one or more cationic or polycationic compounds, and the at least one coding RNA, more preferably an mRNA, is administered as free RNA or is administered as RNA that is complexed with one or more lipids, thereby forming liposomes, lipid nanoparticles and/or lipoplexes.
78. The isRNA for use according to any of items 1 to 77, wherein the treatment comprises chemotherapy, radiation therapy and/or surgery.
79. Pharmaceutical composition comprising an immunostimulatory RNA (isRNA) and a pharmaceutically acceptable carrier and/or vehicle for use in the treatment or prophylaxis of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy, wherein the pharmaceutical composition is administered intratumorally.
80. The pharmaceutical composition for use according to item 79, wherein the isRNA is as defined in items 1 to 12.
81. The pharmaceutical composition for use according to item 79 or 80, wherein the treatment comprises administration of at least one additional pharmaceutically active ingredient, preferably as defined in any of items 13 to 68.
82. Kit or kit of parts comprising an immunostimulatory RNA (isRNA), preferably the isRNA as defined in items 1 to 12, or the pharmaceutical composition as defined in any of items 79 to 81, and optionally technical instructions with information on the administration and dosage for administration,
   for use in the treatment of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy,
   wherein the pharmaceutical composition is administered intratumorally.
83. Use of an isRNA, preferably the isRNA as defined according to any of items 1 to 12, the pharmaceutical composition as defined in any of items 79 to 81, or the kit or kit of parts as defined in item 82, for use in the manufacture of a medicament for treatment of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy, for intratumoral, peritumoral or locoregional administration, preferably for intratumoral administration.
84. Method of treating or preventing a disorder selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the disorder is preferably at an advanced stage and/or refractory to standard therapy, wherein the method comprises administering, preferably intratumorally, to a subject in need thereof an effective amount of an isRNA, preferably the isRNA as defined according to any of items 1 to 12, the pharmaceutical composition as defined in any of items 79 to 81, or the kit or kit of parts as defined in item 82.
85. Combination of an isRNA and at least one coding RNA, wherein the at least one coding RNA encodes at least one peptide or protein comprising IL-12, CD40L, a decoy PD-1 receptor, an anti-CTLA4 antibody, and/or a tumor antigen, or a fragment or variant of each of these proteins.
86. The combination according to item 85, wherein the isRNA is an isRNA as defined in any of items 4 to 12.
87. The combination according to item 85 or 86, wherein the at least one coding RNA is a coding RNA as defined in any of items 13 to 78.
88. The combination according to any of items 85 to 87, wherein the isRNA and the at least one coding RNA are formulated together or separately.
89. The combination according to any of items 85 to 88, wherein the isRNA and the at least one coding RNA are administered concomitantly.
90. The combination according to any of items 85 to 89, wherein the isRNA and the at least one coding RNA are administered at the same site.
91. The combination according to any of items 85 to 90 for use in the treatment or prophylaxis of a disease selected from the group consisting of tumor and cancer disease, infectious diseases, allergies and autoimmune diseases.
92. The combination according to item 91, wherein the combination is for use in the treatment or prophylaxis of a tumor or a cancer disease.
93. The combination according to item 92, wherein the combination is for use in the treatment or prophylaxis of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy.
94. The combination according to item 93, wherein the combination is administered intratumorally
95. The combination according to item 93 or 94, wherein the treatment or prophylaxis comprises administration of at least one additional pharmaceutically active ingredient.
96. The combination according to item 95, wherein the at least one additional pharmaceutically active ingredient is a compound as defined in items 14 to 18.
97. The combination according to item 96, wherein the at least one additional pharmaceutically active ingredient is a PD-1 inhibitor or a PD-L1 inhibitor, preferably an antagonistic antibody directed against PD-1 or PD-L1, or a fragment or variant thereof.
98. The combination according to item 96, wherein the at least one additional pharmaceutically active ingredient is an anti-CTLA4 antibody, or a fragment or variant thereof.
99. Coding RNA encoding a protein comprising at least one peptide or protein comprising IL-12, CD40L, a decoy PD-1 receptor, an anti-CTLA4 antibody, and/or a tumor antigen, or a fragment or variant of any of these proteins.
100. The coding RNA according to item 99, which is a coding RNA as defined in any of items 13 to 78.
101. The coding RNA according to item 99 or 100 for use in the treatment or prophylaxis of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy.
102. The coding RNA for use according to item 101, wherein the coding RNA comprises at least one coding sequence encoding a peptide or protein comprising IL-12 or a fragment or variant thereof.
103. The coding RNA for use according to any of items 99 to 102, wherein the at least one coding RNA comprises at least one coding sequence encoding a peptide or protein comprising CD40L or a fragment or variant thereof and
   at least one coding sequence encoding a peptide or protein comprising IL-12 or a fragment or variant thereof.
104. The coding RNA for use according to any of items 99 to 103, wherein the at least one coding RNA comprises at least one coding sequence encoding a peptide or protein comprising CD40L or a fragment or variant thereof, and
   at least one coding sequence encoding a peptide or protein comprising a decoy PD-1 receptor or a fragment or variant thereof.
105. The coding RNA for use according to any of items 99 to 104, wherein the at least one coding RNA comprises at least one coding sequence encoding a peptide or protein comprising IL-12 or a fragment or variant thereof, and
   at least one coding sequence encoding a peptide or protein comprising a decoy PD-1 receptor or a fragment or variant thereof.
106. The coding RNA for use according to any of items 99 to 105, wherein the at least one coding RNA comprises at least one coding sequence encoding a peptide or protein comprising IL-12 or a fragment or variant thereof, and
   at least one coding sequence encoding at least one tumor antigen or a fragment or variant thereof.
107. The coding RNA for use according to any of items 99 to 106, wherein the at least one coding RNA comprises at least one coding sequence encoding a peptide or protein comprising CD40L or a fragment or variant thereof, and
   at least one coding sequence encoding a peptide or protein comprising an anti-CTLA4 antibody, or a fragment or variant thereof.
108. The coding RNA for use according to any of items 99 to 107, wherein the at least one coding RNA comprises at least one coding sequence encoding a peptide or protein comprising IL-12 or a fragment or variant thereof, and
   at least one coding sequence encoding a peptide or protein comprising an anti-CTLA4 antibody, or a fragment or variant thereof.
109. The coding RNA for use according to any of items 99 to 108, wherein the at least one coding RNA comprises at least one coding sequence encoding a peptide or protein comprising a decoy PD-1 receptor or a fragment or variant thereof, and
   at least one coding sequence encoding a peptide or protein comprising an anti-CTLA4 antibody, or a fragment or variant thereof.
110. The coding RNA for use according to any of items 99 to 109, wherein the at least one coding RNA comprises at least one coding sequence encoding a peptide or protein comprising IL-12 or a fragment or variant thereof, at least one coding sequence encoding a peptide or protein comprising CD40L or a fragment or variant thereof, and
   at least one coding sequence encoding a peptide or protein comprising an anti-CTLA4 antibody, or a fragment or variant thereof.
111. The coding RNA for use according to any of items 99 to 110, wherein the treatment or prophylaxis comprises administration of a second coding RNA, and/or a third coding RNA, wherein
   the coding RNA comprises at least one coding sequence encoding a peptide or protein comprising IL-12 or a fragment or variant thereof,
   the second coding RNA comprises at least one coding sequence encoding a peptide or protein comprising CD40L or a fragment or variant thereof,
   the third coding RNA comprises at least one coding sequence encoding a peptide or protein comprising an anti-CTLA4 antibody, or a fragment or variant thereof.
112. The coding RNA for use according to item 111, whereint the treatment or prophylaxis further comprises administration of a fourth coding RNA, wherein the fourth coding RNA comprises at least one coding sequence encoding a peptide or protein comprising a decoy PD-1 receptor or a fragment or variant thereof
113. The coding RNA for use according to item 111 or 112, wherein the second RNA, the third RNA, and/or the fourth RNA is a coding RNA as defined in any of items 13 to 78.
114. The coding RNA for use according to any of items 101 to 113, wherein the treatment or prophylaxis comprises administration of an isRNA.
115. The coding RNA for use according to item 114, wherein the isRNA is an isRNA as defined in any of items 4 to 12.
116. The coding RNA for use according to any of items 101 to 115, wherein the at least one coding RNA is administered intratumorally,
117. The coding RNA for use according to any of items 101 to 116, wherein the treatment or prophylaxis comprises administration of at least one additional pharmaceutically active ingredient, preferably a compound that is conventionally used in the treatment of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy.
118. The coding RNA for use according to item 117, wherein the at least one additional pharmaceutically active ingredient is a compound as defined in any of items 14 to 33.
119. The coding RNA for use according to item 117 or 118, wherein the at least one additional pharmaceutically active ingredient is a PD-1 inhibitor or a PD-L1 inhibitor, preferably an antagonistic antibody directed against PD-1 or PD-L1, or a fragment or variant thereof.
120. The coding RNA for use according to any one of items 117 to 119, wherein the at least one additional pharmaceutically active ingredient is an anti-CTLA4 antibody, or a fragment or variant thereof.

### EXAMPLES

The Examples shown in the following are merely illustrative and shall describe the present invention in a further way. These Examples shall not be construed to limit the present invention thereto.

Preparation of DNA, mRNA Constructs and Immunostimulatory RNA (RNAdjuvant)

1. Preparation of DNA and RNA Constructs

TABLE 3
RNA constructs

| RNA | Description | SEQ ID NO: |
|---|---|---|
| R2025 R2391 | Non-coding immunostimulatory RNA | 433 |
| | (RNAdjuvant) | |
| R2763 | mRNA encoding murine IL-12 (IL-12 (GC)) | 430 |
| R3971 | mRNA encoding murine soluble PD-1 | 431 |
| | (soluble PD-1 (GC)) | |
| R3571 | mRNA encoding murine CD40L | 432 |
| | (CD40L (GC)) | |
| R5417 | mRNA encoding heavy chain (HC) of anti-CTLA4 antibody | 594 |
| | (HC anti-CTLA4Ab (GC)) | |
| R5418 | mRNA encoding light chain (LC) of anti-CTLA4 antibody | 595 |
| | (HLC anti-CTLA4 Ab (GC)) | |

The constructs of IL-12 (GC), soluble PD-1 (GC) (solPD1), CD40L (GC) and two anti-CTLA4 antibody chains were prepared by a stabilizing sequence derived from the albumin-3′-UTR, a stretch of 64 adenosines (poly(A)-sequence), a stretch of 30 cytosines (poly(C)-sequence), and a histone stem loop. Most DNA sequences were prepared by modifying the wild type encoding DNA sequences by introducing a GC-optimized sequence for stabilization, using an in silico algorithm that increase the GC content of the respective coding sequence compared to the wild type coding sequence. The mRNAs expressing human IL-12, soluble PD-1 receptor, CD40L and anti-CTLA4 antibody are prepared in analogous manner by using the corresponding human coding sequences.

For the present example, a DNA sequence encoding the non-coding immunostimulatory RNA (isRNA) R2025 was prepared and used for subsequent RNA in vitro transcription reactions.

2. RNA In Vitro Transcription

The respective DNA plasmids prepared according to section 1 above were transcribed in vitro using T7 polymerase. The RNA in vitro transcription reactions of the IL-12, CD40L, soluble PD-1 and anti-CTLA4 antibody encoding constructs were performed in the presence of a CAP analog (m7GpppG). The isRNA R2025 was prepared without CAP analog. Subsequently, the RNA was purified using PureMessenger® (CureVac, Tübingen, Germany; WO2008077592).

3. Preparation of the Polymeric Cargo Complex (“RNAdjuvant”)

The following cationic peptide as cationic component of the polymeric carrier was used (Cys-Arg12-Cys or CR12C) according to SEQ ID NO: 579 or SEQ ID NO: 580.

For synthesis of the polymeric carrier cargo complexes, an RNA molecule having the RNA sequence R2025 as defined in section 1 above was mixed with the cationic CR12C (SEQ ID NO: 579) peptide component as defined above. The specified amount of the RNA was mixed with the respective cationic component in mass ratios as indicated below, thereby forming a complex. If polymerizing cationic components were used according to the present invention, polymerization of the cationic components took place simultaneously to complexation of the nucleic acid cargo. Afterwards, the resulting solution was adjusted with water to a final volume of 50 μl and incubated for 30 minutes at room temperature. Further details are described in WO2012013326.

The mass ratio of peptide:RNA was 1:3,7. The polymeric carrier cargo complex is formed by the disulfide-crosslinked cationic peptide CR12C (SEQ ID NO: 579) as carrier and the immunostimulatory R₂₀₂₅ as nucleic acid cargo. This polymeric carrier cargo complex R2025/CR12C (SEQ ID NO: 579) (R₂₃₉₁) was used as adjuvant in the following examples (referred to as “RNAdjuvant”)

4. Preparation of the RNA for Administration

IL-12 mRNA (R1328), soluble PD-1 mRNA (R3971) and CD40L mRNA (R3571) were administered in Ringer's Lactate (RiLa) solution. The co-formulation of naked mRNAs and the polymeric carrier cargo complex “RNAdjuvant” (R2391) were also administered in Ringer's Lactate (RiLa) after mixing of both components directly before injection.

### Example 1: Intratumoral Treatment with an Immunostimulating RNA (“RNAdjuvant”) and an mRNA Encoding Soluble PD-1 and an mRNA Encoding IL-12

Balb/c mice (see Table 4) were injected subcutaneously (s.c.) with 1×10⁶ CT26 cells (colon carcinoma cell line) per mouse (in a volume of 100 μl PBS) on the right flank on day 0 of the experiment. At day 9 after tumor challenge, mice were sorted according to the tumor size to obtain groups with a mean tumor volume of approximately 50 mm³. Intratumoral (i.t.) therapy started at day 9 and continued twice a week for three weeks. Mice were injected with a combination of RNAdjuvant (25 μg of R2391), mRNA-encoded IL-12 (25 μg of R2763) and mRNA-encoded soluble PD-1 (R3971) (group A according to Table 2) or mRNA-encoded IL-12 (25 μg of R2763) (group B according to Table 2) alone or RNAdjuvant (25 μg of R2391) alone (group C according to Table 2). To control for anti-tumor effects due to injection procedure, mice were injected with buffer (RiLa, group D according to Table 4), respectively.

Tumor growth was monitored by measuring the tumor size in three dimensions using a calliper. Tumor volume was calculated according to the following formula:

$$volume ( {mm}^{3} ) = \frac{length ( mm ) \times π \times {width}^{2} ( {mm}^{2} )}{6}$$

On days 9, 11, 14, 17 and 21 of the experiment mice were injected intratumorally (i.t.) with RNA according to the Table 4 below. The volume for intratumoral injection was 50 μl.

Table 4 summarizes the treatment as used in the present example. RNAdjuvant and the mRNA constructs encoding IL-12 and soluble PD-1 were administered intratumorally (i.t.). In CT26 tumor challenged mice, survival rates and median tumor growth were analyzed.

TABLE 4
Groups, treatment and RNA dilution

| | No. of | | Amount | Vaccination |
|---|---|---|---|---|
| Group | mice | Constructs | of RNA (μg) | schedule |
| A | 10 | RNAdjuvant + IL-12 + | 25 each | 2× week |
| | | soluble PD-1 | | |
| B | 10 | IL-12 | 25 | 2× week |
| C | 10 | RNAdjuvant | 25 | 2× week |
| D | 10 | RiLa | — | 2× week |

Tumor Challenge and Administration of the Inventive Composition:

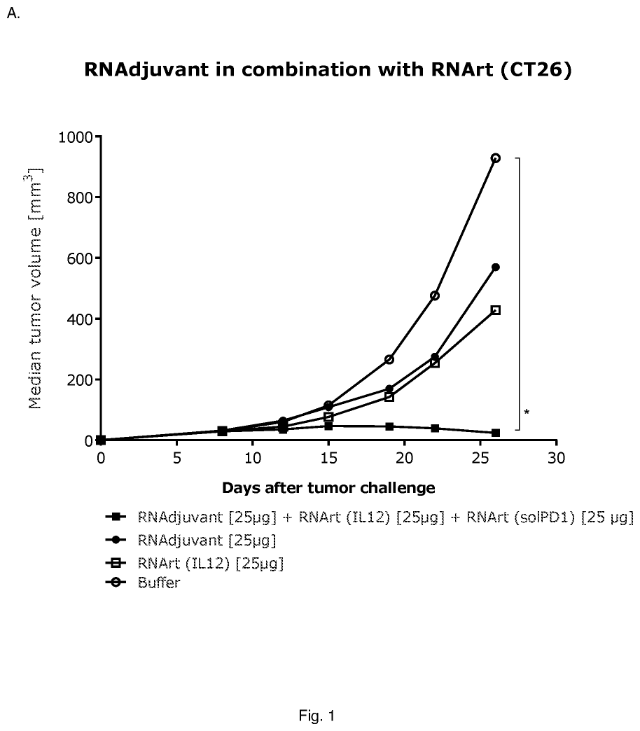

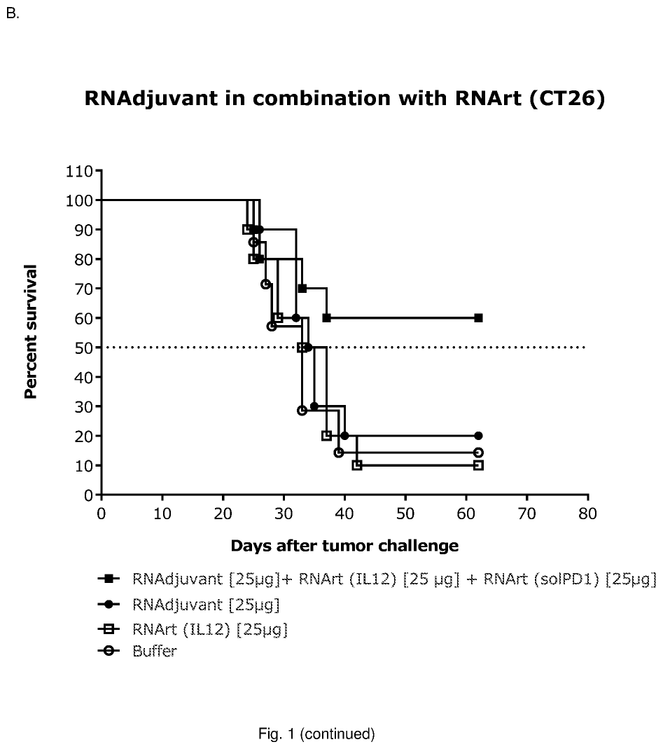

Mice were injected according to the indicated scheme shown in Table 4. Median tumor growth was determined according to formula above. The results of the experiment are shown in FIG. 1, wherein FIG. 1A shows the effect of the inventive composition on tumor growth and FIG. 1B shows the effect of the inventive composition on survival.

Results:

The results in FIG. 1A show that the inventive composition comprising an mRNA encoding IL-12 and mRNA encoding soluble PD-1 in combination with RNAdjuvant (group A according to Table 4) strongly decreased the median tumor volume compared to the other treatments (groups B-D according to Table 4). In addition, the results in FIG. 1B show that the inventive composition comprising an mRNA encoding IL-12 and mRNA encoding soluble PD-1 in combination with RNAdjuvant (group “A” according to Table 4) strongly increased the survival of tumor challenged mice compared to the other treatments (groups B-D according to Table 4).

### Example 2: Treatment with an Immunostimulating RNA (“RNAdjuvant”) in Combination with a Checkpoint Inhibitor Anti PD-1 Antibody

Table 5 summarizes the treatment as used in the present example. In addition to RNAdjuvant (administered i.t.), a checkpoint inhibitor anti PD-1 (BioXCell) was administered intraperitoneal (i.p.) in CT26 tumor challenged mice, survival rates were analyzed.

TABLE 5
Groups, treatment and RNA
dilution/antibody dilution

| | No. of | Construct | Amount of | Antibody (i.p. | Vaccination |
|---|---|---|---|---|---|
| Group | mice | (i.t. treatment) | RNA (μg) | treatment) | schedule |
| A | 8 | RiLa | — | — | 2× week |
| B | 8 | RNAdjuvant | 25 | Control Ab | 2× week |
| | | | | (100 μg) | |
| C | 7 | RNAdjuvant | 25 | Anti-PD-1 | 2× week |
| | | | | (200 μq) | |
| D | 6 | RiLa | — | Anti-PD-1 | 2× week |
| | | | | (200 μg) | |

Tumor Challenge and Administration of the Inventive Composition:

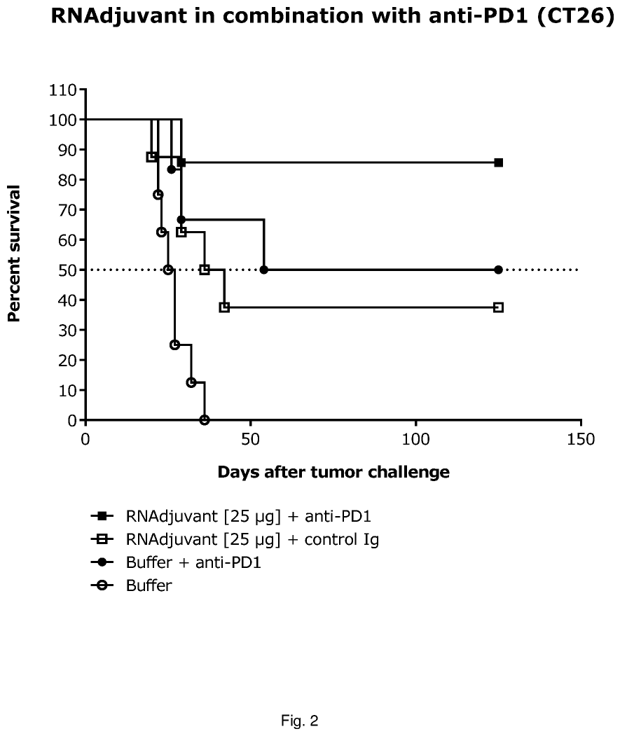

The tumor challenge was performed according to the previous experiment (see Example 1). Mice were injected according to the indicated scheme shown in Table 5. The results of the experiment are shown in FIG. 2.

Results:

FIG. 2 shows that the intratumoral (i.t.) treatment with RNAdjuvant® (R2391) in combination with an i.p. administration of anti PD-1 antibody (Group “C” according to Table 5) resulted in an increase in survival compared to the relevant control group that only received the checkpoint inhibitor anti PD-1 antibody (Group “D” according to Table 5) and in an increase in survival rates compared to the treatment with RNAdjuvant and a control antibody (anti hamster IgG, BioXCell) (Group “B” according to Table 5).

### Example 3: Intratumoral Treatment with an Immunostimulating RNA (“RNAdjuvant”) and an mRNA Encoding CD40 Ligand (CD40L) and an mRNA Encoding IL-12

Table 6 summarizes the treatment as used in the present example. RNAdjuvant and the mRNA constructs encoding IL-12 and murine CD40L were administered intratumorally (i.t.). In CT26 tumor challenged mice, survival rates were analysed.

TABLE 6
Groups, treatment and RNA dilution

| | No. of | | Vaccination |
|---|---|---|---|
| Group | mice | Constructs (amount of RNA) | schedule |
| A | 10 | RNAdjuvant (50 μg) + IL-12 (75 μg) + | 2× week |
| | | CD40L (75 μg) | |
| B | 10 | RNAdjuvant (100 μg) | 2× week |
| C | 10 | RiLa | 2× week |

Tumor Challenge and Administration of the Inventive Composition:

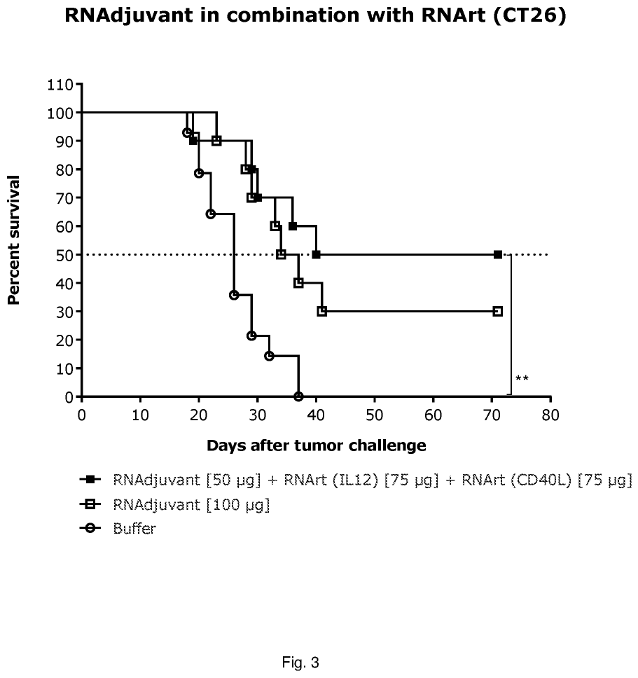

The tumor challenge was performed according to the previous experiments (see Example 1). Mice were injected according to the indicated scheme shown in Table 6. The results of the experiment are shown in FIG. 3.

Results:

The results in FIG. 3 show that the inventive composition comprising an mRNA encoding IL-12 and an mRNA encoding CD40L in combination with RNAdjuvant (group A according to Table 6) strongly increased the median survival of tumor challenged mice compared to the other treatments (groups B to C according to Table 6).

### Example 4: Induction of Systemic Anti-Tumoral Memory Response by Combinination of Intratumoral Treatment with an Immunostimulating RNA (“RNAdjuvant”) and Systemic Anti-PD-1 Treatment

Table 7 summarizes the treatment as used in the present example. RNAdjuvant (administered i.t.) and systemic treatment with a checkpoint inhibitor anti PD-1 (BioXCell) was evaluated by performing re-challenge of mice completely eradicating the primary CT-26 tumor after first treatment, survival rates were analyzed.

TABLE 7
Groups, treatment and RNA dilution

| | No. of | Constructs of first tumor challenge |
|---|---|---|
| Group | mice | (amount of RNA/Ab + route of treatment) |
| A | 7 | RNAdjuvant (100 μg, i.t.) + Anti-PD-1 (200 μg, i.p.) |
| B | 3 | RNAdjuvant (100 μg, i.t.) |
| C | 7 | RiLa |

Tumor Re-Challenge after Administration of the Inventive Composition:

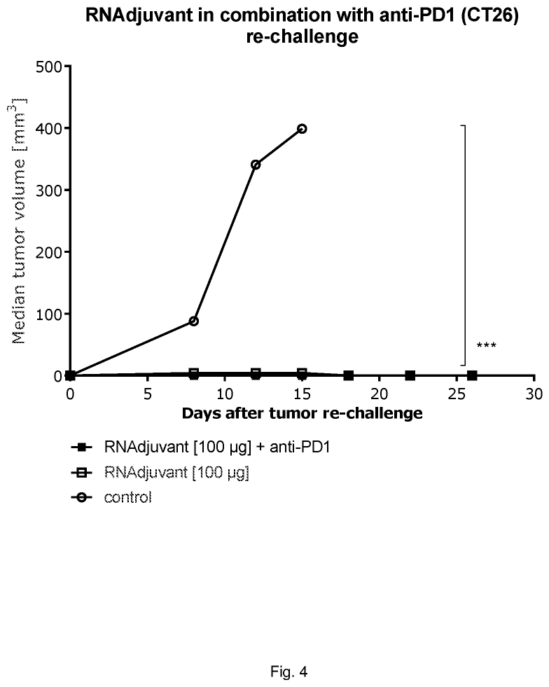

First tumor challenge was performed by s.c. injection of CT-26 tumor cells on the right flank in Balb/C mice, whereas re-challenge with 1×10⁶ syngeneic CT26 colon carcinoma cells was performed on the left flank at day 113 after first tumor challenge. Challenge of naïve animals served as control. Tumor eradication of the primary tumor was noted in a lower percentage of animals with intratumoral RNAdjuvant alone (3 out of 10 mice) as compared to the combination of systemic anti-PD-1 with intratumoral RNAdjuvant (7 out of 9 mice). The results of the experiment are shown in FIG. 4.

Results:

The results in FIG. 4 show that all mice which have eradicated the first tumor were completely protected against the second tumor challenge demonstrating the induction of systemic memory response. Systemic memory response was also induced by intratumoral RNAdjuvant treatment alone. The induction of a systemic memory response is remarkable as no vaccine inducing an adaptive immune response was administered. Therefore administration of the inventive isRNA, particularly in combination with systemic anti-PD-1 treatment, was sufficient to induce a systemic immune response which could not have been expected.

### Example 5: Induction of Systemic Anti-Tumoral Memory Response by Combination of Intratumoral Treatment with an mRNA Encoding CD40 Ligand (CD40L) and an mRNA Encoding IL-12

Table 8 summarizes the treatment as used in the present example. RNAdjuvant and the mRNA constructs encoding IL-12 and murine CD40L (administered i.t.) were evaluated by performing re-challenge of mice completely eradicating the primary CT-26 tumor after treatment, survival rates were analyzed.

TABLE 8
Groups, treatment and RNA dilution

| | No. of | Constructs of first tumor challenge |
|---|---|---|
| Group | mice | (amount of RNA) |
| A | 5 | RNAdjuvant (50 μg) + CD40L (75 μg) + IL-12 (75 μg) |
| B | 3 | RNAdjuvant (100 μg) |
| C | 7 | RiLa |

Tumor Re-Challenge after Administration of the Inventive Composition:

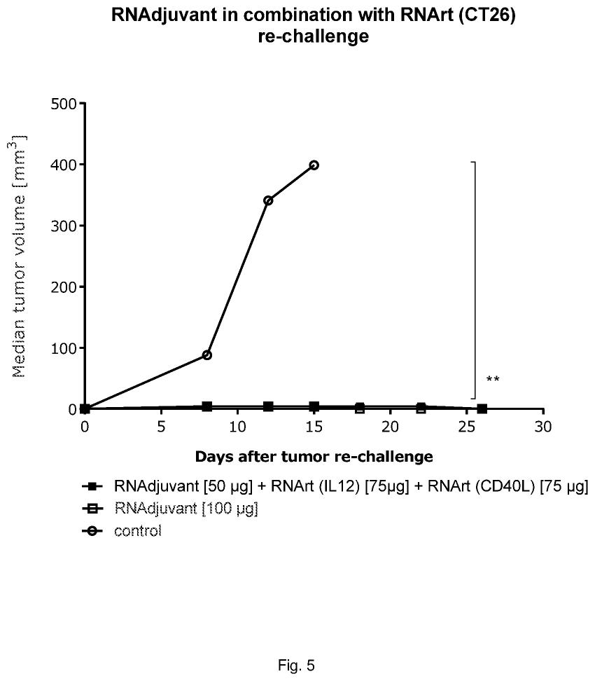

First tumor challenge was performed by s.c. injection of CT-26 tumor cells on the right flank in Balb/C mice. After intratumorally treatment with RNAdjuvant alone or in combination with an mRNA encoding CD40 ligand (CD40L) and an mRNA encoding IL-12 treatment, re-challenge with 1×10⁶ syngeneic CT26 colon carcinoma cells was performed on the left flank at day 113 after the first tumor challenge. Challenge of naïve animals served as control. Tumor eradication of the primary tumor was noted in a lower percentage of animals with intratumoral RNAdjuvant alone (3 out of 10 mice) as compared to the combination of mRNA encoding CD40 ligand (CD40L) and an mRNA-encoded IL-12 with intratumoral RNAdjuvant (5 out of 10 mice). The results of the experiment are shown in FIG. 5.

Results:

The results in FIG. 5 show that all mice which have eradicated the first tumor were completely protected against the second tumor challenge demonstrating the induction of systemic memory response. Systemic memory response was also induced by intratumoral RNAdjuvant treatment alone.

### Example 6: Phase I/II Study of Intratumoral Application of RNAdjuvant (CV8102) in Patients with Advanced Cutaneous Melanoma (cMEL), Cutaneous Squamous Cell Carcinoma (cSCC), Head and Neck Squamous Cell Carcinoma (hnSCC), or Adenoid Cystic Carcinoma (ACC)

Part 1:

Phase I, open label, cohort based dose escalation & expansion study of intratumorally administered RNAdjuvant, with or without systemic anti-PD-1 treatment, in patients having advanced cutaneous melanoma (cMEL), cutaneous squamous cell carcinoma (cSCC), head and neck squamous cell carcinoma (hnSCC), or adenoid cystic carcinoma (ACC).

Intratumoral injection of RNAdjuvant: CV8102 is administered to cutaneous, subcutaneous, or readily accessible lymph node lesions that can be injected using direct visualization or imaging-guidance (ultrasound) as clinically determined.

Schedule: Patients receive a maximum of 8 intratumoral administrations of CV8102, unless disease progression requiring initiation of next-line therapy or unacceptable toxicity occurs:

The first 5 administrations are performed in weekly intervals (Days 1, 8, 15, 22, 29). For patients on anti-PD-1 treatment in Cohorts C and D, CV8102 treatment is initiated on a day of anti-PD-1 treatment and will follow the anti-PD-1 treatment schedule after day 29.

The subsequent 3 administrations of CV8102 are performed with 2-week intervals (Cohorts A and B). For patients on anti-PD-1 treatment in study Cohorts C and D, the administrations of CV8102 after Day 29 follow the anti-PD-1 treatment schedule and are performed on the day of anti-PD-1 administration; i.e., patients on nivolumab receive CV8102 every 2nd week; patients on pembrolizumab receive CV8102 every 3rd week.

Part A: Dose Escalation of Single Agent RNAdjuvant

Part A of the study uses a 2 parameter Bayesian logistic regression model with overdose control for dose escalation. Cohorts of at least 1 (starting dose level) or 2 patients (any other dose level) with advanced cMEL, cSCC, hnSCC, or ACC are treated at escalating doses of intratumorally administered RNAdjuvant until identification of the maximum tolerated dose (MTD) and determination of the recommended dose (RD). A minimum of 7 patients should be enrolled in Part A.

The starting dose in Part A is 25 μg of RNAdjuvant. The further dose levels are listed in Table 9 below.

TABLE 9
Dose levels of RNAdjuvant
Dose Levels evaluated in Part A and C

| Dose Level | RNAdjuvant | No of Patients⁹ |
|---|---|---|
| Level -1 | 25μg | ≥1 |
| Level 2* | 50 μg | ≥2 |
| Level 3 | 100 μg | ≥2 |
| Level 4 | 150 μq | ≥2 |

*Starting dose level Part A; potential starting dose level Part C, based on review of Part A data
^{§}A minimum of 3 patients will be treated per cohort

Inclusion Criteria:

Cohort A:

Patients with histologically confirmed advanced (unresectable or metastatic) cMEL or cSCC who failed approved standard therapy or for whom no standard therapy is indicated

Not amenable to surgical resection or locoregional radiation therapy with curative intent

Progression on/after at least one line of therapy

or

Histologically confirmed recurrent or metastatic hnSCC

Not amenable to surgical resection or locoregional radiation therapy with curative intent

Progression documented radiologically (documentation of a new or progressive lesion on/after at least one line of therapy.

or

Histologically or cytologically confirmed ACC

Not amenable to surgical resection or locoregional radiation therapy with curative intent

Progression documented radiologically; on/after at least one line of therapy

Part B: Expansion Cohort

After completion of Part A, patients with advanced cSCC or ACCare enrolled to separate expansion cohorts at the previously defined recommended dose to further characterize the tolerability and safety profile of intratumorally administered RNAdjuvant in these patient populations and to collect pre-liminary evidence of anti-tumor activity. Part B should enrol up to 10 patients per expansion cohort. Inclusion criteria:

Cohort B:

Expansion cohorts B1 and B2

Patients with histologically confirmed advanced (unresectable/metastatic) cSCC (B1) or ACC (B2) who failed standard therapy or for whom no standard therapy is indicated

Not amenable to curative locoregional treatments

Progression on/after at least one line of therapy (radiological documentation required for ACC)

Part C: Dose Escalation of RNAdjuvant in Combination with Anti-PD-1 Therapy in Patients with Advanced cMEL or HNSCC

Part C enrols patients with advanced cMEL or HNSCC currently receiving anti-PD-1 therapy. Patients must have stable disease or slowly progressive disease after at least 12 weeks of anti-PD-1 therapy prior to application of RNAjuvant. Cohorts of at least 2 patients are treated sequentially at escalating doses of RNAdjuvant. The dose escalation and the determination of the MTD and RCD (“recommended combination dose”) are guided by a 5 parameter bayesian logistic regression model with overdose control.

Dose escalation in Part C starts as soon as at least 3 doses of RNAdjuvant have been evaluated in Part A. Starting dose in Part C is one dose level below the highest dose level considered tolerable from part A at the time Part C is commenced (see Table 9).

Inclusion Criteria:

Cohorts C and D:

Histologically confirmed advanced (unresectable or metastatic) cMEL in Cohorts C and D1

Currently receiving standard anti-PD-1 therapy according to the Summary of Product Characteristics (SPC) Stable or slowly progressing disease after at least 12 weeks of anti-PD-1 therapy according to investigator assessment

Intention to continue current anti-PD-1 therapy due to an assumed clinical benefit by continued anti-PD-1 therapy according to investigators judgement

Histologically confirmed recurrent or metastatic hnSCC in Cohorts C and D2

Currently receiving standard anti-PD-1 therapy according to the SPC

Stable or slowly progressing disease after at least 12 weeks of anti-PD-1 therapy, defined as follows:

Stable disease according to irRECIST

Intention to continue current anti-PD-1 therapy due to an assumed clinical benefit according to investigators judgement

Part D: Expansion Cohort of RNAdjuvant in Combination with Anti-PD-1 Therapy in Patients with Advanced cMEL or HNSCC.

Once the RCD (“recommended combination dose”) has been established in Part C, the expansion Part D enrols additional patients with advanced cMEL or HNSCC on treatment with a PD-1 antagonist (refer to eligibility requirements Part C) to further characterize the tolerability and the safety profile and to evaluate the anti-tumor activity of the combination therapy. Part D should enrol about 21 patients.

Part 2 (May Alternatively be Performed as Separate Clinical Trial)

Phase I, open label, cohort based dose escalation & expansion study of intratumorally and ministered RNAdjuvant and RNArt with or without systemic anti-PD-1 treatment in patients advanced malignant melanoma, squamous cell carcinoma of the skin (SCCs), adenocystic carcinoma (ACC), cutaneous T-cell lymphoma, or squamous cell carcinoma of the head and neck (HNSCC).

In Part 2 of the phase I clinical trial, Part 1 is repeated, but a fixed dose combination of RNArt and RNAdjuvant is investigated. Dose escalation methodology and cohort definitions including clinical indications are similar to Part 1. Please refer to the previous section for details.

However, for Part 2, information gained from Part 1 is considered that may lead to a change of the study design of Part 1 and/or 2. RNArt comprises 3 compounds based on optimized RNA that encodes IL-12, PD-1 decoy receptor and CD-40L.

### Example 7: Intratumoral Treatment with an Immunostimulating RNA (“RNAdjuvant”) and an mRNA Encoding CD40 Ligand (CD40L) and mRNA Encoding Soluble PD1 (solPD1) in Combination with a Checkpoint Inhibitor Anti CTLA-4 Antibody

Table 10 summarizes the treatment as used in the present example. RNAdjuvant and an mRNA encoding soluble PD1 and CD40L in combination with a checkpoint inhibitor anti-CTLA4 antibody (BioXcell, clone 9H10) were administered intratumorally (i.t.) in CT26 tumor challenged mice, median tumor growth were analyzed.

TABLE 10
Groups, treatment and RNA dilution/antibody dilution

| | RNAdjuvant (i.t.) | RNArt (i.t.) | anti-CTLA4 (i.t) | No. of |
|---|---|---|---|---|
| Group | (25 μg) | (25 μg) | (50 μg) | mice |
| A | RNAdjuvant | solPD1 + CD40L | anti-CTLA4 | 10 |
| B | RNAdjuvant | IL12 + solPD1+ CD40L | | 10 |
| C | Buffer | | | 9 |

Tumor Challenge and Administration of the Inventive Composition:

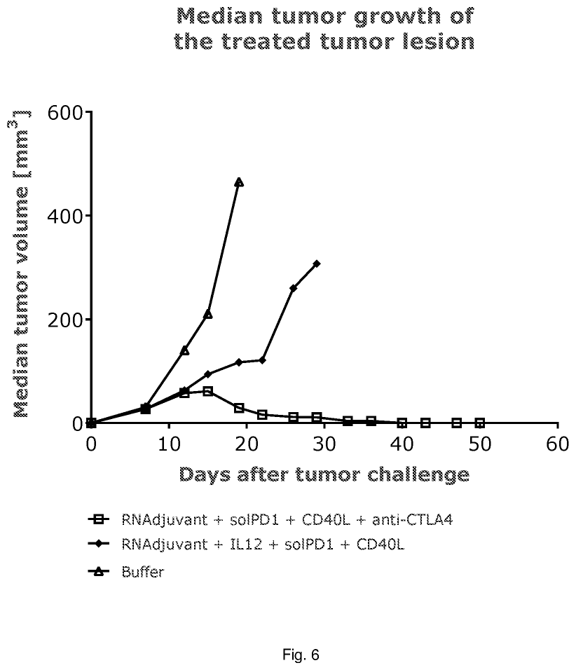

The tumor challenge was performed according to the previous experiments (see Example 1). Mice were injected according to the indicated scheme shown in Table 10. The results of the experiment are shown in FIG. 6.

Results:

The results in FIG. 6 show that the inventive composition comprising RNAdjuvant and an mRNA encoding soluble PD1 and CD40L in combination with a checkpoint inhibitor anti-CTLA4 antibody (group A according to Table 10) strongly decreased the median tumor volume.

### Example 8: Abscopal Effect of Intratumoral Treatment with an Immunostimulating RNA (RNAdjuvant) and an mRNA Encoding CD40 Ligand (CD40L) and mRNA Encoding Soluble PD1 (solPD1) in Combination with a Checkpoint Inhibitor Anti CTLA-4 Antibody

Tumor challenge and administration of the inventive composition: Balb/c mice were injected subcutaneously (s.c.) with 1×10⁶ CT26 cells (colon carcinoma cell line) per mouse (in a volume of 100 μl PBS) on the right flank on day 0 of the experiment. On day 5 of the experiment mice were injected subcutaneously (s.c.) with 1×10⁶ CT26 cells (in a volume of 100 μl PBS) on the left flank to observe an abscopal effect (effect on the untreated tumor) of the inventive composition. Table 10 of Example 7 summarizes the treatment as used in the present example. RNAdjuvant and an mRNA encoding soluble PD1 and CD40L in combination with an anti-CTLA4 checkpoint inhibitor were administered intratumorally (i.t.) in CT26 tumor bearing mice (right flank), median tumor growth of the untreated tumor (left flank) were analyzed.

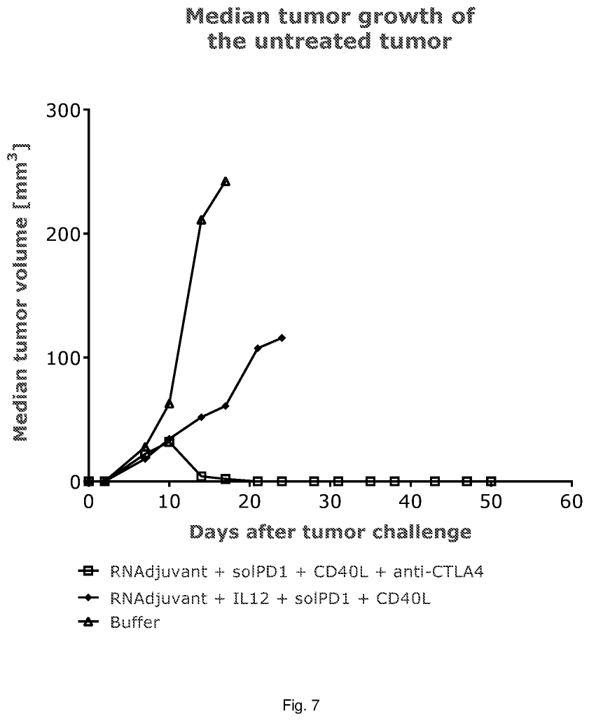

Mice were injected according to the indicated scheme shown in Table 10 of Example 7. Median tumor growth of the untreated tumor (left flank) was analyzed. Results of the experiment are shown in FIG. 7.

Results:

Intratumoral treatment of one lesion with anti-CTLA4 antibody in combination with RNAdjuvant, mRNA encoding soluble PD1 and mRNA encoding CD40 ligand induces a systemic effect and inhibits tumor growth of the untreated tumor.

### Example 9: Intratumoral Treatment with an Immunostimulating RNA (RNAdjuvant) and mRNA Encoding CD40 Ligand (CD40L), mRNA Encoding IL12, mRNA Encoding Soluble PD1 (solPD1) and Anti-CTLA4 Antibody in Combination with Anti-PD1 Antibody (Administered i.p.)

Tumor Challenge and Administration of the Inventive Composition:

The tumor challenge is performed according to the previous experiments (see Example 1). Mice are injected according to the indicated scheme shown in Table 11.

Table 11 summarizes the treatment as use in the present example. RNAdjuvant and an mRNA encoding CD40L, mRNA encoding soluble PD1, mRNA encoding IL12, mRNA encoding CD40L and mRNA encoding the checkpoint inhibitor anti-CTLA4 antibody are administered intratumorally (i.t.) in combination with checkpoint inhibitor anti PD1 antibody in CT26 tumor challenged mice, median tumor growth and survival rates are analyzed.

TABLE 11
Groups, treatment and RNA dilution/antibody dilution

| | | | | anti-PD1 | No. of |
|---|---|---|---|---|---|
| Group | RNAdjuvant (i.t.) | RNArt (i.t.) | anti-CTLA4 (i.t.) | (i.p.) | mice |
| 1 | RNAdjuvant | IL12 + CD40L | anti-CTLA4 | anti-PD1 | 10 |
| 3 | RNAdjuvant | IL12 + CD40L + solPD1 | anti-CTLA4 | | 10 |
| 2 | RNAdjuvant | IL12 + CD40L | anti-CTLA4 | | 10 |
| 4 | RNAdjuvant | IL12 + CD40L | | anti-PD1 | 10 |
| 5 | RNAdjuvant | IL12 + CD40L + solPD1 | | | 10 |
| 6 | RNAdjuvant | IL12 + CD40L | | | 10 |
| 7 | | | anti-CTLA4 | anti-PD1 | 10 |
| 8 | | | anti-CTLA4 | | 10 |
| 9 | | | | anti-PD1 | 10 |
| 10 | Puffer | | | | 8 |

### Example 10: Intratumoral Treatment with an Immunostimulating RNA (RNAdjuvant) and an mRNA Encoding for an Antigen (RNActive) Administered Intradermally (i.d.) in Combination with Anti-PD1 Antibody (Administered i.p.)

C57BL/6 mice were inoculated s.c. with 3×10⁵ E.G7-OVA tumor cells in the right flank. Treatment was start at a mean tumor size of 30 mm³. Mice were treated i.t. with immunostimulatory RNAdjuvant and vaccinated i.d. with OVA RNActive (mRNA encoding ovalbumine) in combination with an anti PD1 antibody (administered i.p.). mRNA encoding Photinus pyralis Luciferase (PpLuc) or buffer were used as unspecific control. On days 7, 11, 14, 17, and 20 of the experiment mice were treated according to Table 12 below. Median tumor growth and survival rates were analyzed.

TABLE 12
Groups, treatment and RNA dilution/antibody dilution

| | No. of | Anti-PD1 (i.p.) | RNAdjuvant (i.t.) | RNActive (i.d.) |
|---|---|---|---|---|
| Group | mice | (200 μg) | (25 μg) | (32 μg) |
| A | 10 | Anti-PD1 | RNAdjuvant | OVA |
| B | 10 | Anti-PD1 | RNAdjuvant | |
| C | 10 | | | OVA |
| D | 10 | | PpLuc | PpLuc |
| E | 10 | | Buffer | Buffer |

Tumor Challenge and Administration of the Inventive Composition:

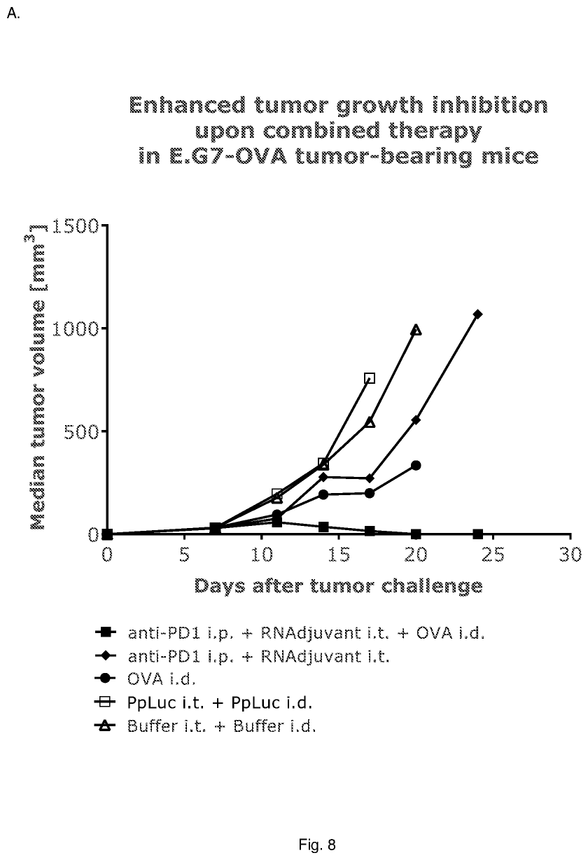

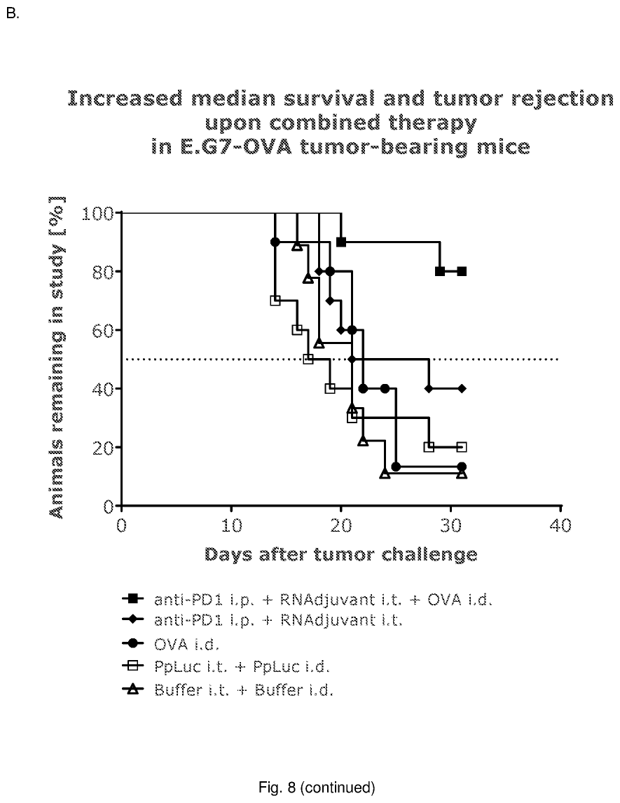

Mice were injected according to the indicated scheme shown in Table 12. The results of the experiment are shown in FIG. 8.

Results:

The results in FIG. 8 show that the immunostimulatory RNA (RNAdjuvant) administered i.t. in combination with mRNA encoding the tumor antigen ovalbumine (OVA) and in combination with an anti PD1 antibody strongly decreased the tumor growth compared to the other treatments (groups B-E according to Table 12). Remarkably, the results in FIG. 8 show that the immunostimulatory RNAdjuvant in combination with a checkpoint inhibitor PD1 antibody and mRNA vaccination (OVA, administered i.d.) induced complete tumor remission and significantly increased the survival of tumor challenged mice compared to the other treatments (groups B-E according to Table 13).

### Example 11: Intratumoral Treatment with an Immunostimulating RNA (RNAdjuvant) and an mRNA Encoding IL12 Administered i.t. In Combination with an mRNA Encoding an Antigen (RNActive) Administered i.d

C57BL/6 mice were inoculated s.c. with 3×105 E.G7-OVA tumor cells in the right flank. Treatment was started at a mean tumor size of 30 mm³. Mice were treated i.t. with immunostimulatory RNAdjuvant and vaccinated i.d. with mRNA encoding the tumor antigen ovalbumine (OVA RNActive) in combination with an mRNA encoding IL12 (administered i.t.). mRNA encoding Photinus pyralis luciferase (PpLuc RNActive) or buffer were used as unspecific control. On days 7, 11, 14, 17, and 20 of the experiment mice were treated according to Table 13 below. Median tumor growth and survival rates were analyzed.

TABLE 13
Groups, treatment and RNA dilution

| | No. | RNAdjuvant (i.t.) | RNArt (i.t.) | RNActive (i.d.) |
|---|---|---|---|---|
| Group | of mice | 25 μg | 25 μg | 32 μg |
| A | 10 | RNAdjuvant | IL12 | OVA |
| B | 10 | RNAdjuvant | IL12 | |
| C | 10 | | | OVA |
| D | 10 | | PpLuc | PpLuc |
| E | 10 | | Buffer | Buffer |

Tumor Challenge and Administration of the Inventive Composition:

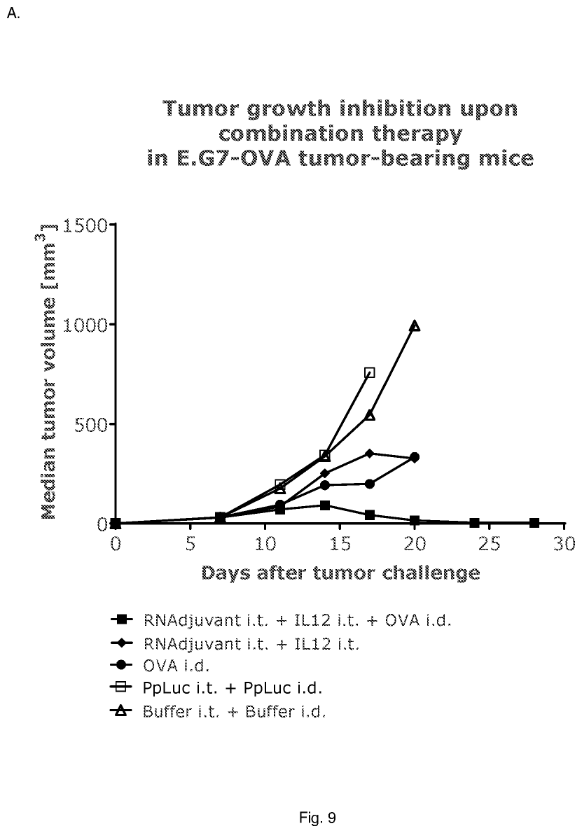

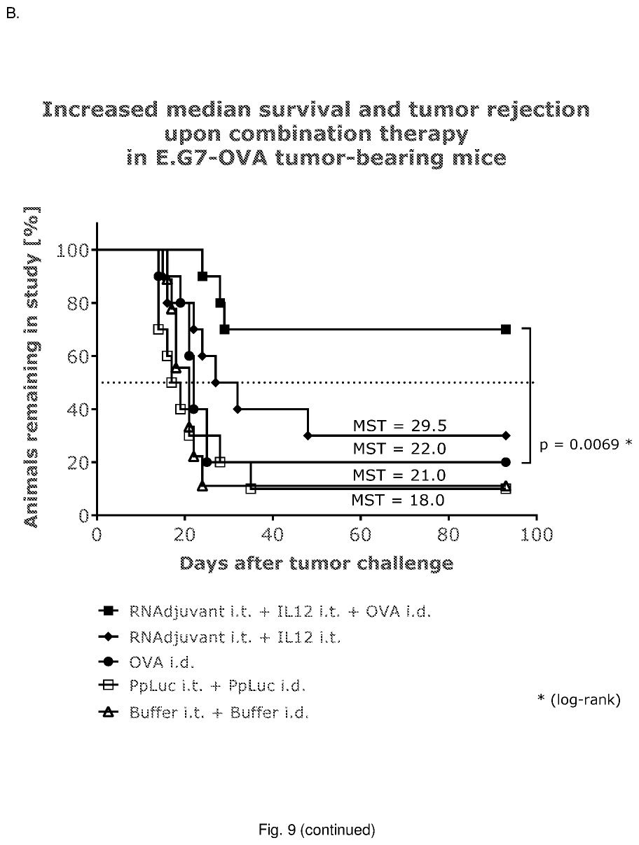

Mice were injected according to the indicated scheme shown in Table 13. The results of the experiment are shown in FIG. 9.

Results:

The results in FIG. 9 show that intratumoral treatment with immunostimulatory RNA (RNAdjuvant) and an mRNA encoding IL12 in combination with intradermal vaccination with OVA RNActive strongly decreased the median tumor volume compared to the other treatments (groups B-E according to Table 13). In addition, the results in FIG. 9 show that the inventive composition comprising an immunostimulatory RNA (RNAdjuvant) and an mRNA encoding IL12 combined with mRNA vaccination (OVA RNActive) strongly increased the survival of tumor challenged mice compared to the other treatments (groups B-E according to Table 13).

### Example 12: Phase I/II Study of Intratumoral Application of RNAdjuvant (CV8102) in Patients with Advanced Cutaneous Melanoma (cMEL), Cutaneous Squamous Cell Carcinoma (cSCC), Head and Neck Squamous Cell Carcinoma (hnSCC), Adenoid Cystic Carcinoma (ACC), Vulvar Squamous Cell Carcinoma (VSCC), or Cutaneous T-Cell Lymphoma, Mycosis Fungoides Subtype (CTCL-MF)

Part 1:

Phase I, open label, cohort based dose escalation & expansion study of intratumorally administered RNAdjuvant, with or without systemic anti-PD-1 treatment, in patients having advanced cutaneous melanoma (cMEL), cutaneous squamous cell carcinoma (cSCC), head and neck squamous cell carcinoma (hnSCC), adenoid cystic carcinoma (ACC), vulvar squamous cell carcinoma (VSCC), or cutaneous T-cell lymphoma, mycosis fungoides subtype (CTCL-MF).

Intratumoral injection of RNAdjuvant: CV8102 is administered to cutaneous, subcutaneous, or readily accessible lymph node lesions that can be injected using direct visualization or imaging-guidance (ultrasound) as clinically determined.

Schedule: Patients receive a maximum of 8 intratumoral administrations of CV8102, unless disease progression requiring initiation of next-line therapy or unacceptable toxicity occurs:

The first 5 administrations are performed in weekly intervals (Days 1, 8, 15, 22, 29). For patients on anti-PD-1 treatment in Cohorts C and D, CV8102 treatment is initiated on a day of anti-PD-1 treatment and will follow the anti-PD-1 treatment schedule after day 29.

The subsequent 3 administrations of CV8102 are performed with 2-week intervals (Cohorts A and B). For patients on anti-PD-1 treatment in study Cohorts C and D, the administrations of CV8102 after Day 29 follow the anti-PD-1 treatment schedule and are performed on the day of anti-PD-1 administration; i.e., patients on nivolumab receive CV8102 every 2nd week; patients on pembrolizumab receive CV8102 every 3rd week.

Part A: Dose Escalation of Single Agent RNAdjuvant

Part A of the study uses a 2 parameter Bayesian logistic regression model with overdose control for dose escalation. Cohorts of at least 1 (starting dose level) or 2 patients (any other dose level) with advanced cMEL, cSCC, hnSCC, or ACC are treated at escalating doses of intratumorally administered RNAdjuvant until identification of the maximum tolerated dose (MTD) and determination of the recommended dose (RD). A minimum of 7 patients should be enrolled in Part A.

The starting dose in Part A is 25 μg of RNAdjuvant. The further dose levels are listed in Table 14 below.

TABLE 14
Dose levels of RNAdjuvant
Dose Levels evaluated in Part A and C

| Dose Level | RNAdjuvant | No of Patients^{§} |
|---|---|---|
| Level -1 | 25 μg | ≥1 |
| Level 2 | 50 μg | ≥2 |
| Level 3 | 100 μg | ≥2 |
| Level 4 | 150 μg | ≥2 |

^{§}A minimum of 2 patients will be treated per cohort

Inclusion Criteria:

Cohort A:

Patients with histologically confirmed advanced (unresectable or metastatic) cMEL or cSCC who failed approved standard therapy or for whom no standard therapy is indicated

Not amenable to surgical resection or locoregional radiation therapy with curative intent

Progression on/after at least one line of therapy

or

Histologically confirmed recurrent or metastatic hnSCC

Not amenable to surgical resection or locoregional radiation therapy with curative intent

Progression documented radiologically (documentation of a new or progressive lesion on/after at least one line of therapy.

or

Histologically or cytologically confirmed ACC

Not amenable to surgical resection or locoregional radiation therapy with curative intent

Progression documented radiologically; on/after at least one line of therapy

or

Histologically confirmed recurrent or metastatic VSCC

Not amenable to surgery, radio- or radiochemotherapy with curative intent

Not a candidate for standard systemic therapies

or

Relapsed or refractory CTCL, mycosis fungoides (MF) subtype

Diagnosis based upon standard staging classification system

MF without CD30⁺ large cell transformation and no evidence of visceral involvement

Relapsed, refractory or progressed after at least one prior treatment

Part B: Expansion Cohort

After completion of Part A, patients with advanced cSCC, ACC, VSCC, or CTCL-MF are enrolled to separate expansion cohorts at the previously defined recommended dose to further characterize the tolerability and safety profile of intratumorally administered RNAdjuvant in these patient populations and to collect pre-liminary evidence of anti-tumor activity. Part B should enrol up to 10 patients per expansion cohort. Inclusion criteria:

Cohort B:

Expansion cohorts B1, B2

Patients with histologically confirmed advanced (unresectable/metastatic) cSCC (B1) or ACC (B2) who failed standard therapy or for whom no standard therapy is indicated

Not amenable to curative locoregional treatments

Progression on/after at least one line of therapy (radiological documentation required for ACC)

Expansion cohort B3

Histologically confirmed recurrent or metastatic VSCC

Not amenable to surgery, radio- or radiochemotherapy with curative intent

Not a candidate for standard systemic therapies

Expansion cohort B4

Relapsed or refractory CTCL, mycosis fungoides (MF) subtype

Diagnosis based upon standard staging classification system

MF without CD30+large cell transformation and no evidence of visceral involvement

Relapsed, refractory or progressed after at least one prior treatment

Part C: Dose Escalation of RNAdjuvant in Combination with Anti-PD-1 Therapy in Patients with Advanced cMEL or HNSCC

Part C enrols patients with advanced cMEL or HNSCC currently receiving anti-PD-1 therapy. Patients must have stable disease or slowly progressive disease after at least 12 weeks of anti-PD-1 therapy prior to application of RNAjuvant. Cohorts of at least 2 patients are treated sequentially at escalating doses of RNAdjuvant. The dose escalation and the determination of the MTD and RCD (“recommended combination dose”) are guided by a 5 parameter bayesian logistic regression model with overdose control.

Dose escalation in Part C starts as soon as at least 3 doses of RNAdjuvant have been evaluated in Part A. Starting dose in Part C is 25 μg (see Table 14).

Inclusion Criteria:

Cohorts C and D:

Histologically confirmed advanced (unresectable or metastatic) cMEL in Cohorts C and D1

Currently receiving standard anti-PD-1 therapy according to the Summary of Product Characteristics (SPC)

Stable or slowly progressing disease after at least 12 weeks of anti-PD-1 therapy according to investigator assessment Intention to continue current anti-PD-1 therapy due to an assumed clinical benefit by continued anti-PD-1 therapy according to investigators judgement

Histologically confirmed recurrent or metastatic hnSCC in Cohorts C and D2

Currently receiving standard anti-PD-1 therapy according to the SPC

Stable or slowly progressing disease after at least 12 weeks of anti-PD-1 therapy, defined as follows:

Stable disease according to irRECIST

Stable disease requires a less than or equal 5% decrease in disease (defined as 5% regression in measurable dimension of disease) during an interval of at least 12 weeks prior to Day 1

Intention to continue current anti-PD-1 therapy due to an assumed clinical benefit according to investigators judgement

Part D: Expansion Cohort of RNAdjuvant in Combination with Anti-PD-1 Therapy in Patients with Advanced cMEL or HNSCC.

Once the RCD (“recommended combination dose”) has been established in Part C, the expansion Part D enrols additional patients with advanced cMEL or HNSCC on treatment with a PD-1 antagonist (refer to eligibility requirements Part C) to further characterize the tolerability and the safety profile and to evaluate the anti-tumor activity of the combination therapy. Part D should enrol about 21 patients.

Part 2 (Will be Performed as Separate Clinical Trial)

Phase I, open label, cohort based dose escalation & expansion study of intratumorally administered RNAdjuvant and RNArt with or without systemic anti-PD-1 treatment in patients percutaneously accessible solid tumors or lymphoma.

Basically, the design of the phase I clinical trial of RNArt+RNadjuvnat corresponds to the design of the Phase I study of RNAdjuvant (Part 1). The range of tolerated dose combinations will first be investigated using a dose-escalation procedure (cohort A). RNart, comprising 5 RNA compounds encoding IL-12, CD40L, soluble PD-1 an the two chains of anti-CTLA-4, will be increased stepwise while RNADjuvnat will be kept at fixed dose of 25 μg. RNArt and RNadjuvnat will be given simultaneously to the same tumor lesion. Dose escalation methodology is similar to Part 1, Cohort A (Bayesian dose-escalation approach). The administration schedule corresponds to the schedule of RNADjuvant described above (i.e 5×weekly followed by 3 addition injectgions Q2W).

The dose escalation part (Cohort A) includes the indications listed in Part 1, plus the following additional indications: Patients with

Human Papilloma virus related advanced tumors, including advanced, recurrent or metastatic vulvar squamous cell carcinoma, cervical cancer, or vaginal cancer

Not amenable to surgery, radio- or radiochemotherapy with curative intent

Not a candidate for standard systemic therapies

or

Follicular low-grade Non-Hodgkin's lymphoma

Either treatment naïve or relapsed or refractory following at least one prior treatment

Not requiring active therapy (asymptomatic, watchful waiting patients)

or

Nodal marginal zone B cell lymphoma

Either treatment naïve or relapsed or refractory following at least one prior treatment

Not requiring active therapy (asymptomatic, watchful waiting patients)

or

Primary cutaneous anaplastic large-cell lymphoma

Relapsed, refractory or progressed after at least one prior treatment or for whom no other therapy options are available or

Histologically confirmed advanced, recurrent or metastatic adult soft tissue sarcoma

Not amenable to surgery or other treatment options with curative intent

At least one line of prior systemic therapy or sarcoma histological subtypes for which there is no known standard systemic therapy

or

Histologically confirmed advanced (unresectable or metastatic) basal cell carcinoma of the skin

Not amendable to surgery of any other treatment options with curative intent

Not a Candidate for Systemic Therapy

Cohort B: Expansion Cohort

For indications please refer to Part 1, Expansion cohort B

Cohort C: Dose Escalation of RNart+RNAdjuvant in Combination with Anti-PD-1 Therapy in Patients with Advanced cMEL, HNSCC or cSCC

As in the Phase I study with RNAdjuvant, Part C enrols patients with advanced cMEL or HNSCC. Depending on whether an anti-PD-1 antobidy will be approved for treatment of advanced cSCC at the time of start of Cohort C, patients with cSCC will be also enrolled into this cohort. Patients must have stable disease or slowly progressive disease after at least 12 weeks of anti-PD-1 therapy prior to application of RNArt+RNAjuvant. RNart, comprising 4 RNA compounds encoding IL-12, CD40L and the two chains of anti-CTLA-4, will be increased stepwise while RNAdjuvnat will be kept at fixed dose of 25 μg. For the dose escalation methodology please refer to Part 1, Dohort C.

Inclusion Criteria:

Cohorts C and D:

Include the indications listed in Part 1, Cohorts C and D. Depending on the approval status of anti-PD-1 antibodies, the following patients will be enrolled to these cohorts:

Histologically confirmed advanced (unresectable or metastatic) cSCC

Currently receiving standard anti-PD-1 therapy according to the Summary of Product Characteristics (SPC)

Stable or slowly progressing disease after at least 12 weeks of anti-PD-1 therapy according to investigator assessment Intention to continue current anti-PD-1 therapy due to an assumed clinical benefit by continued anti-PD-1 therapy according to investigators judgement

Experiment 13: Expression of Single RNArt Constructs in A375 Human Melanoma Cells

A375 cells were seeded in 24-well plates at a density of 50 000 cells/well. After 24 hours cells were transfected with the indicated amounts of RNA and lipofectamine 2000 (Invitrogen) in a constant RNA:lipofectamine ratio of 1:2. After 5 hours the supernatants were collected and 1 ml fresh medium was added. After further 24 hours of incubation supernatants were again collected. Translated mRNA Products of IL12, solPD1 and anti-CTLA4 antibody were measured in the supernatant.

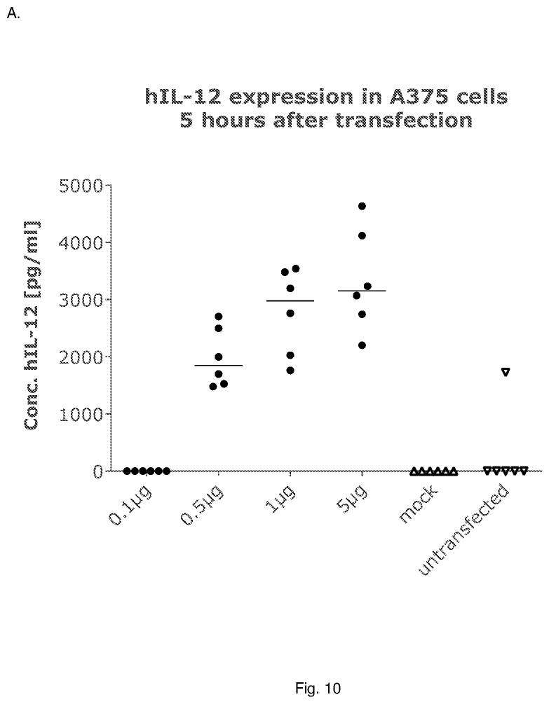

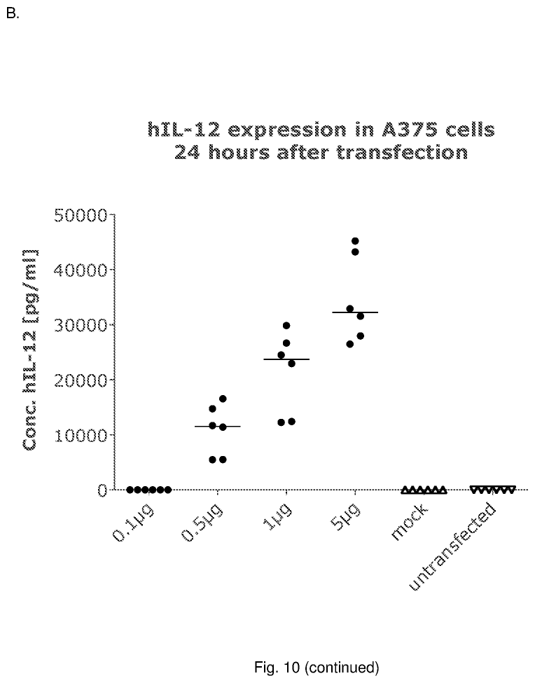

Human IL12 content in the supernatants was measured with the Human IL-12p70 DuoSet Kit, R&D Systems, Cat: DY1270 according to the manufacturer's protocol using 1:10, 1:100 and 1:1000 dilutions of the supernatants (see FIG. 10).

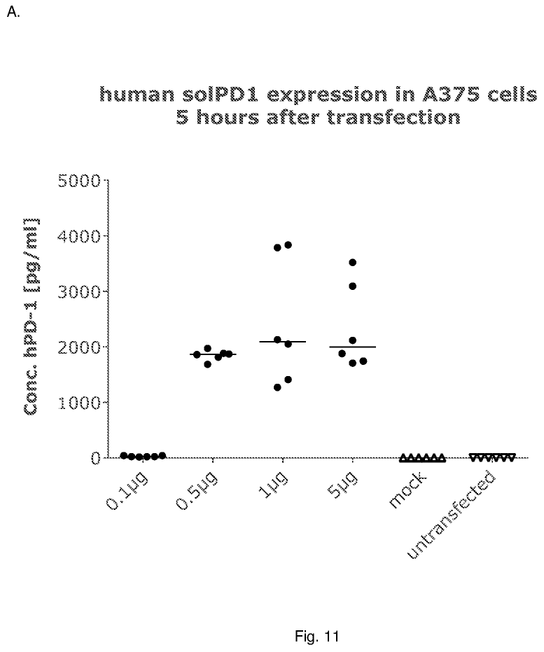

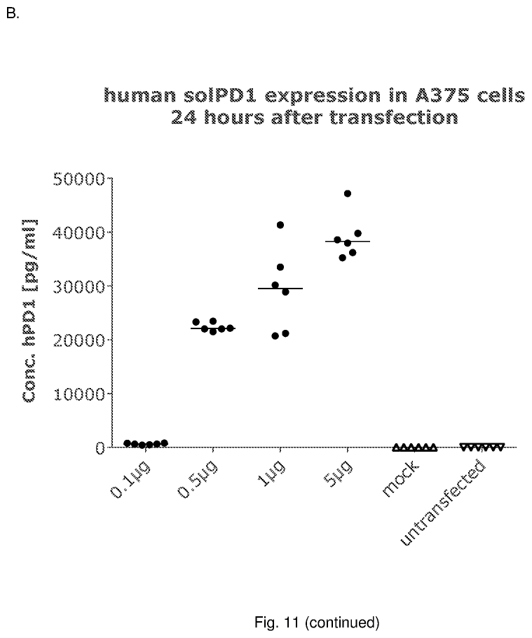

Human PD1 ELISA is done in duplicates with the Human PD1 DuoSet Kit, R&D Systems, Cat: DY1086, according to the manufacturer's protocol using 1:10, 1:100 and 1:1000 dilutions of the supernatants (see FIG. 11).

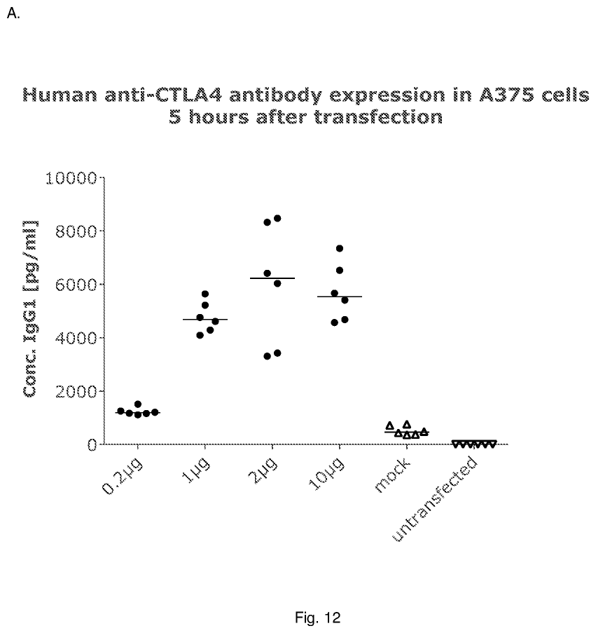

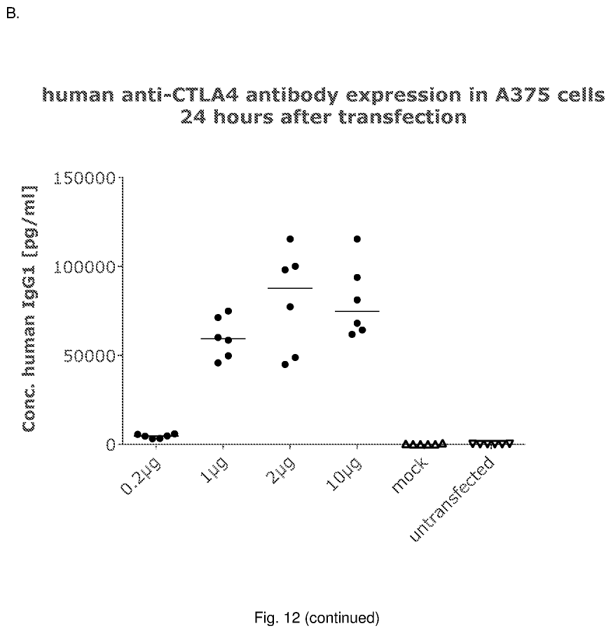

Human anit-CTLA4 IgG1 ELISA is done in duplicates with Rituximab Antibody for standard (Goat Anti-Human IgG (SouthernBiotech Cat. No: 2044-01, c=1 mg/ml, 1:1000) and Goat Anti-Human IgG Biotin (Dianova Cat. No: 109065088-01, 1:20000) using 1:10, 1:100 and 1:1000 dilutions of the supernatants (see FIG. 12).

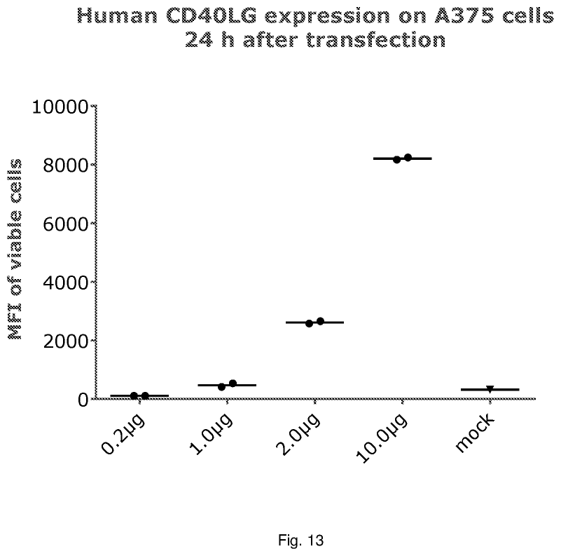

For detection of membrane bound CD40LG protein A375 cells were seeded in 6-well plates at a density of 130 000 cells/well. After 24 hours cells were transfected with the indicated amounts of RNA and lipofectamine 2000 (Invitrogen) in a constant RNA:lipofectamine ratio of 1:2. After 24 hours of incubation cells were collected and cell surface staining was done with CD154 anti-human APC (BD Pharmingen). Finally cell surface expression of CD40LG was analyzed by flow cytometry (see FIG. 13).

TABLE 15
RNA constructs

| RNA | Description | SEQ ID NO: |
|---|---|---|
| R2025 | Non-coding immunostimulatory RNA | 433 |
| | (RNAdjuvant) | |
| R5448/R5939 | mRNA encoding human IL-12 | 598 |
| | (hIL-12 (GC)) | |
| R5447/R5938 | mRNA encoding human soluble PD-1 | 597 |
| | (human solPD1 (GC)) | |
| R5446/R5990 | mRNA encoding human CD40LG | 596 |
| | (CD40LG (GC)) | |
| R5417 | mRNA encoding heavy chain (HC) of anti-CTLA4 antibody | 594 |
| | (HC anit-CTLA4 Ab (GC)) | |
| R5418 | mRNA encoding light chain (LC) of anti-CTLA4 antibody | 595 |
| | (HLC anit-CTLA4 Ab (GC)) | |

1. A method for the treatment or prophylaxis of a tumor or cancer disease selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy comprising administering to a subject in need thereof an immunostimulatory RNA (isRNA).

2. The method according to claim 1, wherein the isRNA is administered intratumorally, intradermally, intramuscularly or subcutaneously.

3. (canceled)

4. The method according to claim 1, wherein the isRNA is a non-coding RNA.

5. The method according to claim 1, wherein the isRNA comprises a nucleic acid sequence according to formula (I)
(GₗXₘGₙ),
wherein:
G is guanosine (guanine), uridine (uracil) or an analogue of guanosine (guanine) or uridine (uracil);
X is guanosine (guanine), (uridine) uracil, adenosine (adenine), thymidine (thymine), cytidine (cytosine) or an analogue of the above-mentioned nucleotides (nucleosides);
l is an integer from 1 to 40,
wherein
when l=1 G is guanosine (guanine) or an analogue thereof,
when l>1 at least 50% of the nucleotides (nucleosides) are guanosine (guanine) or an analogue thereof;
m is an integer and is at least 3;
wherein
when m=3 X is uridine (uracil) or an analogue thereof,
when m>3 at least 3 successive uridines (uracils) or analogues of uridine (uracil) occur;
n is an integer from 1 to 40,
wherein
when n=1 G is guanosine (guanine) or an analogue thereof,
when n>1 at least 50% of the nucleotides (nucleosides) are guanosine (guanine) or an analogue thereof; or
a nucleic acid sequence according to formula (III)
(NᵤGₗXₘGₙNᵥ)ₐ
wherein:
G is guanosine (guanine), uridine (uracil) or an analogue of guanosine (guanine) or uridine (uracil), preferably guanosine (guanine) or an analogue thereof;
X is guanosine (guanine), uridine (uracil), adenosine (adenine), thymidine (thymine), cytidine (cytosine), or an analogue of these nucleotides (nucleosides), preferably uridine (uracil) or an analogue thereof;
N is a nucleic acid sequence having a length of about 4 to 50, preferably of about 4 to 40, more preferably of about 4 to 30 or 4 to 20 nucleic acids, each N independently being selected from guanosine (guanine), uridine (uracil), adenosine (adenine), thymidine (thymine), cytidine (cytosine) or an analogue of these nucleotides (nucleosides);
a is an integer from 1 to 20, preferably from 1 to 15, most preferably from 1 to 10;
l is an integer from 1 to 40,
wherein when l=1, G is guanosine (guanine) or an analogue thereof,
when l>1, at least 50% of these nucleotides (nucleosides) are guanosine (guanine) or an analogue thereof;
m is an integer and is at least 3;
wherein when m=3, X is uridine (uracil) or an analogue thereof, and
when m>3, at least 3 successive uridines (uracils) or analogues of uridine (uracil) occur;
n is an integer from 1 to 40,
wherein when n=1, G is guanosine (guanine) or an analogue thereof,
when n>1, at least 50% of these nucleotides (nucleosides) are guanosine (guanine) or an analogue thereof;
u,v may be independently from each other an integer from 0 to 50,
preferably wherein when u=0, v≥1, or when v=0, u≥1;
wherein the nucleic acid molecule of formula (III) has a length of at least 50 nucleotides, preferably of at least 100 nucleotides, more preferably of at least 150 nucleotides, even more preferably of at least 200 nucleotides and most preferably of at least 250 nucleotides.

6. The method according to claim 1, wherein the isRNA comprises at least one nucleic acid sequence according to any one of SEQ ID NOs: 433 to 437, 1014 to 1016.

7. The method according to claim 1, wherein the isRNA is complexed with a cationic or polycationic compound, preferably with a cationic or polycationic polymer, a cationic or polycationic peptide or protein, a cationic or polycationic polysaccharide and/or a cationic or polycationic lipid.

8. The method according to claim 7, wherein the cationic or polycationic compound is a polymeric carrier.

9. The method according to claim 8, wherein the polymeric carrier is formed by a disulfide-crosslinked cationic component, preferably a disulfide-crosslinked cationic peptide, wherein the disulfide-crosslinked cationic peptide preferably comprises a peptide according to formula V
(Arg)ₗ;(Lys)ₘ;(His)ₙ;(Orn)ₒ;(Xaa)ₓ, (formula (V),
wherein l+m+n+o+x=8-15, and l, m, n or o independently of each other may be any number selected from 0, 1, 2, 3, 4, 5, 6, 7, 8, 9, 10, 11, 12, 13, 14 or 15, provided that the overall content of Arg, Lys, His and Orn represents at least 50% of all amino acids of the oligopeptide; and Xaa may be any amino acid selected from native (=naturally occurring) or non-native amino acids except of Arg, Lys, His or Orn; and x may be any number selected from 0, 1, 2, 3 or 4, provided, that the overall content of Xaa does not exceed 50% of all amino acids of the oligopeptide;
a peptide according to formula Va
{(Arg)ₗ;(Lys)ₘ;(His)ₙ;(Orn)ₒ;(Xaa′)ₓ(Cys)_{y}} formula (Va),
wherein (Arg)ₗ;(Lys)ₘ;(His)ₙ;(Orn)ₒ; and x are as defined for formula V, Xaa′ is any amino acid selected from native (=naturally occurring) or non-native amino acids except of Arg, Lys, His, Orn or Cys and y is any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80 and 81-90, provided that the overall content of Arg (Arginine), Lys (Lysine), His (Histidine) and Orn (Ornithine) represents at least 10% of all amino acids of the oligopeptide;
a peptide according to formula Vb
Cys1{(Arg)ₗ;(Lys)ₘ;(His)ₙ;(Orn)ₒ;(Xaa)ₓ}Cys2 formula (Vb)
wherein empirical formula {(Arg)ₗ;(Lys)ₘ;(His)ₙ;(Orn)ₒ;(Xaa)ₓ} is as defined for formula (V) and forms a core of an amino acid sequence according to (semiempirical) formula (V) and wherein Cys1 and Cys2 are cysteines proximal to, or terminal to (Arg)ₗ;(Lys)ₘ;(His)ₙ;(Orn)ₒ;(Xaa)ₓ; and/or

10. The method according to claim 8, wherein the polymeric carrier comprises at least one of the disulfide-crosslinked cationic peptides Cys-Arg₁₂ or Cys-Arg₁₂-Cys.

11. (canceled)

12. The method according to claim 1, wherein the isRNA is complexed with one or more lipids, thereby forming liposomes, lipid nanoparticles and/or lipoplexes.

13. The method according to claim 1, wherein the treatment comprises administration of at least one additional pharmaceutically active ingredient.

14. The method according to claim 13, wherein the at least one additional pharmaceutically active ingredient is a compound that is used in the treatment of a tumor or cancer disease preferably selected from the group consisting of cutaneous melanoma (cMEL), cutaneous squamous cell carcinoma (cSCC), head and neck squamous cell carcinoma (HNSCC), adenoid cystic carcinoma (ACC), cutaneous T-cell lymphoma, preferably cutaneous T-cell lymphoma of mycosis fungoides subtype, and vulvar squamous cell cancer (VSCC), wherein the tumor or the cancer disease is preferably at an advanced stage and/or refractory to standard therapy.

15. The method according to claim 13, wherein the at least one additional pharmaceutically active ingredient is a checkpoint modulator.

16. The method according to claim 15, wherein the checkpoint modulator is selected from the group consisting of a PD-1 inhibitor, a PD-L1 inhibitor, a CTLA-4 inhibitor, a LAG3 inhibitor, a TIM3 inhibitor, a TIGIT-inhibitor an OX40 stimulator, a 4-1BB stimulator, a CD40L stimulator, a CD28 stimulator and a GITR stimulator.

17. The method according to claim 16, wherein the checkpoint modulator is a PD 1 inhibitor or a PD-L1 inhibitor.

18-19. (canceled)

20. The method according to claim 1, wherein the treatment comprises administration of at least one coding RNA, preferably at least one mRNA.

21. The method according to claim 20, wherein the at least one coding RNA comprises at least one coding sequence encoding at least one peptide or protein comprising at least one peptide or protein selected from the group consisting of
IL-12,
CD40L,
a decoy PD-1 receptor, and
an antagonistic antibody directed against CTLA4.

22-48. (canceled)

49. The method according to claim 20, wherein the at least one coding RNA comprises at least one coding sequence encoding a peptide or protein comprising a tumor antigen.

50-66. (canceled)

67. The method according to claim 1, wherein the treatment comprises chemotherapy, radiation therapy and/or surgery.

68-73. (canceled)
